(19) United States
(12) Patent Application Publication
(10) Pub. No.: US 2020/0101068 A1
(43) Pub. Date: Apr. 2, 2020

(54) Heteroaryl Derivatives as PARP Inhibitors

(71) Applicant: Lupin Limited, Mumbai, Maharashtra (IN)

(72) Inventors: Navnath Popat Karche, Pune (IN); Ajay Ramchandra Tilekar, Pune (IN); Sanjay Pralhad Kurhade, Pune (IN); Ganesh Rajaram Jadhav, Pune (IN); Nishant Ramniwasji Gupta, Pune (IN); Neelima Sinha, Pune (IN); Venkata P. Palle, Pune (IN); Rajender Kumar Kamboj, Pune (IN)

(73) Assignee: Lupin Limited, Mumbai, Maharashtra (IN)

(21) Appl. No.: 15/746,967

(22) PCT Filed: Aug. 13, 2016
(86) PCT No.: PCT/IB2016/054886

§ 371 Date: Jan. 23, 2018

(30) Foreign Application Priority Data
Aug. 17, 2015 (IN) 3111/MUM/2015
Sep. 21, 2015 (IN) 3588/MUM/2015
Jan. 8, 2016 (IN) 201621000832

(51) Int. Cl. A61K 31/496; A61K 45/06; C07D 471/04; A61K 31/4995; C07D 519/00; A61K 31/519; C07D 487/04; A61K 31/4985

(52) U.S. Cl. CPC A61K 31/496; A61K 45/06; C07D 471/04; A61K 31/4985; C07D 519/00; A61K 31/519; C07D 487/04; A61K 31/4995

(57) ABSTRACT

Disclosed are compounds of formula (I), their tautomeric forms, stereoisomers, and pharmaceutically acceptable salts thereof, wherein ring Ar, ring B, R¹-R⁵, X, Y, p, q, r, and s are as defined in the specification, pharmaceutical compositions including a compound, tautomer, stereoisomer, or salt thereof, and methods of treating or preventing diseases or disorders, for example, cancer, that are amenable to treatment or prevention by inhibiting the PARP enzyme of a subject.

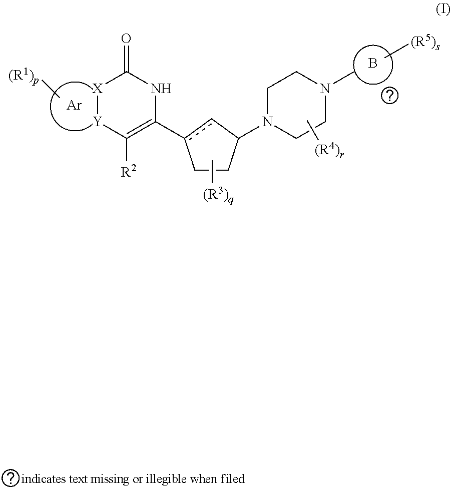

### FIELD OF THE INVENTION

The present invention relates to heteroaryl derivatives, their tautomeric forms, their stereoisomers, their pharmaceutically acceptable salts, combinations with suitable medicament, pharmaceutical compositions containing them, methods of making of heteroaryl derivatives, and their use as PARP inhibitors.

### CROSS-REFERENCE TO RELATED APPLICATIONS

The present application claims the benefit of Indian Provisional Patent Application Number 3111/MUM/2015, filed on 17 Aug. 2015, Indian Provisional Patent Application Number 3588/MUM/2015, filed on 21 Sep. 2015, and Indian Provisional Patent Application Number 201621000832, filed on 8 Jan. 2016, the disclosures of which are incorporated herein by reference in their entirety for all purposes.

### BACKGROUND OF THE INVENTION

Poly (ADP-ribose) Polymerase (PARP; 113 kDa) is an enzyme that catalyzes the addition of ADP-ribose residues to various target proteins. The reaction requires NAD⁺ as substrate. As many as 18 isoforms of PARP are known. PARP1 and PARP2 are the closest relatives [60% identical in PARP1 is activated by SSB (single-strand breaks) in DNA]. ADP-ribosylation occurs at the carboxylate groups of glutamic acid or aspartic acid residues in acceptor proteins and results in the modulation of catalytic activity and protein-protein interactions of the target proteins (e.g., modulation of chromatin structure, DNA synthesis, DNA repair (Base Excision Repair or BER), transcription, and/or cell cycle progression. PARP binds to DNA single strand as well as double strand breaks. The binding of PARP to damaged DNA leads to activation of the enzyme. PARP carries out ADP ribosylation of proteins involved in DNA repair (e.g., BER) including itself. Automodification of PARP results in its release from DNA which allows the DNA repair machinery to access the DNA damage site and carry out the repair process.

Overactivation of PARP leads to necrotic cell death as a result of NAD⁺ and ATP depletion. Cancer patients who have undergone radiotherapy or have been treated with chemotherapeutic agents that damage DNA (e.g., cisplatin, irinotecan, temozolomide) harbour DNA strand breaks. Activation of PARP in such cases allows the repair of the damaged DNA, thus leading to an undesirable resistance to the chemotherapeutic agents (and the consequent inefficacy). In such a scenario, treatment with a PARP inhibitor is expected to make the repair process inefficient and cause cell death.

BRCA1 and BRCA2 play an important role in HR (Homologous Recombination). DNA breaks arising during DNA replication can only be repaired by HR. Continuous exposure of BRCA1/BRCA2 deficient cells to PARP inhibitor results in accumulation of DNA DSB, followed by apoptosis (Synthetic Lethality). Triple Negative Breast Cancers (TNBC) are also acutely sensitive to PARP since they also harbor defects in the DNA repair machinery. Recently, cancer cells deficient in USP11 and endometrial cancer cells deficient in PTEN have also been shown to be sensitive to PARP inhibitors. PARP inhibitors thus have immense potential to be used for anticancer chemotherapy. [Biochem. J., (1999) 342, 249-268; Ann. Rev. Biochem., 1977, 46:95-116; E. Journal Cancer 4 6 (2010) 9-20].

Additionally, PARP has been implicated in a number of disease conditions other than cancer. These include disorders such as stroke, traumatic brain injury, Parkinson's disease, meningitis, myocardial infarction, ischaemic cardiomyopathy and other vasculature-related disorders. In animal experiments, PARP−/−mice demonstrated improved motor and memory function after CCI (Controlled Cortical Impact) versus PARP+/+ mice (J Cereb Blood Flow Metab. 1999, Vol. 19. No. 8, 835).

While attempts have been made to develop PARP inhibitors for treating cancer and other diseases, satisfactory treatment has not been achieved. Therefore, there exists an unmet need for new PARP inhibitors and treatment regimen therewith.

International patent application publications WO2002/090334, WO2002/036576, WO 2003/055865, WO2002/094790, WO2003/063874, WO2013/143663, WO2014/009872 and WO2016/012956 describe certain PARP inhibitors.

### BRIEF SUMMARY OF THE INVENTION

In one aspect, the present invention provides a compound of formula (I), its tautomeric form, its stereoisomer, its pharmaceutically acceptable salt, its combination with suitable medicament, its pharmaceutical composition and its use as PARP inhibitor,

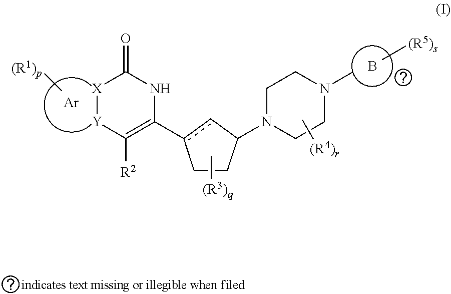

wherein, [symbol]
is either a single or a double bond;
X and Y independently represent carbon or nitrogen;
ring Ar is selected from
a) 6 membered heteroaromatic ring containing 1 to 2 nitrogen atoms, with X and Y being carbon; and
b) 5 membered heteroaromatic ring containing 1 to 2 heteroatoms selected from nitrogen, oxygen, and sulphur, wherein both X and Y are not selected as nitrogen at the same time;
   R¹ is independently selected at each occurrence from halogen, nitro, cyano, perhaloalkyl, substituted- or unsubstituted-alkyl, substituted- or unsubstituted-cyclopropyl, —NH₂, —N(H)CH₃, —OH, and —OCH₃;
   R² is selected from hydrogen, halogen, nitro, cyano, —NH₂, —N(H)CH₃, —OH, —OCH₃, substituted- or unsubstituted-cyclopropyl, and substituted- or unsubstituted-alkyl;
   R³ is independently selected at each occurrence from halogen, and substituted- or unsubstituted-alkyl, or two R³ on the same carbon form an oxo (═O), or two R³ groups together with the carbon atom(s) to which they are attached form a substituted- or unsubstituted-carbocycle;
   R⁴ is independently selected at each occurrence as substituted- or unsubstituted-alkyl, or two R⁴ on the same carbon form an oxo (═O), or two R⁴ groups together with the carbon atom(s) to which they are attached form a substituted- or unsubstituted-carbocycle or substituted- or unsubstituted-heterocycle;
   ring B is selected from cycloalkyl, heterocyclyl, aryl, and heteroaryl;
   R⁵ is independently selected at each occurrence from halogen, nitro, cyano, perhaloalkyl, substituted- or unsubstituted-alkyl, C(═O)R^{1a}, —C(═O)OR^{1b}, —C(═O)NR^{1b}R^{1c}, —NR^{1d}R^{1e}, and —OR^{1f};
   R^{1a} is selected from substituted- or unsubstituted-alkyl, and substituted- or unsubstituted-cycloalkyl;
   R^{1b} and R^{1e} are each independently selected from hydrogen, substituted- or unsubstituted-alkyl, and substituted- or unsubstituted-cycloalkyl;
   R^{1d} and R^{1e} are each independently selected from hydrogen, —C(═O)alkyl, substituted- or unsubstituted-alkyl, and substituted- or unsubstituted-cycloalkyl;
   R^{1f} is selected from hydrogen, —C(═O)alkyl, substituted- or unsubstituted-alkyl, perhaloalkyl, and substituted- or unsubstituted-cycloalkyl;
   p is selected from 0, 1, and 2;
   q is selected from 0, 1, 2, and 3;
   r is selected from 0, 1, 2, and 3; and
   s is selected from 0, 1, 2, and 3.

In a second aspect, the invention provides a pharmaceutical composition comprising the compound of formula (I) and a pharmaceutically acceptable carrier.

In a third aspect, the invention provides a method of treating or preventing a disorder responsive to the inhibition of PARP activity in a mammal suffering therefrom, comprising administering to the mammal in need of such treatment a therapeutically effective amount of a compound of formula (I).

### DETAILED DESCRIPTION OF THE INVENTION

The present invention provides a compound of the general formula (I), its tautomeric form, its stereoisomer, its pharmaceutically acceptable salt, its combination with suitable medicament, its pharmaceutical composition, process and intermediates for the preparation of the above compound,

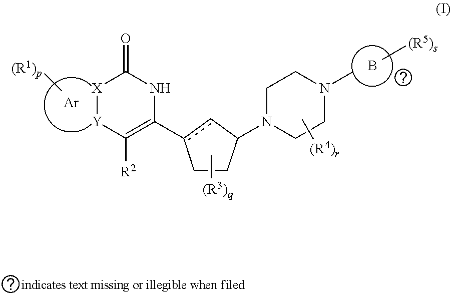

wherein, [symbol]
is either a single or a double bond;
X and Y independently represent carbon or nitrogen;
ring Ar is selected from
a) 6 membered heteroaromatic ring containing 1 to 2 nitrogen atoms, with X and Y being carbon; and
b) 5 membered heteroaromatic ring containing 1 to 2 heteroatoms selected from nitrogen, oxygen, and sulphur, wherein both X and Y are not selected as nitrogen at the same time;
   R¹ is independently selected at each occurrence from halogen, nitro, cyano, perhaloalkyl, substituted- or unsubstituted-alkyl, substituted- or unsubstituted-cyclopropyl, —NH₂, —N(H)CH₃, —OH, and —OCH₃;
   R² is selected from hydrogen, halogen, nitro, cyano, —NH₂, —N(H)CH₃, —OH, —OCH₃, substituted- or unsubstituted-cyclopropyl, and substituted- or unsubstituted-alkyl;
   R³ is independently selected at each occurrence from halogen, and substituted- or unsubstituted-alkyl, or two R³ on the same carbon form an oxo (═O), or two R³ groups together with the carbon atom(s) to which they are attached form a substituted- or unsubstituted-carbocycle;
   R⁴ is independently selected at each occurrence as substituted- or unsubstituted-alkyl, or two R⁴ on the same carbon form an oxo (═O), or two R⁴ groups together with the carbon atom(s) to which they are attached form a substituted- or unsubstituted-carbocycle or substituted- or unsubstituted-heterocycle;
   ring B is selected from cycloalkyl, heterocyclyl, aryl, and heteroaryl;
   R⁵ is independently selected at each occurrence from halogen, nitro, cyano, perhaloalkyl, substituted- or unsubstituted-alkyl, C(═O)R^{1a}, —C(═O)OR^{1b}, —C(═O)NR^{1b}R^{1c}, —NR^{1d}R^{1e}, and —OR^{1f};
   R^{1a} is selected from substituted- or unsubstituted-alkyl, and substituted- or unsubstituted-cycloalkyl;
   R^{1b} and R^{1c} are each independently selected from hydrogen, substituted- or unsubstituted-alkyl, and substituted- or unsubstituted-cycloalkyl;
   R^{1d} and R^{1e} are each independently selected from hydrogen, —C(═O)alkyl, substituted- or unsubstituted-alkyl, and substituted- or unsubstituted-cycloalkyl;
   R^{1f} is selected from hydrogen, —C(═O)alkyl, substituted- or unsubstituted-alkyl, perhaloalkyl, and substituted- or unsubstituted-cycloalkyl;
   p is selected from 0, 1, and 2;
   q is selected from 0, 1, 2, and 3;
   r is selected from 0, 1, 2, and 3;
   s is selected from 0, 1, 2, and 3;
   when ‘alkyl’ is substituted, it is substituted with 1 to 3 substituents independently selected from oxo (═O), halogen, nitro, cyano, perhaloalkyl, cycloalkyl, cycloalkenyl, heterocyclyl, —OR^{6b}, —SO₂R^{6a}, —C(═O)OR^{6a}, —OC(═O)R^{6a}, —C(═O)N(H)R⁶, —C(═O)N(alkyl)R⁶, —N(H)C(═O)R^{6a}, —N(H)R⁶, and —N(alkyl)R⁶;
   when ‘cycloalkyl’ and ‘carbocycle’ are substituted, each is substituted with 1 to 3 substituents independently selected from oxo (═O), halogen, nitro, cyano, alkyl, alkenyl, perhaloalkyl, heterocyclyl, —OR^{6b}, —SO₂R^{6a}, —C(═O)OR^{6a}, —OC(═O)R^{6a}, —C(═O)N(H)R⁶, —C(═O)N(alkyl)R⁶, —N(H)C(═O)R^{6a}, —N(H)R⁶, and —N(alkyl)R⁶;
   when the ‘heterocycle’ is substituted, it is substituted either on one or more ring carbon atoms or on one or more ring hetero atoms, and when it is substituted on ring carbon atom(s), it is substituted with 1 to 3 substituents independently selected from oxo (═O), halogen, cyano, alkyl, alkenyl, perhaloalkyl, —OR⁶, —SO₂(alkyl), —C(═O)O(alkyl), —C(═O)N(H)R⁶, —C(═O)N(alkyl)R⁶, —N(H)C(═O)(alkyl), —N(H)R⁶, and —N(alkyl)₂; and when the heterocyclic group is substituted on ring nitrogen atom(s), it is substituted with a substituent or substituents independently selected from alkyl, alkenyl, cycloalkyl, cycloalkenyl, —SO₂(alkyl), —C(═O)(alkyl), C(═O)O(alkyl), —C(═O)N(H)R⁶, and —C(═O)N(alkyl)R⁶;
   each R⁶ is independently selected from hydrogen, alkyl, alkenyl, cycloalkyl, cycloalkenyl, and heterocyclyl;
   each R^{6a} is independently selected from alkyl, alkenyl, perhaloalkyl, cycloalkyl, cycloalkenyl, and heterocyclyl; and
   R^{6b} is selected from hydrogen, alkyl, alkenyl, perhaloalkyl, cycloalkyl, cycloalkenyl, and heterocyclyl.

In an embodiment, ring Ar is

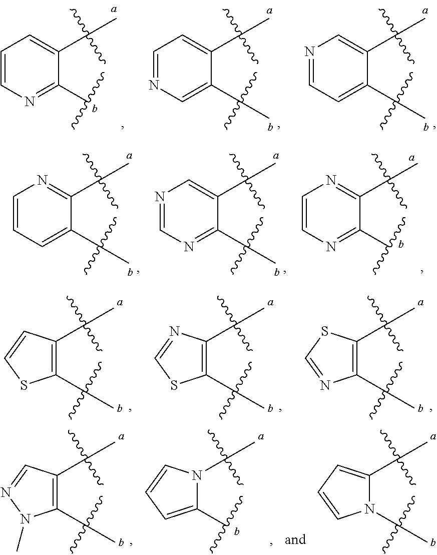

wherein a and b represent the points of attachment of the C═O and CR₂ moieties of the adjoining dihydropyridinone ring.

In any of the above embodiments, R¹ is independently selected at each occurrence from halogen, substituted- or unsubstituted-alkyl, and —NH₂.

In another embodiment, R¹ is independently selected at each occurrence from fluorine, methyl, and amino.

In any of the above embodiments, p is 0 or 1.

In any of the above embodiments, R² is selected from hydrogen, nitro, and substituted- or unsubstituted-alkyl.

In another embodiment, R² is selected from hydrogen, nitro, and methyl.

In any of the above embodiments, q is 0.

In any of the above embodiments, R⁴ is independently selected at each occurrence as substituted- or unsubstituted-alkyl, or two R⁴ on the same carbon form an oxo (═O), or two R⁴ groups together with the carbon atoms to which they are attached form a substituted- or unsubstituted-heterocycle.

In another embodiment, R⁴ is independently selected at each occurrence as methyl, or two R⁴ on the same carbon form an oxo (═O), or two R⁴ groups together with the carbon atoms to which they are attached form a 2,5-diazabicyclo[2.2.1]heptane.

In any of the above embodiments, r is selected from 0, 1, and 2.

In any of the above embodiments, ring B is selected from aryl and heteroaryl.

In an embodiment, ring B is selected from phenyl, pyridinyl, thiazolyl, 2,3-dihydro-indene-5-yl, 2,3-dihydro-1-indenone-5-yl, 1-isoindolinone-5-yl, and 2,3-dihydro-1-isobenzofuranone-5-yl.

More particularly, ring B is selected from

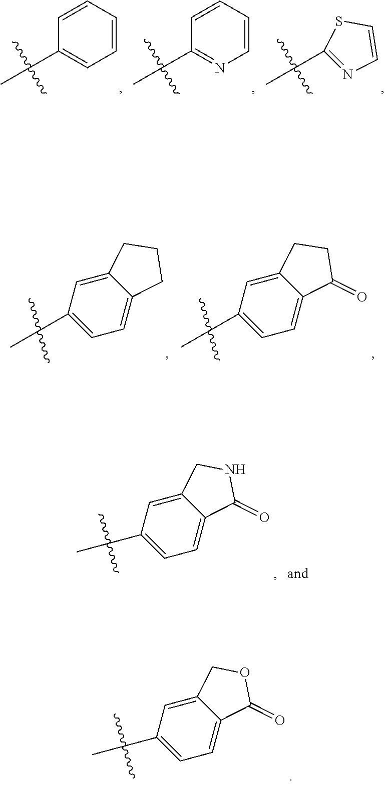

In any of the above embodiments, R⁵ is independently selected at each occurrence from halogen, cyano, perhaloalkyl, substituted- or unsubstituted-alkyl, C(═O)R^{1a}, —C(═O)OR^{1b}, —C(═O)NR^{1b}R^{1c}, —NR^{1d}R^{1e}, and —OR^{1f}, wherein R^{1a} is substituted- or unsubstituted-alkyl; R^{1b} and R^{1e} are each independently selected from hydrogen, and substituted- or unsubstituted-alkyl; R^{1d} and R^{1e} are each independently selected from hydrogen, and substituted- or unsubstituted-alkyl; and R^{1f} is substituted- or unsubstituted-alkyl.

In another embodiment, R⁵ is independently selected at each occurrence from fluorine, chlorine, cyano, trifluoromethyl, methyl, —C(═O)CH₃, —C(═O)OCH₂CH₃, —C(═O)NHCH₃, —C(═O)NH₂, —NHCH₃, and —OCH₃.

In any of the above embodiments, s is selected from 0, 1, and 2.

In another embodiment, ring Ar is

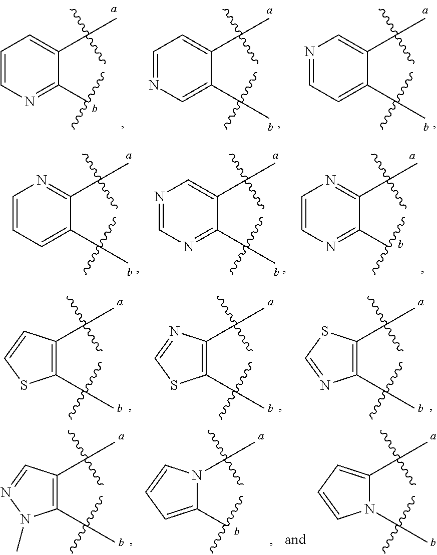

wherein a and b represent the points of attachment of the C═O and CR₂ moieties of the adjoining dihydropyridinone ring;

R¹ is independently selected at each occurrence from halogen, substituted- or unsubstituted-alkyl, and —NH₂;

R² is selected from hydrogen, nitro, and substituted- or unsubstituted-alkyl;

R⁴ is independently selected at each occurrence as substituted- or unsubstituted-alkyl, or two R⁴ on the same carbon form an oxo (═O), or two R⁴ groups together with the carbon atoms to which they are attached form a substituted- or unsubstituted-heterocycle;

ring B is selected from aryl and heteroaryl;

R⁵ is independently selected at each occurrence from halogen, cyano, perhaloalkyl, substituted- or unsubstituted-alkyl, C(═O)R^{1a}, —C(═O)OR^{1b}, —C(═O)NR^{1b}R^{1c}, —NR^{1d}R^{1e}, and —OR^{1f}, wherein R^{1a} is substituted- or unsubstituted-alkyl; R^{1b} and R^{1e} are each independently selected from hydrogen, and substituted- or unsubstituted-alkyl; R^{1d} and R^{1e} are each independently selected from hydrogen, and substituted- or unsubstituted-alkyl; and R^{1f} is substituted- or unsubstituted-alkyl;

p is 0 or 1;

q is 0;

r is selected from 0, 1, and 2; and

s is selected from 0, 1, and 2.

In yet another embodiment, ring Ar is

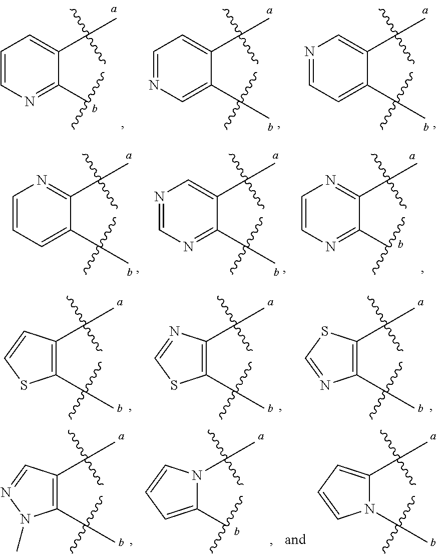

wherein a and b represent the points of attachment of the C═O and CR₂ moieties of the adjoining dihydropyridinone ring;

R¹ is independently selected at each occurrence from fluorine, methyl, and amino;

R² is selected from hydrogen, nitro, and methyl;

R⁴ is independently selected at each occurrence as methyl, or two R⁴ on the same carbon form an oxo (═O), or two R⁴ groups together with the carbon atoms to which they are attached form a 2,5-diazabicyclo[2.2.1]heptane;

ring B is selected from phenyl, pyridinyl, thiazolyl, 2,3-dihydro-indene-5-yl, 2,3-dihydro-1-indenone-5-yl, 2,3-dihydro-1-isobenzofuranone-5-yl, and 1-isoindolinone-5-yl;

R⁵ is independently selected at each occurrence from fluorine, chlorine, cyano, trifluoromethyl, methyl, —C(═O)CH₃, —C(═O)OCH₂CH₃, —C(═O)NHCH₃, —C(═O)NH₂, —NH(CH₃), and —OCH₃;

p is 0 or 1;

q is 0;

r is selected from 0, 1, and 2; and

s is selected from 0, 1, and 2.

In a further embodiment, the compound of formula (I) has the structure of formula (Ia):

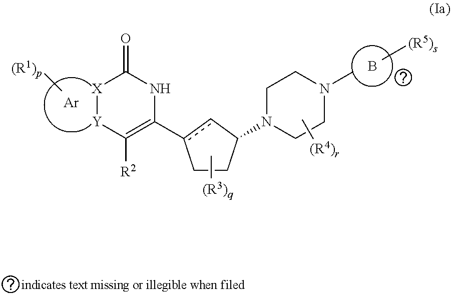

wherein R¹-R⁵, ring Ar, ring B, X, Y, p, q, r and s are as defined in formula (I).

In another embodiment, the compound of formula (I) has the structure of formula (Ib):

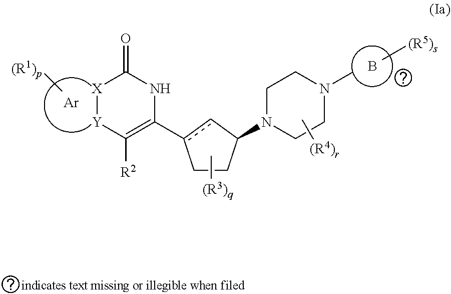

wherein R¹-R⁵, ring Ar, ring B, X, Y, p, q, r and s are as defined in formula (I).
General terms used in formula can be defined as follows; however, the meaning stated should not be interpreted as limiting the scope of the term per se.

The term ‘alkyl’, as used herein, means a straight chain or branched hydrocarbon containing from 1 to 20 carbon atoms. Preferably, the alkyl group contains 1 to 10 carbon atoms. More preferably, the alkyl group contains up to 6 carbon atoms. Representative examples of alkyl groups include, but are not limited to, methyl, ethyl, n-propyl, iso-propyl, n-butyl, sec-butyl, iso-butyl, tert-butyl, n-pentyl, isopentyl, neopentyl, and n-hexyl.

The term ‘substituted alkyl’, as defined hereinabove refers to an alkyl group which is substituted with 1 to 3 substituents independently selected from oxo (═O), halogen, nitro, cyano, perhaloalkyl, cycloalkyl, cycloalkenyl, heterocyclyl, —OR^{6b}, —SO₂R^{6a}, —C(═O)OR^{6a}, —OC(═O)R^{6a}, —C(═O)N(H)R⁶, —C(═O)N(alkyl)R⁶, —N(H)C(═O)R^{6a}, —N(H)R⁶, and —N(alkyl)R⁶; each R⁶ is independently selected from hydrogen, alkyl, alkenyl, cycloalkyl, cycloalkenyl, and heterocyclyl; each R^{6a} is independently selected from alkyl, alkenyl, perhaloalkyl, cycloalkyl, cycloalkenyl, and heterocyclyl; and R^{6b} is selected from hydrogen, alkyl, alkenyl, perhaloalkyl, cycloalkyl, cycloalkenyl, and heterocyclyl.

The term ‘perhaloalkyl’, as used herein, means an alkyl group as defined hereinabove wherein all the hydrogen atoms of the said alkyl group are substituted with halogen. The perhaloalkyl group is exemplified by trifluoromethyl, pentafluoroethyl, and the like.

The term ‘cycloalkyl’ and ‘carbocycle’ as used herein, means a monocyclic, bicyclic, or tricyclic non-aromatic ring system containing from 3 to 14 carbon atoms, preferably monocyclic cycloalkyl ring containing 3 to 6 carbon atoms. Examples of monocyclic ring systems include cyclopropyl, cyclobutyl, cyclopentyl, cyclohexyl, cycloheptyl, and cyclooctyl. Bicyclic ring systems include monocyclic ring system fused across a bond with another cyclic system which may be an alicyclic ring or an aromatic ring. Bicyclic rings also include spirocyclic systems wherein the second ring gets annulated on a single carbon atom. Bicyclic ring systems are also exemplified by a bridged monocyclic ring system in which two non-adjacent carbon atoms of the monocyclic ring are linked by an alkylene bridge. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane, bicyclo[3.3.2]decane, bicyclo[3.1.0]hexane, bicyclo[4.1.0]heptane, bicyclo[3.2.0]heptanes, octahydro-1H-indene, spiro[2.5]octane, spiro[4.5]decane, spiro[bicyclo[4.1.0]heptane-2,1′-cyclopentane], hexahydro-2′H-spiro[cyclopropane-1,1′-pentalene]. Tricyclic ring systems are the systems wherein the bicyclic systems as described above are further annulated with third ring, which may be an alicyclic ring or aromatic ring. Tricyclic ring systems are also exemplified by a bicyclic ring system in which two non-adjacent carbon atoms of the bicyclic ring are linked by a bond or an alkylene bridge. Representative examples of tricyclic-ring systems include, but are not limited to, tricyclo[3.3.1.0^{3.7}]nonane, and tricyclo[3.3.1.1^{3.7}]decane (adamantane).

The term ‘alkenyl’, as used herein, means an alkyl group containing at least one carbon-carbon double bond. The term ‘cycloalkenyl’, as used herein, means a cycloalkyl group containing at least one carbon-carbon double bond.

The term ‘substituted cycloalkyl’, or ‘substituted carbocycle’ as defined hereinabove is a cycloalkyl group which is substituted with 1 to 3 substituents independently selected from oxo (═O), halogen, nitro, cyano, alkyl, alkenyl, perhaloalkyl, heterocyclyl, —OR^{6b}, —SO₂R^{6a}, —C(═O)OR^{6a}, —OC(═O)R^{6a}, —C(═O)N(H)R⁶, —C(═O)N(alkyl)R⁶, —N(H)C(═O)R^{6a}, —N(H)R⁶, and —N(alkyl)R⁶; each R⁶ is independently selected from hydrogen, alkyl, alkenyl, cycloalkyl, cycloalkenyl, and heterocyclyl; each R^{6a} is independently selected from alkyl, alkenyl, perhaloalkyl, cycloalkyl, cycloalkenyl, and heterocyclyl; and R^{6b} is selected from hydrogen, alkyl, alkenyl, perhaloalkyl, cycloalkyl, cycloalkenyl, and heterocyclyl.

The term ‘heterocycle’ or ‘heterocyclic’ as used herein, means a ‘cycloalkyl’ group wherein one or more of the carbon atoms replaced by heteroatom selected from N, S and O. The heterocycle may be connected to the parent molecular moiety through any carbon atom and/or any nitrogen atom contained within the heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. Representative examples of bicyclic heterocycle include, but are not limited to, 1,2,3,4-tetrahydroisoquinolin-2-yl, 1,2,3,4-tetrahydroquinolin-1-yl, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, and 1,2,3,4-tetrahydroquinolinyl. The term ‘heterocycle’ or ‘heterocyclic’ also includes bridged and spiro heterocyclic systems such as azabicyclo[3.2.1]octane, azabicyclo[3.3.1]nonane, 8-oxa-3-azabicyclo[3.2.1]octan-3-yl, 3-oxa-8-azabicyclo[3.2.1]octan-8-yl, 6-oxa-3-azabicyclo[3.1.1]heptan-3-yl, 8-azabicyclo[3.2.1]octan-8-yl, 3-azabicyclo[3.2.1]octan-3-yl, 3-azabicyclo[3.1.0]hexan-3-yl, 6-azaspiro[2.5]octan-6-yl, 5-azaspiro[2.5]octan-5-yl, 4-azaspiro[2.4]heptan-4-yl, 2,5-diazabicyclo[2.2.1]heptane and the like.

The term ‘substituted heterocycle’ or ‘substituted heterocyclic’ as defined hereinabove, each of them is substituted either on a ring carbon atoms or on a ring hetero atoms, and when it is substituted on a ring carbon atom(s), it is substituted with 1 to 3 substituents independently selected from oxo (═O), halogen, cyano, alkyl, alkenyl, perhaloalkyl, —OR⁶, —SO₂(alkyl), —C(═O)O(alkyl), —C(═O)N(H)R⁶, —C(═O)N(alkyl)R⁶, —N(H)C(═O)(alkyl), —N(H)R⁶, and —N(alkyl)₂; and when the heterocyclic group is substituted on a ring nitrogen atoms(s), it is substituted with one or more substituents independently selected from alkyl, alkenyl, cycloalkyl, cycloalkenyl, —SO₂(alkyl), —C(═O)(alkyl), C(═O)O(alkyl), —C(═O)N(H)R⁶, and —C(═O)N(alkyl)R⁶; wherein each R⁶ is independently selected from hydrogen, alkyl, alkenyl, cycloalkyl, cycloalkenyl, and heterocyclyl.

The term ‘aryl’, as used herein, refers to a monovalent monocyclic, bicyclic or tricyclic aromatic hydrocarbon ring system. Examples of aryl groups include phenyl, naphthyl, anthracenyl, fluorenyl, indenyl, azulenyl, and the like. The term ‘aryl’ as used herein, also includes partially saturated bicyclic and tricyclic aromatic hydrocarbons optionally substituted with oxo (═O), e.g., tetrahydro-naphthalene, 2,3-dihydro-indene-5-yl, and 2,3-dihydro-1-indenone-5-yl.

The term ‘heteroaryl’, as used herein, refers to a 5-14 membered monocyclic, bicyclic, or tricyclic ring system having 1-4 ring heteroatoms selected from O, N, or S, and the remainder ring atoms being carbon (with appropriate hydrogen atoms unless otherwise indicated), wherein at least one ring in the ring system is aromatic. Thus the term ‘heteroaryl’ as used herein, also includes a 5-14 membered partially saturated bicyclic and tricyclic aromatic ring system having 1-4 ring heteroatoms selected from O, N, or S, and the said heteroaryl is optionally substituted with oxo (═O). Examples of heteroaryl groups include, but not limited to, pyridyl, 1-oxo-pyridyl, furanyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, benzoxazolyl, benzofuranyl, indolizinyl, imidazopyridyl, tetrazolyl, benzimidazolyl, benzothiazolyl, benzothiadiazolyl, benzoxadiazolyl, indolyl, azaindolyl, imidazopyridyl, quinazolinyl, purinyl, pyrrolo[2,3]pyrimidinyl, pyrazolo[3,4]pyrimidinyl, and benzo(b)thienyl, 2,3-thiadiazolyl, 1H-pyrazolo[5,1-c]-1,2,4-triazolyl, pyrrolo[3,4-d]-1,2,3-triazolyl, cyclopentatriazolyl, 3H-pyrrolo[3,4-c] isoxazolyl, 2,3-dihydro-benzo[1,4]dioxin-6-yl, 2,3-dihydro-benzo[1,4]dioxin-5-yl, 2,3-dihydro-benzofuran-5-yl, 2,3-dihydro-benzofuran-4-yl, 2,3-dihydro-benzofuran-6-yl, 2,3-dihydro-benzofuran-6-yl, 2,3-dihydro-isobenzofuran-5-yl, 2,3-dihydro-1-isobenzofuranone-5-yl, 2,3-dihydro-1H-indol-5-yl, 2,3-dihydro-1H-indol-4-yl, 2,3-dihydro-1H-indol-6-yl, 2,3-dihydro-1H-indol-7-yl, 1-isoindolinone-5-yl, benzo[1,3]dioxol-4-yl, benzo[1,3]dioxol-5-yl, 1,2,3,4-tetrahydroquinolinyl, 1,2,3,4-tetrahydroisoquinolinyl, 2,3-dihydrobenzothien-4-yl, 2-oxoindolin-5-yl and the like.

The term ‘oxo’ means a divalent oxygen (═O) attached to the parent group. For example, an oxo attached to carbon forms a carbonyl, an oxo substituted on cyclohexane forms a cyclohexanone, and the like.

The term ‘annulated’ means the ring system under consideration is either annulated with another ring at a carbon atom(s) of the cyclic system or across a bond of the cyclic system as in the case of fused or spiro ring systems.

The term ‘bridged’ means the ring system under consideration contain an alkylene bridge having 1 to 4 methylene units joining two non-adjacent ring atoms.

Whenever a range of the number of atoms in a structure is indicated (e.g., a C₁ to C₂₀ alkyl, C₂ to C₂₀ alkenyl etc.), it is specifically contemplated that any sub-range or individual number of carbon atoms falling within the indicated range also can be used. Thus, for instance, the recitation of a range of 1-6 carbon atoms (e.g., C₁ to C₆), 2-6 carbon atoms (e.g., C₂ to C₆), 3-6 carbon atoms (e.g., C₃ to C₆), as used with respect to any chemical group (e.g., alkyl, alkenyl, etc.) referenced herein encompasses and specifically describes 1, 2, 3, 4, 5, and/or 6 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 2-3 carbon atoms, 2-4 carbon atoms, 2-5 carbon atoms, 2-6 carbon atoms, 3-4 carbon atoms, 3-5 carbon atoms, 3-6 carbon atoms, 4-5 carbon atoms, 4-6 carbon atoms as appropriate).

In accordance with an embodiment, the invention provides a compound, its tautomeric form, its stereoisomers, racemates, and pharmaceutically acceptable salt thereof as described hereinabove wherein the compound of general formula (I) is selected from:
(R)-4-(4-(3-(5-oxo-5,6-dihydro-1,6-naphthyridin-7-yl)cyclopent-2-en-1-yl)piperazin-1-yl)benzonitrile (Compound 1);
(R)-4-(4-(3-(3-fluoro-5-oxo-5,6-dihydro-1,6-naphthyridin-7-yl)cyclopent-2-en-1-yl)piperazin-1-yl)benzonitrile (Compound 2);
(R)-7-(3-(4-(o-tolyl)piperazin-1-yl)cyclopent-1-en-1-yl)-1,6-naphthyridin-5(6H)-one (Compound 3);
(S)-4-(4-(3-(5-oxo-5,6-dihydro-1,6-naphthyridin-7-yl)cyclopent-2-en-1-yl)piperazin-1-yl)benzonitrile (Compound 4);
(S)-4-(4-(3-(3-fluoro-5-oxo-5,6-dihydro-1,6-naphthyridin-7-yl)cyclopent-2-en-1-yl)piperazin-1-yl)benzonitrile (Compound 5);
(R)-4-(4-(3-(2-methyl-5-oxo-5,6-dihydro-1,6-naphthyridin-7-yl)cyclopent-2-en-1-yl)piperazin-1-yl)benzonitrile (Compound 6);
(R)-4-(4-(3-(3-amino-5-oxo-5,6-dihydro-1,6-naphthyridin-7-yl)cyclopent-2-en-1-yl)piperazin-1-yl)benzonitrile (Compound 7);
(R)-4-(4-(3-(8-nitro-5-oxo-5,6-dihydro-1,6-naphthyridin-7-yl)cyclopent-2-en-1-yl)piperazin-1-yl)benzonitrile (Compound 8);
(R)-4-(4-(3-(8-methyl-5-oxo-5,6-dihydro-1,6-naphthyridin-7-yl)cyclopent-2-en-1-yl)piperazin-1-yl)benzonitrile (Compound 9);
(S)-4-(4-(3-(8-methyl-5-oxo-5,6-dihydro-1,6-naphthyridin-7-yl)cyclopent-2-en-1-yl)piperazin-1-yl)benzonitrile (Compound 10);
4-(4-((1R,3S/3R)-3-(5-oxo-5,6-dihydro-1,6-naphthyridin-7-yl)cyclopentyl)piperazin-1-yl)benzonitrile (Compound 11);
4-(4-((1R,3R/3S)-3-(5-oxo-5,6-dihydro-1,6-naphthyridin-7-yl)cyclopentyl)piperazin-1-yl)benzonitrile (Compound 12);
(R)-4-(2-oxo-4-(3-(5-oxo-5,6-dihydro-1,6-naphthyridin-7-yl)cyclopent-2-en-1-yl)piperazin-1-yl)benzonitrile (Compound 13);
4-((R)-3-methyl-4-((R/S)-3-(5-oxo-5,6-dihydro-1,6-naphthyridin-7-yl)cyclopent-2-en-1-yl)piperazin-1-yl)benzonitrile (Compound 14);
4-((R)-3-methyl-4-((S/R)-3-(5-oxo-5,6-dihydro-1,6-naphthyridin-7-yl)cyclopent-2-en-1-yl)piperazin-1-yl)benzonitrile (Compound 15);
4-((1S,4S)-5-((R/S)-3-(5-oxo-5,6-dihydro-1,6-naphthyridin-7-yl)cyclopent-2-en-1-yl)-2,5-diazabicyclo[2.2.1]heptan-2-yl)benzonitrile (Compound 16);
4-((1S,4S)-5-((S/R)-3-(5-oxo-5,6-dihydro-1,6-naphthyridin-7-yl)cyclopent-2-en-1-yl)-2,5-diazabicyclo[2.2.1]heptan-2-yl)benzonitrile (Compound 17);
(R)—N-methyl-4-(4-(3-(5-oxo-5,6-dihydro-1,6-naphthyridin-7-yl)cyclopent-2-en-1-yl)piperazin-1-yl)benzamide (Compound 18);
(R)-4-(4-(3-(5-oxo-5,6-dihydro-1,6-naphthyridin-7-yl)cyclopent-2-en-1-yl)piperazin-1-yl)benzamide (Compound 19);
Ethyl(R)-4-(4-(3-(5-oxo-5,6-dihydro-1,6-naphthyridin-7-yl)cyclopent-2-en-1-yl)piperazin-1-yl)benzoate (Compound 20);
(R)-7-(3-(4-phenylpiperazin-1-yl)cyclopent-1-en-1-yl)-1,6-naphthyridin-5(6H)-one (Compound 21);
(R)-7-(3-(4-(4-fluorophenyl)piperazin-1-yl)cyclopent-1-en-1-yl)-1,6-naphthyridin-5(6H)-one (Compound 22);
(R)-3-fluoro-4-(4-(3-(5-oxo-5,6-dihydro-1,6-naphthyridin-7-yl)cyclopent-2-en-1-yl)piperazin-1-yl)benzonitrile (Compound 23);
(R)-7-(3-(4-(4-chlorophenyl)piperazin-1-yl)cyclopent-1-en-1-yl)-1,6-naphthyridin-5(6H)-one (Compound 24);
(R)-7-(3-(4-(4-methoxyphenyl)piperazin-1-yl)cyclopent-1-en-1-yl)-1,6-naphthyridin-5(6H)-one (Compound 25);
(R)-7-(3-(4-(p-tolyl)piperazin-1-yl)cyclopent-1-en-1-yl)-1,6-naphthyridin-5(6)-one (Compound 26);
(R)-7-(3-(4-(4-(methylamino)phenyl)piperazin-1-yl)cyclopent-1-en-1-yl)-1,6-naphthyridin-5(6H)-one (Compound 27);
(R)-7-(3-(4-(4-acetylphenyl) piperazin-1-yl)cyclopent-1-en-1-yl)-1,6-naphthyridin-5(6H)-one (Compound 28);
(R)-7-(3-(4-(1-oxo-2,3-dihydro-1H-inden-5-yl)piperazin-1-yl)cyclopent-1-en-1-yl)-1,6-naphthyridin-5(6H)-one (Compound 29);
(R)-7-(3-(4-(2,3-dihydro-1H-inden-5-yl)piperazin-1-yl)cyclopent-1-en-1-yl)-1,6-naphthyridin-5(6H)-one (Compound 30);
(R)-7-(3-(4-(1-oxo-1,3-dihydroisobenzofuran-5-yl)piperazin-1-yl)cyclopent-1-en-1-yl)-1,6-naphthyridin-5(6H)-one (Compound 31);
(R)-7-(3-(4-(1-oxoisoindolin-5-yl)piperazin-1-yl)cyclopent-1-en-1-yl)-1,6-naphthyridin-5(6H)-one (Compound 32);
(R)-7-(3-(4-(4-(trifluoromethyl)phenyl)piperazin-1-yl)cyclopent-1-en-1-yl)-1,6-naphthyridin-5(6H)-one (Compound 33);
(R)-6-(4-(3-(5-oxo-5,6-dihydro-1,6-naphthyridin-7-yl)cyclopent-2-en-1-yl)piperazin-1-yl)nicotinonitrile (Compound 34);
(R)-2-(4-(3-(5-oxo-5,6-dihydro-1,6-naphthyridin-7-yl)cyclopent-2-en-1-yl)piperazin-1-yl)thiazole-5-carbonitrile (Compound 35);
(R)-4-(4-(3-(1-oxo-1,2-dihydro-2,6-naphthyridin-3-yl)cyclopent-2-en-1-yl)piperazin-1-yl)benzonitrile (Compound 36);
(R)-4-(4-(3-(8-oxo-7,8-dihydro-1,7-naphthyridin-6-yl)cyclopent-2-en-1-yl)piperazin-1-yl)benzonitrile (Compound 37);
(R)-4-(4-(3-(1-oxo-1,2-dihydro-2,7-naphthyridin-3-yl)cyclopent-2-en-1-yl)piperazin-1-yl)benzonitrile (Compound 38);
(R)-7-(3-(4-(2,4-difluorophenyl)piperazin-1-yl)cyclopent-1-en-1-yl)-1,6-naphthyridin-5(6H)-one (Compound 39);
(R)-4-(4-(3-(5-oxo-5,6-dihydropyrido[4,3-d]pyrimidin-7-yl)cyclopent-2-en-1-yl)piperazin-1-yl)benzonitrile (Compound 40);
(R)-4-(4-(3-(5-oxo-5,6-dihydropyrido[3,4-b]pyrazin-7-yl)cyclopent-2-en-1-yl)piperazin-1-yl)benzonitrile (Compound 41);
(R)-4-(4-(3-(4-oxo-4,5-dihydrothieno[3,2-c]pyridin-6-yl)cyclopent-2-en-1-yl)piperazin-1-yl)benzonitrile (Compound 42);
(R)-4-(4-(3-(4-oxo-4,5-dihydrothiazolo[5,4-c]pyridin-6-yl)cyclopent-2-en-1-yl)piperazin-1-yl)benzonitrile (Compound 43);
(R)-4-(4-(3-(4-oxo-4,5-dihydrothiazolo[4,5-c]pyridin-6-yl)cyclopent-2-en-1-yl)piperazin-1-yl)benzonitrile (Compound 44);
(S)-4-(4-(3-(4-oxo-4,5-dihydrothieno[3,2-c]pyridin-6-yl)cyclopent-2-en-1-yl)piperazin-1-yl)benzonitrile (Compound 45);
(S)-4-(4-(3-(4-oxo-4,5-dihydrothiazolo[5,4-c]pyridin-6-yl)cyclopent-2-en-1-yl)piperazin-1-yl)benzonitrile (Compound 46);
(R)-6-(3-(4-(4-fluorophenyl)piperazin-1-yl)cyclopent-1-en-1-yl)thieno[3,2-c]pyridin-4(5H)-one (Compound 47);
(R)-6-(3-(4-phenylpiperazin-1-yl)cyclopent-1-en-1-yl)thieno[3,2-c]pyridin-4(5H)-one (Compound 48);
(R)—N-methyl-4-(4-(3-(4-oxo-4,5-dihydrothieno[3,2-c]pyridin-6-yl)cyclopent-2-en-1-yl)piperazin-1-yl)benzamide (Compound 49);
(R)-6-(4-(3-(4-oxo-4,5-dihydrothieno[3,2-c]pyridin-6-yl)cyclopent-2-en-1-yl)piperazin-1-yl)nicotinonitrile (Compound 50);
(R)-6-(3-(4-(thiazol-2-yl)piperazin-1-yl)cyclopent-1-en-1-yl)thieno[3,2-c]pyridin-4(5H)-one (Compound 51);
(R)-3-fluoro-4-(4-(3-(4-oxo-4,5-dihydrothiazolo[5,4-c]pyridin-6-yl)cyclopent-2-en-1-yl)piperazin-1-yl)benzonitrile (Compound 52);
(R)-4-(4-(3-(1-methyl-4-oxo-4,5-dihydro-1H-pyrazolo[4,3-c]pyridin-6-yl)cyclopent-2-en-1-yl)piperazin-1-yl)benzonitrile (Compound 53);
(R)-4-(4-(3-(1-oxo-1,2-dihydropyrrolo[1,2-c]pyrimidin-3-yl)cyclopent-2-en-1-yl)piperazin-1-yl)benzonitrile (Compound 54);
(R)-3-(3-(4-(4-fluorophenyl)piperazin-1-yl)cyclopent-1-en-1-yl)pyrrolo[1,2-c]pyrimidin-1 (2H)-one (Compound 55); and
(R)-4-(4-(3-(1-oxo-1,2-dihydropyrrolo[1,2-a]pyrazin-3-yl)cyclopent-2-en-1-yl)piperazin-1-yl)benzonitrile (Compound 56).
   According to an embodiment of the present invention, the compounds of general formula (I) where all the symbols are as defined earlier, can be prepared by methods given in Schemes 1-15 and the examples. Representative procedures are shown below, however; these synthetic methods should not be construed as limiting the invention in any way, which lies in the whole genus described by the compound of formula (I) as disclosed hereinabove.

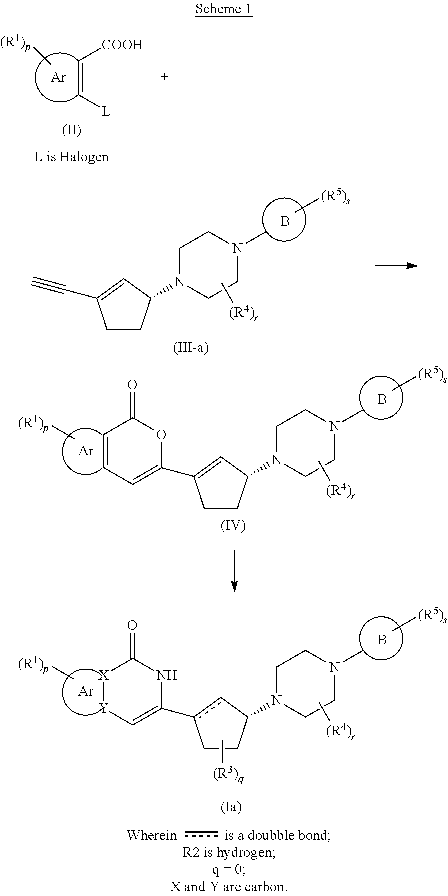

Scheme 1 shows a method of preparation of the compounds of formula represented as (Ia) in accordance with an embodiment. The compounds of formula (Ia), wherein R² is hydrogen, q=0, [symbol] is double bond, X and Y are carbon, and all other symbols are as defined under formula (I), can be prepared from compounds of formula (III-a), wherein R⁴ and R⁵ are as defined under formula (I).

The compounds of formula (II), wherein, L is halogen or trifluoromethanesulfonate (OTf), and all other symbols are as defined under formula (I), are subjected to Sonogashira coupling with compounds of formula (III-a) wherein R⁴ and R⁵ are defined earlier in formula (I), followed by in situ cyclization to obtain the compounds of formula (IV). Sonogashira coupling can be carried out under different coupling conditions and in a suitable solvent or solvents, for example, a halogenated hydrocarbon such as dichloromethane or chloroform, an aromatic hydrocarbon such as xylene, toluene, or benzene, an ether type solvent such as diethyl ether, tetrahydrofuran or 1,4-dioxane, an aprotic solvent such as dimethylformamide, dimethylsulfoxide, acetonitrile, or N-methyl-2-pyrrolidinone, in the presence of a suitable base such as potassium carbonate, triethylamine, diethylisopropylamine, diisopropylethylamine or the like, and a palladium catalyst such as bis(triphenylphosphine)palladium (II) dichloride [(PPh₃)₂PdCl₂], bis(triphenylphosphine)palladium (II) diacetate [(PPh₃)₂Pd(OAc)₂] combined with a co-catalytic amount of copper(I)iodide (CuI), as well known in the art (Review article by R. Chinchilla and C. Nejera; Chem. Soc. Rev., 2011, 40, 5084) at a temperature of 0-120° C. over a period of 1-12 hr to give compounds of formula (IV). Preferably, the Sonogashira reaction is carried out in anhydrous acetonitrile in the presence of bis(triphenylphosphine)palladium (II) chloride, using diisopropylethylamine or triethylamine as base at 60-65° C. under nitrogen for 3 hr.

The compounds of formula (IV), where all symbols are as defined under formula (I), can be treated with ammonia to obtain compounds of formula (Ia); where R² is hydrogen, q=0, [symbol] is double bond, X and Y are carbon, and all other symbols are as defined under formula (I). Preferably, the reaction is carried out in the presence of methanolic ammonia at 85° C. for 3 h.

The compounds of formula (Ib), wherein R² is hydrogen, q=0, [symbol] is double bond, X and Y are carbon, and all other symbols are as defined under formula (I), can be prepared from compounds of formula (III-b), wherein R⁴ and R⁵ are as defined under formula (I), using similar process as described in Scheme-1.

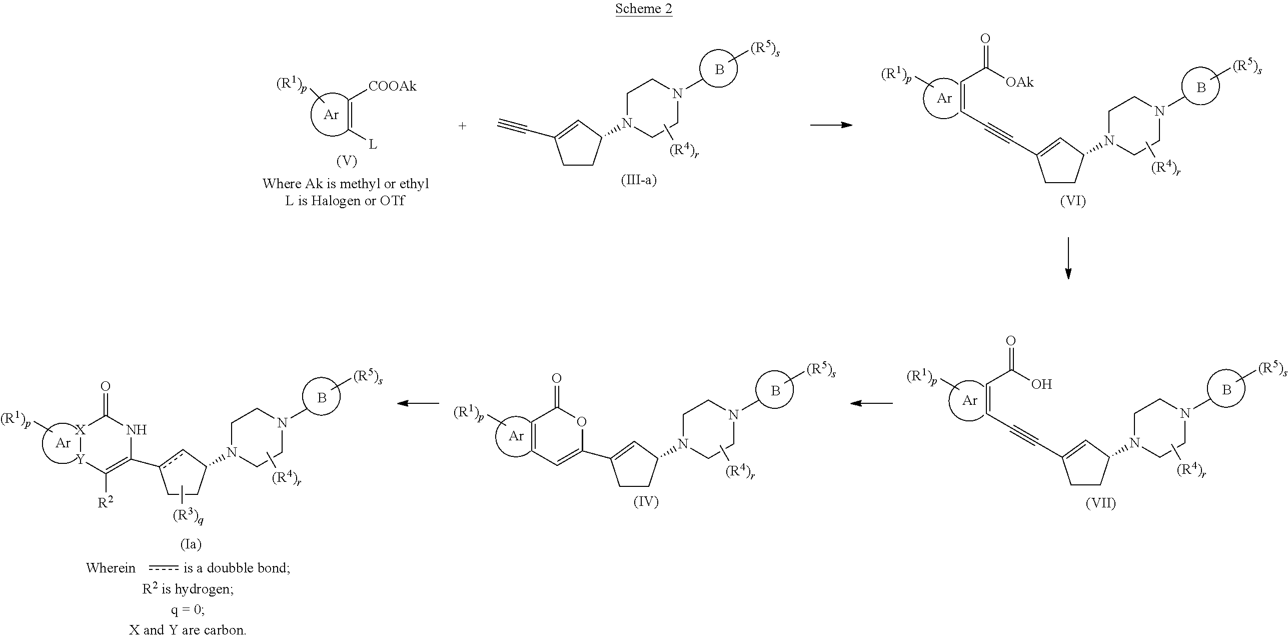

Scheme 2 shows a method of preparation of compounds of formula (Ia) in accordance with an embodiment. Compounds of formula (Ia), where R² is hydrogen, q=0, [symbol] is double bond, X and Y are carbon, and all other symbols are as defined under formula (I), can be prepared from compounds of formula (III-a), where R⁴ and R⁵ are as defined under formula (I).

The compounds of formula (V), wherein L is halogen, or trifluoromethanesulfonate (OTf), and all other symbols are as defined under formula (I), are subjected to Sonogashira coupling with compound of formula (III-a), where R⁴ and R⁵ are defined earlier in formula (I), to obtain compounds of formula (VI). Preferably, the Sonogashira reaction is carried out in anhydrous acetonitrile in the presence of bis(triphenylphosphine)palladium (II) chloride, using diisopropylethyl amine or triethylamine as base at 60-80° C. under nitrogen for 3-18 hours.

The compounds of formula (VI), where all symbols are as defined under formula (I), are hydrolyzed using sodium hydroxide in water and methanol to obtain compounds of formula (VII); and further cyclized to obtain compounds of formula (IV) using lewis acid such as trifluoromethane sulphonic acid.

The compounds of formula (IV), where all symbols are as defined under formula (I), can be treated with ammonia to obtain compounds of formula (Ia); where R² is hydrogen, q=0, [symbol] is double bond, X and Y are carbon, and all other symbols are as defined under formula (I). Preferably, the reaction is carried out in the presence of methanolic ammonia at 85° C. for 3 h.

The compounds of formula (Ib), wherein R² is hydrogen, q=0, [symbol] is double bond, X and Y are carbon, and all other symbols are as defined under formula (I), can be prepared from compounds of formula (III-b), wherein R⁴ and R⁵ are as defined under formula (I), using similar process as described in Scheme 2.

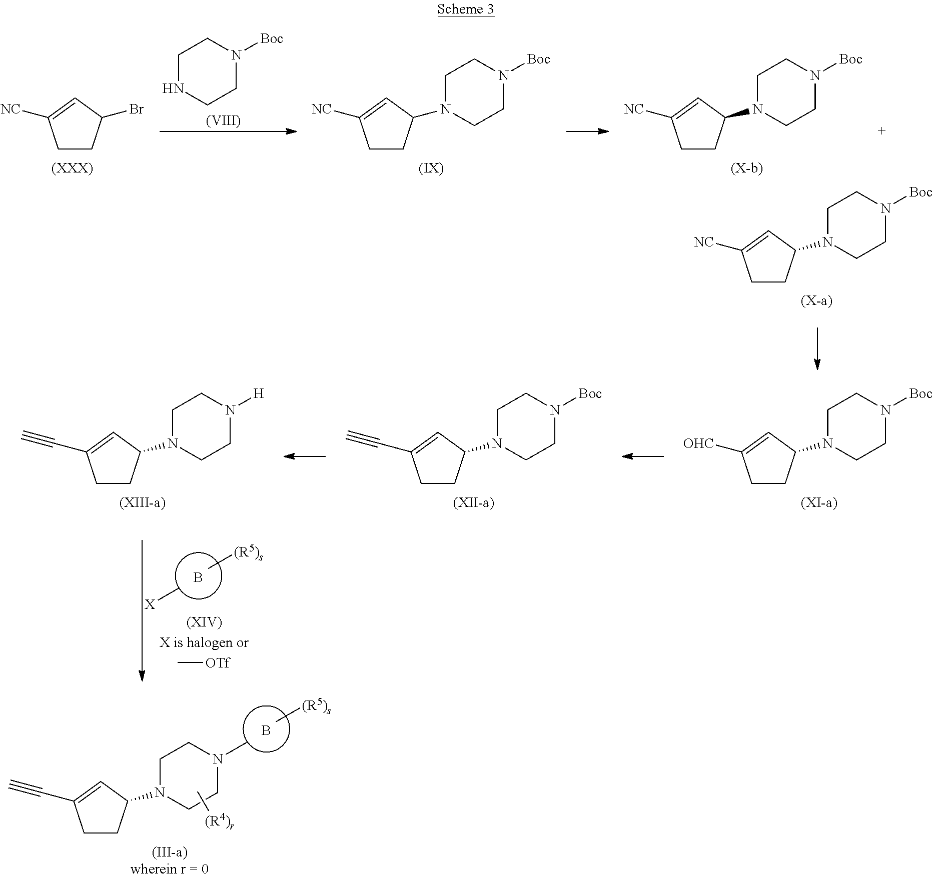

Scheme 3 shows a method of preparation of enantiopure compounds of formula (III-a). The compounds of formula (III-a), wherein r=0 and all other symbols are as defined under compounds of formula (I), can be prepared from a compound (IX). The compound (IX) is prepared from compound (XXX) and (VIII) according to the procedure reported in WO20149872.

Racemic compound of formula (IX) can be subjected to preparative chiral HPLC to separate two enantiomers compound (X-b) and compound (X-a). Enantiopure compound of formula (III-a) can be synthesized starting from enantiopure compound of formula (X-a).

Compound of formula (X-a) can be treated with diisobutyl aluminium hydride (DIBAL-H) in a suitable solvent or mixture of solvents, for example, tetrahydrofuran, toluene, chloroform, dichloromethane or the like, at a temperature of −78° C. to 50° C. over a period of 1-16 hr to give a compound of formula (XI-a).

The compound of formula (XI-a) can be treated with trimethylsilyldiazomethane solution (2M in diethyl ether or in hexane) in a suitable solvent, for example, tetrahydrofuran or the like, in the presence of base n-butyl lithium or the like at a temperature of −78° C. to 50° C. over a period of 1-20 hr to give a compound of formula (XII-a).

The compound of formula (XII-a) is subjected to deprotection of N-protecting group to obtain a compound of formula (XIII-a). Deprotection reaction of N-protecting groups can be carried out using standard procedures generally used in synthetic organic chemistry or well known in the literature e.g., Greene T. W. et al., 1999. Preferably, reaction is carried out in dichloromethane using hydrochloric acid in 1,4-dioxane.

The compound of formula (XIII-a) is reacted with the compounds of formula (XIV), where X═F, Cl, Br, I, or OTf, either in nucleophilic substitution reaction condition or Buchwald coupling method to obtain the compounds of formula (III-a), wherein r=0 and all other symbols are as defined under compounds of formula (I). The reaction may be carried out in a suitable solvent such as dimethylsulfoxide, N,N-dimethylformamide, 1,4-dioxane, acetonitrile, dichloromethane, methanol, or ethanol in the presence of a base such as potassium carbonate, sodium bicarbonate, triethylamine or the like, at a temperature of 25° C.-150° C. over a period of 30 min to 20 hr to obtain the compounds of formula (III-a). Preferably, reaction is carried out in N,N-dimethylformamide using potassium carbonate as base. On the other hand, Buchwald coupling can be carried out in a solvent such as toluene, tert-butanol, dimethylformamide, iso-propyl alcohol, 1,4-dioxane, 1,2-dimethoxyethane, tetrahydrofuran, and/or acetonitrile, in the presence of a base such as potassium phosphate, potassium carbonate, sodium tert-butoxide, cesium carbonate, lithium hexamethyl disilazane or the like, palladium catalysts such as palladium (II) acetate (Pd(OAc)₂), tris(dibenzyllideneacetone)dipalladium (0), [Pd₂(dba)₃], at a temperature of 50-160° C. and ligand such as 2,2′-Bis(diphenylphosphino)-1,1′-binaphthyl (BINAP), 2-Dicyclohexylphosphino-2′,4′,6′-triisopropylbiphenyl (XPhos), 2-Dicyclohexylphosphino-2′-(N,N-dimethylamino) biphenyl (DavePhos), (2-Biphenyl)di-tert-butylphosphine (JohnPhos), 2-Dicyclohexylphosphino-2′,6′-dimethoxybiphenyl (SPhos), 2-Dicyclohexylphosphino-2′-methylbiphenyl (MePhos) or the like.

Enantiopure compound of formula (III-b), wherein r=0 and all other symbols are as defined under compounds of formula (I) can be synthesized from enantiopure compound of formula (X-b). Enantiopure compound of formula (X-b) is synthesized by following methods described in the Scheme 3 which can be further converted to a compound of formula (III-b), wherein r=0 and all other symbols are as defined under compounds of formula (I); by following methods described in Scheme 3 for the synthesis of enantiopure compound of formula (III-a).

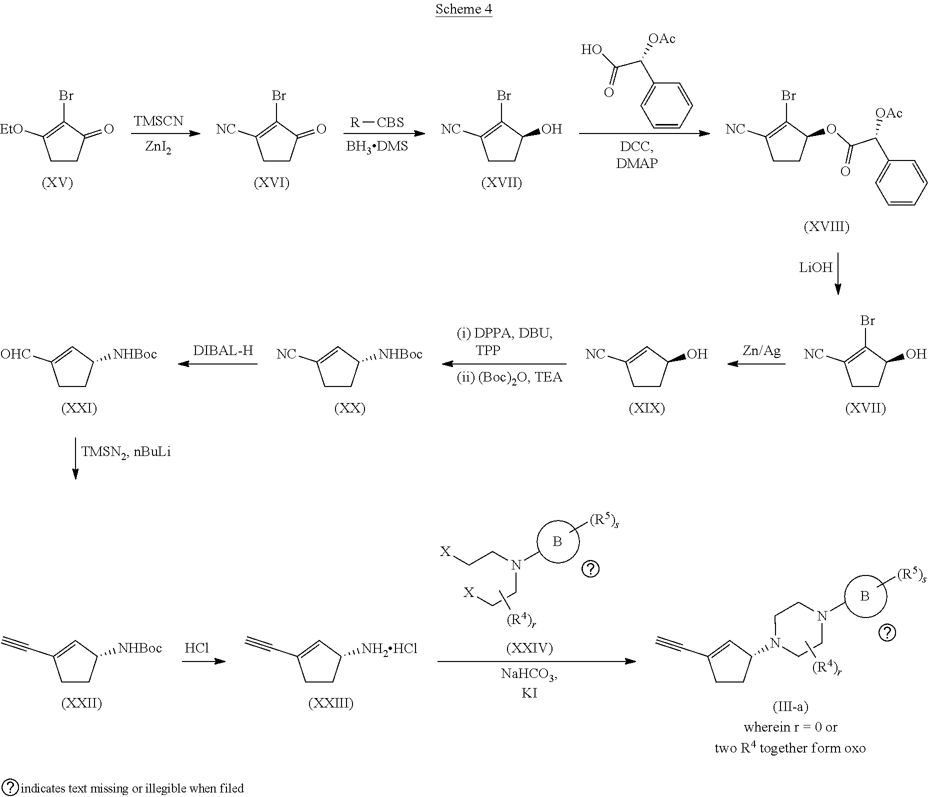

Scheme 4 shows a method of preparation of the compound of formula (III-a), wherein r=0 or two R⁴ together can form oxo and all other symbols are as defined under the compounds of formula (I), from a compound of formula (XV). The compound of formula (XV) can be prepared according to the procedure described in Journal of Medicinal Chemistry, 1999, 42, 7, 1274-1281.

The compound of formula (XV) is reacted with trimethylsilylcyanide (TMSCN) and zinc iodide, in the presence of an acid or zinc iodide in dichloromethane to obtain a compound of formula (XVI). The compound of formula (XVI) is reacted with (R)-1,3a-dimethyl-3,3-diphenylhexahydropyrrolo [1,2c][1,3,2]oxaborole (R-CBS) (1M solution in toluene) and Borane dimethyl sulphide complex (BH₃.DMS) in Tetrahydrofuran (THF) to obtain a compound of formula (XVII) with an enantiomeric excess ˜94.0%.

The compound of formula (XVII) as obtained in the previous step is subjected to coupling with (2R)-2-acetoxy-2-phenylacetic acid to obtain a compound of formula (XVIII) to enrich the enantiomeric excess. The coupling reaction can be carried out according to the conditions known for converting carboxylic acids to esters, to a person skilled in the art. The reaction may be carried out in an organic solvent, for example, N,N-dimethyl formamide, tetrahydrofuran, a halogenated hydrocarbon such as chloroform or dichloromethane, an aromatic hydrocarbon such as xylene, benzene, toluene, or mixtures thereof, or the like, in the presence of suitable base such as triethylamine, diisopropylethylamine, pyridine, dimethyl amino pyridine or the like at a temperature of 0-50° C. using reagents such as 1-(3-dimethylaminopropyl)-3-ethylcarbodiimide hydrochloride (EDCI), 1,3-dicyclohexylcarbodiimide (DCC), and auxiliary reagents such as 1-hydroxy-7-azabenzotriazole (HOAT), hydroxybenzotriazole hydrate (HOBT) or the like. Preferably, the coupling is carried out in dichloromethane using DCC and dimethyl amino pyridine as base. Further ester hydrolysis of the compound (XVIII) using LiOH in THF-water provides a compound of formula (XVII) with enantiomeric excess ˜98.5%.

The compound of formula (XVII) is reacted with Zn—Ag couple to obtain the de-brominated product as a compound of formula (XIX). The compound of formula (XIX) is reacted with [azido(phenoxy)phosphoryl]oxybenzene in tetrahydrofuran; the resulting intermediate is treated with triphenyl phospine, Boc-anhydride and triethylamine to obtain a compound of formula (XX).

The compound of formula (XX) is subjected to reduction using di-isobutyl aluminium hydride (DIBAL-H) in dichloromethane to obtain a compound of formula (XXI); which in turn is treated with trimethylsilyldiazomethane and n-butyl lithium in tetrahydrofuran to obtain a compound of formula (XXII).

The compound of formula (XXII) is treated with hydrochloric acid in dichloromethane or dioxane to obtain a compound of formula (XXIII) as hydrochloride salt; which in turn is reacted with a compound of formula (XXIV); wherein r=0 or two R⁴ together can form oxo and all other symbols are as defined under compounds of formula (I) and X is halogen, tosylate (OTs), mesylate (OMs), or any other leaving group, to obtain the compounds of formula (III-a); wherein r=0 or two R⁴ together can form oxo and all other symbols are as defined under compounds of formula (I).

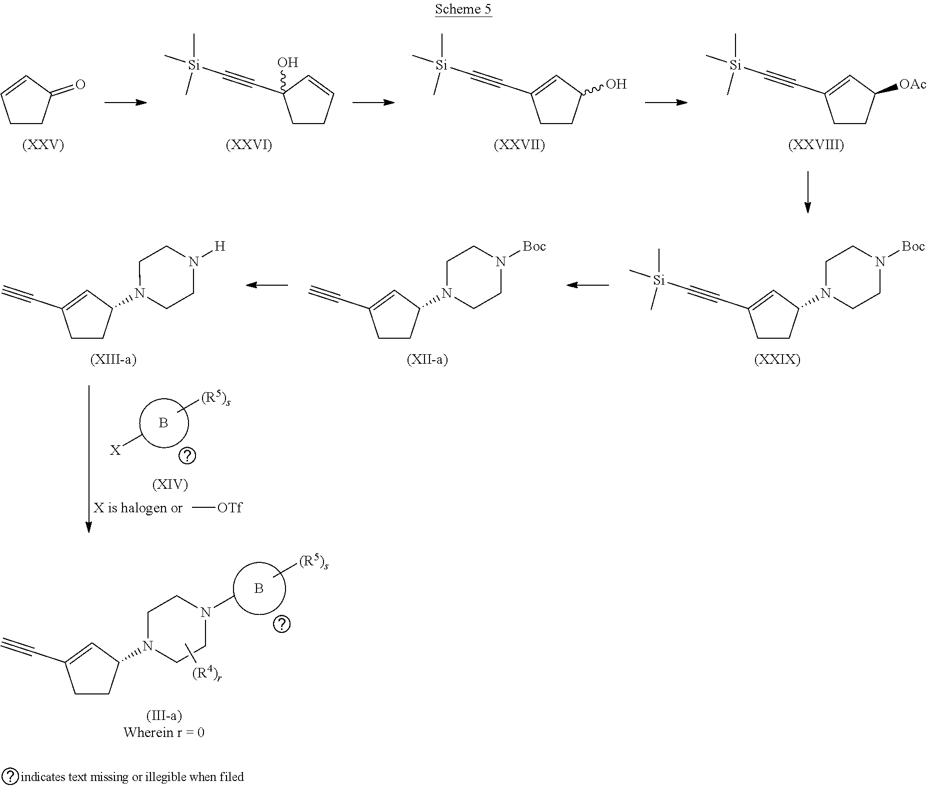

Scheme 5 shows a method of preparation of the compounds of formula (III-a); wherein r=0 and all other symbols are as defined under compounds of formula (I), from a compound represented by formula (XXV). The compound of formula (XXV) is commercially available.

The compound of formula (XXV) is reacted with Trimethylsilylacetylene in the presence of a base such as n-butyl lithium in tetrahydrofuran to obtain a compound of formula (XXVI). The compound of formula (XXVI) is treated with aqueous sulphuric acid to obtain a migrated product as (XXVII).

The compound of formula (XXVII) as obtained in the previous step is subjected to enantioselective acylation reaction with vinyl acetate in the presence of an enzyme such as Lipase PS “Amano” SD to obtain a compound of formula (XXVIII).

The compound of formula (XXVIII) is reacted with piperazine derivative in presence of a palladium catalyst such as Tetrakis(triphenyl phosphine) Pd(O) to obtain the coupled product as compound of formula (XXIX).

The compound of formula (XXIX) is subjected to deprotection reaction using tetrabutyl ammonium fluoride (TBAF) to obtain the compound of formula (XII-a). The compound of formula (XII-a) can be converted into the compound of formula (III-a) by following the procedure described in Scheme 3.

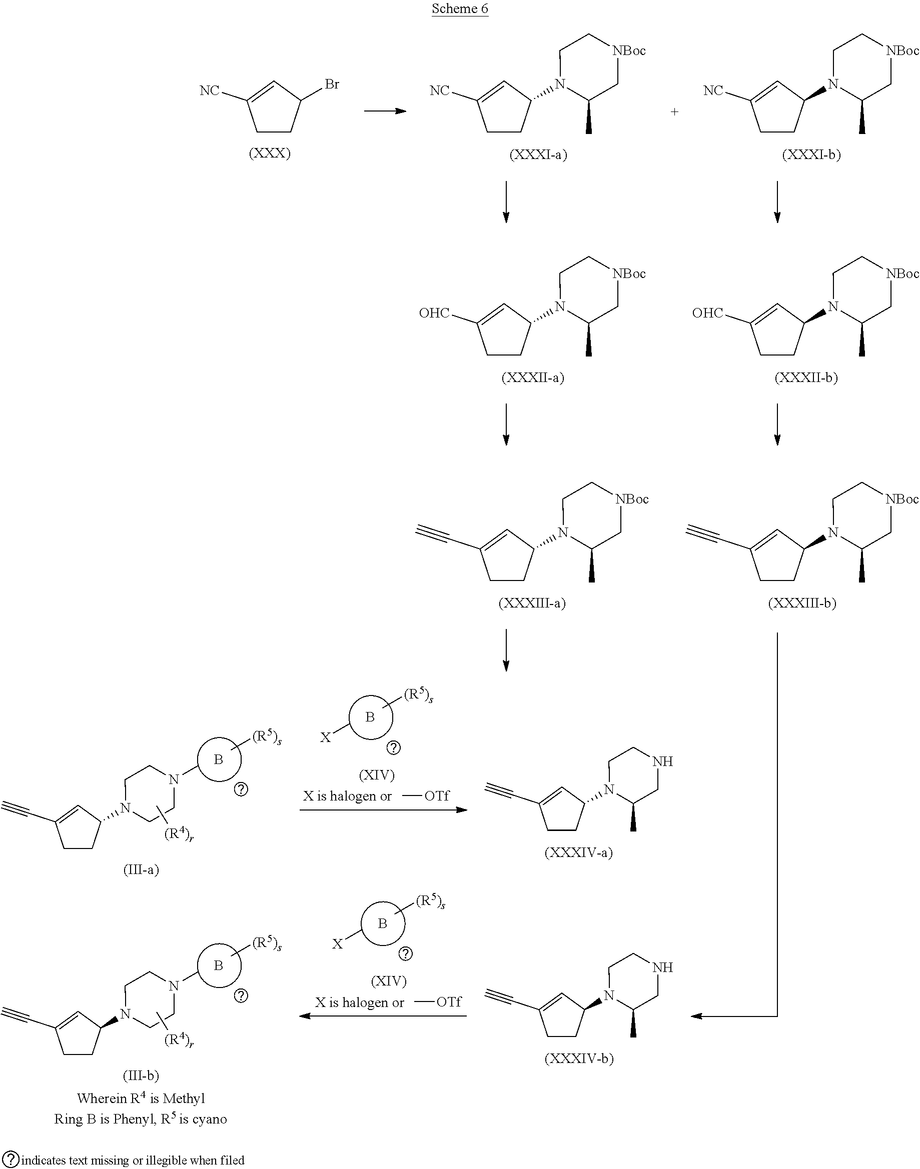

Scheme 6 shows a method of preparation of compounds of formula (III-a) and (III-b), wherein R⁴ is methyl, Ring B is phenyl and R⁵ is cyano and all other symbols are as defined under the compounds of formula (I) from a compound of formula (XXX).

The compound of formula (XXX) is reacted with tert-butyl (R)-3-methylpiperazine-1-carboxylate in the presence of a base such as potassium carbonate in acetonitrile as a solvent to obtain the compound as diastereomeric mixture. The mixture of compounds is separated by column chromatography to obtain two diastereomers of compound of formulas (XXXI-a) and (XXXI-b). The compounds of formula (III-a) and (III-b), where R⁴ is methyl, Ring B is phenyl and R⁵ is cyano and all other symbols are as defined under compound of formula (I), can be synthesized following the methods described in Scheme 3, starting from compounds of formula (XXXI-a) and compound of formula (XXXI-b) respectively.

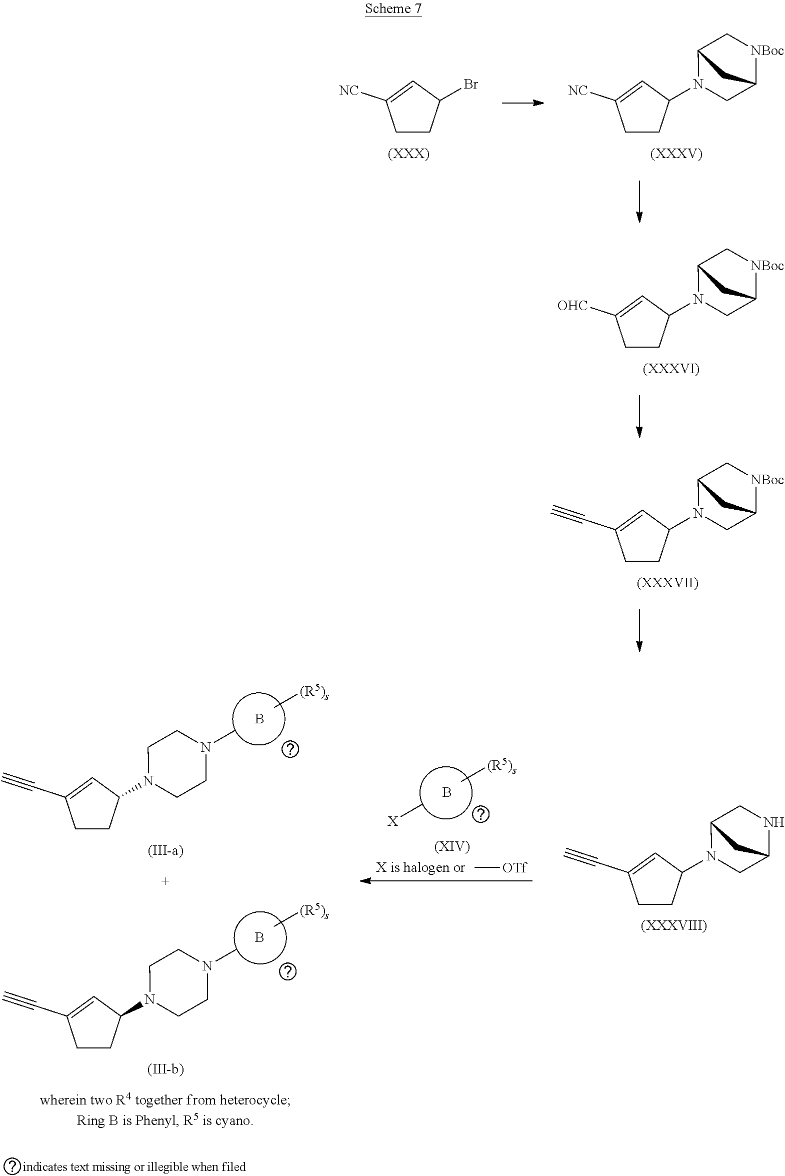

Scheme 7 shows a method of preparation of compounds of formula (IIIa) and (III-b), wherein two R⁴ together form bridged heterocycle ring, Ring B is phenyl and R⁵ is cyano and all other symbols are as defined under compound of formula (I), from the compound of formula (XXX).

The compound of formula (XXX) is reacted with tert-butyl (1S,4S)-2,5-diazabicyclo[2.2.1]heptane-2-carboxylate in the presence of a base such as potassium carbonate, in acetonitrile as a solvent to obtain the compound as diastereomeric mixture (XXXV). The compounds of formula (III-a) and (III-b); where two R⁴ together form (1S,4S)-2,5-diazabicyclo[2.2.1]heptane bridged heterocycle ring, Ring B is phenyl and R⁵ is cyano and all other symbols are as defined under compound of formula (I); can be synthesized starting from compounds of formula (XXXV); following the methods described in Scheme 3 and separation by chiral HPLC.

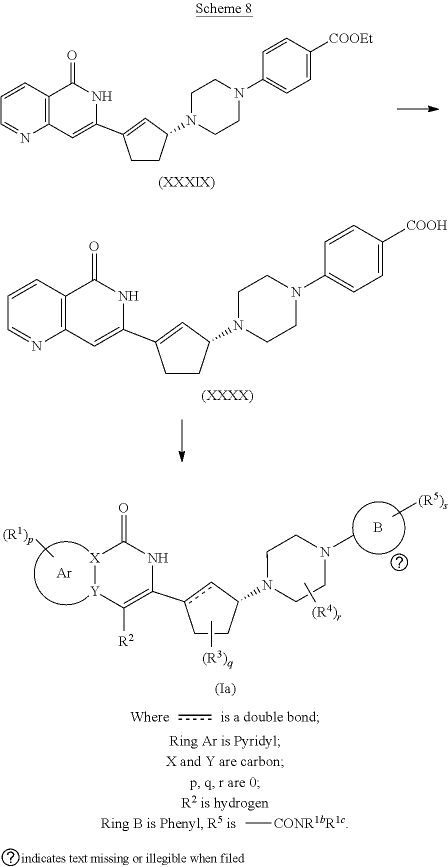

Scheme 8 shows a method of preparation of the compounds of formula (Ia) wherein R² is hydrogen, p, q, and r=0, [symbol] is double bond, Ring Ar is Pyridyl, X and Y are carbon, Ring B is Phenyl, R⁵ is —CONR^{1b}R^{1c}, from a compound of formula (XXXIX). The compounds of formula (XXXIX) can be prepared by the method described in Scheme 1.

The compound of formula (XXXIX) can be converted to a compound of formula (XXXX) according to reaction conditions known in the art for converting carboxylic esters to carboxylic acids. Preferably, the reaction is carried out using sodium hydroxide as a base and water-ethanol as a solvent.

The compound of formula (XXXX) is reacted with alkylamine hydrochloride. The reaction can be carried out using the conditions generally used for synthesis of amides from acids. The reaction may be carried out in suitable solvents such as dimethyl sulfoxide (DMSO), N,N-dimethylformamide, tetrahydrofuran, chloroform, dichloromethane, xylene, benzene or mixtures thereof or the like in the presence of a base such as methylamine, triethylamine, diisopropylethylamine, pyridine or the like at a temperature between 0-100° C. using reagent(s) such as thionyl chloride, phosphorous chloride, oxalyl chloride, alkyl chloroformate, 1-ethyl-3-(3-dimethylaminopropyl)carbodiimide (EDCI), N,N-dicyclohexylcarbodiimide (DCC), auxiliary reagents such as Hydroxybenzotriazole (HOBt), 1-hydroxy-7-azabenzotriazole (HOAt), N,N,N′,N′-Tetramethyl-O-(1H-benzotriazol-1-yl)uronium hexafluorophosphate (HBTU), (1-[Bis(dimethylamino)methylene]-1H-1,2,3-triazolo[4,5-b]pyridinium 3-oxid hexafluorophosphate) (HATU) or the like. Preferably, the coupling is carried out in DMSO using HATU and Diisopropyl ethyl amine (DIPEA) as base.

The compounds of formula (Ib), wherein R² is hydrogen, p, q, and r=0, [symbol] is double bond, Ring Ar is Pyridyl, X and Y are carbon, Ring B is Phenyl, R⁵ is —CONR^{1b}R^{1c} can be prepared from compounds of formula (III-b), wherein q=0, r=0, Ring B is Phenyl, R⁵ is ˜COOEt, using similar process as described in Scheme-8.

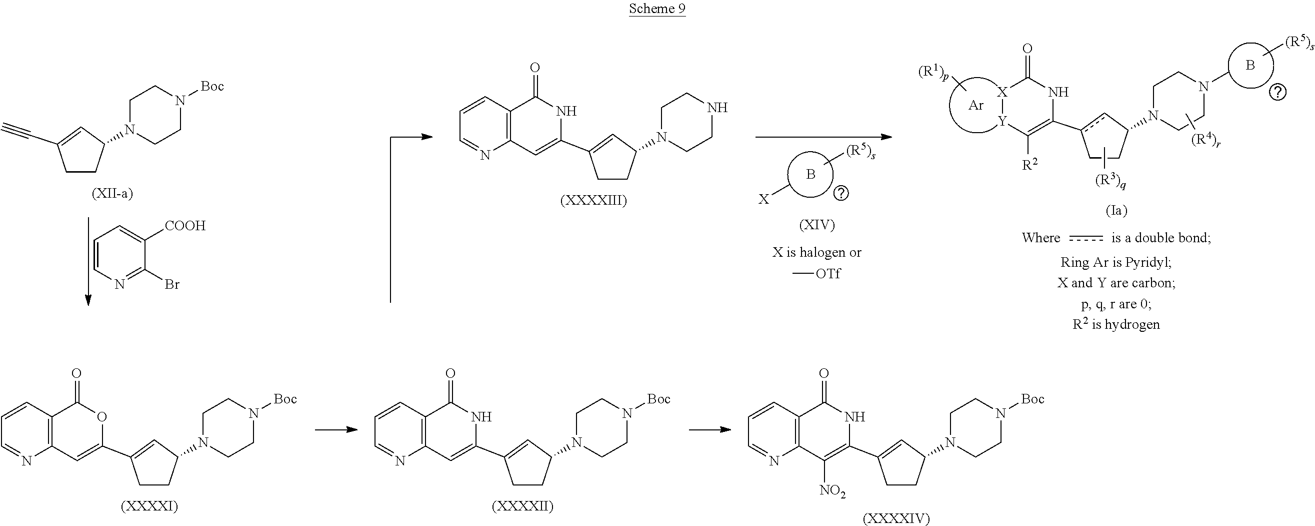

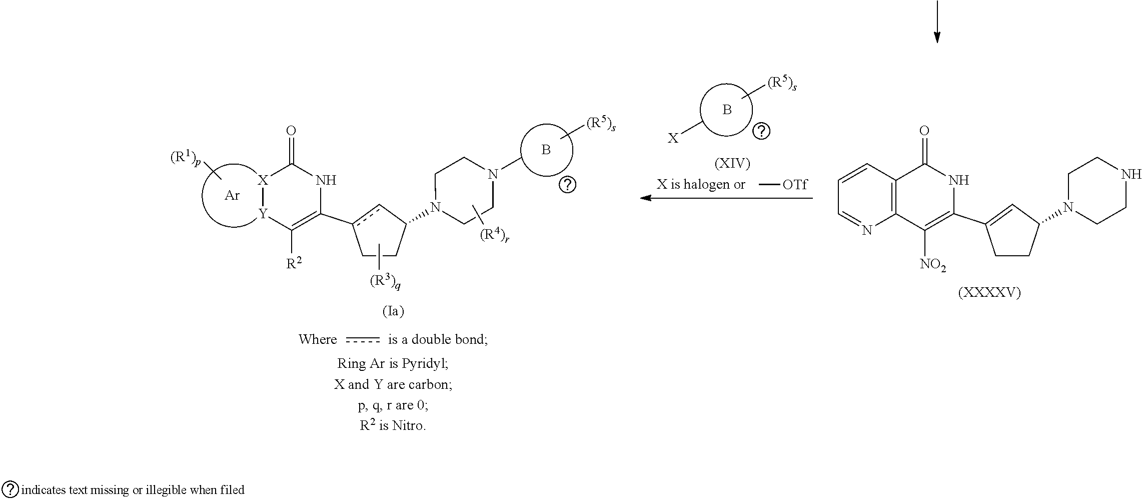

Scheme 9 shows a method of preparation of the compound of formula (Ia); wherein R² is hydrogen, p, q, and r are 0, [symbol] is double bond, Ring Ar is Pyridyl, X and Y are carbon, and all other symbols are as defined under compound of formula (I), from the compound of formula (XII-a). The compound of formula (XII-a) can be prepared by the method described in the Scheme 3 or Scheme 5.

The compound of formula (XII-a) is subjected to Sonogashira coupling with 2-bromonicotinic acid, followed by in situ cyclization to obtain a compound of formula (XXXXI). Preferably, the Sonogashira coupling reaction is carried out in anhydrous acetonitrile in the presence of bis(triphenylphosphine)palladium (II) chloride, using diisopropylethylamine or triethylamine as a base at 60-85° C. under nitrogen for 3-16 hr.

The compounds of formula (XXXXI) can be treated with ammonia to obtain a compound of formula (XXXXII). Preferably, the reaction is carried out in the presence of methanolic ammonia at 85° C. for 3-24 hrs.

The compound of formula (XXXXII) is subjected to deprotection of the N-protecting group to obtain a compound of formula (XXXXIII). The deprotection reaction of N-protecting groups can be carried out using standard procedures generally used in synthetic organic chemistry or well known in the literature e.g., Greene T. W. et al., 1999. Preferably, the reaction is carried out in dichloromethane using hydrochloric acid in 1,4-dioxane.

The compound of formula (XXXXIII) is reacted with the compounds of formula (XIV), wherein X═F, Cl, Br, I, or OTf, either in nucleophilic substitution reaction condition or Buchwald coupling method to obtain the compounds of formula (Ia), wherein R² is hydrogen, p, q, r=0, Ring Ar is Pyridyl, X and Y are carbon, and all other symbols are as defined under the compounds of formula (I). The reaction may be carried out in a suitable solvent such as dimethylsulfoxide, N,N-dimethylformamide, 1,4-dioxane, acetonitrile, dichloromethane, methanol, or ethanol in the presence of a base such as potassium carbonate, sodium bicarbonate, triethylamine or the like, at a temperature of 25° C.-150° C. over a period of 30 min to 20 hr to obtain compound of formula (I). Preferably, the nucleophilic substitution reaction is carried out in N,N-dimethylformamide using potassium carbonate as base. On the other hand, Buchwald coupling can be carried out in a solvent such as toluene, tert-butanol, dimethylformamide, iso-propyl alcohol, 1,4-dioxane, 1,2-dimethoxyethane, tetrahydrofuran, and/or acetonitrile, in the presence of a base such as potassium phosphate, potassium carbonate, sodium tert-butoxide, cesium carbonate, lithium hexamethyl disilazane or the like, palladium catalysts such as palladium (II) acetate (Pd(OAc)₂), tris(dibenzylideneacetone)dipalladium (0), [Pd₂(dba)₃], at a temperature of 50-160° C. and ligand such as 2,2′-Bis(diphenylphosphino)-1,1′-binaphthyl (BINAP), 2-Dicyclohexylphosphino-2′,4′,6′-triisopropylbiphenyl (XPhos), 2-Dicyclohexylphosphino-2′-(N,N-dimethylamino)biphenyl (DavePhos), (2-Biphenyl)di-tert-butylphosphine (JohnPhos), 2-Dicyclohexylphosphino-2′,6′-dimethoxybiphenyl (SPhos), 2-Dicyclohexylphosphino-2′-methylbiphenyl (MePhos) or the like.

The compound of formula (XXXXII) is reacted with sulphuric acid and nitric acid to obtain the compound of formula (XXXXIV); which on deprotection and 5 coupling with the compounds of formula (XIV), where X═F, Cl, Br, I, or OTf, either in a nucleophilic substitution reaction condition or Buchwald coupling method, to give the compounds of formula (Ia), wherein p, q, and r are 0, R² is nitro, [symbol] is double bond, Ring Ar is Pyridyl, X and Y are carbon, and all other symbols are as defined under compound of formula (I).

The compounds of formula (Ib), wherein R² is hydrogen or nitro, p, q, and r are 0, [symbol] is double bond, Ring Ar is Pyridyl, X and Y are carbon, and all other symbols are as defined under compound of formula (I), from the compound of formula (XII-b). The compound of formula (XII-b) can be prepared by the method described in the Scheme 3.

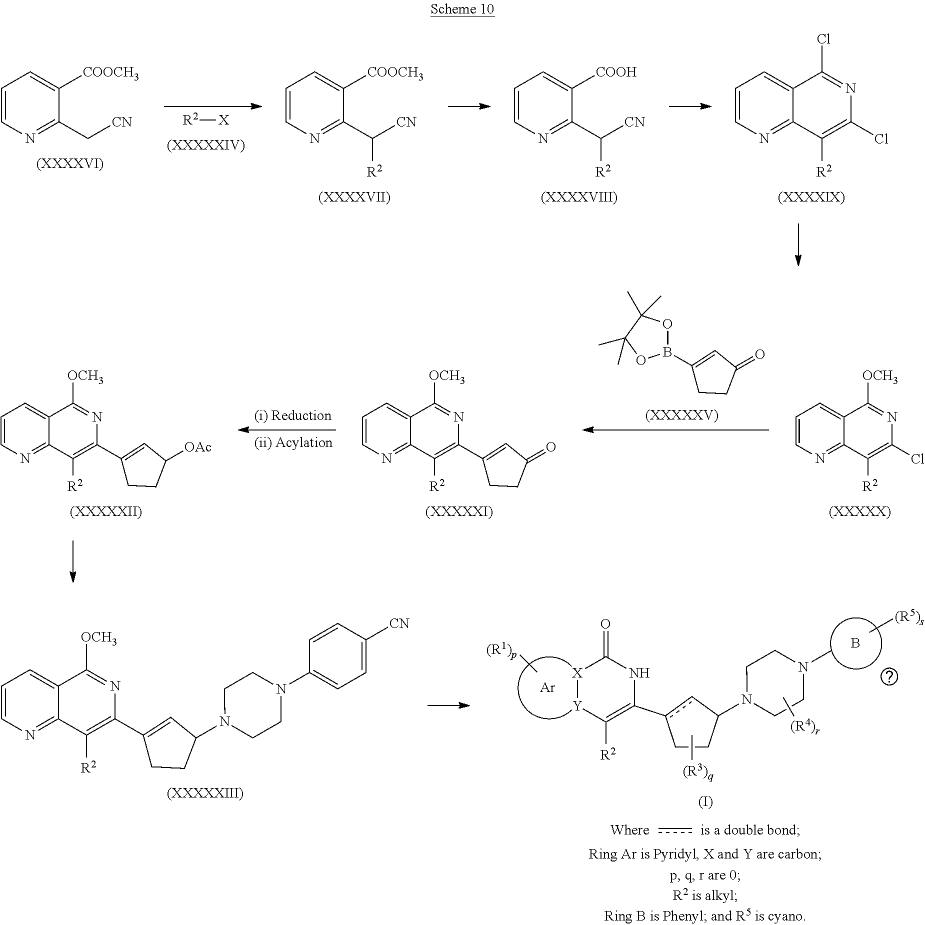

Scheme 10 shows a method of preparation of the compounds of formula (I), wherein p, q, r are 0, R² is alkyl, [symbol] is double bond, Ring Ar is Pyridyl, X and Y are carbon, Ring B is Phenyl and R⁵ is cyano, from a compound of formula (XXXXVI). The compound of formula (XXXXVI) can be prepared according to the procedure described in WO2015/200677.

The compound of formula (XXXXVI) is reacted with a halide of formula (XXXXXIV); where R² is alkyl, and X is halogen; in the presence of a base like sodium ethoxide, sodium hydride, potassium t-butoxide, potassium carbonate, or cesium carbonate in solvents such as acetonitrile, DMF, THF, or acetone to obtain the compounds of formula (XXXXVII), where R² is alkyl. Preferably, the alkylation reaction is carried out in DMF in the presence of sodium hydride as base.

Ester hydrolysis of the compounds of formula (XXXXVII) gives the compounds of formula (XXXXVIII). Ester hydrolysis may be carried out using standard procedure generally used in synthetic organic chemistry or well known in the art with reagents such as sodium hydroxide, potassium hydroxide, lithium hydroxide or the like in solvents such as alcohol, THF, water or the like or a mixture thereof. Preferably, an aqueous solution of sodium hydroxide and methanol is used for the reaction.

The compounds of formula (XXXXVIII) so obtained are reacted with phosphoryl chloride or phosphorus pentachloride to obtain the dichlorinated compounds of formula (XXXXIX) under heating conditions; the resulting products treated with sodium methoxide in methanol to obtain the compounds of formula (XXXXX). Reactions can be carried out using procedures reported in the literature (e.g., US2004199024 and WO201387805).

The compounds of formula (XXXXX) obtained in the previous step are subjected to Suzuki coupling with boronic ester (prepared according to the procedure reported in the literature, US2012/77814) represented by formula (XXXXXV), to obtain compounds of formula (XXXXXI). Suzuki coupling with boronic ester can be carried out by following procedures well known in the art.

The compounds of formula (XXXXXI) as obtained in the previous step are treated with reducing agents such as sodium borohydride in the presence of Cerium(III) chloride, followed by acylation using acetic anhydride in the presence of base such as triethyl amine and DMAP (4-Dimethylaminopyridine) to obtain the compounds of formula (XXXXXII).

The compounds of formula (XXXXXII) as obtained are reacted with 4-(piperazin-1-yl)benzonitrile in the presence of Palladium catalyst such as Tetrakis(triphenylphosphine)Pd(0) (Pd(PPh₃)₄) to obtain the coupled product as compounds of formula (XXXXXIII).

The compounds of formula (XXXXXIII) so obtained in the previous step are reacted with trimethylsilyl chloride (TMS-Cl) and sodium iodide to obtain the compounds of formula (I); wherein p, q, and r are 0, R² is alkyl, Ring Ar is Pyridyl, X and Y are carbon, [symbol] is double bond, Ring B is Phenyl, and R⁵ is cyano.

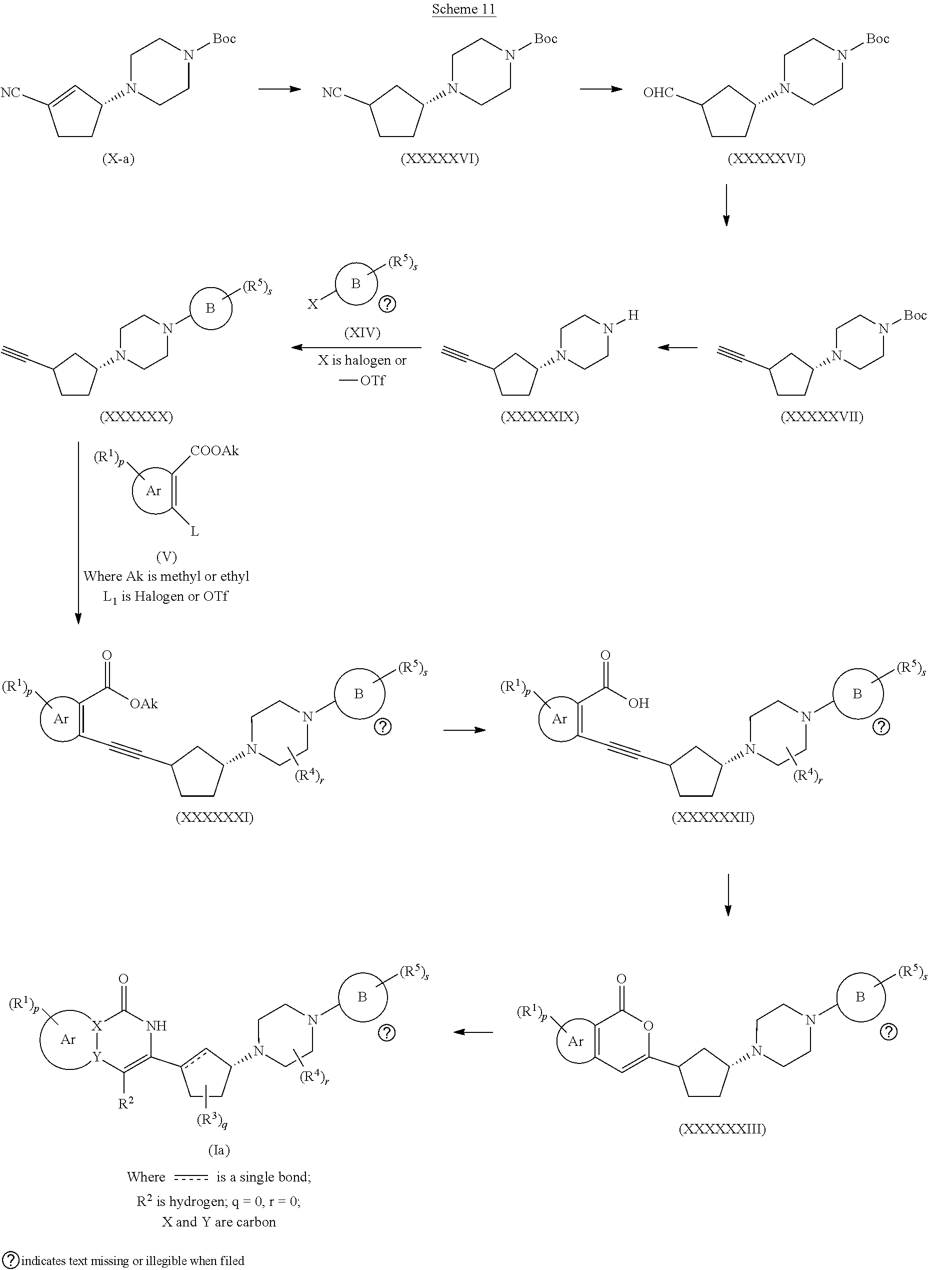

Scheme 11 shows a method of preparation of the compounds of formula (Ia), where R² is hydrogen, q=0, r=0, X and Y are carbon, [symbol] is single bond, and all other symbols are as defined in general formula (I), from the compound of formula (X-a). The compound of formula (X-a) can be prepared according to the procedure described in Scheme 3.

The compound of formula (X-a) is reduced to a compound of formula (XXXXXVI). The compound of formula (XXXXXVI) is further converted to the compounds of formula (Ia), wherein R² is hydrogen, q=0 r=0, X and Y are carbon, [symbol] is single bond, and all other symbols are as defined in general formula (I), by following the procedures described in Schemes 2 and 3.

The compounds of formula (Ib), where R² is hydrogen, q=0, r=0, X and Y are carbon, [symbol] is single bond, and all other symbols are as defined in general formula (I), from the compound of formula (X-b). The compound of formula (X-b) can be prepared according to the procedure described in Scheme 3.

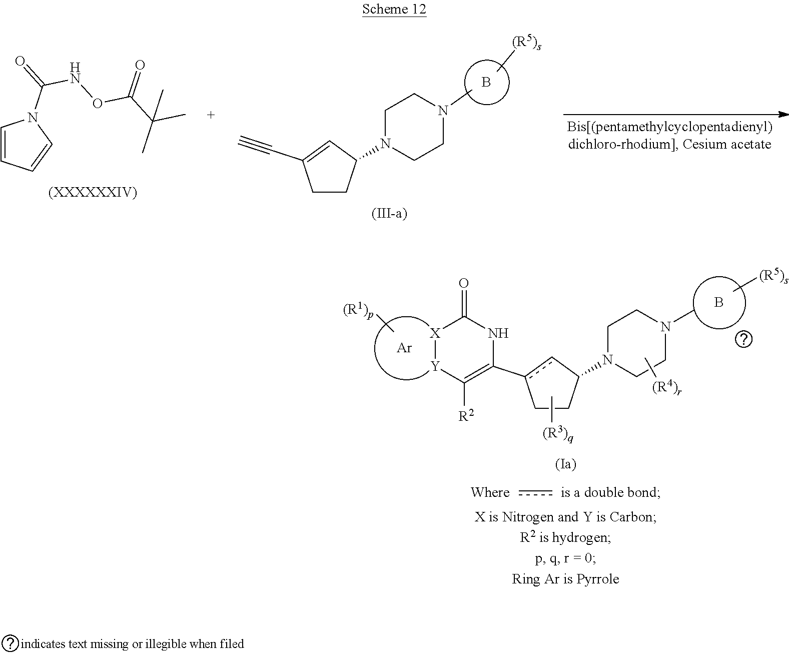

Scheme 12 shows a method of preparation of compounds of formula (Ia) in accordance with an embodiment. The compounds of formula (Ia), wherein X is nitrogen, Y is carbon, R² is hydrogen, p, q, and r are 0, [symbol] is double bond, Ring Ar is Pyrrole and all symbols are as defined under formula (I), can be prepared from compounds of formula (III-a), wherein ring B, R⁵ and s are as defined under formula (I). The compounds of formula (III-a) can be prepared by the procedures described in Scheme 3.

The compounds of formula (XXXXXXIV) are subjected to Rh(III) catalyzed coupling with compound of formula (III-a), where all symbols are as defined under formula (I), to obtain the compounds of formula (Ia). The reaction may be carried out in the presence of an organic solvent, for example, methanol, acetonitrile, N,N-dimethyl formamide, tetrahydrofuran, a halogenated hydrocarbon such as chloroform or dichloromethane, an aromatic hydrocarbon such as xylene, benzene, toluene, or the like or mixtures thereof. Preferably, the coupling reaction is carried out in methanol in the presence of bis[(pentamethylcyclopentadienyl)dichloro-rhodium], using cesium acetate at 30° C. under nitrogen.

The compounds of formula (Ib), wherein X is nitrogen, Y is carbon, R² is hydrogen, p, q, and r are 0, [symbol] is double bond, Ring Ar is Pyrrole and all symbols are as defined under formula (I), can be prepared from compound of formula (III-b), wherein ring B, R⁵ and s are as defined under formula (I). The compounds of formula (III-b) can be prepared by the procedures described in Scheme 3.

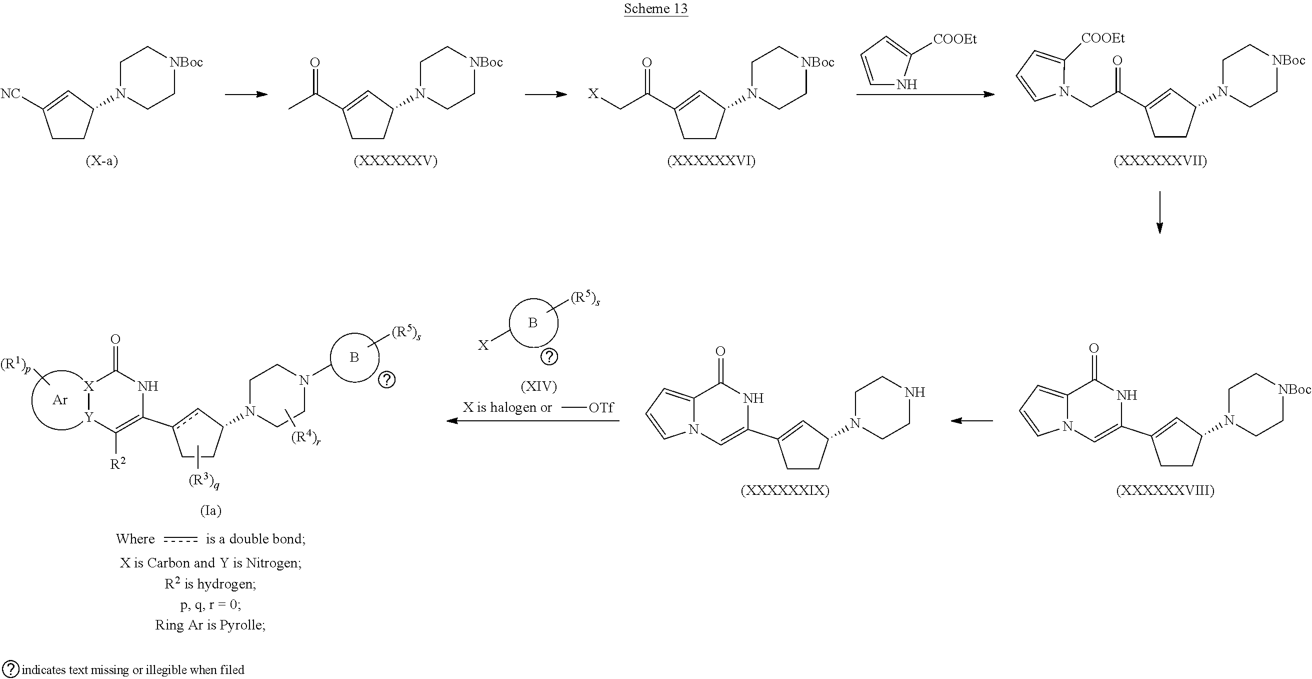

Scheme 13 shows a method of preparation of compounds of formula (Ia) in accordance with an embodiment. The compounds of formula (I), wherein X is carbon, Y is nitrogen, R² is hydrogen, p, q, and r are 0, [symbol] is double bond, Ring Ar is Pyrrole and all symbols are as defined under formula (I), can be prepared from the compound of formula (X-a), The compound of formula (X-a) can be prepared by the procedures described in Scheme 3.

The compound of formula (X-a) is reacted with methyl lithium in THF to obtain a compound of formula (XXXXXXV). The compound of formula (XXXXXXV) so obtained is reacted under halogenation condition generally used in the synthetic organic chemistry using halogenating agents such as N-bromosuccinimide, N-chlorosuccinimide, bromine, phosphorous tribromide and aluminium tribromide. In an embodiment, chlorination is carried out using N-chlorosuccinimide, in tetrahydrofuran to obtain compounds of formula (XXXXXXVI) wherein X is halogen. The compounds of formula (XXXXXXVI) as obtained in the previous step are reacted with ethyl 1H-pyrrole-2-carboxylate in the presence of base such as cesium carbonate in DMF as solvent to obtain a compound of formula (XXXXXXVII).

The compound of formula (XXXXXXVII) so obtained is reacted with ammonia in methanol to obtain the cyclized product as compound of formula (XXXXXXVIII). The compound of formula (XXXXXXVIII) as obtained in the previous step is subjected to deprotection in HCl in dioxane and dichloromethane to obtain a compound of formula (XXXXXXIX). The compound of formula (XXXXXXIX) is reacted with the compounds of formula (XIV), wherein X═F, Cl, Br, I, or OTf, and all other symbols are as defined under formula (I), either in nucleophilic substitution reaction condition or Buchwald coupling method to obtain the compounds of formula (Ia) wherein X is carbon, Y is nitrogen, R² is hydrogen, p, q and r=0, [symbol] is double bond and all symbols are as defined under formula (I). The reaction is carried out depending on nature of X and R⁵ in compound of formula (XIV) in a suitable solvent such as dimethylsulfoxide, N,N-dimethylformamide, 1,4-dioxane, acetonitrile, dichloromethane, methanol, or ethanol in the presence of a suitable base such as potassium carbonate, sodium bicarbonate, triethylamine or the like at temperature between 25° C.-150° C. over a period of 30 min to 20 hr to obtain the compounds of formula (I). On the other hand, Buchwald coupling can be carried out under reaction conditions known in the art. Preferably, the Buchwald coupling is carried out in a solvent such as toluene, tert-butanol, dimethylformamide, iso-propyl alcohol, 1,4-dioxane, 1,2-dimethoxyethane, tetrahydrofuran, and/or acetonitrile, in the presence of a base such as potassium phosphate, potassium carbonate, sodium tert-butoxide, cesium carbonate, lithium hexamethyl disilazane or the like, and a palladium catalyst such as palladium (II) acetate (Pd(OAc)₂), tris(dibenzyllideneacetone)dipalladium (0), [Pd₂(dba)₃], at a temperature between 50-160° C. and a ligand such as 2,2′-Bis(diphenylphosphino)-1,1′-binaphthyl (BINAP), 2-Dicyclohexylphosphino-2′,4′,6′-triisopropylbiphenyl (XPhos), 2-Dicyclohexylphosphino-2′-(N,N-dimethylamino) biphenyl (DavePhos), (2-Biphenyl)di-tert-butylphosphine (JohnPhos), 2-Dicyclohexylphosphino-2′,6′-dimethoxybiphenyl (SPhos), 2-Dicyclohexylphosphino-2′-methylbiphenyl (MePhos) or the like.

The compounds of formula (Ib), wherein X is carbon, Y is nitrogen, R² is hydrogen, p, q, and r are 0, [symbol] is double bond, Ring Ar is Pyrrole and all symbols are as defined under formula (I), can be prepared from the compound of formula (X-b). The compound of formula (X-b) can be prepared by the procedures described in Scheme 3.

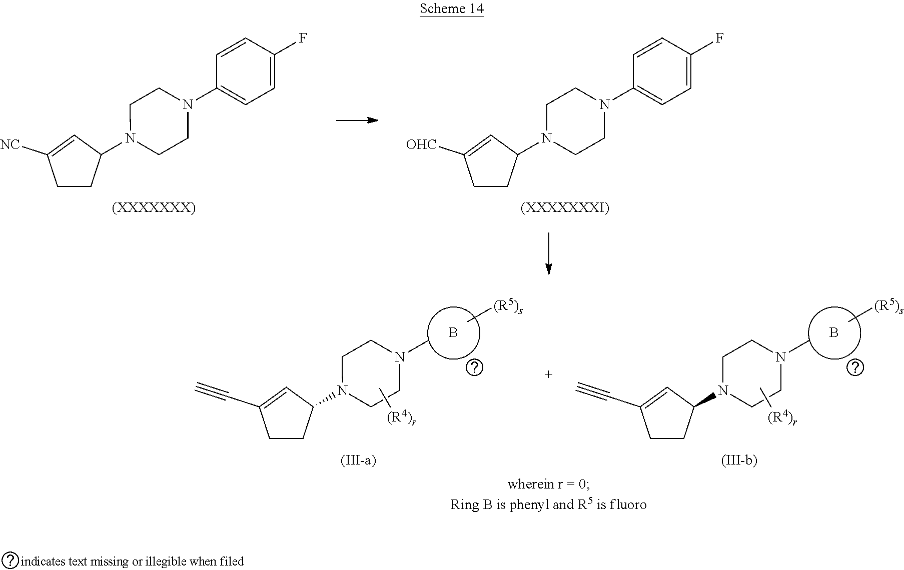

The enantiopure compound of formula (IIIa and III-b), wherein r=0, Ring B is phenyl and R⁵ is fluoro, and all other symbols are as defined under compounds of formula (I), can be synthesized from a compound of formula (XXXXXXX). Compounds of formula (XXXXXXX) can be synthesized by following methods described in literature (WO20149872); which can be further converted to a compound of formula (III-a and III-b), wherein r=0, Ring B is phenyl and R⁵ is fluoro, and all other symbols are as defined under compounds of formula (I), by following methods described in Scheme 3 for the synthesis of enantiopure compound of formula (III-a) followed by chiral separation using chiral preparative HPLC.

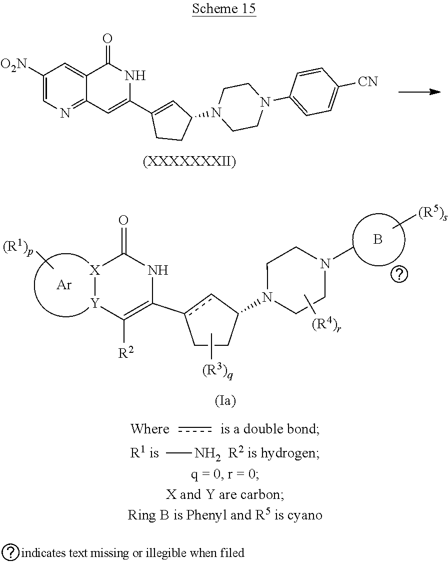

Scheme 15 shows a method of preparation of the compounds of formula represented as (Ia) in accordance with an embodiment. The compounds of formula (Ia), wherein R¹ is —NH₂, R² is hydrogen, q=0, r=0, [symbol] is double bond, X and Y are carbon, Ring B is phenyl and R⁵ is cyano, and all other symbols are as defined under formula (I), can be prepared from compound of formula (XXXXXXXII), which can be prepared according to the method described in Scheme 1.

Compound of formula (XXXXXXXII) is converted to compounds of formula (Ia), wherein R¹ is —NH₂, R² is hydrogen, q=0, r=0, [symbol] is double bond, X and Y are carbon, Ring B is phenyl and R⁵ is cyano, and all other symbols are as defined under formula (I), by reduction of the nitro group to amino group using iron and acetic acid in ethanol.

The intermediates and the compounds of the present invention can be obtained in a pure form in a manner known per se, for example, by distilling off the solvent in vacuum and/or re-crystallizing the residue obtained from a suitable solvent, such as pentane, diethyl ether, isopropyl ether, chloroform, dichloromethane, ethyl acetate, acetone or their combinations or subjecting them to one of the purification methods, such as column chromatography (e.g. flash chromatography) on a suitable support material such as alumina or silica gel using an eluent such as dichloromethane, ethyl acetate, hexane, methanol, acetone and their combinations. Preparative LC-MS method can also be used for the purification of molecules described herein.

Unless otherwise stated, work-up includes distribution of the reaction mixture between the organic and aqueous phases indicated within parentheses, separation of layers and drying the organic layer over sodium sulphate, filtration and evaporation of the solvent. Purification, unless otherwise mentioned, includes purification by silica gel chromatographic techniques, generally using a mobile phase with suitable polarity.

Salts of compound of formula (I) can be obtained by dissolving the compound in a suitable solvent, for example in a chlorinated hydrocarbon, such as methyl chloride or chloroform or a low molecular weight aliphatic alcohol, for example, ethanol or isopropanol, which was then treated with the desired acid or base as described in Berge S. M. et al., “Pharmaceutical Salts, a review article in Journal of Pharmaceutical sciences volume 66, page 1-19 (1977)” and in “Handbook of Pharmaceutical Salts—Properties, Selection, and Use,” by P. H. Einrich Stahland Camille G. wermuth, Wiley-VCH (2002). Lists of suitable salts can also be found in Remington's Pharmaceutical Sciences, 18th ed., Mack Publishing Company, Easton, Pa., 1990, p. 1445, and Journal of Pharmaceutical Science, 66, 2-19 (1977). For example, the salt can be of an alkali metal (e.g., sodium or potassium), alkaline earth metal (e.g., calcium), or ammonium.

The compound of the invention or a composition thereof can potentially be administered as a pharmaceutically acceptable acid-addition, base neutralized or addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, potassium hydroxide. The conversion to a salt is accomplished by treatment of the base compound with at least a stoichiometric amount of an appropriate acid. Typically, the free base is dissolved in an inert organic solvent such as diethyl ether, ethyl acetate, chloroform, ethanol, methanol, and the like, and the acid is added in a similar solvent. The mixture is maintained at a suitable temperature (e.g., between 0° C. and 50° C.). The resulting salt precipitates spontaneously or can be brought out of solution with a less polar solvent.

The stereoisomers of the compounds of formula (I) of the present invention may be prepared by stereospecific syntheses or resolution of racemic compound using an optically active amine, acid or complex forming agent, and separating the diastereomeric salt/complex by fractional crystallization or by column chromatography.

The compounds of formula (I) of the present invention can exist in tautomeric forms, such as keto-enol tautomers. Such tautomeric forms are contemplated as an aspect of the present invention and such tautomers may be in equilibrium or predominant in one of the forms.

The present invention further provides a pharmaceutical composition, containing the compounds of the general formula (I) as defined above, its tautomeric forms, its stereoisomers, its pharmaceutically acceptable salts in combination with one or more of pharmaceutically acceptable carriers, diluents, excipients, and the like.

The pharmaceutically acceptable carrier or excipient is preferably one that is chemically inert to the compound of the invention and one that has no detrimental side effects or toxicity under the conditions of use. Such pharmaceutically acceptable carriers or excipients include saline (e.g., 0.9% saline), Cremophor EL® (which is a derivative of castor oil and ethylene oxide available from Sigma Chemical Co., St. Louis, Mo.) (e.g., 5% Cremophor EL/5% ethanol/90% saline, 10% Cremophor EL/90% saline, or 50% Cremophor EL/50% ethanol), propylene glycol (e.g., 40% propylene glycol/10% ethanol/50% water), polyethylene glycol (e.g., 40% PEG 400/60% saline), and alcohol (e.g., 40% ethanol/60% water). A preferred pharmaceutical carrier is polyethylene glycol, such as PEG 400, and particularly a composition comprising 40% PEG 400 and 60% water or saline. The choice of carrier will be determined in part by the particular compound chosen, as well as by the particular method used to administer the composition. Accordingly, there is a wide variety of suitable formulations of the pharmaceutical composition of the present invention.

The following formulations for oral, aerosol, parenteral, subcutaneous, intravenous, intraarterial, intramuscular, intrathecal, intraperitoneal, rectal, and vaginal administration are merely exemplary and are in no way limiting.

The pharmaceutical compositions can be administered parenterally, e.g., intravenously, intraarterially, subcutaneously, intradermally, intrathecally, or intramuscularly. Thus, the invention provides compositions for parenteral administration that comprise a solution of the compound of the invention dissolved or suspended in an acceptable carrier suitable for parenteral administration, including aqueous and non-aqueous, isotonic sterile injection solutions.

Overall, the requirements for effective pharmaceutical carriers for parenteral compositions are well known to those of ordinary skill in the art. See Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986). Such compositions include solutions containing anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol (for example in topical applications), or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, and synthetic oils. Specific examples of oils useful in such formulations include peanut, soybean, sesame, cottonseed, corn, olive, petrolatum, and mineral oil. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl-(3-aminopropionates, and 2-alkyl-imidazoline quaternary ammonium salts, and (e) mixtures thereof.

The parenteral formulations typically will contain from about 0.5% or less to about 25% or more by weight of a compound of the invention in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Topical formulations, including those that are useful for transdermal drug release, are well known to those of skill in the art and are suitable in the context of the present invention for application to skin.

Formulations suitable for oral administration can consist of (a) liquid solutions, such as an effective amount of a compound of the invention dissolved in diluents, such as water, saline, or orange juice; (b) capsules, sachets, tablets, lozenges, and troches, each containing a pre-determined amount of the compound of the invention, as solids or granules; (c) powders; (d) suspensions in an appropriate liquid; and (e) suitable emulsions. Liquid formulations can include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the compound ingredient in a flavor, usually sucrose and acacia or tragacanth, as well as pastilles comprising a compound of the invention in an inert base, such as gelatin and glycerin, or sucrose and acacia, emulsions, gels, and the like containing, in addition to the compound of the invention, such excipients as are known in the art.

A compound of the present invention, alone or in combination with other suitable components, can be made into aerosol formulations to be administered via inhalation. A compound or epimer of the invention is preferably supplied in finely divided form along with a surfactant and propellant. Typical percentages of the compounds of the invention can be about 0.01% to about 20% by weight, preferably about 1% to about 10% by weight. The surfactant must, of course, be nontoxic, and preferably soluble in the propellant. Representative of such surfactants are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides can be employed. The surfactant can constitute from about 0.1% to about 20% by weight of the composition, preferably from about 0.25% to about 5%. The balance of the composition is ordinarily propellant. A carrier can also be included as desired, e.g., lecithin, for intranasal delivery. These aerosol formulations can be placed into acceptable pressurized propellants, such as dichlorodifluoromethane, propane, nitrogen, and the like. They also can be formulated as pharmaceuticals for non-pressured preparations, such as in a nebulizer or an atomizer. Such spray formulations can be used to spray mucosa.

Additionally, the compound of the invention can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the compound ingredient, such carriers as are known in the art to be appropriate.

The concentration of the compound in the pharmaceutical formulations can vary, e.g., from less than about 1% to about 10%, to as much as about 20% to about 50% or more by weight, and can be selected primarily by fluid volumes, and viscosities, in accordance with the particular mode of administration selected.

For example, a typical pharmaceutical composition for intravenous infusion could be made up to contain 250 ml of sterile Ringer's solution, and 100 mg of at least one compound of the invention. Actual methods for preparing parenterally administrable compounds of the invention will be known or apparent to those skilled in the art and are described in more detail in, for example, Remington's Pharmaceutical Science (17^{th} ed., Mack Publishing Company, Easton, Pa., 1985).

It will be appreciated by one of ordinary skill in the art that, in addition to the afore-described pharmaceutical compositions, the compound of the invention can be formulated as inclusion complexes, such as cyclodextrin inclusion complexes, or liposomes. Liposomes can serve to target a compound of the invention to a particular tissue, such as lymphoid tissue or cancerous hepatic cells. Liposomes can also be used to increase the half-life of a compound of the invention. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

The compounds of the invention can be administered in a dose sufficient to treat the disease, condition or disorder. Such doses are known in the art (see, for example, the Physicians' Desk Reference (2004)). The compounds can be administered using techniques such as those described in, for example, Wasserman et al., Cancer, 36, pp. 1258-1268 (1975) and Physicians' Desk Reference, 58th ed., Thomson PDR (2004).

Suitable doses and dosage regimens can be determined by conventional range-finding techniques known to those of ordinary skill in the art. Generally, treatment is initiated with smaller dosages that are less than the optimum dose of the compound of the present invention. Thereafter, the dosage is increased by small increments until the optimum effect under the circumstances is reached. The present method can involve the administration of about 0.1 μg to about 50 mg of at least one compound of the invention per kg body weight of the individual. For a 70 kg patient, dosages of from about 10 μg to about 200 mg of the compound of the invention would be more commonly used, depending on a patient's physiological response.

By way of example and not intending to limit the invention, the dose of the pharmaceutically active agent(s) described herein for methods of treating or preventing a disease or condition as described above can be about 0.001 to about 1 mg/kg body weight of the subject per day, for example, about 0.001 mg, 0.002 mg, 0.005 mg, 0.010 mg, 0.015 mg, 0.020 mg, 0.025 mg, 0.050 mg, 0.075 mg, 0.1 mg, 0.15 mg, 0.2 mg, 0.25 mg, 0.5 mg, 0.75 mg, or 1 mg/kg body weight per day. The dose of the pharmaceutically active agent(s) described herein for the described methods can be about 1 to about 1000 mg/kg body weight of the subject being treated per day, for example, about 1 mg, 2 mg, 5 mg, 10 mg, 15 mg, 0.020 mg, 25 mg, 50 mg, 75 mg, 100 mg, 150 mg, 200 mg, 250 mg, 500 mg, 750 mg, or 1000 mg/kg body weight per day.

PARP inhibitors of the present invention can be used for the treatment of diseases and/or disorders that include but are not limited to cancer, stroke, traumatic brain injury, Parkinson's disease, meningitis, myocardial infarction, ischaemic cardiomyopathy, vascular disease, septic shock, ischemic injury, reperfusion injury, neurotoxicity, inflammatory disease, and haemorrhagic shock. PARP inhibitors mentioned herein can be used as single agents and/or in combination with other chemotherapeutic agents so that they can potentiate the effects of the standard chemotherapeutic agents.

Cancers that can be treated with PARP inhibitors include but are not, limited to breast cancer, glioblastoma, pancreatic cancer, ovarian cancer, prostate cancer, melanoma, colon cancer, leukaemia and lymphoma.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, 100%, 90%, 80%, 70%, 60%, 50%, 40%, 30%, 20%, or 10%. Furthermore, the treatment, prevention, amelioration, or inhibition provided by the inventive method can include treatment, prevention, amelioration, or inhibition of one or more conditions or symptoms of the disorder, e.g., cancer. Also, for purposes herein, “treatment,” “prevention,” “amelioration,” or “inhibition” can encompass delaying the onset of the disorder, or a symptom or condition thereof.

The terms “effective amount” or “therapeutically effective amount,” as used herein, refer to a sufficient amount of an agent or a compound being administered which will relieve to some extent one or more of the symptoms of the disease or condition being treated. In some embodiments, the result is a reduction and! or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. In some embodiments, an appropriate “effective” amount in any individual case is determined using techniques, such as a dose escalation study.

The terms “potentiation” or “potentiating,” as used herein, means to increase or prolong either in potency or duration a desired effect. Thus, in regard to potentiating the effect of therapeutic agents/regimen, the term “potentiating” refers to the ability to increase or prolong, either in potency or duration, the effect of other therapeutic agents on a system.

In accordance with the invention, the term subject includes an “animal” which in turn includes a mammal such as, without limitation, the order Rodentia, such as mice, and the order Lagomorpha, such as rabbits. In one aspect, the mammals are from the order Carnivora, including Felines (cats) and Canines (dogs). In another aspect, the mammals are from the order Artiodactyla, including Bovines (cows) and Swine (pigs) or of the order Perssodactyla, including Equines (horses). In a further aspect, the mammals are of the order Primates, Ceboids, or Simoids (monkeys) or of the order Anthropoids (humans and apes). In yet another aspect, the mammal is human.

The term “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

Another aspect of the present invention is a pharmaceutical composition of compound of formula (I) in combination with at least one other known anticancer agent, or a pharmaceutically acceptable salt of said agent.

Any suitable anticancer agent can be used. In an embodiment, the anticancer agent used in combination is selected from the group consisting of busulfan, melphalan, chlorambucil, cyclophosphamide, ifosfamide, temozolomide, bendamustine, cisplatin, mitomycin C, bleomycin, carboplatin, camptothecin, irinotecan, topotecan, doxorubicin, epirubicin, aclarubicin, mitoxantrone, elliptinium, etoposide, 5-azacytidine, gemcitabine, 5-fluorouracil, methotrexate, 5-fluoro-2′-deoxy-uridine, fludarabine, nelarabine, ara-C, alanosine, pralatrexate, pemetrexed, hydroxyurea, thioguanine, colchicine, vinblastine, vincristine, vinorelbine, paclitaxel, ixabepilone, cabazitaxel, docetaxel, campath, imatinib, gefitinib, erlotinib, lapatinib, sorafenib, sunitinib, nilotinib, dasatinib, pazopanib, temsirolimus, everolimus, vorinostat, romidepsin, tamoxifen, letrozole, fulvestrant, mitoguazone, octreotide, retinoic acid, arsenic trioxide, zoledronic acid, bortezomib, thalidomide and lenalidomide.

In another embodiment, the invention provides a method of treatment or prevention of a disorder responsive to the inhibition of PARP activity in a mammal suffering therefrom, comprising administering to the mammal in need of such treatment or prevention, an effective amount of a compound of formula (I).

In an embodiment, the disorder as stated above is cancer, which includes liver cancer, melanoma, Hodgkin's disease, non-Hodgkin's lymphomas, acute or chronic lymphocytic leukemia, multiple myeloma, neuroblastoma, breast carcinoma, ovarian carcinoma, lung carcinoma, Wilms' tumor, cervical carcinoma, testicular carcinoma, soft-tissue sarcoma, chronic lymphocytic leukemia, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, malignant melanoma, chorio carcinoma, mycosis fungo ide, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma.

The invention further provides a method of potentiating the efficacy of chemotherapeutic regimen for a patient undergoing chemotherapeutic treatment comprising co-administering to the patient an effective amount of a compound of the present invention, wherein, the compound of the invention may be co-administered simultaneously, sequentially, or cyclically with the anticancer agent.

The chemotherapeutic agent mentioned above is selected form busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, elliptinium, etoposide, 5-azacytidine, gemcitabine, 5-fluorouracil, methotrexate, 5-fluoro-2′-deoxy-uridine, fludarabine, nelarabine, ara-C, alanosine, pralatrexate, pemetrexed, hydroxyurea, thioguanine, colchicine, vinblastine, vincristine, vinorelbine, paclitaxel, ixabepilone, cabazitaxel, docetaxel, campath, panitumumab, ofatumumab, bevacizumab, trastuzumab, adalimumab, imatinib, gefitinib, erlotinib, lapatinib, sorafenib, sunitinib, nilotinib, dasatinib, pazopanib, temsirolimus, everolimus, vorinostat, romidepsin, tamoxifen, letrozole, fulvestrant, mitoguazone, octreotide, retinoic acid, arsenic trioxide, zoledronic acid, bortezomib, thalidomide or lenalidomide.

Overactivation of PARP leads to necrotic cell death as a result of NAD⁺ and ATP depletion. Cancer patients who have undergone radiotherapy or have been treated with chemotherapeutic agents that damage DNA harbour DNA strand breaks. Activation of PARP in such cases allows the repair of the damaged DNA, thus leading to an undesirable resistance to the chemotherapeutic agents (and the consequent inefficacy). In such a scenario, treatment with a PARP inhibitor is expected to make the repair process inefficient and cause cell death.

In a further embodiment, the invention provides a method for sensitizing a patient who has developed or likely to develop resistance for chemotherapic agents comprising administering an effective amount of a compound of the present invention.

### EXAMPLES

The following examples further illustrate a method of preparation of representative compounds embodied in formula (I); however, the same should not be constructed as limiting the scope of the invention.

### Example 1: Synthesis of (R)-4-(4-(3-(5-oxo-5,6-dihydro-1,6-naphthyridin-7-yl)cyclopent-2-en-1-yl)piperazin-1-yl)benzonitrile (Compound 1)

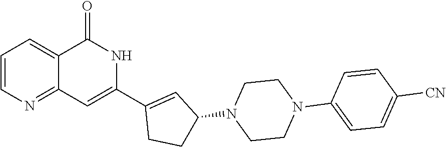 [SMILES: N#Cc1ccc(N2CCN([C@H]3C=C(c4cc5ncccc5c(=O)[nH]4)CC3)CC2)cc1]

### Step 1: 2-bromo-3-oxocyclopent-1-enecarbonitrile (Compound 1a)

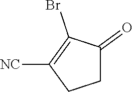

To a stirred solution of 2-bromo-3-ethoxycyclopent-2-enone (Prepared according to the procedure reported in Journal of Medicinal Chemistry, 1999, 42, 7, 1274-1281, 185.00 g, 0.90 mol) in dichloromethane (1200 ml) was added zinc iodide (28.80 g, 0.09 mol) and trimethylsilyl cyanide (179.00 g, 242.0 ml, 1.80 mol) under nitrogen atmosphere at 0° C. and reaction mixture was stirred at 25° C. for 0.5 hr and at room temperature for 18 hr. The progress of the reaction was monitored by TLC. The reaction mixture was slowly quenched with aqueous 1M hydrochloric acid solution (500 ml). The organic layer was separated and washed with aqueous sodium bicarbonate solution (2×500 ml). The organic layer was dried over sodium sulphate and was concentrated to obtain crude product; which was purified by column chromatography over silica gel (100-200 mesh) using 25% ethyl acetate in hexane as an eluent to obtain the title compound (128.00 g, 76.0%). 1H NMR (400 MHz, CDCl₃) δ 2.91 (t, J=6.8 Hz, 2H), 2.71 (t, J=6.8 Hz, 2H).

MS: m/z 186 (M+1).

### Step 2: (S)-2-bromo-3-hydroxycyclopent-1-enecarbonitrile (Compound 1b)

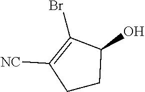

To a stirred solution of 2-bromo-3-oxocyclopent-1-enecarbonitrile (Compound 1a, 110.00 g, 0.59 mol) in tetrahydrofuran (700 ml) was added (R)-1,3a-dimethyl-3,3-diphenylhexahydropyrrolo[1,2c][1,3,2]oxaborole (118.0 ml 1M solution in Toluene, 0.12 mol) under nitrogen atmosphere at 0° C. Stirring was continued over a period of 20 min. Borane dimethylsulfide complex (31.4 gm, 39.3 ml, 0.41 mol) was added to the reaction mixture at 0° C. in drop wise manner in 20 min and reaction mixture was stirred at 0° C. for 1 hr. The progress of the reaction was monitored by TLC. The reaction mixture was quenched with methanol (50 ml). The organic layer was dried over sodium sulphate and was concentrated to obtain crude product. A column of silica gel (100-200 mesh) was loaded in hexane and crude compound was adsorbed over silica gel (100-200 mesh). The eluent used for column was hexane to 25% ethyl acetate and the desired product was eluted in 20-22% ethyl acetate in hexane to obtain the title compound (93.4 g, 84.0%, % ee=94.0% confirmed by chiral HPLC).

¹H NMR (400 MHz, CDCl₃) δ 4.83-4.85 (m, 1H), 2.69-2.74 (m, 1H), 2.51-2.56 (m, 2H), 2.48 (brs-exchanges with D₂O, 1H), 1.96-2.04 (m, 1H).

MS: m/z 188.2 (M+1).

### Step 3: (R)-(S)-2-bromo-3-cynocyclopent-2-en-1-yl 2-acetoxy-2-phenylacetate (Compound 1c)

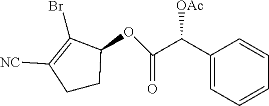 [SMILES: CC(=O)O[C@@H](C(=O)O[C@H]1CCC(C#N)=C1Br)c1ccccc1]

To a stirred solution of (S)-2-bromo-3-hydroxycyclopent-1-enecarbonitrile (Compound 1b, 145.0 g, 0.77 mol) in dichloromethane (1000 ml) was added (2R)-2-acetoxy-2-phenylacetic acid (150.0 g, 0.77 mol) and dimethyl amino pyridine (4.7 g, 38.6 mmol) at 0° C. To this N, N′-Dicyclohexyl dicarbodiimide (175.0 g, 0.85 mol) was added in portions at 0° C. The reaction mixture was stirred over a period of 4 hr at room temperature (white solid separates out). The progress of the reaction was monitored by TLC. The reaction mixture was filtered and organic layer was washed with 5% aqueous hydrochloric acid, saturated aqueous sodium bicarbonate solution and was dried over sodium sulphate. The organic layer was concentrated to obtain crude product which was again dissolved in ether (1500 ml) and filtered; filtrate was concentrated up to 200 ml of ether and then triturated with hexane (3000 ml) to form the precipitated off white title product (232.0 g, 82.0%).

¹H NMR (400 MHz, CDCl₃) δ 7.47-7.50 (m, 2H), 7.38-7.42 (m, 3H), 5.93 (s, 1H), 5.83-4.86 (m, 1H), 2.22 (s, 3H), 2.47-2.64 (m, 3H), 1.74-1.77 (m, 1H).

MS: m/z 366.1 (M+1).

### Step 4: (S)-2-bromo-3-hydroxycyclopent-1-enecarbonitrile (Compound 1d)

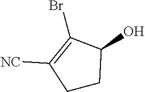

To a stirred solution of (R)-(S)-2-bromo-3-cynocyclopent-2-en-1-yl 2-acetoxy-2-phenylacetate (Compound 1c, 115.0 g, 0.30 mol) in tetrahydrofuran: Water (600:300 ml) was added lithium hydroxide (22.6 g, 0.94 mol) and the reaction mixture was stirred at room temperature for 2 hr. The progress of the reaction was monitored by TLC. The reaction mixture was quenched with water (300 ml) and extracted with dichloromethane (2×500 ml). The organic layer was separated and washed with aqueous 10% hydrochloric acid (300 ml). The organic layer was dried over sodium sulphate and was concentrated to obtain title product (45.0 g, Yield=76.0%, % ee=98.5% confirmed by chiral HPLC).

¹H NMR (400 MHz, CDCl₃) δ 4.83-4.85 (m, 1H), 2.69-2.74 (m, 1H), 2.51-2.56 (m, 2H), 2.48 (bs-exchanges with D₂O, 1H), 1.96-2.04 (m, 1H).

MS: m/z 188 (M+1).

### Step 5: (S)-3-hydroxycyclopent-1-enecarbonitrile (Compound 1e)

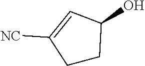

Aqueous 10% hydrochloric acid (750 ml) was added to zinc (272.0 g, 4.10 mol) with stirring at room temperature. After 5 min, hydrochloric acid was decanted and zinc was washed with acetone (2×100 ml), and diethyl ether (2×100 ml). Zinc was dried under vacuum (vacuum was released under nitrogen); free flowing zinc was added to a suspension of silver acetate in boiling acetic acid. After 1 min supernatant was decanted and the black Zn—Ag couple was washed with acetic acid (200 ml), ether (4×100 ml) and methanol (2×100 ml). To a moist Zn—Ag couple was added a solution of (S)-2-bromo-3-hydroxycyclopent-1-enecarbonitrile (Compound 1d, 130.0 g, 0.69 mol) in methanol (600 ml) at 25° C. and was stirred at 25° C. for 24.0 hr. The progress of the reaction was monitored by TLC. The reaction mixture was filtered and washed with methanol (50 ml), filtrate was concentrated and then portioned between ether (1000 ml) and 30% aqueous hydrochloric acid (300 ml). The ether layer was separated, dried over sodium sulphate and concentrated to obtain a crude product. The crude product was purified by column chromatography over silica gel (100-200 mesh) using 20-22% ethyl acetate in hexane as an eluent to obtain the title compound (64.1 g, 85.0%).

¹H NMR (400 MHz, CDCl₃) δ 6.64 (s, 1H), 4.99-5.03 (m, 1H), 2.74-2.79 (m, 1H), 2.51-2.56 (m, 1H), 2.46-2.49 (m, 1H), 1.95 (bs-exchanges with D₂O, 1H), 1.83-1.87 (m, 1H).

MS: m/z 108 (M+1).

### Step 6: (R)-tert-butyl (3-cyanocyclopent-2-en-1-yl)carbamate (Compound 1f)

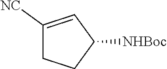 [SMILES: CC(C)(C)OC(=O)N[C@H]1C=C(C#N)CC1]

To a stirred solution of (S)-3-hydroxycyclopent-1-enecarbonitrile (Compound 1e, 64.0 g, 0.58 mol) in tetrahydrofuran (500 ml), was added [azido(phenoxy)phosphoryl]oxybenzene (210.0 g, 164.9 ml, 0.76 mol) at 0° C. in drop wise manner. The reaction mixture was stirred at 0° C. for 10 min and 1,8-diazabicyclo[5.4.0]undec-7-ene (116.0 g, 115.0 ml, 0.76 mol) was added to reaction mixture at 0° C. The reaction mixture was allowed to stir at 0° C. for 2 hr. The progress of the reaction was monitored by TLC. Triphenyl phosphine (169.0 g, 0.64 mol) and water (140 ml) were added at 0° C. and reaction mixture was stirred at room temperature for 18 hrs. The progress of the reaction was monitored by TLC. Boc anhydride (141.0 g, 150 ml, 0.64 mol) was added to the reaction mixture at 0° C. followed by addition of triethyl amine (89.0 g, 123.0 ml, 0.88 mol), the reaction mixture was gradually warmed to room temperature, and stirred for 3 hrs. The progress of the reaction was monitored by TLC. The reaction mixture was quenched with water (50 ml). The reaction mixture was concentrated; and to the residue saturated aqueous ammonium chloride solution (100 ml) was added and extracted with ethyl acetate (2×250 ml). The organic layer was separated, dried over sodium sulphate and concentrated to obtain the crude product; which was purified by flash column chromatography using 10% ethyl acetate in hexane as an eluent to obtain the title compound (0.14 g, 45.0%).

¹H NMR (400 MHz, CDCl₃) δ 6.57 (s, 1H), 4.88-4.90 (m, 1H), 4.63 (brs-exchangeable with D₂O, 1H), 2.45-2.70 (m, 3H), 1.65-1.69 (m, 1H), 1.46 (s, 9H).

MS: m/z 207 (M+1).

### Step 7: (R)-tert-butyl (3-formylcyclopent-2-en-1-yl)carbamate (Compound 1g)

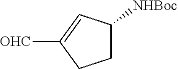 [SMILES: CC(C)(C)OC(=O)N[C@H]1C=C(C=O)CC1]

To a stirred solution of (R)-tert-butyl (3-cyanocyclopent-2-en-1-yl)carbamate (Compound if, 10.0 g, 48.0 mmol) in dichloromethane (100 ml), diisobutyl aluminium hydride (72 ml 1M solution in toluene, 72.0 mmol) was added at −40° C. A cooling bath was removed and reaction mixture was allowed to warm up to room temperature and stirred for 2 hr. The progress of the reaction was monitored by TLC. The reaction mixture was re-cooled to 0° C. and was quenched with saturated aqueous ammonium chloride solution (30 ml) at 0° C. The reaction mixture was diluted with 10% methanol in dichloromethane (100 ml) and stirred for 10 min and filtered through a Celite bed. The Celite bed was washed with 10% methanol in dichloromethane (100 ml). The combined filtrate was concentrated under reduced pressure to obtain crude product; which was purified by flash column chromatography using 25% ethyl acetate in hexane as an eluent to obtain the title compound (0.050 g, 43.1%).

¹H NMR (400 MHz, CDCl₃) δ 9.83 (s, 1H), 6.75 (s, 1H), 4.89-4.92 (m, 1H), 4.60 (brs-exchangeable with D₂O, 1H), 2.62-2.65 (m, 1H), 2.40-2.51 (m, 2H), 1.64-1.67 (m, 1H), 1.49 (s, 9H).

### Step 8: (R)-tert-butyl (3-ethynylcyclopent-2-en-1-yl)carbamate (Compound 1h)

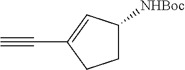

To the stirred solution of trimethylsilyldiazomethane (12.3 ml 2M solution in diethyl ether, 24.6 mmol) in tetrahydrofuran (15 ml) was added n-Butyl lithium (15.5 ml, 1.6 M solution in hexane) at −78° C. in drop wise manner and stirred for 30 min. (R)-tert-butyl (3-formylcyclopent-2-en-1-yl)carbamate (Compound 1g, 4.0 g, 18.9 mmol) in tetrahydrofuran (15 ml) was added to the reaction mixture and stirred for 10 min. The cooling bath was removed and reaction mixture was allowed to stir at room temperature for 2 hr. The progress of the reaction was monitored by TLC. The reaction mixture was diluted with ethyl acetate (100 ml), organic layer was washed with water (20 ml) and dried over anhydrous sodium sulphate. The organic layer was concentrated under reduced pressure to obtain crude product; which was purified by flash column chromatography using 15% ethyl acetate in hexane as an eluent to obtain the title compound (2.8 g, 70.5%).

¹H NMR (400 MHz, CDCl₃) δ 6.04 (q, J=2.1 Hz, 1H), 4.91-4.72 (m, 1H), 4.56 (bs, exchanges with D₂O, 1H), 3.07 (s, 1H), 2.62-2.48 (m, 1H), 2.48-2.32 (m, 2H), 1.71-1.53 (m, 1H), 1.40 (s, 9H).

MS: m/z 207 (M+1).

### Step 9: (R)-3-ethynylcyclopent-2-enamine hydrochloride (Compound 1i)

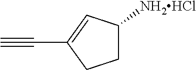

To a stirred solution of (R)-tert-butyl (3-ethynylcyclopent-2-en-1-yl)carbamate (Compound 1h, 1.5 g, 7.24 mmol) in dichloromethane (10 ml), hydrochloric acid (2.2 ml 4M solution in dioxane, 72.4 mmol) was added at 0° C. The reaction mixture was stirred at room temperature for 1 hr. The progress of the reaction was monitored by TLC. The reaction mixture was concentrated under reduced pressure to dryness. A residue was co-evaporated with toluene to obtain the title product (0.95 gm, 95.5%).

¹H NMR (400 MHz, DMSO-d₆) δ 8.26 (bs-exchanges with D₂O, 2H), 6.05 (s, 1H), 4.24-4.26 (m, 1H), 3.40 (s, 1H), 2.59-2.62 (m, 1H), 2.41-2.42 (m, 1H), 2.24-2.27 (m, 1H), 1.79-1.82 (m, 1H).

### Step 10: (R)-4-(4-(3-ethynylcyclopent-2-en-1-yl)piperazin-1-yl)benzonitrile (Compound 1j)

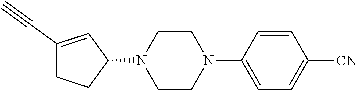 [SMILES: C#CC1=C[C@H](N2CCN(c3ccc(C#N)cc3)CC2)CC1]

To the (R)-3-ethynylcyclopent-2-enamine hydrochloride (Compound 1i, 6.8 g, 47.3 mmol) and 4-(bis(2-chloroethyl)amino)benzonitrile (Prepared according to the procedure reported in U.S. Pat. No. 6,455,528, 14.53 g, 61.6 mmol) were added sodium bicarbonate (19.9 g, 237.0 mmol), potassium iodide (19.6 g, 118.0 mmol), and n-butanol (70 ml) at room temperature under nitrogen atmosphere. The reaction mixture was heated at 110° C. for 18 hrs under nitrogen atmosphere on pre-heated oil bath. The progress of the reaction was monitored by TLC. The reaction mixture was cooled to room temperature and diluted with ethyl acetate (50 ml). The reaction mixture was filtered through Celite, and washed with ethyl acetate (40 ml). The combined filtrate was concentrated under reduced pressure to obtain a crude product which was purified by flash column chromatography using 15% ethyl acetate in hexane as an eluent to obtain the title compound (10.5 g, 82.0%).

¹H NMR (400 MHz, CDCl₃) δ 7.56-7.47 (m, 2H), 6.91-6.84 (m, 2H), 6.18 (q, J=2.2 Hz, 1H), 4.00-3.88 (m, 1H), 3.41-3.26 (m, 4H), 3.08 (s, 1H), 2.74-2.61 (m, 4H), 2.62-2.42 (m, 2H), 2.14-1.85 (m, 2H).

MS: m/z 278 (M+1).

### Step 11: (R)-4-(4-(3-(5-oxo-5,6-dihydro-1,6-naphthyridin-7-yl)cyclopent-2-en-1-yl)piperazin-1-yl)benzonitrile (Compound 1)

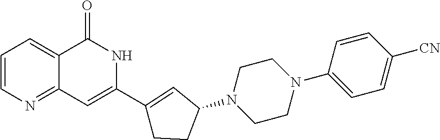 [SMILES: N#Cc1ccc(N2CCN([C@H]3C=C(c4cc5ncccc5c(=O)[nH]4)CC3)CC2)cc1]

To a solution of 2-bromonicotinic acid (0.947 g, 4.69 mmol) in anhydrous acetonitrile (10 ml, degassed by nitrogen gas) was added bis(triphenylphosphine) palladium (II) chloride (0.253 g, 0.361 mmol) at 25° C. The reaction mixture was heated and stirred at 70° C. for 10 min and to this warmed reaction mixture was added diisopropylethylamine (3.78 ml, 21.63 mmol) followed by the addition of a solution of (R)-4-(4-(3-ethynylcyclopent-2-en-1-yl)piperazin-1-yl)benzonitrile (Compound 1j, 1.0 g, 3.61 mmol) in acetonitrile (5 ml) and the reaction mixture was heated at same temperature for 3 hrs. The progress of the reaction was monitored by TLC. The reaction mixture was cooled to room temperature and diluted with ethyl acetate (200 ml), washed with water (100 ml). The aqueous layer was again extracted with ethyl acetate (100 ml) and the combined organic layer was dried over sodium sulfate, filtered and concentrated under reduced pressure to give crude intermediate; which was dissolved in anhydrous tetrahydrofuran (10 ml). To this solution of crude intermediate was added ammonia in methanol (50 ml 7M solution in methanol, 361.0 mmol) at 25° C. and was heated at 90° C. for 2 hr. The progress of the reaction was monitored by TLC. The reaction mixture was cooled to room temperature, filtered and filtrate was concentrated under reduced pressure to obtain crude product which was purified by flash column chromatography over silica gel (100-200 mesh) using 0-5% methanol in dichloromethane as eluent to obtain title compound (0.110 g, 7.68% yield).

¹H NMR (400 MHz, DMSO-d₆) δ 11.47 (brs-exchangeable with D₂O, 1H), 8.90 (dd, J=8.8, 2.0 Hz, 1H), 8.47 (dd, J=8.0, 2.0 Hz, 1H), 7.59 (d, J=8.8 Hz, 2H), 7.48 (dd, J=8.0, 2.0 Hz, 1H), 7.04 (d, J=8.8 Hz, 2H), 6.96 (d, J=2.0 Hz, 1H), 6.59 (s, 1H), 3.97-3.86 (m, 1H), 3.43-3.35 (m, 4H), 2.82-2.70 (m, 1H), 2.68-2.55 (m, 4H), 2.15-2.01 (m, 1H), 1.98-1.80 (m, 1H), 1.38-1.13 (m, 1H).

MS: m/z 398.3 (M+1).

### Step 12: (R)-4-(4-(3-(5-oxo-5,6-dihydro-1,6-naphthyridin-7-yl)cyclopent-2-en-1-yl)piperazin-1-yl)benzonitrile (Compound 1—hydrochloride salt)

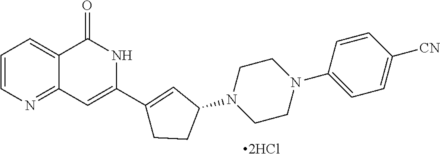 [SMILES: N#Cc1ccc(N2CCN([C@H]3C=C(c4cc5ncccc5c(=O)[nH]4)CC3)CC2)cc1]

A solution of (R)-4-(4-(3-(5-oxo-5,6-dihydro-1,6-naphthyridin-7-yl)cyclopent-2-en-1-yl)piperazin-1-yl)benzonitrile (Compound 1, 90 mg, 0.226 mmol) in tetrahydrofuran (2 ml) and methanol (2 ml) was heated at 65° C. and was added hydrochloric acid in methanol (0.830 ml, 0.498 mmol, 3M solution) at same temperature in small portions over a period of 5 min. The reaction mixture was then stirred for 30 min at 25° C. The reaction mixture was cooled to room temperature, diluted with diethyl ether (10 ml), and product was collected upon filtration. The resulting solid was washed with diethyl ether (10 ml) and dried under reduced pressure for 3 hr at 40° C. to obtain the title compound (0.095 g, 89% yield).

¹H NMR (400 MHz, DMSO-d₆) δ 11.73 (brs-exchangeable with D₂O, 1H), 11.49 (brs-exchangeable with D₂O, 1H), 9.00 (dd, J=8.8, 2.0 Hz, 1H), 8.62 (dd, J=8.0, 2.0 Hz, 1H), 7.68 (d, J=8.8 Hz, 2H), 7.62 (dd, J=8.0, 2.0 Hz, 1H), 7.15 (d, J=8.8 Hz, 2H), 6.89 (d, J=2.0 Hz, 1H), 6.82 (s, 1H), 4.73-4.53 (m, 2H), 4.15 (d, J=12.4 Hz, 2H), 3.59 (t, J=11.6 Hz, 2H), 3.35 (t, J=11.6 Hz, 2H), 3.12 (dd, J=20.0, 9.6 Hz, 2H), 2.90 (q, J=7.6 Hz, 2H), 2.40 (q, J=7.6 Hz, 2H).

MS: m/z 398.2 (M+1).

The following compounds were prepared using the procedure described above in Example 1 with appropriate changes to the reactants and reaction conditions.

### (R)-7-(3-(4-phenylpiperazin-1-yl)cyclopent-1-en-1-yl)-1,6-naphthyridin-5(6H)-one (Compound 21—hydrochloride salt)

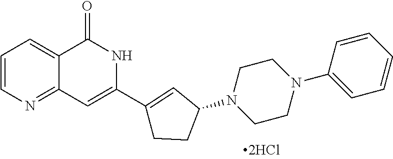 [SMILES: O=c1[nH]c(C2=C[C@H](N3CCN(c4ccccc4)CC3)CC2)cc2ncccc12]

¹H NMR (400 MHz, DMSO-d₆) δ 11.75 (brs-exchangeable with D₂O, 1H), 11.16 (brs-exchangeable with D₂O, 1H), 9.03-8.95 (m, 1H), 8.65-8.57 (m, 1H), 7.66-7.57 (m, 1H), 7.28 (t, J=7.8 Hz, 2H), 7.03 (d, J=8.2 Hz, 2H), 6.92 (s, 1H), 6.88 (t, J=7.2 Hz, 1H), 6.85-6.79 (m, 1H), 4.69 (s, 1H), 3.92-3.84 (m, 2H), 3.64-3.54 (m, 2H), 3.27-3.08 (m, 4H), 2.94-2.87 (m, 2H), 2.46-2.35 (m, 2H).

MS: m/z 373.0 (M+1).

### (R)-7-(3-(4-(o-tolyl)piperazin-1-yl)cyclopent-1-en-1-yl)-1,6-naphthyridin-5(6H)-one (Compound 3)

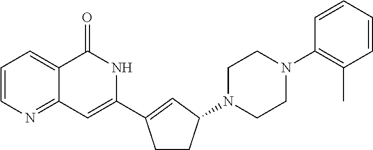 [SMILES: Cc1ccccc1N1CCN([C@H]2C=C(c3cc4ncccc4c(=O)[nH]3)CC2)CC1]

¹H NMR (400 MHz, DMSO-d₆) δ 11.49 (brs-exchangeable with D₂O, 1H) 8.95-8.87 (m, 1H), 8.52-8.44 (m, 1H), 7.52-7.44 (m, 1H), 7.21-7.13 (m, 2H), 7.06-6.91 (m, 3H), 6.60 (s, 1H), 3.99-3.83 (m, 1H), 2.95-2.81 (m, 4H), 2.80-2.56 (m, 6H), 2.26 (s, 3H), 2.14-2.05 (m, 1H), 1.98-1.88 (m, 1H).

MS: m/z 386.8 (M+1).

### (R)-4-(2-oxo-4-(3-(5-oxo-5,6-dihydro-1,6-naphthyridin-7-yl)cyclopent-2-en-1-yl)piperazin-1-yl)benzonitrile (Compound 13—hydrochloride salt)

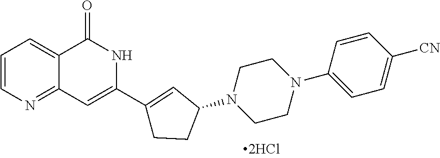 [SMILES: N#Cc1ccc(N2CCN([C@H]3C=C(c4cc5ncccc5c(=O)[nH]4)CC3)CC2)cc1]

¹H NMR (400 MHz, DMSO-d₆) δ 12.45 (brs-exchangeable with D₂O, 1H), 11.69 (s brs-exchangeable with D₂O, 1H), 8.99 (dd, J=4.8, 1.7 Hz, 1H), 8.61 (dd, J=8.1, 1.7 Hz, 1H), 7.96 (d, J=8.4 Hz, 2H), 7.66-7.58 (m, 3H), 6.87 (s, 1H), 6.83 (s, 1H), 4.73-4.61 (m, 2H), 4.35-4.22 (m, 1H), 4.12 (s, 2H), 4.00-3.85 (m, 1H), 3.65-3.48 (m, 1H), 3.03-2.86 (m, 2H), 2.47-2.36 (m, 2H).

MS: m/z 412.1 (M+1).

### (R)-7-(3-(4-(4-fluorophenyl)piperazin-1-yl)cyclopent-1-en-1-yl)-1,6-naphthyridin-5(6H)-one (Compound 22—hydrochloride salt)

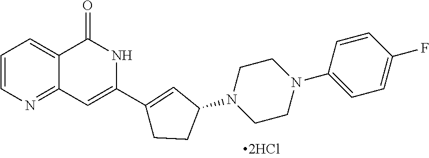 [SMILES: O=c1[nH]c(C2=C[C@H](N3CCN(c4ccc(F)cc4)CC3)CC2)cc2ncccc12]

¹H NMR (400 MHz, DMSO-d₆) δ 11.89 (brs-exchangeable with D₂O, 1H), 11.52 (brs-exchangeable with D₂O, 1H), 9.04 (s, 1H), 8.73 (d, J=8.0 Hz, 1H), 7.70 (dd, J=8.0, 5.0 Hz, 1H), 7.14-7.10 (m, 2H), 7.07-7.03 (m, 3H), 6.96 (s, 1H), 6.88 (s, 1H), 4.69 (s, 1H), 3.84-3.75 (m, 2H), 3.62-3.54 (m, 2H), 3.30-3.11 (m, 4H), 2.94-2.86 (m, 2H), 2.44-2.38 (m, 2H).

MS: m/z 391.2 (M+1).

### (R)-7-(3-(4-(4-chlorophenyl)piperazin-1-yl)cyclopent-1-en-1-yl)-1,6-naphthyridin-5(6H)-one (Compound 24—hydrochloride salt)

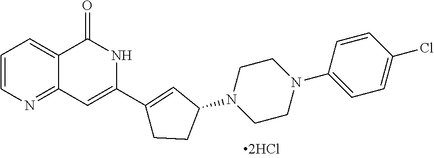 [SMILES: O=c1[nH]c(C2=C[C@H](N3CCN(c4ccc(Cl)cc4)CC3)CC2)cc2ncccc12]

¹H NMR (400 MHz, DMSO-d₆) δ 11.79 (brs-exchangeable with D₂O, 1H), 11.43 (brs-exchangeable with D₂O, 1H), 9.01 (d, J=4.8 Hz, 1H), 8.65 (d, J=8.0 Hz, 1H), 7.65 (dd, J=8.0, 4.8 Hz, 1H), 7.31 (d, J=8.6 Hz, 2H), 7.05 (d, J=8.6 Hz, 2H), 6.93 (s, 1H), 6.84 (s, 1H), 4.69-4.67 (m, 1H), 3.90-3.87 (m, 2H), 3.60-3.55 (m, 2H), 3.26-3.12 (m, 4H), 2.98-2.83 (m, 2H), 2.44-2.36 (m, 2H).

MS: m/z 407.1 (M+1).

### (R)-7-(3-(4-(4-methoxyphenyl)piperazin-1-yl)cyclopent-1-en-1-yl)-1,6-naphthyridin-5(6H)-one (Compound 25—hydrochloride salt)

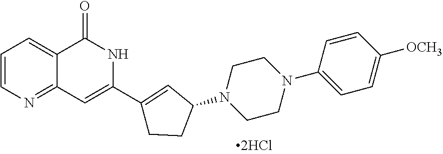 [SMILES: COc1ccc(N2CCN([C@H]3C=C(c4cc5ncccc5c(=O)[nH]4)CC3)CC2)cc1]

¹H NMR (400 MHz, DMSO-d₆) δ11.96 (brs-exchangeable with D₂O, 1H), 11.62 (brs-exchangeable with D₂O, 1H), 9.06 (d, J=5.0 Hz, 1H), 8.77 (d, J=8.0 Hz, 1H), 7.73 (dd, J=8.0, 5.0 Hz, 1H), 7.04 (d, J=8.0 Hz, 2H), 6.99 (s, 1H), 6.91-6.81 (m, 3H), 4.70 (s, 1H), 3.70 (s, 3H), 3.64-3.50 (m, 2H), 3.33-3.14 (m, 2H), 3.12-3.02 (m, 2H), 2.93-2.87 (m, 2H), 2.49-2.42 (s, 2H), 2.37-2.31 (m, 2H).

MS: m/z 403.1 (M+1).

### (R)-7-(3-(4-(p-tolyl)piperazin-1-yl)cyclopent-1-en-1-yl)-1,6-naphthyridin-5(6H)-one (Compound 26—hydrochloride salt)

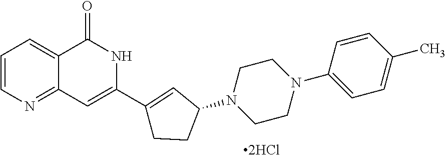 [SMILES: Cc1ccc(N2CCN([C@H]3C=C(c4cc5ncccc5c(=O)[nH]4)CC3)CC2)cc1]

¹H NMR (400 MHz, DMSO-d₆) δ 11.80 (brs-exchangeable with D₂O, 1H), 11.34 (brs-exchangeable with D₂O, 1H), 9.02 (d, J=4.8 Hz, 1H), 8.66 (d, J=8.0 Hz, 1H), 7.65 (dd, J=8.1, 4.8 Hz, 1H), 7.09 (d, J=8.2 Hz, 2H), 6.96-6.91 (m, 3H), 6.84 (s, 1H), 4.67-4.65 (m, 1H), 3.81-3.79 (m, 2H), 3.59-3.55 (m, 2H), 3.28-3.08 (m, 4H), 2.94-2.85 (m, 2H), 2.44-2.31 (m, 2H), 2.23 (s, 3H).

MS: m/z 387.1 (M+1).

### (R)-7-(3-(4-(2,3-dihydro-1H-inden-5-yl)piperazin-1-yl)cyclopent-1-en-1-yl)-1,6-naphthyridin-5(6H)-one (Compound 30—hydrochloride salt)

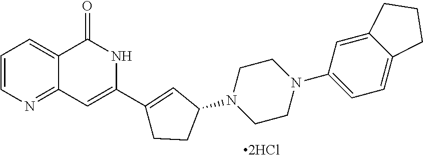 [SMILES: O=c1[nH]c(C2=C[C@H](N3CCN(c4ccc5c(c4)CCC5)CC3)CC2)cc2ncccc12]

¹H NMR (400 MHz, DMSO-d₆) δ 11.81 (brs-exchangeable with D₂O, 1H), 11.31 (brs-exchangeable with D₂O, 1H), 9.02 (s, 1H), 8.66 (d, J=7.8 Hz, 1H), 7.74-7.56 (m, 1H), 7.12 (d, J=8.4 Hz, 1H), 6.94-6.92 (m, 2H), 6.83-6.80 (m, 2H), 4.86 (s, 1H), 4.68 (s, 1H), 3.79-3.76 (m, 2H), 3.59-3.56 (m, 2H), 3.28-3.11 (m, 5H), 2.97-2.84 (m, 2H), 2.83-2.71 (m, 3H), 2.41-2.39 (m, 1H), 2.09-1.92 (m, 2H).

MS: m/z 414.2 (M+1).

### (R)-7-(3-(4-(2,4-difluorophenyl)piperazin-1-yl)cyclopent-1-en-1-yl)-1,6-naphthyridin-5(6H)-one (Compound 39—hydrochloride salt)

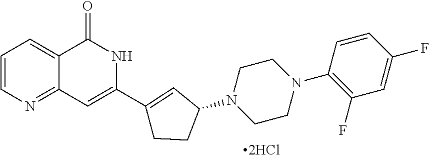 [SMILES: O=c1[nH]c(C2=C[C@H](N3CCN(c4ccc(F)cc4F)CC3)CC2)cc2ncccc12]

¹H NMR (400 MHz, DMSO-d₆) δ 11.79 (brs-exchangeable with D₂O, 1H), 11.45 (brs-exchangeable with D₂O, 1H), 9.01 (dd, J=4.5, 1.5 Hz, 1H), 8.65 (d, J=8.0 Hz, 1H), 7.63-7.66 (m, 1H), 7.26-7.32 (m, 1H), 7.15-7.21 (m, 1H), 7.08-7.03 (m, 1H), 6.93 (s, 1H), 6.83 (s, 1H), 4.72 (s, 1H), 3.60-3.54 (m, 2H), 3.48-3.45 (m, 3H), 3.15-3.32 (m, 4H), 2.99-2.85 (m, 2H), 2.43-2.38 (m, 2H).

MS: m/z 409.1 (M+1).

### (R)-6-(3-(4-phenylpiperazin-1-yl)cyclopent-1-en-1-yl)thieno[3,2-c]pyridin-4(5H)-one (Compound 48—hydrochloride salt)

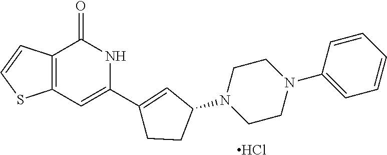 [SMILES: O=c1[nH]c(C2=C[C@H](N3CCN(c4ccccc4)CC3)CC2)cc2sccc12 ; Cl]

¹H NMR (400 MHz, DMSO-d₆) δ 11.41 (brs-exchangeable with D₂O, 1H), 11.36 (brs-exchangeable with D₂O, 1H), 7.69 (d, J=5.3 Hz, 1H), 7.51 (d, J=5.3 Hz, 1H), 7.28 (t, J=7.7 Hz, 2H), 7.10 (s, 1H), 7.03 (d, J=8.2 Hz, 2H), 6.88 (t, J=7.3 Hz, 1H), 6.79 (s, 1H), 4.63 (s, 1H), 3.92-3.80 (m, 2H), 3.64-3.49 (m, 2H), 3.26-3.09 (m, 4H), 2.94-2.74 (m, 2H), 2.41-2.32 (m, 2H).

MS: m/z 378.1 (M+1).

### Example 2: Synthesis of (R)-3-fluoro-4-(4-(3-(5-oxo-5,6-dihydro-1,6-naphthyridin-7-yl)cyclopent-2-en-1-yl)piperazin-1-yl)benzonitrile (Compound 23)

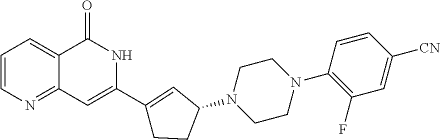 [SMILES: N#Cc1ccc(N2CCN([C@H]3C=C(c4cc5ncccc5c(=O)[nH]4)CC3)CC2)c(F)c1]

### Step 1: 3-bromocyclopent-1-enecarbonitrile (Compound 23a)

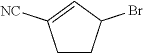

To a stirred solution of cyclopent-1-enecarbonitrile (50 g, 537 mmol) in tetrachloromethane (400 ml) at 25° C. was added N-bromosuccinimide (96 g, 537 mmol) under nitrogen atmosphere. The resulting mixture was refluxed for 2 hr. The progress of reaction was monitored by TLC. The reaction mixture was cooled to 25° C. and filtered through Celite. The filtrate was concentrated under reduced pressure to obtain a crude product, which was purified by column chromatography over silica gel (100-200 mesh) using 1% ethyl acetate in hexane as an eluent to obtain the title compound (60.0 g, 65%).

¹HNMR (400 MHz, CDCl₃): δ 6.77-6.73 (m, 1H), 5.12-5.09 (m, 1H) 2.95-2.86 (m, 1H) 2.67-2.42 (m, 3H).

### Step 2: tert-butyl 4-(3-cyanocyclopent-2-en-1-yl)piperazine-1-carboxylate (Compound 23b)

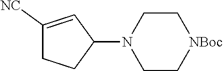 [SMILES: CC(C)(C)OC(=O)N1CCN(C2C=C(C#N)CC2)CC1]

To a stirred solution of tert-butyl piperazine-1-carboxylate (59.5 g, 320 mmol) in dimethyl formamide (400 ml) was added triethylamine (134 ml, 959 mmol) at 25° C. and stirred the reaction mixture for 10 minutes. To the above mixture was added 3-bromocyclopent-1-enecarbonitrile (Compound 23a, 55 g, 320 mmol) and the reaction mixture was stirred for 3 hr. The progress of the reaction was monitored by TLC. The reaction mixture was then concentrated under reduced pressure. The residue obtained was diluted with water (250 ml) and extracted with ethyl acetate (3×250 ml). The combined organic layer was dried over anhydrous sodium sulphate. The solvent in the organic layer was evaporated under reduced pressure to obtain a crude product. The crude product was purified by flash column chromatography over silica gel (100-200 mesh) using 40% ethyl acetate in hexane as an eluent to obtain the title compound (35.0 g, 39.5% yield).

¹H NMR (400 MHz, CDCl₃): δ 6.66-6.64 (m, 1H) 3.97-3.93 (m, 1H), 3.45-2.42 (m, 4H), 2.65-2.57 (m, 2H), 2.50-2.40 (m, 4H), 2.11-2.04 (m, 1H) 1.97-1.89 (m, 1H) 1.47 (s, 9H).

A chiral separation of racemic tert-butyl 4-(3-cyanocyclopent-2-en-1-yl)piperazine-1-carboxylate (Compound 23b-racemic, 30 g) was carried out using chiral column to obtain

### (R) tert-butyl 4-(3-cyanocyclopent-2-en-1-yl)piperazine-1-carboxylate (Compound 23b′; 12 g)

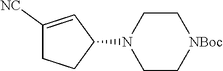 [SMILES: CC(C)(C)OC(=O)N1CCN([C@H]2C=C(C#N)CC2)CC1]

¹H NMR (400 MHz, CDCl₃): δ 6.66-6.64 (m, 1H) 3.97-3.93 (m, 1H), 3.45-2.42 (m, 4H), 2.65-2.57 (m, 2H), 2.50-2.40 (m, 4H), 2.11-2.04 (m, 1H) 1.97-1.89 (m, 1H) 1.47 (s, 9H).

and

### (S) tert-butyl 4-(3-cyanocyclopent-2-en-1-yl)piperazine-1-carboxylate (compound 23b″; 11.5 g)

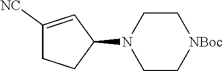 [SMILES: CC(C)(C)OC(=O)N1CCN([C@@H]2C=C(C#N)CC2)CC1]

¹H NMR (400 MHz, CDCl₃): δ 6.66-6.64 (m, 1H) 3.97-3.93 (m, 1H), 3.45-2.42 (m, 4H), 2.65-2.57 (m, 2H), 2.50-2.40 (m, 4H), 2.11-2.04 (m, 1H) 1.97-1.89 (m, 1H) 1.47 (s, 9H).

### Step 3: tert-butyl (R)-4-(3-formylcyclopent-2-en-1-yl)piperazine-1-carboxylate (Compound 23c)

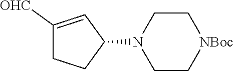 [SMILES: CC(C)(C)OC(=O)N1CCN([C@H]2C=C(C=O)CC2)CC1]

To a stirred solution of (R) tert-butyl 4-(3-cyanocyclopent-2-en-1-yl)piperazine-1-carboxylate (Compound 23b′, 10 g, 36.1 mmol) in dry dichloromethane (100 ml) was added di-isobutyl aluminium hydride (DIBAL-H) (43.3 ml, 1M solution in toluene, 43.3 mmol) under nitrogen atmosphere at −78° C. over a period of 30 min. The reaction mixture was slowly warmed to room temperature and stirred over a period of 16 hr. The progress of the reaction was monitored by TLC. The reaction mixture was diluted with ethyl acetate (250 ml), quenched with saturated aqueous ammonium chloride solution (100 ml) and the reaction mixture was stirred for 15 min. The reaction mass was filtered through a Celite bed and the residue was washed with ethyl acetate (100 ml). The separated organic layer was dried over sodium sulfate, and filtered. The filtrate was concentrated under reduced pressure to obtain a crude product, which was purified by flash column chromatography over silica gel (100-200 mesh) using 35-40% ethyl acetate in hexane as an eluent to obtain the title compound (4.0 g, 39.6%).

¹H NMR (400 MHz, CDCl₃): δ 9.84 (s, 1H), 6.85 (s, 1H), 3.99 (dt, J=6.4, 3.2 Hz, 1H), 3.46 (t, J=4.8 Hz, 4H), 2.66-2.38 (m, 6H), 2.19-2.06 (m, 1H), 2.00-1.85 (m, 1H), 1.47 (s, 9H).

### Step 4: tert-butyl (R)-4-(3-ethynylcyclopent-2-en-1-yl)piperazine-1-carboxylate (Compound 23d)

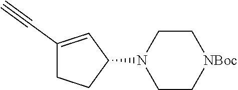 [SMILES: C#CC1=C[C@H](N2CCN(C(=O)OC(C)(C)C)CC2)CC1]

To a stirred solution of trimethylsilyldiazomethane (10.70 ml, 21.40 mmol) in dry tetrahydrofuran (10 ml) was added n-butyl lithium (8.56 ml, 21.40 mmol, 1.6 M solution in hexane) under nitrogen atmosphere at −78° C. The resulting mixture was stirred for 30 min. To this reaction mixture a solution of tert-butyl (R)-4-(3-formylcyclopent-2-en-1-yl)piperazine-1-carboxylate (Compound 23c, 5.0 g, 17.83 mmol) in tetrahydrofuran (25 ml) was added slowly at the same temperature. The reaction mixture was allowed to stir at room temperature for 20 h. The progress of the reaction was monitored by TLC. The reaction mixture was diluted with ethyl acetate (50 ml) and was washed with water (10 ml). The organic layer was dried over sodium sulfate, and filtered. The filtrate was concentrated under reduced pressure to obtain a crude product which was purified by flash column chromatography over silica gel (100-200 mesh) using 45-50% ethyl acetate in hexane as an eluent to obtain the title compound (2.5 g, 50.7%).

¹H NMR (400 MHz, CDCl₃): δ 6.15 (q, J=2.2 Hz, 1H), 3.95-3.85 (m, 1H), 3.52 (s, 4H), 3.06 (s, 1H), 2.61-2.38 (m, 6H), 2.05-1.82 (m, 2H), 1.47 (s, 9H).

### Step 5: (R)-1-(3-ethynylcyclopent-2-en-1-yl)piperazine hydrochloride (Compound 23e)

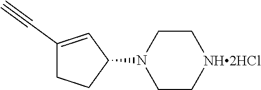

To a solution of tert-butyl (R)-4-(3-ethynylcyclopent-2-en-1-yl)piperazine-1-carboxylate (Compound 23d, 2 g, 7.24 mmol) in dry dichloromethane (250 ml) was added hydrochloric acid (12.06 ml, 36.2 mmol, 4M solution in 1,4-dioxane) in a drop-wise manner at 0-5° C. The reaction mixture was stirred at room temperature for 1-2 hrs. The progress of the reaction was monitored by TLC. The reaction mixture was concentrated under reduced pressure. The residue was washed with diethyl ether (10 ml), and dried under reduced pressure to obtain the title compound.

¹H NMR (400 MHz, DMSO-d₆): δ 12.19 (brs-exchangeable with D₂O, 1H), 9.73 (brs-exchangeable with D₂O, 1H), 6.23 (q, J=2.1 Hz, 1H), 4.58-4.49 (m, 1H), 3.79-3.20 (m, 9H), 2.72-2.60 (m, 1H), 2.51-2.39 (m, 1H), 2.35-2.11 (m, 2H).

### Step 6: (R)-4-(4-(3-ethynylcyclopent-2-en-1-yl)piperazin-1-yl)-3-fluorobenzonitrile (Compound 23f)

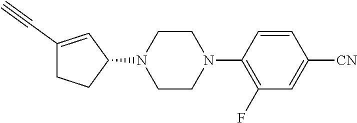 [SMILES: C#CC1=C[C@H](N2CCN(c3ccc(C#N)cc3F)CC2)CC1]

To a solution of (R)-1-(3-ethynylcyclopent-2-en-1-yl)piperazine hydrochloride (Compound 23e, 2.5 g, 14.18 mmol) in N,N-dimethylformamide (20 ml) were added 3,4-difluorobenzonitrile (1.960 g, 14.18 mmol) in N,N-dimethylformamide (5 ml) and potassium carbonate (5.88 g, 42.6 mmol) at room temperature. The reaction mixture was heated at 120-125° C. for 18-20 hr under a nitrogen atmosphere. The progress of the reaction was monitored by TLC. The reaction mixture was cooled to room temperature and quenched with water (50 ml). The aqueous layer was extracted with ethyl acetate (2×100 ml). The combined organic layer was dried over sodium sulfate, filtered and concentrated under reduced pressure to obtain crude product which was purified over flash chromatography over silica gel (100-200 mesh) using 20-30% ethyl acetate as an eluent to obtain the title compound (1.2 g, 30.5%).

¹H NMR (400 MHz, DMSO-d₆) δ 7.74-7.66 (m, 1H), 7.61-7.55 (m, 1H), 7.117-7.08 (m, 1H), 6.18 (d, J=2.0 Hz, 1H), 4.12 (s, 1H), 3.86-3.78 (m, 1H), 3.20-3.11 (m, 4H), 2.64-2.52 (m, 4H), 2.46-2.29 (m, 2H), 2.00-1.87 (m, 1H), 1.86-1.75 (m, 1H).

MS: m/z 296 (M+1).

### Step 7: (R)-3-fluoro-4-(4-(3-(5-oxo-5,6-dihydro-1,6-naphthyridin-7-yl)cyclopent-2-en-1-yl)piperazin-1-yl)benzonitrile (Compound 23)

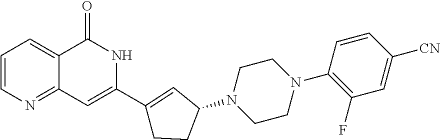 [SMILES: N#Cc1ccc(N2CCN([C@H]3C=C(c4cc5ncccc5c(=O)[nH]4)CC3)CC2)c(F)c1]

To a solution of 2-bromonicotinic acid (0.947 g, 4.69 mmol) in anhydrous acetonitrile (10 ml, degassed by nitrogen gas) was added bis (triphenylphosphine) palladium (II) chloride (0.253 g, 0.361 mmol) at 25° C. The reaction mixture was heated and stirred at 70° C. for 10 min and to this warmed reaction mixture was added diisopropylethylamine (3.78 ml, 21.63 mmol) followed by the addition of a solution of (R)-4-(4-(3-ethynylcyclopent-2-en-1-yl)piperazin-1-yl)-3-fluorobenzonitrile (Compound 23f, 1.0 g, 3.61 mmol) in acetonitrile (5 ml) and the reaction mixture was heated at same temperature for 3 hrs. The progress of the reaction was monitored by TLC. The reaction mixture was cooled to room temperature and diluted with ethyl acetate (200 ml), washed with water (100 ml). The aqueous layer was again extracted with ethyl acetate (100 ml) and the combined organic layer was dried over sodium sulfate, filtered and concentrated under reduced pressure to give crude intermediate; which was dissolved in anhydrous tetrahydrofuran (10 ml). To this solution of crude intermediate was added ammonia in methanol (50 ml, 361.0 mmol) at 25° C. and was heated at 90° C. for 2 hr. The progress of the reaction was monitored by TLC. The reaction mixture was cooled to room temperature, filtered and filtrate was concentrated under reduced pressure to obtain crude product which was purified by flash column chromatography over silica gel (100-200 mesh) using 0-5% methanol in dichloromethane as eluent to obtain title compound (0.110 g, 7.68% yield).

MS: m/z 415 (M+1).

### Step 8: (R)-3-fluoro-4-(4-(3-(5-oxo-5,6-dihydro-1,6-naphthyridin-7-yl)cyclopent-2-en-1-yl)piperazin-1-yl)benzonitrile (Compound 23—hydrochloride salt)

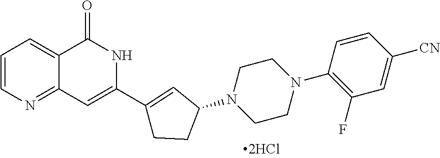 [SMILES: N#Cc1ccc(N2CCN([C@H]3C=C(c4cc5ncccc5c(=O)[nH]4)CC3)CC2)c(F)c1]

A solution of (R)-3-fluoro-4-(4-(3-(5-oxo-5,6-dihydro-1,6-naphthyridin-7-yl)cyclopent-2-en-1-yl)piperazin-1-yl)benzonitrile (Compound 23, 90 mg, 0.226 mmol) in tetrahydrofuran (2 ml) and methanol (2 ml) was heated at 65° C. and was added hydrochloric acid in methanol (0.830 ml, 0.498 mmol, 3M solution) at same temperature in small portions over a period of 5 min. The reaction mixture was then stirred for 30 min at 25° C. The reaction mixture was cooled to room temperature, diluted with diethyl ether (10 ml), and product was collected upon filtration. The solid was washed with diethyl ether (10 ml) and dried under reduced pressure for 3 hr at 40° C. to obtain the title compound (0.095 g, 89% yield).

¹H NMR (400 MHz, DMSO-d₆) δ 11.86 (brs-exchangeable with D₂O, 2H), 9.04 (dd, J=5.0, 1.7 Hz, 1H), 8.84-8.60 (m, 1H), 7.80 (dd, J=13.1, 1.9 Hz, 1H), 7.69 (dd, J=8.1, 5.0 Hz, 1H), 7.64 (dd, J=8.4, 1.9 Hz, 1H), 7.27 (t, J=8.7 Hz, 1H), 6.95 (d, J=2.6 Hz, 1H), 6.87 (s, 1H), 4.70 (s, 1H), 3.76-3.73 (m, 2H), 3.61-3.55 (m, 2H), 3.46-3.40 (m, 2H), 3.31-0.16 (m, 2H), 2.98-2.81 (m, 2H), 2.44-2.38 (m, 2H).

MS: m/z 415.9 (M+1).

The following compounds were prepared using the procedure described above in Example 2 with appropriate changes to the reactants, if required stereoisomer (compound 23b″) and to the reaction conditions.

### (R)-4-(4-(3-(5-oxo-5,6-dihydro-1,6-naphthyridin-7-yl)cyclopent-2-en-1-yl)piperazin-1-yl)benzonitrile (Compound 1—hydrochloride salt)

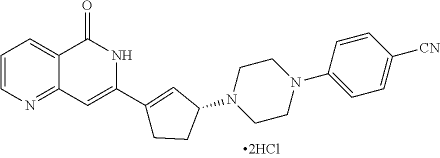 [SMILES: N#Cc1ccc(N2CCN([C@H]3C=C(c4cc5ncccc5c(=O)[nH]4)CC3)CC2)cc1]

¹H NMR (400 MHz, DMSO-d₆) δ 11.73 (brs-exchangeable with D₂O, 1H), 11.49 (brs-exchangeable with D₂O, 1H), 9.00 (dd, J=8.8, 2.0 Hz, 1H), 8.62 (dd, J=8.0, 2.0 Hz, 1H), 7.68 (d, J=8.8 Hz, 2H), 7.62 (dd, J=8.0, 2.0 Hz, 1H), 7.15 (d, J=8.8 Hz, 2H), 6.89 (d, J=2.0 Hz, 1H), 6.82 (s, 1H), 4.73-4.53 (m, 1H), 4.15 (d, J=12.4 Hz, 2H), 3.59 (t, J=11.6 Hz, 2H), 3.35 (t, J=11.6 Hz, 2H), 3.12 (dd, J=20.0, 9.6 Hz, 2H), 2.90 (q, J=7.6 Hz, 2H), 2.40 (q, J=7.6 Hz, 2H).

MS: m/z 398.3 (M+1).

### (S)-4-(4-(3-(5-oxo-5,6-dihydro-1,6-naphthyridin-7-yl)cyclopent-2-en-1-yl)piperazin-1-yl)benzonitrile (Compound 4—hydrochloride salt)

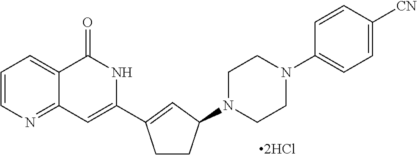 [SMILES: N#Cc1ccc(N2CCN([C@@H]3C=C(c4cc5ncccc5c(=O)[nH]4)CC3)CC2)cc1]

¹H NMR (400 MHz, DMSO-d₆) δ 11.73 (brs-exchangeable with D₂O, 1H), 11.49 (brs-exchangeable with D₂O, 1H), 9.00 (dd, J=8.8, 2.0 Hz, 1H), 8.62 (dd, J=8.0, 2.0 Hz, 1H), 7.68 (d, J=8.8 Hz, 2H), 7.62 (dd, J=8.0, 2.0 Hz, 1H), 7.15 (d, J=8.8 Hz, 2H), 6.89 (d, J=2.0 Hz, 1H), 6.82 (s, 1H), 4.73-4.53 (m, 2H), 4.15 (d, J=12.4 Hz, 2H), 3.59 (t, J=11.6 Hz, 2H), 3.35 (t, J=11.6 Hz, 2H), 3.12 (dd, J=20.0, 9.6 Hz, 2H), 2.90 (q, J=7.6 Hz, 2H), 2.40 (q, J=7.6 Hz, 2H).

MS: m/z 398.1 (M+1).

### Ethyl (R)-4-(4-(3-(5-oxo-5,6-dihydro-1,6-naphthyridin-7-yl)cyclopent-2-en-1-yl)piperazin-1-yl)benzoate (Compound 20—hydrochloride salt)

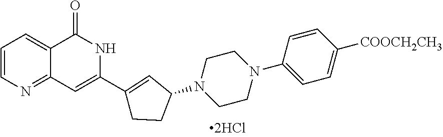

¹H NMR (400 MHz, DMSO-d₆) δ 11.71 (brs-exchangeable with D₂O, 1H), 11.28 (brs-exchangeable with D₂O, 1H), 8.99 (dd, J=4.8, 1.8 Hz, 1H), 8.61-8.59 (m, 1H), 7.85 (d, J=8.5 Hz, 2H), 7.61 (dd, J=8.1, 4.8 Hz, 1H), 7.10 (d, J=8.5 Hz, 2H), 6.89 (s, 1H), 6.81 (s, 1H), 4.69 (s, 1H), 4.26 (q, J=7.0 Hz, 2H), 4.12 (d, J=13.0 Hz, 2H), 3.60 (t, J=10.5 Hz, 2H), 3.41-3.13 (m, 4H), 2.91 (d, J=7.0 Hz, 2H), 2.40 (m, 2H), 1.30 (t, J=7.0 Hz, 3H).

MS: m/z 445.1 (M+1).

### (R)-7-(3-(4-(1-oxo-2,3-dihydro-1H-inden-5-yl)piperazin-1-yl)cyclopent-1-en-1-yl)-1,6-naphthyridin-5(6H)-one (Compound 29—hydrochloride salt)

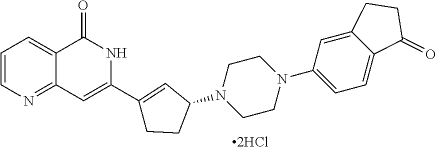 [SMILES: O=C1CCc2cc(N3CCN([C@H]4C=C(c5cc6ncccc6c(=O)[nH]5)CC4)CC3)ccc21]

¹H NMR (400 MHz, DMSO-d₆) δ 11.76 (brs-exchangeable with D₂O, 1H), 11.52 (brs-exchangeable with D₂O, 1H), 9.01 (d, J=4.8 Hz, 1H), 8.64 (d, J=8.0 Hz, 1H), 7.64 (dd, J=8.0, 4.8 Hz, 1H), 7.51 (d, J=8.8 Hz, 1H), 7.09 (m, 2H), 6.91 (s, 1H), 6.83 (s, 1H), 4.69 (s, 1H), 4.51 (s, 1H), 4.19-4.16 (m, 1H), 3.62-3.56 (m, 2H), 3.42-3.35 (m, 2H), 3.20-3.11 (m, 2H), 3.00-2.87 (m, 2H), 2.90-2.87 (m, 2H), 2.57-2.55 (m, 2H), 2.45-2.32 (m, 2H).

MS: m/z 427.3 (M+1).

### (R)-7-(3-(4-(1-oxo-1,3-dihydroisobenzofuran-5-yl)piperazin-1-yl)cyclopent-1-en-1-yl)-1,6-naphthyridin-5(6H)-one (Compound 31—hydrochloride salt)

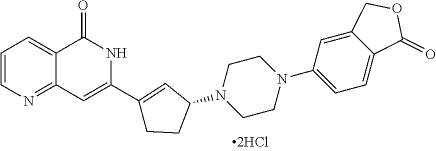 [SMILES: O=C1OCc2cc(N3CCN([C@H]4C=C(c5cc6ncccc6c(=O)[nH]5)CC4)CC3)ccc21]

¹H NMR (400 MHz, DMSO-d₆) δ 11.66 (brs-exchangeable with D₂O, 1H), 11.12 (brs-exchangeable with D₂O, 1H), 9.02-8.95 (m, 1H), 8.57 (d, J=8.0 Hz, 1H), 7.71 (d, J=8.6 Hz, 1H), 7.59 (dd, J=8.0, 4.0 Hz, 1H), 7.25 (d, J=9 Hz, 1H), 7.19 (s, 1H), 6.88 (s, 1H), 6.80 (s, 1H), 5.31 (s, 2H), 4.70 (s, 1H), 3.62 (t, J=12 Hz, 2H), 3.34 (t, J=12 Hz, 2H), 3.24-3.16 (m, 2H), 2.95-2.88 (m, 2H), 2.58-2.54 (m, 2H), 2.46-2.37 (m, 2H).

MS: m/z 429.1 (M+1).

### (R)-7-(3-(4-(1-oxoisoindolin-5-yl)piperazin-1-yl)cyclopent-1-en-1-yl)-1,6-naphthyridin-5(6H)-one (Compound 32—hydrochloride salt)

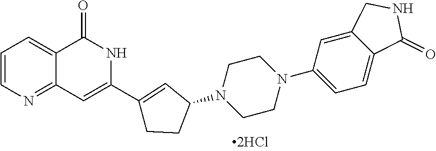 [SMILES: O=C1NCc2cc(N3CCN([C@H]4C=C(c5cc6ncccc6c(=O)[nH]5)CC4)CC3)ccc21]

¹H NMR (400 MHz, DMSO-d₆) δ 11.58 (brs-exchangeable with D₂O, 1H), 10.40 (brs-exchangeable with D₂O, 1H), 8.96 (s, 1H), 8.52 (d, J=8.1 Hz, 1H), 8.27 (s, 1H), 7.63-7.51 (m, 2H), 7.11-7.17 (m, 1H), 6.87 (s, 1H), 6.80 (s, 1H), 4.71 (s, 1H), 4.30 (s, 2H), 4.07 (d, J=12.2 Hz, 2H), 3.63 (s, 2H), 3.11-3.19 (m, 4H), 2.93 (s, 2H), 2.41-2.44 (m, 2H).

MS: m/z 428.1 (M+1).

### (R)-7-(3-(4-(4-(trifluoromethyl)phenyl)piperazin-1-yl)cyclopent-1-en-1-yl)-1,6-naphthyridin-5(6H)-one (Compound 33—hydrochloride salt)

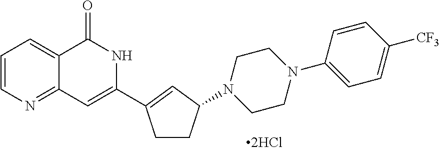 [SMILES: O=c1[nH]c(C2=C[C@H](N3CCN(c4ccc(C(F)(F)F)cc4)CC3)CC2)cc2ncccc12]

¹H NMR (400 MHz, DMSO-d₆) δ 11.69 (brs-exchangeable with D₂O, 1H), 11.30 (brs-exchangeable with D₂O, 1H), 8.99 (d, J=4.5 Hz, 1H), 8.59 (d, J=8.0 Hz, 1H), 7.64-7.54 (m, 3H), 7.18 (d, J=8.5 Hz, 2H), 6.89 (s, 1H), 6.81 (s, 1H), 4.9-4.61 (m, 1H), 4.11-4.07 (m, 2H), 3.68-3.54 (m, 2H), 3.34-3.30 (m, 2H), 3.22-3.12 (m, 2H), 2.92-2.82 (m, 2H), 1.54-1.52 (m, 2H).

MS: m/z 441.3 (M+1).

### (R)-6-(4-(3-(4-oxo-4,5-dihydrothieno[3,2-c]pyridin-6-yl)cyclopent-2-en-1-yl)piperazin-1-yl)nicotinonitrile (Compound 50—hydrochloride salt)

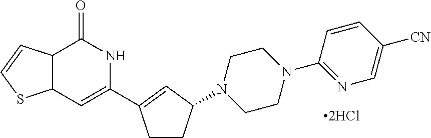 [SMILES: N#Cc1ccc(N2CCN([C@H]3C=C(C4=CC5SC=CC5C(=O)N4)CC3)CC2)nc1]

¹H NMR (400 MHz, DMSO-d₆) δ 11.56 (brs-exchangeable with D₂O, 1H), 11.35 (brs-exchangeable with D₂O, 1H), 8.58 (d, J=2.0 Hz, 1H), 7.99 (dd, J=8.0, 2.0 Hz, 1H), 7.69 (d, J=5.0 Hz, 1H), 7.51 (d, J=5.0 Hz, 1H), 7.10 (d, J=8.0 Hz, 2H), 6.73 (s, 1H), 4.68-4.61 (m, 3H), 3.61-3.45 (m, 4H), 3.17-3.00 (m, 2H), 2.89-2.7 (m, 2H), 2.39-2.37 (m, 2H).

MS: m/z 404.3 (M+1).

### Example 3: Synthesis of (R)—N-methyl-4-(4-(3-(5-oxo-5,6-dihydro-1,6-naphthyridin-7-yl)cyclopent-2-en-1-yl)piperazin-1-yl)benzamide (Compound 18)

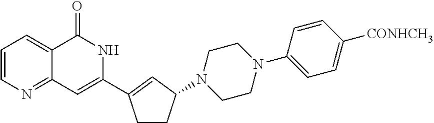

### Step 1: (R)-4-(4-(3-(5-oxo-5,6-dihydro-1,6-naphthyridin-7-yl)cyclopent-2-en-1-yl)piperazin-1-yl)benzoic acid (Compound 18a)

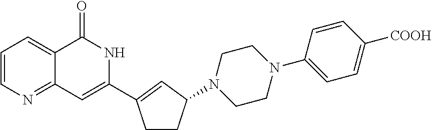 [SMILES: O=C(O)c1ccc(N2CCN([C@H]3C=C(c4cc5ncccc5c(=O)[nH]4)CC3)CC2)cc1]

To the stirred solution of ethyl (R)-4-(4-(3-(5-oxo-5,6-dihydro-1,6-naphthyridin-7-yl)cyclopent-2-en-1-yl)piperazin-1-yl)benzoate (Compound 20, 1.6 g, 3.60 mmol) in ethanol (20 ml), tetrahydrofuran (3 ml) was added NaOH (0.576 g, 14.40 mmol) in water (5 ml) and the reaction mixture was stirred at room temperature for 15 min and heated at 70° C. for 16 h. The progress of the reaction was monitored by TLC. The reaction was cooled to room temperature and the solvent was evaporated under vacuum. Water (10 ml) was added to the reaction followed by 10% HCl (till acidic pH). The solid obtained was filtered. The residue was washed with water and azeotropped with toluene to afford Ig (66%) of the titled compound as white solid.

MS: m/z 417 (M+1).

### Step 2: (R)—N-methyl-4-(4-(3-(5-oxo-5,6-dihydro-1,6-naphthyridin-7-yl)cyclopent-2-en-1-yl)piperazin-1-yl)benzamide (Compound 18)

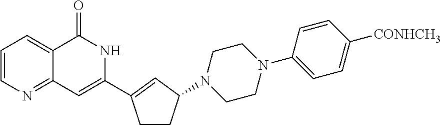

To the cooled (10° C.) and stirred solution of (R)-4-(4-(3-(5-oxo-5,6-dihydro-1,6-naphthyridin-7-yl)cyclopent-2-en-1-yl)piperazin-1-yl)benzoic acid (Compound 18a, 0.5 g, 1.20 mmol) in dimethyl sulphoxide (15 ml) was added HATU (1.14 g, 3.00 mmol), DIPEA (0.839 ml, 4.80 mmol). The reaction mixture was warmed to room temperature and stirred for 0.5 hr. The reaction mixture was cooled to 0° C. and methylamine (2.4 ml, 4.80 mmol) was added and the reaction was stirred at room temperature for 16 hrs. The progress of the reaction was monitored by TLC. Ice cold water (20 ml) was added and reaction mass was filtered. The residue obtained was washed with water; dried under vacuum to afford 300 mg (58%) of the titled compound as yellow solid.

¹H NMR (400 MHz, DMSO-d₆) δ 11.48 (brs-exchangeable with D₂O, 1H), 8.91 (dd, J=4.5, 2.0 Hz, 1H), 8.48 (d, J=8.0 Hz, 1H), 8.19-8.11 (m, 1H), 7.75-7.69 (m, 2H), 7.51-7.44 (m, 1H), 7.0-6.92 (m, 2H), 6.60 (s, 1H), 3.98-3.88 (m, 1H), 3.27 (d, J=5.2 Hz, 3H), 2.92-2.86 (m, 1H), 2.83-2.57 (m, 7H), 2.55 (s, 2H), 2.15-2.04 (m, 1H), 1.98-1.85 (m, 1H).

MS: m/z 430.1 (M+1).

### Step 3: (R)—N-methyl-4-(4-(3-(5-oxo-5,6-dihydro-1,6-naphthyridin-7-yl)cyclopent-2-en-1-yl)piperazin-1-yl)benzamide (Compound 18—hydrochloride salt)

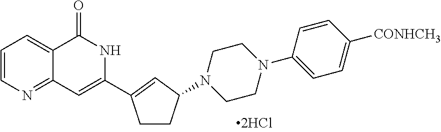

To the stirred suspension of (R)—N-methyl-4-(4-(3-(5-oxo-5,6-dihydro-1,6-naphthyridin-7-yl)cyclopent-2-en-1-yl)piperazin-1-yl)benzamide (Compound 18, 0.300 g, 0.698 mmol) in methanol (5 ml) and DCM (5 ml) was added dropwise HCl (1.397 ml, 5.59 mmol) 4M in Dioxane at room temperature. The reaction mixture was stirred for 1 hr. To the reaction mixture, diethyl ether (10 mL) was added. The solid obtained was filtered and dried under vacuum to afford 280 mg (80%) of the titled compound as white solid.

¹H NMR (400 MHz, DMSO-d₆) δ11.67 (brs-exchangeable with D₂O, 1H), 10.99 (brs-exchangeable with D₂O, 1H), 8.98 (d, J=4.5 Hz, 1H), 8.57 (d, J=8.1 Hz, 1H), 8.25 (d, J=4.5 Hz, 1H), 7.78 (d, J=8.5 Hz, 2H), 7.59 (dd, J=8.1, 4.7 Hz, 1H), 7.06 (d, J=8.5 Hz, 2H), 6.89 (s, 1H), 6.80 (s, 1H), 4.69 (s, 1H), 4.54 (s, 2H), 4.06 (d, J=9.6 Hz, 2H), 3.58 (d, J=11.0 Hz, 2H), 3.19 (s, 2H), 2.91 (s, 2H), 2.76 (d, J=4.0 Hz, 3H), 2.42-2.38 (m, 2H).

MS: m/z 430.1 (M+1).

The following compounds were prepared using the procedure described above in Example 3 with appropriate changes to the reactants and reaction conditions.

### (R)-4-(4-(3-(5-oxo-5,6-dihydro-1,6-naphthyridin-7-yl)cyclopent-2-en-1-yl)piperazin-1-yl)benzamide (Compound 19—hydrochloride salt)

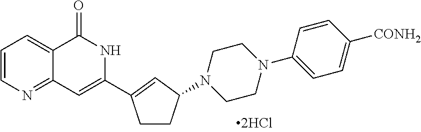 [SMILES: NC(=O)c1ccc(N2CCN([C@H]3C=C(c4cc5ncccc5c(=O)[nH]4)CC3)CC2)cc1]

¹H NMR (400 MHz, DMSO-d₆) δ 11.73 (brs-exchangeable with D₂O, 1H), 11.34 (brs-exchangeable with D₂O, 1H), 8.99 (dd, J=5.0, 1.7 Hz, 1H), 8.62 (d, J=8.0 Hz, 1H), 7.81 (d, J=8.5 Hz, 2H), 7.62 (dd, J=8.0, 5.0 Hz, 1H), 7.05 (d, J=8.5 Hz, 2H), 6.91 (s, 1H), 6.82 (m, 3H), 4.69 (s, 1H), 4.05 (d, J=12.3 Hz, 2H), 3.58 (t, J=10.6 Hz, 2H), 3.29-3.13 (m, 4H), 2.92-2.88 (m, 2H), 2.41 (d, J=8.0 Hz, 2H).

MS: m/z 416.3 (M+1).

### (R)—N-methyl-4-(4-(3-(4-oxo-4,5-dihydrothieno[3,2-c]pyridin-6-yl)cyclopent-2-en-1-yl)piperazin-1-yl)benzamide (Compound 49—hydrochloride salt)

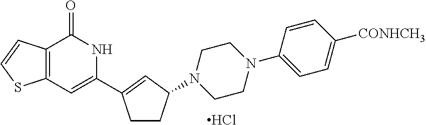

¹H NMR (400 MHz, DMSO-d₆) δ 11.41 (brs-exchangeable with D₂O, 1H), 8.29 (brs-exchangeable with D₂O, 1H), 7.78 (d, J=8.5 Hz, 2H), 7.69 (d, J=5.0 Hz, 1H), 7.51 (d, J=5.0 Hz, 1H), 7.10 (s, 1H), 7.05 (d, J=8.5 Hz, 2H), 6.79 (s, 1H), 4.63 (s, 1H), 4.03 (d, J=12.0 Hz, 2H), 3.57 (t, J=13 Hz, 2H), 3.22-3.32 (m, 2H), 3.20-3.08 (m, 2H), 2.92-2.79 (m, 2H), 2.76 (s, 3H), 2.40-2.35 (m, 2H).

MS: m/z 435.2 (M+1).

### Example 4: Synthesis of (R)-4-(4-(3-(1-oxo-1,2-dihydro-2,6-naphthyridin-3-yl)cyclopent-2-en-1-yl)piperazin-1-yl)benzonitrile (Compound 36)

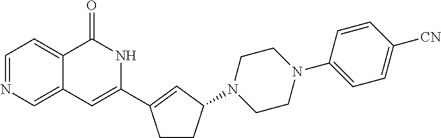 [SMILES: N#Cc1ccc(N2CCN([C@H]3C=C(c4cc5cnccc5c(=O)[nH]4)CC3)CC2)cc1]

### Step 1: (R)-methyl 3-((3-(4-(4-cyanophenyl)piperazin-1-yl)cyclopent-1-en-1-yl)ethynyl)isonicotinate (Compound 36a)

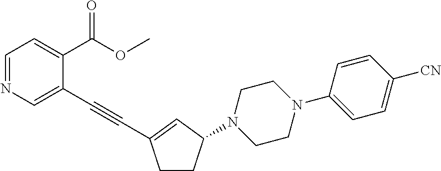 [SMILES: COC(=O)c1ccncc1C#CC1=C[C@H](N2CCN(c3ccc(C#N)cc3)CC2)CC1]

To a solution of methyl-3-bromoisonicotinate (2.337 g, 10.82 mmol) in anhydrous acetonitrile (100 ml, degassed by nitrogen gas) was added bis(triphenylphosphine) palladium (II) chloride (0.633 g, 0.901 mmol) at 25° C. The reaction mixture was heated and stirred at 80° C. for 10 min and to this warmed reaction mixture was added diisopropylethyl amine (9.45 ml, 54.10 mmol) followed by the addition of a solution of (R)-4-(4-(3-ethynylcyclopent-2-en-1-yl)piperazin-1-yl)benzonitrile (Compound 1j-Prepared according to the procedure given in Example 1; step 10, 2.5 g, 9.01 mmol) in acetonitrile (25 ml). The reaction mixture was heated at same temperature for 18 hrs. The progress of the reaction was monitored by TLC. The reaction mixture was cooled to room temperature and diluted with ethyl acetate (200 ml), washed with water (100 ml). The aqueous layer was again extracted with ethyl acetate (100 ml) and the combined organic layer was dried over sodium sulfate, filtered and concentrated under reduced pressure to give crude intermediate, which was purified by flash column chromatography over silica gel (100-200 mesh) using 70-100% ethyl acetate in hexane as eluent to obtain title compound (1.5 g, 40.3% yield).

¹H NMR (400 MHz, CDCl₃) δ 8.89-8.81 (m, 1H), 8.70-8.60 (m, 1H), 7.81-7.76 (m, 1H), 7.53 (d, J=8.6 Hz, 2H), 6.89 (d, J=8.4 Hz, 2H), 6.31 (s, 1H), 3.99 (s, 3H), 3.54-3.38 (m, 4H), 2.85-2.63 (m, 6H), 2.24-2.02 (m, 3H).

MS: m/z 413 (M+1).

### Step 2: (R)-3-((3-(4-(4-cyanophenyl)piperazin-1-yl)cyclopent-1-en-1-yl)ethynyl) isonicotinic acid (Compound 36b)

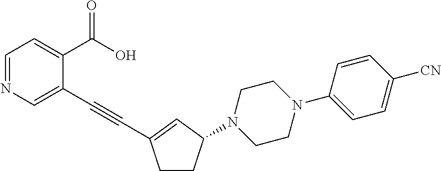 [SMILES: N#Cc1ccc(N2CCN([C@H]3C=C(C#Cc4cnccc4C(=O)O)CC3)CC2)cc1]

To a stirred solution of (R)-methyl 3-((3-(4-(4-cyanophenyl)piperazin-1-yl)cyclopent-1-en-1-yl)ethynyl)isonicotinate (Compound 36a, 1.5 g, 3.64 mmol) in methanol (100 ml) was added aqueous sodium hydroxide (0.582 g, 14.55 mmol) in water (10 ml), at 25-30° C. The reaction mixture was stirred for 2 hrs at the same temperature. The progress of the reaction was monitored by TLC. The reaction mixture was distilled under reduced pressure completely till dryness. The sticky solid obtained was dissolved in water (50 ml), a clear solution was observed and then washed with ethyl acetate (25 ml) to remove the impurities. The aqueous layer was separated, cooled at 0-5° C. and then the pH was adjusted ˜3 using dilute aqueous hydrochloric acid (1:1) at 0-5° C., the solid compound was precipitated out. The obtained solid compound was stirred for 10-15 min at same temperature and filtered through Buchner funnel, washed with ice cold water (10 ml), dried till dryness to obtain the title compound (1.2 gm, 83.0% yield).

¹H NMR (400 MHz, DMSO-d₆) δ 8.79 (s, 1H), 8.68 (d, J=5.0 Hz, 1H), 7.75 (d, J=5.1 Hz, 1H), 7.61 (d, J=8.5 Hz, 2H), 7.05 (d, J=8.6 Hz, 2H), 6.34 (s, 1H), 4.14 (s, 1H), 3.57-3.42 (m, 6H), 2.92-2.73 (m, 4H), 2.66-2.57 (m, 1H), 2.18-1.95 (m, 2H).

MS: m/z 399 (M+1).

### Step 3: (R)-4-(4-(3-(1-oxo-1H-pyrano[4,3-c]pyridin-3-yl)cyclopent-2-en-1-yl) piperazin-1-yl)benzonitrile (Compound 36c)

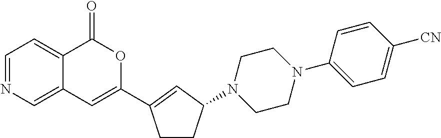 [SMILES: N#Cc1ccc(N2CCN([C@H]3C=C(c4cc5cnccc5c(=O)o4)CC3)CC2)cc1]

To a solution of (R)-3-((3-(4-(4-cyanophenyl)piperazin-1-yl)cyclopent-1-en-1-yl)ethynyl)isonicotinic acid (Compound 36b, 1.1 g, 2.76 mmol) in anhydrous dichloromethane:tetrahydrofuran (100 ml, Ratio: 1:1), was added trifluoromethane sulphonic acid (0.621 g, 4.14 mmol) at 0-5° C. and the reaction mixture was stirred for 48 hrs. The progress of the reaction was monitored by TLC. The reaction mixture was cooled at 0-5° C. and then diluted with diethyl ether (25 ml), a solid compound was precipitated out. The reaction mixture was stirred for 30 min at same temperature and filtered through Buchner funnel, washed with diethyl ether (10 ml), and dried completely to obtain the title compound (490 mg, 44.5% yield).

¹H NMR (400 MHz, DMSO-d₆) δ 9.84 (s, 1H), 9.10 (s, 1H), 8.83 (d, J=5.7 Hz, 1H), 7.68 (d, J=8.5 Hz, 2H), 7.13 (d, J=8.7 Hz, 2H), 6.61 (s, 1H), 4.72 (s, 1H), 4.24-4.07 (m, 2H), 3.75-3.50 (m, 2H), 3.35-3.20 (m, 1H), 3.18-2.99 (m, 3H), 2.96-2.68 (m, 3H), 2.46-2.28 (m, 2H).

MS: m/z 399 (M+1).

### Step 4: (R)-4-(4-(3-(1-oxo-1,2-dihydro-2,6-naphthyridin-3-yl)cyclopent-2-en-1-yl)piperazin-1-yl)benzonitrile (Compound 36)

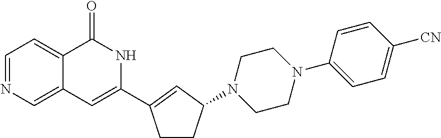 [SMILES: N#Cc1ccc(N2CCN([C@H]3C=C(c4cc5cnccc5c(=O)[nH]4)CC3)CC2)cc1]

To a solution of (R)-4-(4-(3-(1-oxo-1H-pyrano[4,3-c]pyridin-3-yl)cyclopent-2-en-1-yl)piperazin-1-yl)benzonitrile (Compound 36c, 300 mg, 0.753 mmol) in anhydrous tetrahydrofuran (5 ml), was added ammonia in methanol (10.76 ml, 75 mmol) at 25° C. The reaction mixture in steel bomb reactor was stirred for 12 hrs at 80-85° C. The progress of the reaction was monitored by TLC. The reaction mixture was cooled to room temperature and then concentrated under reduced pressure to obtain crude product which was purified by flash column chromatography over silica gel (100-200 mesh) using 2-5% methanol in dichloromethane as eluent to obtain title compound (0.050 g, 16.71% yield).

¹H NMR (400 MHz, DMSO-d₆) δ 11.53 (brs-exchangeable with D₂O, 1H), 9.08 (dd, J=8.8, 2.0 Hz, 1H), 8.61 (dd, J=8.0, 2.0 Hz, 1H), 7.97 (d, J=8.8 Hz, 2H), 7.58 (dd, J=8.0, 2.0 Hz, 1H), 7.03 (d, J=8.8 Hz, 2H), 6.91 (d, J=2.0 Hz, 1H), 6.71 (s, 1H), 3.97-3.86 (m, 1H), 3.43-3.35 (m, 4H), 2.82-2.70 (m, 1H), 2.68-2.55 (m, 4H), 2.15-2.01 (m, 1H), 1.98-1.80 (m, 1H), 1.90-1.75 (m, 1H).

MS: m/z 398.3 (M+1).

### Step 5: (R)-4-(4-(3-(1-oxo-1,2-dihydro-2,6-naphthyridin-3-yl)cyclopent-2-en-1-yl)piperazin-1-yl)benzonitrile (Compound 36—hydrochloride salt)

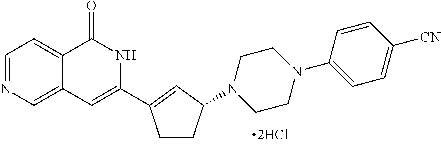 [SMILES: N#Cc1ccc(N2CCN([C@H]3C=C(c4cc5cnccc5c(=O)[nH]4)CC3)CC2)cc1]

A solution of (R)-4-(4-(3-(1-oxo-1,2-dihydro-2,6-naphthyridin-3-yl)cyclopent-2-en-1-yl)piperazin-1-yl)benzonitrile (Compound 36, 40 mg, 0.101 mmol) in dichloromethane (2 ml) and methanol (2 ml) was heated at 65° C. and was added hydrochloric acid in methanol (0.587 ml, 0.352 mmol, 3M solution) at same temperature in small portions over a period of 5 min. The reaction mixture was then stirred for 30 min at 25° C. The reaction mixture was cooled to room temperature, diluted with diethyl ether (10 ml), and the product was collected upon filtration. The solid compound was washed with diethyl ether (10 ml) and dried under reduced pressure for 3 hr at 40° C. to obtain the title compound (0.035 g, 88% yield).

¹H NMR (400 MHz, DMSO-d₆) δ 11.74 (brs-exchangeable with D₂O, s, 1H), 11.46 (brs-exchangeable with D₂O, s, 1H), 9.19 (s, 1H), 8.70 (d, J=5.4 Hz, 1H), 8.10 (d, J=5.4 Hz, 1H), 7.68 (d, J=8.7 Hz, 2H), 7.14 (d, J=8.7 Hz, 2H), 6.93 (s, 1H), 6.83 (s, 1H), 4.67 (s, 1H), 4.14 (d, J=13.4 Hz, 2H), 3.64-3.52 (m, 2H), 3.34 (t, J=13.1 Hz, 2H), 3.23-3.07 (m, 2H), 2.95-2.80 (m, 2H), 2.45-2.35 (m, 2H).

MS: m/z 398.3 (M+1).

The following compounds were prepared using the procedure described above in Example 4 with appropriate changes to the reactants and reaction conditions. If required, compound 23b″ is used as starting material and procedure described in step 3 to step 6 of example 2 is followed to prepare required intermediate.

### (R)-4-(4-(3-(3-fluoro-5-oxo-5,6-dihydro-1,6-naphthyridin-7-yl)cyclopent-2-en-1-yl)piperazin-1-yl)benzonitrile (Compound 2—hydrochloride salt)

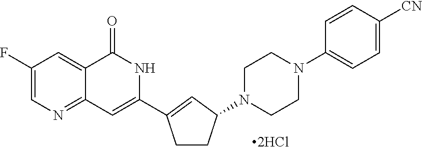 [SMILES: N#Cc1ccc(N2CCN([C@H]3C=C(c4cc5ncc(F)cc5c(=O)[nH]4)CC3)CC2)cc1]

¹H NMR (400 MHz, DMSO-d₆) δ 11.70 (brs-exchangeable with D₂O, 1H), 11.32 (brs-exchangeable with D₂O, 1H), 9.01 (d, J=3.0 Hz, 1H), 8.29 (dd, J=8.5, 3.0 Hz, 1H), 7.68 (d, J=8.4 Hz, 2H), 7.15 (d, J=8.6 Hz, 2H), 6.83 (d, J=13.2 Hz, 2H), 4.67 (d, J=6.9 Hz, 1H), 4.15 (d, J=13.3 Hz, 2H), 3.55-3.60 (m, 2H), 3.40-3.25 (m, 2H), 3.25-3.05 (m, 2H), 2.97-2.83 (m, 2H), 2.38 (s, 2H).

MS: m/z 416.1 (M+1).

### (S)-4-(4-(3-(3-fluoro-5-oxo-5,6-dihydro-1,6-naphthyridin-7-yl)cyclopent-2-en-1-yl)piperazin-1-yl)benzonitrile (Compound 5—hydrochloride salt)

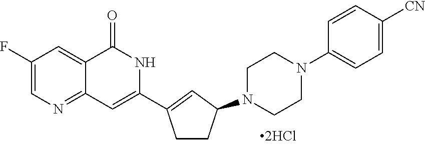 [SMILES: N#Cc1ccc(N2CCN([C@@H]3C=C(c4cc5ncc(F)cc5c(=O)[nH]4)CC3)CC2)cc1]

¹H NMR (400 MHz, DMSO-d₆) δ 11.69 (brs-exchangeable with D₂O, 1H), 11.30 (brs-exchangeable with D₂O, 1H), 9.00 (d, J=3.0 Hz, 1H), 8.29 (dd, J=8.5, 3.0 Hz, 1H), 7.68 (d, J=8.4 Hz, 2H), 7.15 (d, J=8.6 Hz, 2H), 6.83 (d, J=13.2 Hz, 2H), 4.67 (d, J=6.9 Hz, 1H), 4.15 (d, J=13.3 Hz, 2H), 3.55-3.60 (m, 2H), 3.40-3.25 (m, 2H), 3.25-3.05 (m, 2H), 2.96-2.83 (m, 2H), 2.39 (s, 2H).

MS: m/z 416 (M+1).

### (R)-4-(4-(3-(8-oxo-7,8-dihydro-1,7-naphthyridin-6-yl)cyclopent-2-en-1-yl)piperazin-1-yl)benzonitrile (Compound 37—hydrochloride salt)

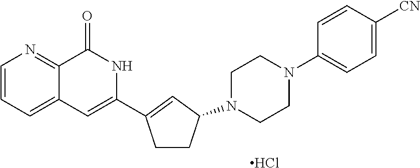 [SMILES: N#Cc1ccc(N2CCN([C@H]3C=C(c4cc5cccnc5c(=O)[nH]4)CC3)CC2)cc1 ; Cl]

¹H NMR (400 MHz, DMSO-d₆): δ 11.83 (brs-exchangeable with D₂O, 1H), 11.60 (brs-exchangeable with D₂O, 1H), 8.87-8.84 (m, 1H), 8.38 (d, J=8.2 Hz, 1H), 7.89-7.85 (m, 1H), 7.69-7.66 (m, 2H), 7.14-7.11 (m, 2H), 6.83-6.85 (m, 2H), 4.62 (s, 1H), 4.14 (d, J=13.3 Hz, 2H), 3.59-3.52 (m, 2H), 3.35-3.25 (m, 2H), 3.20-3.07 (m, 2H), 2.88-2.78 (m, 2H), 2.41-2.34 (m, 2H).

MS: m/z 398.3 (M+1).

### (R)-4-(4-(3-(1-oxo-1,2-dihydro-2,7-naphthyridin-3-yl)cyclopent-2-en-1-yl)piperazin-1-yl)benzonitrile (Compound 38—hydrochloride salt)

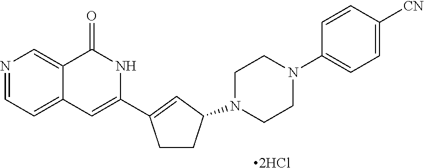 [SMILES: N#Cc1ccc(N2CCN([C@H]3C=C(c4cc5ccncc5c(=O)[nH]4)CC3)CC2)cc1 ; "·2HCl"]

¹H NMR (400 MHz, DMSO-d₆) δ 12.08 (brs-exchangeable with D₂O, 1H), 11.78 (brs-exchangeable with D₂O, 1H), 9.41 (s, 1H), 8.80 (d, J=6.0 Hz, 1H), 7.97 (d, J=6.1 Hz, 1H), 7.68 (d, J=8.6 Hz, 2H), 7.15 (d, J=8.6 Hz, 2H), 7.00 (s, 1H), 6.95 (s, 1H), 4.69 (s, 1H), 4.22-4.08 (m, 2H), 3.66-3.51 (m, 2H), 3.34-3.32 (m, 2H), 3.24-3.06 (m, 2H), 2.99-2.80 (m, 2H), 2.44-2.37 (m, 2H).

MS: m/z 398.4 (M+1).

### (R)-4-(4-(3-(5-oxo-5,6-dihydropyrido[4,3-d]pyrimidin-7-yl)cyclopent-2-en-1-yl)piperazin-1-yl)benzonitrile (Compound 40—hydrochloride salt)

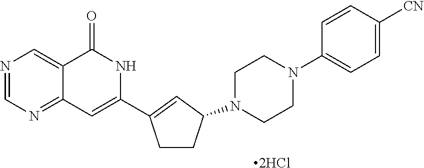 [SMILES: N#Cc1ccc(N2CCN([C@H]3C=C(c4cc5ncncc5c(=O)[nH]4)CC3)CC2)cc1 ; "·2HCl"]

¹H NMR (400 MHz, DMSO-d₆) δ 11.93 (brs-exchangeable with D₂O, 1H), 11.55 (brs-exchangeable with D₂O, 1H), 9.43 (s, 1H), 9.37 (s, 1H), 7.68 (d, J=8.5 Hz, 2H), 7.14 (d, J=8.7 Hz, 2H), 6.94 (s, 1H), 6.72 (s, 1H), 4.75-4.64 (m, 1H), 4.20-4.11 (m, 2H), 3.59-3.53 (m, 2H), 3.37-3.28 (m, 2H), 3.26-3.07 (m, 2H), 2.96-2.84 (m, 2H), 2.45-2.35 (m, 2H).

MS: m/z 399.1 (M+1).

### (R)-4-(4-(3-(5-oxo-5,6-dihydropyrido[3,4-b]pyrazin-7-yl)cyclopent-2-en-1-yl)piperazin-1-yl)benzonitrile (Compound 41—hydrochloride salt)

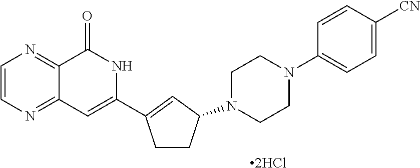 [SMILES: N#Cc1ccc(N2CCN([C@H]3C=C(c4cc5nccnc5c(=O)[nH]4)CC3)CC2)cc1 ; "·2HCl"]

¹H NMR (400 MHz, DMSO-d₆) δ 11.84 (brs-exchangeable with D₂O, 1H), 11.59 (brs-exchangeable with D₂O, 1H), 8.98 (s, 1H), 8.83 (s, 1H), 7.68 (d, J=8.5 Hz, 2H), 7.14 (d, J=8.5 Hz, 2H), 6.89 (s, 1H), 6.78 (s, 1H), 4.68 (s, 1H), 4.14 (d, J=13.5 Hz, 2H), 3.65-3.53 (m, 2H), 3.34 (d, J=13.5 Hz, 2H), 3.19-3.06 (m, 2H), 2.91 (s, 2H), 2.44-2.34 (m, 2H).

MS: m/z 399.1 (M+1).

### (R)-4-(4-(3-(4-oxo-4,5-dihydrothieno[3,2-c]pyridin-6-yl)cyclopent-2-en-1-yl)piperazin-1-yl)benzonitrile (Compound 42—hydrochloride salt)

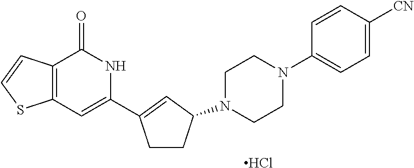 [SMILES: N#Cc1ccc(N2CCN([C@H]3C=C(c4cc5sccc5c(=O)[nH]4)CC3)CC2)cc1 ; Cl]

¹H NMR (400 MHz, DMSO-d₆): δ 11.38 (brs-exchangeable with D₂O, 1H), 11.08 (brs-exchangeable with D₂O, 1H), 7.75-7.64 (m, 3H), 7.51 (d, J=5.3 Hz, 1H), 7.21-7.07 (m, 3H), 6.75 (s, 1H), 4.64 (s, 1H), 4.14 (d, J=13.3 Hz, 2H), 3.59-3.52 (m, 2H), 3.35-3.25 (m, 2H), 3.19-3.07 (m, 2H), 2.90-2.78 (m, 2H), 2.42-2.31 (m, 2H).

MS: m/z 403.1 (M+1).

### (R)-4-(4-(3-(4-oxo-4,5-dihydrothiazolo[5,4-c]pyridin-6-yl)cyclopent-2-en-1-yl)piperazin-1-yl)benzonitrile (Compound 43—hydrochloride salt)

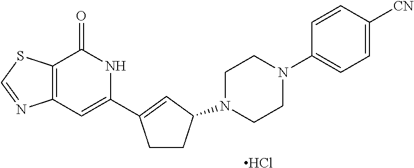 [SMILES: N#Cc1ccc(N2CCN([C@H]3C=C(c4cc5ncsc5c(=O)[nH]4)CC3)CC2)cc1 ; Cl]

¹H NMR (400 MHz, DMSO-d₆): δ 11.81 (brs-exchangeable with D₂O, 1H), 10.98 (brs-exchangeable with D₂O, 1H), 9.60 (s, 1H), 7.68 (d, J=8.2 Hz, 1H), 7.19-7.11 (m, 3H), 6.79 (s, 1H), 4.62 (s, 1H), 4.67 (m, 1H), 4.17-4.13 (m, 2H), 3.61-3.57 (m, 2H), 3.37-3.27 (m, 4H), 2.92-2.88 (m, 2H), 2.41-2.37 (m, 2H).

MS: m/z 404.1 (M+1).

### (R)-4-(4-(3-(4-oxo-4,5-dihydrothiazolo[4,5-c]pyridin-6-yl)cyclopent-2-en-1-yl)piperazin-1-yl)benzonitrile (Compound 44—hydrochloride salt)

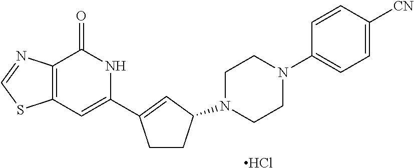 [SMILES: N#Cc1ccc(N2CCN([C@H]3C=C(c4cc5scnc5c(=O)[nH]4)CC3)CC2)cc1 ; Cl]

¹H NMR (400 MHz, DMSO-d₆) δ 11.67 (brs-exchangeable with D₂O, 1H), 10.96 (brs-exchangeable with D₂O, 1H), 9.21 (s, 1H), 7.74-7.65 (m, 2H), 7.20 (s, 1H), 7.19-7.12 (m, 2H), 6.79 (s, 1H), 4.71-4.62 (m, 1H), 4.22-4.11 (m, 2H), 3.62-3.53 (m, 2H), 3.34-3.23 (m, 2H), 3.21-3.07 (m, 2H), 2.90-2.79 (m, 2H), 2.44-2.29 (m, 2H).

MS: m/z 404.2 (M+1).

### (S)-4-(4-(3-(4-oxo-4,5-dihydrothieno[3,2-c]pyridin-6-yl)cyclopent-2-en-1-yl)piperazin-1-yl)benzonitrile (Compound 45—hydrochloride salt)

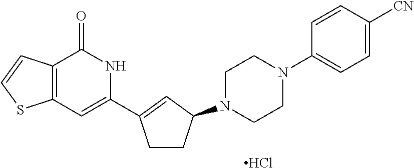 [SMILES: N#Cc1ccc(N2CCN([C@@H]3C=C(c4cc5sccc5c(=O)[nH]4)CC3)CC2)cc1 ; Cl]

¹H NMR (400 MHz, DMSO-d₆): δ 11.39 (brs-exchangeable with D₂O, 1H), 11.07 (brs-exchangeable with D₂O, 1H), 7.75-7.64 (m, 3H), 7.51 (d, J=5.3 Hz, 1H), 7.21-7.07 (m, 3H), 6.75 (s, 1H), 4.64 (s, 1H), 4.14 (d, J=13.3 Hz, 2H), 3.59-3.52 (m, 2H), 3.35-3.25 (m, 2H), 3.19-3.07 (m, 2H), 2.91-2.77 (m, 2H), 2.44-2.30 (m, 2H).

MS: m/z 425.0 (M+23).

### (S)-4-(4-(3-(4-oxo-4,5-dihydrothiazolo[5,4-c]pyridin-6-yl)cyclopent-2-en-1-yl)piperazin-1-yl)benzonitrile (Compound 46—hydrochloride salt)

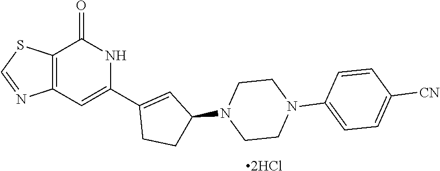 [SMILES: N#Cc1ccc(N2CCN([C@@H]3C=C(c4cc5ncsc5c(=O)[nH]4)CC3)CC2)cc1 ; "·2HCl"]

¹H NMR (400 MHz, DMSO-d₆): δ 11.80 (brs-exchangeable with D₂O, 1H), 10.99 (brs-exchangeable with D₂O, 1H), 9.60 (s, 1H), 7.68 (d, J=8.2 Hz, 1H), 7.19-7.11 (m, 3H), 6.79 (s, 1H), 4.63 (s, 1H), 4.67 (m, 1H), 4.17-4.13 (m, 2H), 3.61-3.57 (m, 2H), 3.37-3.27 (m, 4H), 2.92-2.89 (m, 2H), 2.41-2.38 (m, 2H).

MS: m/z 404.2 (M+1).

### (R)-4-(4-(3-(1-methyl-4-oxo-4,5-dihydro-1H-pyrazolo[4,3-c]pyridin-6-yl)cyclopent-2-en-1-yl)piperazin-1-yl)benzonitrile (Compound 53—hydrochloride salt)

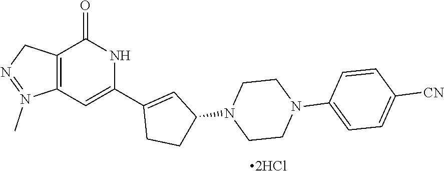

¹H NMR (400 MHz, DMSO-d₆) δ 11.25 (brs-exchangeable with D₂O, 1H), 10.99 (brs-exchangeable with D₂O, 1H), 8.04 (s, 1H), 7.68 (d, J=8.4 Hz, 2H), 7.15 (d, J=8.6 Hz, 2H), 6.83 (s, 1H), 6.76 (s, 1H), 4.65 (s, 1H), 4.18-4.11 (m, 2H), 3.99 (s, 3H), 3.77-3.48 (m, 4H), 3.38-3.26 (m, 2H), 3.22-3.07 (m, 2H), 2.96-2.80 (m, 2H).

MS: m/z 423.1 (M+23).

### Example 5: Synthesis of (R)-7-(3-(4-(4-fluorophenyl)piperazin-1-yl)cyclopent-1-en-1-yl)-1,6-naphthyridin-5(6H)-one (Compound 22)

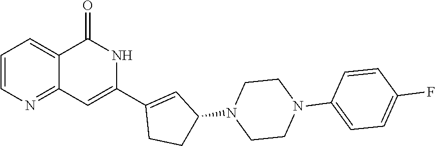 [SMILES: O=c1[nH]c(C2=C[C@H](N3CCN(c4ccc(F)cc4)CC3)CC2)cc2ncccc12]

### Step 1: 3-(4-(4-fluorophenyl)piperazin-1-yl)cyclopent-1-enecarbonitrile (Compound 22a)

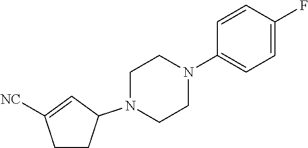 [SMILES: N#CC1=CC(N2CCN(c3ccc(F)cc3)CC2)CC1]

To a stirred solution of 1-(4-fluorophenyl)piperazine (50.3 g, 279 mmol) in acetonitrile (700 ml), was added potassium carbonate (80 g, 581 mmol) at 0° C. and stirred for 30 min at room temperature and followed by 3-bromocyclopent-1-enecarbonitrile (Compound 1a, 40 g, 233 mmol) at 0° C. The reaction mixture was stirred at room temperature for 3 hr. The progress of the reaction was monitored by TLC. The reaction mixture was diluted with water (3 lit) and extracted with ethyl acetate (4×700 ml). The combined organic layer was dried over anhydrous sodium sulfate, filtered and concentrated under reduced pressure to give the crude compound which was purified by column chromatography over silica gel (100-200 mesh) using 20-50% ethyl acetate in hexane as eluent to obtain the title compound (50 g, 79% yield).

¹H NMR (400 MHz, CDCl₃): δ 7.08-7.01 (m, 2H), 6.99 (q, J=2.1 Hz, 1H), 6.96-6.90 (m, 2H), 3.97-3.86 (m, 1H), 3.05 (t, J=4.9 Hz, 4H), 2.71-2.51 (m, 6H), 2.10-1.98 (m, 1H), 1.94-1.79 (m, 1H).

MS: m/z 272.4 (M+1).

### Step 2: 3-(4-(4-fluorophenyl)piperazin-1-yl)cyclopent-1-enecarbaldehyde (Compound 22b)

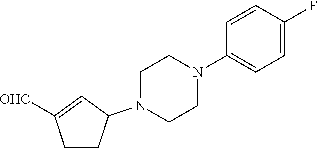 [SMILES: O=CC1=CC(N2CCN(c3ccc(F)cc3)CC2)CC1]

To a stirred solution of 3-(4-(4-fluorophenyl)piperazin-1-yl)cyclopent-1-enecarbonitrile (Compound 22a, 50 g, 184 mmol) in dichloromethane (100 ml) was added di-isobutyl aluminium hydride (221.0 ml, 221.0 mmol, 1M solution in toluene) at −78° C. over a period of 30 min. The reaction mixture was warmed to 25-30° C. and stirred for 18-20 hr. The progress of the reaction was monitored by TLC. The reaction mixture was diluted with ethylacetate (250 ml) and quenched with saturated aqueous solution of ammonium chloride (100 ml). The reaction mass was filtered through a Celite bed, and the Celite bed was washed with ethyl acetate (100 ml). The organic layer was dried over anhydrous sodium sulfate, filtered and concentrated under reduced pressure to give crude product which was purified by column chromatography over silica gel (100-200 mesh) using 45-50% ethyl acetate in hexane as eluent to obtain the title compound (12 g, 23% yield).

¹H NMR (400 MHz, CDCl₃) δ 9.87 (s, 1H), 6.98 (t, J=8.7 Hz, 2H), 6.94-6.86 (m, 3H), 4.10-3.99 (m, 1H), 3.17 (t, J=4.9 Hz, 4H), 3.03-2.87 (m, 1H), 2.83-2.66 (m, 4H), 2.56-2.44 (m, 1H), 2.26-2.14 (m, 1H), 2.07-1.97 (m, 1H).

MS: m/z 274.4 (M+1).

### Step 3: 1-(3-ethynylcyclopent-2-en-1-yl)-4-(4-fluorophenyl)piperazine (Compound 22c)

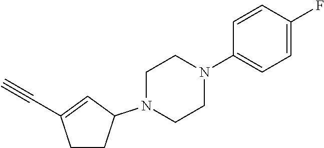 [SMILES: C#CC1=CC(N2CCN(c3ccc(F)cc3)CC2)CC1]

To a stirred solution of trimethylsilyldiazomethane (32.8 ml, 65.6 mmol, 2M solution in hexane) in anhydrous tetrahydrofuran (100 ml) was added n-butyl lithium (41.0 ml, 65.6 mmol) at −78° C. The reaction mixture was stirred for 30 min at the same temperature. To this reaction mixture a solution of 3-(4-(4-fluorophenyl)piperazin-1-yl)cyclopent-1-enecarbaldehyde (Compound 22b, 12 g, 43.7 mmol) in tetrahydrofuran (20 ml) was added at same temperature and warmed to room temperature and stirred for 18-20 hr. The progress of the reaction was monitored by TLC. The reaction mixture was diluted with ethyl acetate (150 ml) and washed with water (2×100 ml). The organic layer was dried over sodium sulfate, filtered and concentrated under reduced pressure to give crude product, which was purified by flash column chromatography over silica gel (100-200 mesh) using 45-50% of ethyl acetate in hexane as eluent to obtain the title compound (6.0 g, 50% yield).

¹H NMR (400 MHz, CDCl₃) δ 7.01-6.95 (m, 2H), 6.89 (dd, J=9.2, 4.6 Hz, 2H), 6.21 (q, J=2.2 Hz, 1H), 3.97 (s, 1H), 3.17 (s, 4H), 3.08 (s, 1H), 2.74 (s, 4H), 2.64-2.43 (m, 2H), 2.16-1.91 (m, 2H).

MS: m/z 271 (M+1).

A chiral separation of racemic 1-(3-ethynylcyclopent-2-en-1-yl)-4-(4-fluorophenyl)piperazine (Compound 22c-racemic, 30 g) was carried out using a chiral column to obtain

### (R)-1-(3-ethynylcyclopent-2-en-1-yl)-4-(4-fluorophenyl)piperazine (Compound 22c′; 12 g)

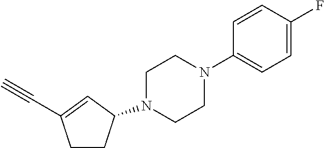 [SMILES: C#CC1=C[C@H](N2CCN(c3ccc(F)cc3)CC2)CC1]

¹H NMR (400 MHz, CDCl₃) δ 7.01-6.95 (m, 2H), 6.89 (dd, J=9.2, 4.6 Hz, 2H), 6.21 (q, J=2.2 Hz, 1H), 3.97 (s, 1H), 3.17 (s, 4H), 3.08 (s, 1H), 2.74 (s, 4H), 2.64-2.43 (m, 2H), 2.16-1.91 (m, 2H).

MS: m/z 271 (M+1).

and

### (S)-1-(3-ethynylcyclopent-2-en-1-yl)-4-(4-fluorophenyl)piperazine (compound 22c″; 11.5 g)

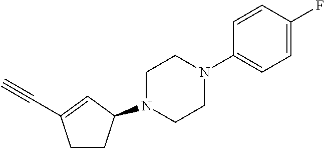 [SMILES: C#CC1=C[C@@H](N2CCN(c3ccc(F)cc3)CC2)CC1]

¹H NMR (400 MHz, CDCl₃) δ 7.01-6.95 (m, 2H), 6.89 (dd, J=9.2, 4.6 Hz, 2H), 6.21 (q, J=2.2 Hz, 1H), 3.97 (s, 1H), 3.17 (s, 4H), 3.08 (s, 1H), 2.74 (s, 4H), 2.64-2.43 (m, 2H), 2.16-1.91 (m, 2H).

MS: m/z 271 (M+1).

### Step 4: (R)-7-(3-(4-(4-fluorophenyl)piperazin-1-yl)cyclopent-1-en-1-yl)-1,6-naphthyridin-5(6H)-one (Compound 22—hydrochloride salt)

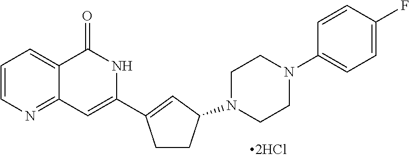 [SMILES: O=c1[nH]c(C2=C[C@H](N3CCN(c4ccc(F)cc4)CC3)CC2)cc2ncccc12]

Synthesis of (R)-7-(3-(4-(4-fluorophenyl)piperazin-1-yl)cyclopent-1-en-1-yl)-1,6-naphthyridin-5(6H)-one (Compound 22—hydrochloride salt) was carried out starting from (R)-1-(3-ethynylcyclopent-2-en-1-yl)-4-(4-fluorophenyl)piperazine (Compound 22c′) following the procedure described for the synthesis of (R)-4-(4-(3-(5-oxo-5,6-dihydro-1,6-naphthyridin-7-yl)cyclopent-2-en-1-yl)piperazin-1-yl)benzonitrile (Compound 1—hydrochloride salt) in Example 1.

¹H NMR (400 MHz, DMSO-d₆) δ 11.89 (brs-exchangeable with D₂O, 1H), 11.52 (brs-exchangeable with D₂O, 1H), 9.04 (s, 1.6 Hz, 1H), 8.73 (d, J=8.0 Hz, 1H), 7.70 (dd, J=8.0, 5.0 Hz, 1H), 7.14-7.10 (m, 2H), 7.07-7.03 (m, 2H), 6.96 (s, 1H), 6.88 (s, 1H), 4.69 (s, 1H), 3.84-3.75 (m, 2H), 3.62-3.54 (m, 2H), 3.30-3.11 (m, 4H), 2.94-2.86 (m, 2H), 2.44-2.38 (m, 2H).

MS: m/z 391.2 (M+1).

The following compound was prepared using the procedure described above in Example 5 with appropriate changes to the reactants and reaction conditions.

### (R)-6-(3-(4-(4-fluorophenyl)piperazin-1-yl)cyclopent-1-en-1-yl)thieno[3,2-c]pyridin-4(5H)-one (Compound 47—hydrochloride salt)

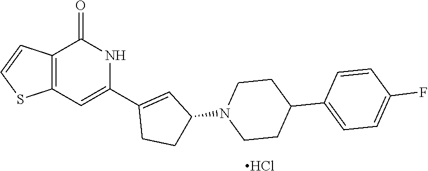 [SMILES: O=c1[nH]c(C2=C[C@H](N3CCC(c4ccc(F)cc4)CC3)CC2)cc2sccc12 ; Cl]

¹H NMR (400 MHz, DMSO-d₆) δ 11.40 (brs-exchangeable with D₂O, 1H), 11.15 (brs-exchangeable with D₂O, 1H), 7.69 (d, J=5.0 Hz, 1H), 7.51 (d, J=5.0 Hz, 1H), 7.14-7.06 (m, 3H), 7.05-7.03 (m, 2H), 4.63 (s, 1H), 3.78 (d, J=10.8 Hz, 2H), 3.56 (t, J=11.9 Hz, 2H), 3.25-3.06 (m, 4H), 2.92-2.75 (m, 2H), 2.37 (d, J=7.4 Hz, 2H).

MS: m/z 396 (M+1).

### Example 6: Synthesis of (R)-4-(4-(3-(2-methyl-5-oxo-5,6-dihydro-1,6-naphthyridin-7-yl)cyclopent-2-en-1-yl)piperazin-1-yl)benzonitrile (Compound 6)

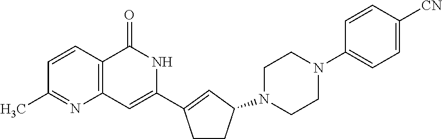 [SMILES: Cc1ccc2c(=O)[nH]c(C3=C[C@H](N4CCN(c5ccc(C#N)cc5)CC4)CC3)cc2n1]

### Step 1: Synthesis of 1-((trimethylsilyl)ethynyl)cyclopent-2-enol (Compound 6a)

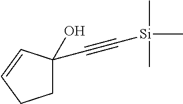

To a stirred solution of Trimethylsilylacetylene (160 ml, 1.139 mol) in tetrahydrofuran (680 ml) was added n-Butyl Lithium (1.6 M in hexane, 712 ml, 1.139 mol) at −78° C. over a period of 30 minutes under N₂ atmosphere and the resulting mixture was allowed to stir over a period of 60 minute at same temperature. Cyclopent-2-enone (85 g, 1035 mmol) was added over a period of 30 min at the same temperature. The reaction mixture was stirred for 2 hrs at same temperature. The progress of the reaction was monitored by TLC. The reaction mixture was warmed to ˜−40° C. and 20% ammonium chloride solution added slowly (635 ml). The organic layer was separated, aqueous layer extracted with Methyl tert-butyl ether (MTBE) (500 ml). The combined organic layer was washed with water (3×500 ml) followed by brine solution (500 ml). The organic layer was separated, dried over sodium sulfate and concentrated under reduced pressure to get oily compound which was purified by high vacuum distillation (Oil bath temp-115-130° C.) to get 101.00 gm (54.1%) of title compound as liquid.

¹H NMR (400 MHz, CDCl₃) δ 6.01 (dt, J=5.0, 2.2 Hz, 1H), 5.82 (dt, J=4.9, 2.1 Hz, 1H), 2.62-2.50 (m, 1H), 2.50-2.37 (m, 2H), 2.24-2.12 (m, 2H), 0.18 (s, 9H).

### Step 2: Synthesis of 3-((trimethylsilyl)ethynyl)cyclopent-2-enol (Compound 6b)

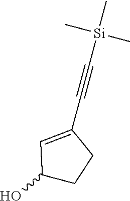

To a stirred solution of 1-((trimethylsilyl)ethynyl)cyclopent-2-enol (Compound 6a, 100 g, 555 mmol) in MTBE (800 ml) was added 3% H₂SO₄ (800 ml) at 10° C. and the resulting biphasic reaction mixture was allowed to stir at ambient temperature for 16 hrs. The progress of reaction was monitored by TLC. The organic layer was separated and aqueous layer was extracted with MTBE (400 ml). The combined organic layer was washed with water (3×400 ml; pH-7) and brine solution (400 ml). The organic layer dried over anhydrous Na₂SO₄, filtered and concentrated to yield 100.00 gm (99.5%) of title compound as liquid.

¹H NMR (400 MHz, CDCl₃) δ 6.11 (q, J=2.1 Hz, 1H), 4.94-4.85 (m, 1H), 2.71-2.55 (m, 1H), 2.47-2.25 (m, 2H), 1.84-1.69 (m, 2H), 0.21 (s, 9H).

### Step 3: Synthesis (R)-3-((trimethylsilyl)ethynyl)cyclopent-2-en-1-yl acetate (Compound 6c)

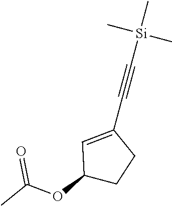

To the stirred solution of 3-((trimethylsilyl)ethynyl)cyclopent-2-enol (Compound 6b, 50 g, 277 mmol) in MTBE (650 ml) were added vinyl acetate (51 ml) and Lipase PS′Amano″SD (10 g, 20% w/w). The above suspension was stirred at 45° C. (internal temperature) for 18 hrs. The reaction was monitored by TLC, which showed 25-30% conversion. Vinyl acetate (15 ml, 166.2 mmol) was added and stirred at same temperature for 6 hrs. Additional Vinyl acetate (15 ml, 166.2 mmol) and 3.0 gm of Lipase PS Amano SD enzyme (6% w/w) were added and stirred at same temperature for 18 hrs and reaction monitored by TLC, which showed approximately 50% conversion. The suspension was filtered through Celite bed and bed was washed with MTBE (300 mL). A crude product was purified by silica (100-200) column chromatography using 5-6% ethyl acetate in n-hexane to yield (23.00 gm, 37.3%) title compound.

¹H NMR (400 MHz, CDCl₃) δ 6.10 (q, J=2.2 Hz, 1H), 5.76-5.67 (m, 1H), 2.74-2.60 (m, 1H), 2.52-2.27 (m, 2H), 2.04 (s, 3H), 1.95-1.82 (m, 1H), 0.22 (s, 9H).

### Step 4: (R)-tert-butyl 4-(3-((trimethylsilyl)ethynyl)cyclopent-2-en-1-yl)piperazine-1-carboxylate (Compound 6d)

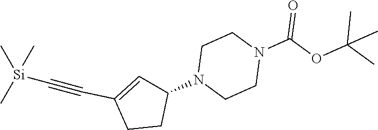 [SMILES: CC(C)(C)OC(=O)N1CCN([C@H]2C=C(C#C[Si](C)(C)C)CC2)CC1]

To a stirred deoxygenated solution (R)-3-((trimethylsilyl)ethynyl)cyclopent-2-en-1-yl acetate (Compound 6c, 23 g, 103 mmol) and tert-butyl piperazine-1-carboxylate (19.27 g, 103 mmol) in 1,4-dioxane: water (370 ml: 95 ml) at 0-5° C., Tetrakis(triphenyl phosphine) Pd(0) (0.896 g, 0.776 mmol) was added. The reaction mixture was stirred at 0-5° C. for 18 hrs. The progress of the reaction was monitored by TLC. The reaction mass was filtered to remove the heterogeneous mass. The filtrate was diluted with n-hexane (120 ml) and quenched with water (120 ml). The organic layer was separated, and the aqueous layer was further extracted with n-hexane (120 ml). The combined organic layer was washed with water (120 ml), brine (100 ml), dried over anhydrous Na₂SO₄, and evaporated under reduced pressure to afford the crude product. The obtained crude product was further dissolved in n-heptane (230 ml) and activated carbon (4 gm) was added and stirred at 25-30° C. for additional 1 hr. It was filtered through Celite bed and the filtrate was evaporated to dryness under reduced pressure to yield (35.00 gm, 97.00%) title compound.

¹H NMR (400 MHz, CDCl₃) δ 6.11 (q, J=2.2 Hz, 1H), 4.06-3.88 (m, 1H), 3.60-3.42 (m, 4H), 2.71-2.50 (m, 4H), 2.18-1.86 (m, 2H), 1.47 (s, 9H), 1.37-1.19 (m, 2H), 0.22 (s, 9H).

MS: m/z-349.11 (M+1).

### Step 5: (R)-tert-butyl 4-(3-ethynylcyclopent-2-en-1-yl)piperazine-1-carboxylate (Compound 23d)

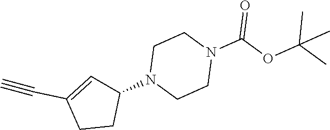 [SMILES: C#CC1=C[C@H](N2CCN(C(=O)OC(C)(C)C)CC2)CC1]

TBAF (7.53 ml, 7.53 mmol) was added slowly to a deoxygenated solution of (R)-tert-butyl 4-(3-((trimethylsilyl)ethynyl)cyclopent-2-en-1-yl)piperazine-1-carboxylate (Compound 6d, 5 g, 100 mmol) in tetrahydrofuran (350 ml) at 25-30° C. over a period of 15 min. The reaction mixture was allowed to stir at the same temperature for 15-20 min. Water (200 ml) was added to the reaction mixture and the product was extracted with n-hexane (200 ml). The organic layer was separated, and the aqueous layer was further extracted with n-hexane (200 ml). The combined organic layer was washed with water (100 ml) and then with brine (100 ml). The separated organic layer was dried over anhydrous Na₂SO₄, and evaporated under reduced pressure to afford the crude product. The obtained crude product was further dissolved in n-heptane (350 ml) and treated with activated carbon (4 g) for 30 min. It was filtered through Celite bed and the filtrate was evaporated to dryness under reduced pressure to yield crude (23.00 gm with 81% ee).

### Process for Enhancement of Enantiomeric Excess (ee) Through Crystallization

The crude was dissolved in n-heptane (70 ml) at 60-70° C. and then slowly cooled to 0° C. over a period of 30 min. The solution was stirred at 0° C. for 3h to observe selective crystallization of major enantiomer. The solid was separated, filtered and washed with cold (−30 to −40° C.) n-heptane (20 ml). The filtered solid was dried at atmospheric pressure to yield title compound (13.50 gm, 48.6%).

¹H NMR (400 MHz, CDCl₃) δ 6.15 (q, J=2.2 Hz, 1H), 3.94-3.85 (m, 1H), 3.50-3.42 (m, 4H), 3.06 (s, 1H), 2.59-2.39 (m, 6H), 2.10-1.96 (m, 1H), 1.95-1.81 (m, 1H), 1.47 (s, 9H).

MS: m/z-277.58 (M+1).

### Step 6: (R)-1-(3-ethynylcyclopent-2-en-1-yl)piperazine dihydrochloride (Compound 23e)

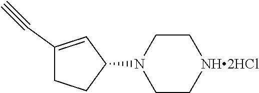

To a solution of (R)-tert-butyl 4-(3-ethynylcyclopent-2-en-1-yl)piperazine-1-carboxylate (Compound 23d, 57 g, 206 mmol) in dichloromethane (300 ml) was added, hydrochloric acid in 1,4 dioxane (516 ml, 2062 mmol, 4M solution in 1,4 dioxane) at 0-5° C. The reaction mixture was warmed to room temperature and stirred for 2 h. The progress of the reaction was monitored by TLC. The reaction mixture was evaporated under reduced pressure to obtain solid product which was co-evaporated with diethylether (150 ml), followed by toluene (150 ml) to obtain the title product (51.0 gm, 99.0%) as a white solid.

¹H NMR (400 MHz, DMSO-d₆) δ 12.18 (brs-exchangeable with D₂O, 1H), 9.70 (brs-exchangeable with D₂O, 1H), 6.23 (s, 1H), 4.57-4.50 (m, 1H), 4.47 (s, 1H), 3.51-3.21 (m, 7H), 2.73-2.59 (m, 1H), 2.50-2.40 (m, 2H), 2.36-2.11 (m, 2H).

### Step 7: (R)-4-(4-(3-ethynylcyclopent-2-en-1-yl)piperazin-1-yl)benzonitrile (Compound 1j)

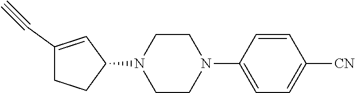 [SMILES: C#CC1=C[C@H](N2CCN(c3ccc(C#N)cc3)CC2)CC1]

To a solution of (R)-1-(3-ethynylcyclopent-2-en-1-yl)piperazine dihydrochloride (Compound 23e, 47 g, 189 mmol) in dimethylsulfoxide (200 ml) was added potassium carbonate (117 g, 849 mmol) followed by the addition of 4-fluoro benzonitrile (29.7 g, 245 mmol) at 25-30° C. The reaction mixture was warmed and stirred at 120° C. for 18 h. The progress of the reaction was monitored by TLC. The reaction mixture was poured into water (1000 ml) and extracted with ethyl acetate (2×400 ml), combined organic layer was washed with water (300 ml) and brine solution (300 ml). The organic layer was dried over sodium sulphate and evaporated under reduced pressure to obtain a crude oily product which was purified by column chromatography over silica gel (100-200 mesh) using 35-40% ethyl acetate in hexane as an eluent to obtain the title product (41.0 gm, 78.0% yield).

¹H NMR (400 MHz, CDCl₃) δ 7.51 (d, J=8.8 Hz, 2H), 6.87 (d, J=8.8 Hz, 2H), 6.18 (d, J=2.2 Hz, 1H), 3.98-3.89 (m, 1H), 3.40-3.27 (m, 4H), 3.08 (s, 1H), 2.73-2.61 (m, 4H), 2.59-2.46 (m, 2H), 2.14-2.00 (m, 1H), 1.99-1.85 (m, 1H).

MS: m/z 277.98 (M+1).

### Step 8: (R)-methyl 2-((3-(4-(4-cyanophenyl)piperazin-1-yl)cyclopent-1-en-1-yl)ethynyl)-6-methylnicotinate (Compound 6e)

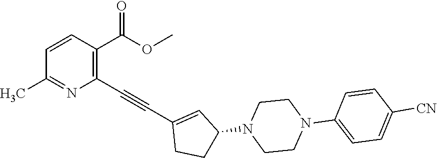 [SMILES: COC(=O)c1ccc(C)nc1C#CC1=C[C@H](N2CCN(c3ccc(C#N)cc3)CC2)CC1]

To a stirred solution of methyl 2-bromo-6-methylnicotinate (US2010144760, 2.79 g, 12.11 mmol) in acetonitrile (50 ml) (degassed by N₂ purge separately) was added bis(triphenylphosphine)palladium(II) chloride (1.063 g, 1.514 mmol). The reaction mixture was heated up to 70° C. and diisopropyl ethyl amine (7.83 g, 60.6 mmol) was added slowly, followed by a solution of (R)-4-(4-(3-ethynylcyclopent-2-en-1-yl)piperazin-1-yl)benzonitrile (Compound 1j, 2.8 g, 10.10 mmol) in acetonitrile (20 ml) was added slowly at the same temperature. The reaction mixture was heated and stirred at 80-85° C. for 14 hrs. The progress of the reaction was monitored by TLC. The reaction mixture was distilled under vacuum to dryness to obtain a crude product which was purified by column chromatography over silica gel (100-200 mesh) using 60-80% ethyl acetate in hexane as an eluent to obtain the title product (0.9 gm, 20.90% yield).

¹H NMR (400 MHz, CDCl₃) δ 8.17 (d, J=8.1 Hz, 1H), 7.52 (d, J=8.5 Hz, 2H), 7.20 (d, J=8.2 Hz, 1H), 6.90-6.87 (m, 2H), 6.36 (d, J=2.3 Hz, 1H), 4.08-4.03 (m, 1H), 3.96 (s, 3H), 3.40-3.35 (m, 4H), 2.75-2.67 (m, 6H), 2.64 (s, 3H), 2.13-2.09 (m, 1H), 2.03-1.95 (m, 1H).

MS: m/z 427.24 (M+1).

### Step 9: (R)-2-((3-(4-(4-cyanophenyl)piperazin-1-yl)cyclopent-1-en-1-yl)ethynyl)-6-methylnicotinic acid (Compound 6f)

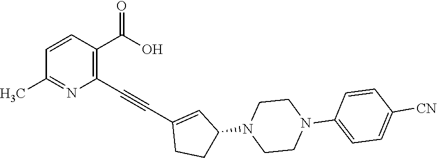 [SMILES: Cc1ccc(C(=O)O)c(C#CC2=C[C@H](N3CCN(c4ccc(C#N)cc4)CC3)CC2)n1]

To a stirred solution of (R)-methyl 2-((3-(4-(4-cyanophenyl)piperazin-1-yl)cyclopent-1-en-1-yl)ethynyl)-6-methylnicotinate (Compound 6e, 0.9 g, 2.110 mmol) in methanol (10 ml) and tetrahydrofuran (10 ml) was added sodium hydroxide (0.253 g, 6.33 mmol) dissolved in water (3 ml) at room temperature and reaction was stirred at same temperature for 2 hrs. The progress of the reaction was monitored by TLC. The reaction mixture was distilled under vacuum till dryness to obtain a crude product. To this crude product was added water (5 ml) and pH was adjusted to 5 using 10% aqueous hydrochloric acid. The solid precipitated out was filtered off and dried to obtain the title product (0.87 gm, 100% yield).

¹H NMR (400 MHz, DMSO-d₆) δ 11.87 (brs-exchangeable with D₂O, 1H), 8.15 (d, J=8.2 Hz, 1H), 7.67 (d, J=8.6 Hz, 2H), 7.41 (d, J=7.9 Hz, 1H), 7.12 (d, J=8.7 Hz, 2H), 6.46 (s, 1H), 4.63-4.58 (m, 1H), 3.59-3.34 (m, 4H), 3.23-3.15 (m, 1H), 3.13-3.02 (m, 1H), 2.84-2.61 (m, 4H), 2.56 (s, 3H), 2.40-2.33 (m, 2H).

MS: m/z 413.13 (M+1).

### Step 10: (R)-4-(4-(3-(2-methyl-5-oxo-5H-pyrano[4,3-b]pyridin-7-yl)cyclopent-2-en-1-yl)piperazin-1-yl)benzonitrile (Compound 6g)

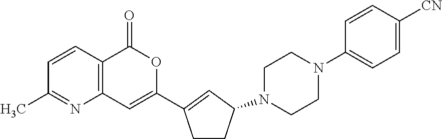 [SMILES: Cc1ccc2c(=O)oc(C3=C[C@H](N4CCN(c5ccc(C#N)cc5)CC4)CC3)cc2n1]

To a stirred solution of (R)-2-((3-(4-(4-cyanophenyl)piperazin-1-yl)cyclopent-1-en-1-yl)ethynyl)-6-methylnicotinic acid (Compound 6f, 0.87 g, 2.109 mmol) in tetrahydrofuran (10 ml) and dichloromethane (10 ml) was added triflic acid (1.266 g, 8.44 mmol) at room temperature and reaction was stirred at same temperature for 48 hr. The progress of the reaction was monitored by TLC. Diethyl ether (20 ml) was added slowly to the reaction mixture and the solid that precipitated out was filtered off and dried under vacuum to obtain the title product (0.8 gm, 92% yield).

¹H NMR (400 MHz, DMSO-d₆) δ 8.40 (d, J=8.1 Hz, 1H), 7.67 (d, J=8.6 Hz, 2H), 7.53 (d, J=8.2 Hz, 1H), 7.12 (d, J=8.7 Hz, 2H), 6.98 (s, 1H), 6.69 (s, 1H), 4.70-4.66 (m, 1H), 4.14-4.06 (m, 2H), 3.66-3.58 (m, 1H), 3.52-3.45 (m, 1H), 3.43-3.32 (m, 2H), 3.31-3.20 (m, 1H), 3.15-3.07 (m, 1H), 2.98-2.89 (m, 1H), 2.80-2.72 (m, 1H), 2.65 (s, 3H), 2.44-2.37 (m, 2H).

MS: m/z 413.0 (M+1).

### Step 11: (R)-4-(4-(3-(2-methyl-5-oxo-5,6-dihydro-1,6-naphthyridin-7-yl)cyclopent-2-en-1-yl)piperazin-1-yl)benzonitrile (Compound 6)

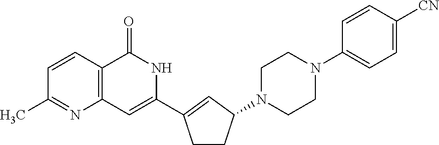 [SMILES: Cc1ccc2c(=O)[nH]c(C3=C[C@H](N4CCN(c5ccc(C#N)cc5)CC4)CC3)cc2n1]

To a solution of (R)-4-(4-(3-(2-methyl-5-oxo-5H-pyrano[4,3-b]pyridin-7-yl)cyclopent-2-en-1-yl)piperazin-1-yl)benzonitrile (Compound 6g, 0.8 g, 1.939 mmol) in anhydrous tetrahydrofuran (5 ml) was added ammonia in methanol (13.65 ml, 97 mmol, 7M solution in methanol) at 25° C. The reaction mixture in steel bomb reactor was stirred at 80-85° C. for 4 hrs. The progress of the reaction was monitored by TLC. The reaction mixture was cooled to room temperature and the solid precipitated out was filtered and dried to obtain the title product (0.65 gm, 81% yield).

¹H NMR (400 MHz, DMSO-d₆) δ 11.38 (brs-exchangeable with D₂O, 1H), 8.35 (d, J=8.2 Hz, 1H), 7.59 (d, J=8.6 Hz, 2H), 7.35 (d, J=8.4 Hz, 1H), 7.04 (d, J=8.6 Hz, 2H), 6.95 (s, 1H), 6.54 (s, 1H), 3.93-3.88 (m, 1H), 3.21-3.34 (m, 4H), 2.76-2.56 (m, 9H), 2.11-2.06 (m, 1H), 1.92-1.87 (m, 1H).

MS: m/z 412.2 (M+1).

### Step 12: (R)-4-(4-(3-(2-methyl-5-oxo-5,6-dihydro-1,6-naphthyridin-7-yl)cyclopent-2-en-1-yl)piperazin-1-yl)benzonitrile (Compound 6—hydrochloride salt)

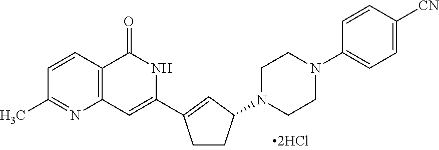 [SMILES: Cc1ccc2c(=O)[nH]c(C3=C[C@H](N4CCN(c5ccc(C#N)cc5)CC4)CC3)cc2n1]

To a suspension of (R)-4-(4-(3-(2-methyl-5-oxo-5,6-dihydro-1,6-naphthyridin-7-yl)cyclopent-2-en-1-yl)piperazin-1-yl)benzonitrile (Compound 6, 0.05 g, 0.122 mmol) in dichloromethane (5 ml) and ethanol (5 ml), was added hydrochloric acid (0.027g, 0.729 mmol, 3M in 1,4-dioxane) at 55-60° C. The reaction mixture was stirred for 30 min at the same temperature. The reaction mixture was then cooled to room temperature, diluted with diethyl ether (10 ml), and product was collected by filtration. The solid compound was washed with diethyl ether (5 ml) and dried under vacuum to obtain the title compound (0.049 g, 90% yield).

¹H NMR (400 MHz, DMSO-d₆) δ 11.86 (brs-exchangeable with D₂O, 1H), 11.59 (brs-exchangeable with D₂O, 1H), 8.64 (d, J=8.4 Hz, 1H), 7.68 (d, J=8.4 Hz, 2H), 7.60 (d, J=8.4 Hz, 1H), 7.15 (d, J=8.4 Hz, 2H), 6.94 (s, 1H), 6.88 (s, 1H), 4.69 (s, 1H), 4.17-4.13 (m, 2H), 3.64-3.54 (m, 2H), 3.42-3.30 (m, 2H), 3.23-3.07 (m, 2H), 2.95-2.79 (m, 2H), 2.74 (s, 3H), 2.46-2.36 (m, 2H).

MS: m/z 412.2 (M+1).

The following compounds were prepared using the procedure described above in Example 6 with appropriate changes to the reactants and reaction conditions.

### (R)-7-(3-(4-(4-(methylamino)phenyl)piperazin-1-yl)cyclopent-1-en-1-yl)-1,6-naphthyridin-5(6H)-one (Compound 27—hydrochloride salt)

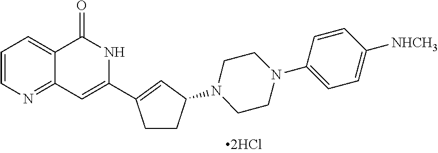 [SMILES: CNc1ccc(N2CCN([C@H]3C=C(c4cc5ncccc5c(=O)[nH]4)CC3)CC2)cc1]

¹H NMR (400 MHz, DMSO-d6) δ 11.65 (brs-exchangeable with D₂O, 1H), 10.94 (brs-exchangeable with D₂O, 1H), 8.98 (dd, J=4.7, 1.7 Hz, 1H), 8.56 (d, J=8.1 Hz, 1H), 7.58 (dd, J=8.1, 4.7 Hz, 2H), 7.43 (d, J=8.5 Hz, 2H), 7.16 (d, J=8.5 Hz, 2H), 6.88 (s, 1H), 6.80 (s, 1H), 4.72-4.70 (m, 1H), 3.97-3.94 (m, 2H), 3.64-3.58 (m, 2H), 3.20.3.13 (m, 4H), 2.92-2.88 (m, 5H), 1.32-1.22 (m, 2H).

MS: m/z 401.5 (M+1).

### (R)-7-(3-(4-(4-acetylphenyl)piperazin-1-yl)cyclopent-1-en-1-yl)-1,6-naphthyridin-5(6H)-one (Compound 28—hydrochloride salt)

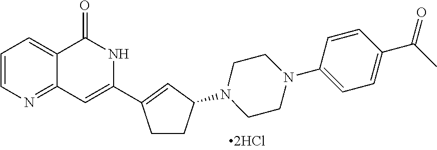 [SMILES: CC(=O)c1ccc(N2CCN([C@H]3C=C(c4cc5ncccc5c(=O)[nH]4)CC3)CC2)cc1]

¹H NMR (400 MHz, DMSO-d6) δ 11.68 (brs-exchangeable with D₂O, 1H), 11.31 (brs-exchangeable with D₂O, 1H), 8.98 (dd, J=4.7, 1.8 Hz, 1H), 8.59 (dd, J=8.1, 1.8 Hz, 1H), 7.87 (d, J=9.0 Hz, 2H), z 7.60 (dd, J=8.1, 4.7 Hz, 1H), 7.10 (d, J=9.0 Hz, 2H), 6.89 (s, 1H), 6.81 (s, 1H), 4.69 (s, 1H), 4.16-4.13 (m, 2H), 3.66-3.55 (m, 2H), 3.40-3.27 (m, 2H), 3.27-3.08 (m, 2H), 2.94-2.89 (m, 2H), 2.49 (s, 3H), 2.46-2.29 (m, 2H).

MS: m/z 415.2 (M+1).

### (R)-6-(3-(4-(thiazol-2-yl)piperazin-1-yl)cyclopent-1-en-1-yl)thieno[3,2-c]pyridin-4(5H)-one (Compound 51—hydrochloride salt)

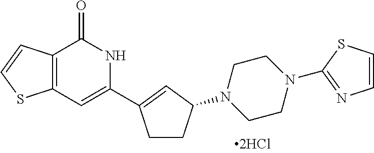 [SMILES: O=c1[nH]c(C2=C[C@H](N3CCN(c4nccs4)CC3)CC2)cc2sccc12]

¹H NMR (400 MHz, DMSO-d6) δ 11.74 (brs-exchangeable with D₂O, 1H), 11.38 (brs-exchangeable with D₂O, 1H), 7.69 (d, J=5.3 Hz, 1H), 7.51 (d, J=5.3 Hz, 1H), 7.33 (d, J=3.8 Hz, 1H), 7.10 (s, 1H), 7.06 (d, J=3.8 Hz, 1H), 6.74 (s, 1H), 4.64 (s, 1H), 4.17-4.13 (m, 2H), 3.77-3.51 (m, 4H), 3.30-3.14 (m, 2H), 2.93-2.76 (m, 2H), 2.40-2.36 (m, 2H).

MS: m/z 385.2 (M+1).

### (R)-3-fluoro-4-(4-(3-(4-oxo-4,5-dihydrothiazolo[5,4-c]pyridin-6-yl)cyclopent-2-en-1-yl)piperazin-1-yl)benzonitrile (Compound 52—hydrochloride salt)

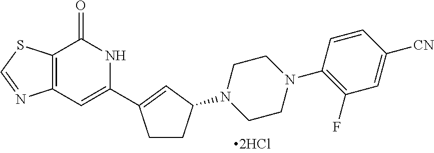 [SMILES: N#Cc1ccc(N2CCN([C@H]3C=C(c4cc5ncsc5c(=O)[nH]4)CC3)CC2)c(F)c1]

¹H NMR (400 MHz, DMSO-d₆) δ 11.85 (brs-exchangeable with D₂O, 1H), 11.48 (brs-exchangeable with D₂O, 1H), 9.61 (s, 1H), 7.81 (dd, J=13.1, 1.9 Hz, 1H), 7.65 (dd, J=8.5, 1.9 Hz, 1H), 7.27 (t, J=8.7 Hz, 1H), 7.11 (s, 1H), 6.81 (d, J=2.5 Hz, 1H), 4.67 (s, 1H), 3.81-3.70 (m, 2H), 3.66-3.52 (m, 2H), 3.47-3.34 (m, 2H), 3.33-3.14 (m, 2H), 2.95-2.85 (m, 2H), 2.43-2.30 (m, 2H).

MS: m/z 422.1 (M+1).

### Example 7: Synthesis of 4-((R)-3-methyl-4-((R/S)-3-(5-oxo-5,6-dihydro-1,6-naphthyridin-7-yl)cyclopent-2-en-1-yl)piperazin-1-yl)benzonitrile (Compound 14)

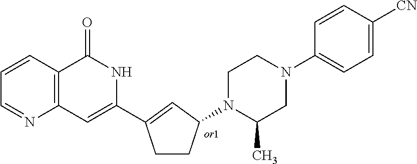 [SMILES: C[C@@H]1CN(c2ccc(C#N)cc2)CCN1[C@H]1C=C(c2cc3ncccc3c(=O)[nH]2)CC1]

### Step 1: 3-bromocyclopent-1-enecarbonitrile (Compound 14a)

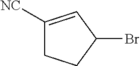

To a stirred solution of cyclopent-1-enecarbonitrile (50 g, 537 mmol) in tetrachloromethane (400 ml) at 25° C. was added N-bromosuccinimide (96 g, 537 mmol) under nitrogen atmosphere. The resulting mixture was refluxed for 2 hrs. The progress of reaction was monitored by TLC. The reaction mixture cooled to 25° C. and filtered through Celite. The filtrate was concentrated under reduced pressure to obtain a crude product, which was purified by column chromatography over silica gel (100-200 mesh) using 1% ethyl acetate in hexane as an eluent to obtain the title compound (60.0 g, 65%).

¹HNMR (400 MHz, CDCl₃): δ 6.77-6.73 (m, 1H), 5.12-5.09 (m, 1H) 2.95-2.86 (m, 1H) 2.67-2.42 (m, 3H).

### Step 2: tert-butyl (3R)-4-(3-cyanocyclopent-2-en-1-yl)-3-methylpiperazine-1-carboxylate (Compound 14b)

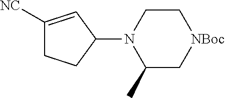 [SMILES: C[C@@H]1CN(C(=O)OC(C)(C)C)CCN1C1C=C(C#N)CC1]

To a stirred solution of tert-butyl (R)-3-methylpiperazine-1-carboxylate (9.0 g, 44.9 mmol) in acetonitrile (100 ml) was added potassium carbonate (18.63 g, 135 mmol) at 25° C. and stirred the reaction mixture for 10 minutes. To the reaction mixture was added, a solution of 3-bromocyclopent-1-enecarbonitrile (Compound 14a, 7.73 g, 44.9 mmol) in acetonitrile (25 ml) and the reaction mixture was stirred for 16 h. The progress of the reaction was monitored by TLC. The reaction mixture was concentrated under reduced pressure. The residue obtained was diluted with water (100 ml) and extracted with ethyl acetate (2×200 ml). The combined organic layer was dried over anhydrous sodium sulphate and evaporated under reduced pressure to obtain crude product. The crude product purified by flash column chromatography over silica gel (100-200 mesh) using 20% ethyl acetate in hexane as an eluent to obtain the title compound (8.2 g, 62.6% yield).

The diastereomers of tert-butyl (3R)-4-(3-cyanocyclopent-2-en-1-yl)-3-methylpiperazine-1-carboxylate was separated by flash column chromatography over silica gel (100-200 mesh) using 10-20% ethyl acetate in hexane as an eluent to obtain two diastereomers separately.

### tert-butyl (R)-4-((R/S)-3-cyanocyclopent-2-en-1-yl)-3-methylpiperazine-1-carboxylate (Compound 14b′)

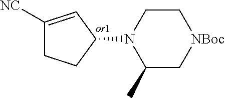 [SMILES: C[C@@H]1CN(C(=O)OC(C)(C)C)CCN1[C@H]1C=C(C#N)CC1]

¹H NMR (400 MHz, CDCl₃) δ 6.58 (s, 1H), 4.52 (s, 1H), 4.00-3.74 (m, 2H), 3.08-2.90 (m, 1H), 2.64 (s, 3H), 2.57-2.40 (m, 1H), 2.36-2.18 (m, 1H), 2.02-1.81 (m, 2H), 1.63 (s, 1H), 1.48 (s, 9H), 1.19-1.03 (m, 3H).

MS: m/z 292.1 (M+1).

and

### tert-butyl (R)-4-((S/R)-3-cyanocyclopent-2-en-1-yl)-3-methylpiperazine-1-carboxylate (Compound 14b″)

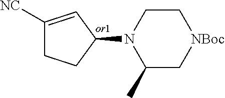 [SMILES: C[C@@H]1CN(C(=O)OC(C)(C)C)CCN1[C@@H]1C=C(C#N)CC1]

¹H NMR (400 MHz, CDCl₃) δ 6.69 (d, J=2.4 Hz, 1H), 4.32 (s, 1H), 3.71 (s, 2H), 3.18 (s, 1H), 2.68 (s, 3H), 2.57 (dt, J=16.3, 7.0 Hz, 1H), 2.24 (s, 2H), 1.92 (s, 1H), 1.63 (s, 1H), 1.48 (s, 9H), 1.13 (d, J=6.2 Hz, 3H).

MS: m/z 292.21 (M+1).

Both these diastereomers were processed further individually to obtain the respective title products.

### Step 3: tert-butyl (R)-4-((R/S)-3-formylcyclopent-2-en-1-yl)-3-methylpiperazine-1-carboxylate (Compound 14c)

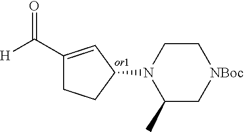 [SMILES: C[C@@H]1CN(C(=O)OC(C)(C)C)CCN1[C@H]1C=C(C=O)CC1]

A stirred solution of tert-butyl (R)-4-((R/S)-3-cyanocyclopent-2-en-1-yl)-3-methylpiperazine-1-carboxylate (Compound 14b′, 4.0 g, 13.73 mmol) in dichloromethane (50 ml) was cooled at −78° C. Diisobutylaluminium hydride (20.59 ml, 20.59 mmol, 1M solution in toluene) was added slowly over 10-15 minutes. The reaction mixture was stirred for 15 min at −78° C. and warmed to room temperature and stirred for 1 h. The progress of the reaction was monitored by TLC. The reaction mixture was quenched by drop wise addition of saturated ammonium chloride solution (20 ml) at 0° C. (carefully: The reaction quenching is exothermic). A gel type reaction mass was observed, Celite (100 g) was added to the reaction mixture and the reaction mixture was diluted with 10% methanol in dichloromethane (0.3 lit) and stirred for 20 min. The reaction mass was filtered through Celite bed and the bed was washed with 1 lit. of 10% methanol in dichloromethane. The combined organic filtrate was dried over sodium sulphate and concentrated under vacuum till dryness to afford the crude product which was purified by column chromatography over silica gel (100-200 mesh) using ethyl acetate in hexane as an eluent to obtain the title product as an yellow solid. (3.05 gm, 75.0% yield).

¹H NMR (400 MHz, CDCl₃) δ 9.85 (s, 1H), 6.78 (s, 1H), 4.56 (s, 1H), 4.00-3.73 (m, 2H), 3.02 (t, J=11.8 Hz, 1H), 2.79-2.44 (m, 5H), 2.33 (t, J=11.2 Hz, 1H), 2.03-1.84 (m, 2H), 1.47 (s, 9H), 1.16 (d, J=6.2 Hz, 3H).

MS: m/z 295.1 (M+1).

### Step 4: tert-butyl (R)-4-((R/S)-3-ethynylcyclopent-2-en-1-yl)-3-methyl piperazine-1-carboxylate (Compound 14d)

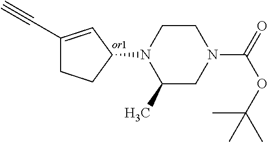 [SMILES: C#CC1=C[C@H](N2CCN(C(=O)OC(C)(C)C)C[C@H]2C)CC1]

To a solution of trimethylsilyldiazomethane (8.66 ml, 17.32 mmol) in tetrahydrofuran (100 ml) at −78° C. was slowly added, n-butyl lithium (9.55 ml, 15.29 mmol) solution in hexane (1.6 M). The reaction mixture was stirred for 30 minute at same temperature. A solution of tert-butyl (R)-4-((R/S)-3-formylcyclopent-2-en-1-yl)-3-methylpiperazine-1-carboxylate (Compound 14c, 3.0 g, 10.19 mmol) in tetrahydrofuran (50 ml) was slowly added to the reaction mixture at −78° C. The reaction mixture was stirred for 30 minute and then warmed to room temperature and further stirred for 2 h. The progress of the reaction was monitored by TLC. The reaction mixture was diluted with ethyl acetate (100 ml) and then washed with water (50 ml). The organic layer was separated and the aqueous layer was again extracted with ethyl acetate (2×100 ml). The combined organic layer was dried over sodium sulphate and evaporated under reduced pressure to obtain crude oily product which was purified by column chromatography over silica gel (100-200 mesh) using ethyl acetate in hexane as an eluent to obtain the title product (1.55 gm, 49.7% yield).

¹H NMR (400 MHz, CDCl₃) δ 6.07 (s, 1H), 4.47 (s, 1H), 4.02-3.64 (m, 2H), 3.10-2.93 (m, 2H), 2.82-2.60 (m, 2H), 2.57-2.40 (m, 3H), 2.22 (d, J=13.8 Hz, 1H), 1.96-1.76 (m, 2H), 1.48 (s, 9H), 1.20-1.06 (m, 3H).

MS: m/z 291.0 (M+1).

### Step 5: (R)-1-((R/S)-3-ethynylcyclopent-2-en-1-yl)-2-methylpiperazine dihydrochloride (Compound 14e)

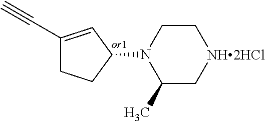

To a solution of tert-butyl (R)-4-((R/S)-3-ethynylcyclopent-2-en-1-yl)-3-methyl piperazine-1-carboxylate (Compound 14d, 1.5 g, 5.17 mmol) in dichloromethane (10 ml) was added, hydrochloric acid in 1,4 dioxane (12.91 ml, 51.7 mmol, 4M solution in 1,4 dioxane) at 0-5° C. The reaction mixture was warmed to room temperature and stirred for 2 h. The progress of reaction was monitored by TLC. The reaction mixture was evaporated under reduced pressure to obtain a solid product which was co-evaporated with diethyl ether (50 ml), followed by toluene (50 ml) to obtain the title product (1.35 gm, 99%) as a white solid.

¹H NMR (400 MHz, DMSO-d₆) δ 6.05 (s, 1H), 4.91 (s, 1H), 4.37 (d, J=2.5 Hz, 1H), 3.62-3.44 (m, 3H), 3.44-3.11 (m, 4H), 2.95-2.54 (m, 2H), 2.36-2.16 (m, 1H), 2.10-1.93 (m, 1H), 1.40 (d, J=6.4 Hz, 3H).

MS: m/z 191.2 (M+1).

### Step 6: 4-((R)-4-((R/S)-3-ethynylcyclopent-2-en-1-yl)-3-methylpiperazin-1-yl)benzonitrile (Compound 14f)

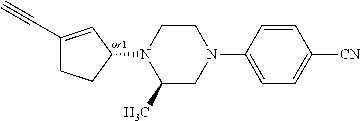 [SMILES: C#CC1=C[C@H](N2CCN(c3ccc(C#N)cc3)C[C@H]2C)CC1]

To a solution of (R)-1-((R/S)-3-ethynylcyclopent-2-en-1-yl)-2-methylpiperazine dihydrochloride (Compound 14e, 1.3 g, 4.94 mmol) in dimethylsulfoxide (10 ml) was added, potassium carbonate (3.07 g, 22.23 mmol) followed by the addition of 4-fluoro benzonitrile (0.778 g, 6.42 mmol) at 25-30° C. The reaction mixture was warmed and stirred at 120° C. for 18 h. The progress of the reaction was monitored by TLC. The reaction mixture was poured into water (25 ml) and extracted with ethyl acetate (2×50 ml) and the organic layer was washed with water (25 ml) and brine solution (25 ml) simultaneously. The organic layer separated was dried over sodium sulphate and evaporated under vacuum to obtain crude oily product which was purified by column chromatography over silica gel (100-200 mesh) using 35-40% ethyl acetate in hexane as an eluent to obtain the title product (1.15 gm, 80.0% yield).

¹H NMR (400 MHz, CDCl₃) δ 7.52 (d, J=8.5 Hz, 2H), 6.87 (d, J=8.5 Hz, 2H), 6.07 (s, 1H), 4.51 (s, 1H), 3.76-3.46 (m, 2H), 3.25-2.88 (m, 2H), 2.91-2.12 (m, 6H), 2.15-1.68 (m, 2H), 1.30-1.02 (m, 3H).

MS: m/z 292.2 (M+1).

### Step 7: 4-((R)-3-methyl-4-((R/S)-3-(5-oxo-5H-pyrano[4,3-b]pyridin-7-yl)cyclopent-2-en-1-yl)piperazin-1-yl)benzonitrile (Compound 14g)

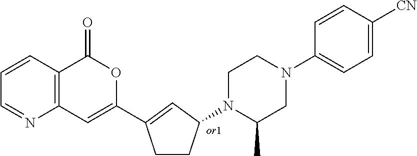 [SMILES: C[C@@H]1CN(c2ccc(C#N)cc2)CCN1[C@H]1C=C(c2cc3ncccc3c(=O)o2)CC1]

To a stirred solution of 2-bromonicotinic acid (0.763 g, 3.78 mmol) in acetonitrile (50 ml) (degassed by N₂ purge) was added bis(triphenylphosphine)palladium(II) chloride (0.265 g, 0.378 mmol). The reaction mixture was heated upto 70° C. and diisopropylethyl amine (2.93 g, 22.65 mmol) was added slowly, followed by a solution of 4-((R)-4-((R/S)-3-ethynylcyclopent-2-en-1-yl)-3-methylpiperazin-1-yl)benzonitrile (Compound 14f, 1.10 g, 3.78 mmol) in acetonitrile (10 ml) was added slowly at same temperature. The mixture was heated and stirred at 80-85° C. for 24 h. The progress of the reaction was monitored by TLC. The reaction mixture was distilled under vacuum to dryness to obtain a crude product which was purified by column chromatography over silica gel (100-200 mesh) using ethyl acetate in hexane (100% ethyl acetate) as an eluent to obtain the title product (0.55 gm, 35.3% yield).

¹H NMR (400 MHz, CDCl₃) δ 8.97-8.90 (m, 1H), 8.58-8.51 (m, 1H), 6 7.62-7.54 (m, 2H), 7.46 (dd, J=11.5, 2.9 Hz, 1H), 6.87 (dd, J=8.8, 3.5 Hz, 2H), 6.66 (d, J=9.8 Hz, 1H), 4.61 (s, 1H), 3.76-3.57 (m, 2H), 3.04 (d, J=17.1 Hz, 1H), 2.90-2.65 (m, 4H), 2.59-2.42 (m, 2H), 2.07 (s, 2H), 1.34-1.15 (m, 4H).

MS: m/z 413.3 (M+1).

### Step 8: 4-((R)-3-methyl-4-((R/S)-3-(5-oxo-5,6-dihydro-1,6-naphthyridin-7-yl)cyclopent-2-en-1-yl)piperazin-1-yl)benzonitrile (Compound 14)

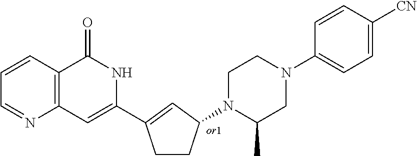 [SMILES: C[C@@H]1CN(c2ccc(C#N)cc2)CCN1[C@H]1C=C(c2cc3ncccc3c(=O)[nH]2)CC1]

To a solution of 4-((R)-3-methyl-4-((R/S)-3-(5-oxo-5H-pyrano[4,3-b]pyridin-7-yl)cyclopent-2-en-1-yl)piperazin-1-yl)benzonitrile (Compound 14g, 0.5 g, 1.212 mmol) in anhydrous tetrahydrofuran (5 ml) was added ammonia in methanol (8.66 ml, 60.6 mmol, 7M solution in methanol) at 25° C., reaction mixture in steel bomb was stirred at 80-85° C. for 24 hrs. The progress of the reaction was monitored by TLC. The reaction mixture was distilled under vacuum. A crude product was purified by chromatography using methanol in dichloromethane. The desired compound was isolated at 3-4% of methanol in dichloromethane to obtain the title compound (0.130 mg, 26.1% yield).

¹H NMR (400 MHz, DMSO-d₆) δ 11.51 (brs-exchangeable with D₂O, 1H), 8.90 (dd, J=4.6, 1.8 Hz, 1H), 8.48 (dd, J=8.0, 1.8 Hz, 1H), 7.57 (d, J=8.6 Hz, 2H), 7.47 (dd, J=8.0, 4.6 Hz, 1H), 7.02 (d, J=8.7 Hz, 2H), 6.74 (s, 1H), 6.57 (s, 1H), 4.48 (s, 1H), 3.73 (d, J=11.7 Hz, 2H), 2.94-2.89 (m, 1H), 2.80-2.54 (m, 5H), 2.40-2.30 (m, 1H), 1.98-1.84 (m, 2H), 1.16 (d, J=5.9 Hz, 3H).

MS: m/z 412.2 (M+1).

### Step 9: 4-((R)-3-methyl-4-((R/S)-3-(5-oxo-5,6-dihydro-1,6-naphthyridin-7-yl)cyclopent-2-en-1-yl)piperazin-1-yl)benzonitrile (Compound 14—hydrochloride salt)

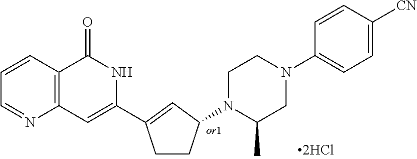 [SMILES: C[C@@H]1CN(c2ccc(C#N)cc2)CCN1[C@H]1C=C(c2cc3ncccc3c(=O)[nH]2)CC1]

A clear solution of 4-((R)-3-methyl-4-((R/S)-3-(5-oxo-5,6-dihydro-1,6-naphthyridin-7-yl)cyclopent-2-en-1-yl)piperazin-1-yl)benzonitrile (Compound 14, 120 mg, 0.292 mmol) in dichloromethane (5 ml) and methanol (5 ml), was warmed and stirred at 55-60° C., then hydrochloric acid in dioxane (0.583 ml, 1.750 mmol, 3M solution in dioxane) was added at same temperature in small portions over a period of 5 minute. The reaction mixture was stirred for 30 min at 55-60° C. The reaction mixture was cooled to room temperature, diluted with diethyl ether (10 ml), and product was collected upon filtration. The solid compound was washed with diethyl ether (10 ml) and dried under reduced pressure for 3 h at 40° C. to obtain the title compound (0.115 g, 81% yield).

¹H NMR (400 MHz, DMSO-d₆) δ 11.94 (brs-exchangeable with D₂O, 1H), 11.87 (brs-exchangeable with D₂O, 1H), 9.06-8.95 (m, 1H), 8.65 (d, J=8.0 Hz, 1H), 7.74-7.56 (m, 3H), 7.14 (d, J=8.6 Hz, 2H), 6.83-6.65 (m, 2H), 5.05 (s, 1H), 4.26-4.03 (m, 2H), 3.57 (s, 1H), 3.45-3.30 (m, 2H), 3.27-3.02 (m, 2H), 2.95-2.78 (m, 2H), 2.40-2.20 (m, 2H), 1.54-1.35 (m, 3H).

MS: m/z 412.1 (M+1).

The following compound was prepared using the procedure described above in Example 7 by using tert-butyl (R)-4-((S/R)-3-cyanocyclopent-2-en-1-yl)-3-methylpiperazine-1-carboxylate (Compound 14b″)

### 4-((R)-3-methyl-4-((S/R)-3-(5-oxo-5,6-dihydro-1,6-naphthyridin-7-yl)cyclopent-2-en-1-yl)piperazin-1-yl)benzonitrile (Compound 15—hydrochloride salt)

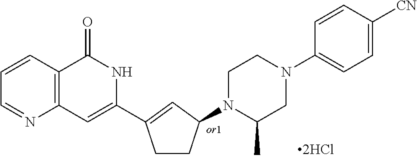 [SMILES: C[C@@H]1CN(c2ccc(C#N)cc2)CCN1[C@@H]1C=C(c2cc3ncccc3c(=O)[nH]2)CC1]

¹H NMR (400 MHz, DMSO-d₆) δ 11.88 (brs-exchangeable with D₂O, 2H), 9.12-9.00 (m, 1H), 8.77 (t, J=9.4 Hz, 1H), 7.73 (dd, J=7.9, 5.3 Hz, 1H), 7.66 (d, J=8.5 Hz, 2H), 7.14 (dd, J=8.6, 5.4 Hz, 2H), 6.91 (s, 1H), 6.85 (s, 1H), 5.06 (s, 1H), 4.20 (d, J=13.3 Hz, 1H), 4.16-3.97 (m, 2H), 3.55-3.06 (m, 5H), 3.03-2.75 (m, 3H), 2.49-2.37 (m, 2H), 1.57 (d, J=6.3 Hz, 2H).

MS: m/z 412.1 (M+1).

### Example 8: Synthesis of 4-((1S,4S)-5-((R/S)-3-(5-oxo-5,6-dihydro-1,6-naphthyridin-7-yl)cyclopent-2-en-1-yl)-2,5-diazabicyclo[2.2.1]heptan-2-yl) benzonitrile (Compound 16)

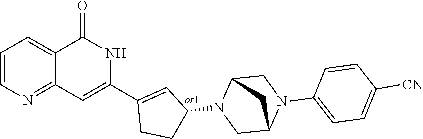 [SMILES: N#Cc1ccc(N2C[C@@H]3C[C@H]2CN3[C@H]2C=C(c3cc4ncccc4c(=O)[nH]3)CC2)cc1]

### Step 1: tert-butyl (1S,4S)-5-(3-cyanocyclopent-2-en-1-yl)-2,5-diazabicyclo[2.2.1]heptane-2-carboxylate (Compound 16a)

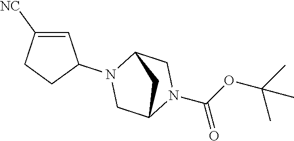 [SMILES: CC(C)(C)OC(=O)N1C[C@@H]2C[C@H]1CN2C1C=C(C#N)CC1]

To a stirred solution of tert-butyl (1S,4S)-2,5-diazabicyclo[2.2.1]heptane-2-carboxylate (10.0 g, 50.4 mmol) in acetonitrile (100 ml) was added potassium carbonate (20.91 g, 151 mmol) at 25° C. and stirred the reaction mixture for 10 minutes. To this was added, a solution of 3-bromocyclopent-1-enecarbonitrile (Compound 1a, 8.68 g, 50.4 mmol) in acetonitrile (25 ml) and the reaction mixture was stirred for 16 hrs. The progress of reaction was monitored by TLC. The reaction mixture was concentrated under reduced pressure. The residue obtained was diluted with water (100 ml) and extracted with ethyl acetate (2×200 ml). The combined organic layer was dried over anhydrous sodium sulphate and evaporated under reduced pressure to obtain a crude product. The crude product was purified by flash column chromatography over silica gel (100-200 mesh) using 20% ethyl acetate in hexane as an eluent to obtain the title compound (10.0 g, 68.5% yield).

¹H NMR (400 MHz, DMSO-d₆) δ 6.89 (d, J=8.1 Hz, 1H), 4.19-4.11 (m, 1H), 3.88-3.78 (m, 1H), 3.57-3.51 (m, 1H), 3.32-3.24 (m, 1H), 3.13-3.02 (m, 1H), 2.89-2.78 (m, 1H), 2.61-2.54 (m, 2H), 2.18-2.09 (m, 1H), 1.72-1.52 (m, 3H), 1.39 (s, 9H), 1.26-1.14 (m, 1H).

MS: m/z 290.0 (M+1).

### Step 2: tert-butyl (1S,4S)-5-(3-formylcyclopent-2-en-1-yl)-2,5-diazabicyclo[2.2.1]heptane-2-carboxylate (Compound 16b)

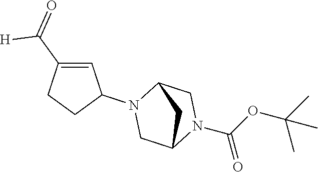 [SMILES: CC(C)(C)OC(=O)N1C[C@@H]2C[C@H]1CN2C1C=C(C=O)CC1]

A solution of tert-butyl (1S,4S)-5-(3-cyanocyclopent-2-en-1-yl)-2,5-diazabicyclo[2.2.1]heptane-2-carboxylate (Compound 16a, 10.0 g, 34.6 mmol) in dichloromethane (50 ml) was cooled at −78° C. Diisobutylaluminium hydride (51.8 ml, 51.8 mmol, 1M solution in toluene) was added slowly within 10-15 minute. The reaction mixture was stirred for 15 min at −78° C. and then warmed at room temperature and stirred for 1 h. The progress of the reaction was monitored by TLC. The reaction mixture was quenched by drop wise addition of saturated ammonium chloride solution (20 ml) at 0° C. (carefully: The reaction quenching is exothermic). A gel type reaction mass was observed, Celite (100 g) was added to the reaction mixture and the reaction mixture was diluted with 10% methanol in dichloromethane (300 ml) and stirred for 20 min. The reaction mass was filtered through Celite bed and the bed was washed with 10% methanol in dichloromethane (300 ml). The combined organic filtrate was dried over sodium sulphate and concentrated under vacuum till dryness to afford the crude product which was purified by column chromatography over silica gel (100-200 mesh) using ethyl acetate in hexane as an eluent to obtain the title product as yellow solid. (5.0 g, 49.5% yield).

¹H NMR (400 MHz, DMSO-d₆) δ 9.77 (s, 1H), 7.01 (d, J=9.9 Hz, 1H), 4.21-4.13 (m, 1H), 3.96-3.83 (m, 1H), 3.58 (s, 1H), 3.33-3.29 (m, 1H), 3.16-3.05 (m, 1H), 2.89-2.81 (m, 1H), 2.65-2.56 (m, 1H), 2.47-2.38 (m, 1H), 2.36-2.22 (m, 1H), 2.21-2.09 (m, 1H), 1.74-1.61 (m, 3H), 1.40 (s, 9H).

MS: m/z 292.4 (M+1).

### Step 3: tert-butyl (1S,4S)-5-(3-ethynylcyclopent-2-en-1-yl)-2,5-diazabicyclo[2.2.1]heptane-2-carboxylate (Compound 16c)

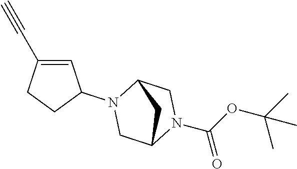 [SMILES: C#CC1=CC(N2C[C@@H]3C[C@H]2CN3C(=O)OC(C)(C)C)CC1]

To a solution of trimethylsilyldiazomethane (12.23 ml, 24.46 mmol) in tetrahydrofuran (50 ml) at −78° C. was slowly added, n-butyllithium (15.28 ml, 24.46 mmol) solution in hexane (1.6 M). The reaction mixture was stirred for 30 minutes at same temperature. A solution of tert-butyl (1S,4S)-5-(3-formylcyclopent-2-en-1-yl)-2,5-diazabicyclo[2.2.1]heptane-2-carboxylate (Compound 16b, 5.5 g, 18.81 mmol) in tetrahydrofuran (50 ml) was slowly added to the reaction mixture at −78° C. The reaction mixture was stirred for 30 minute and then warmed to room temperature and further stirred for 2 h. The progress of the reaction was monitored by TLC. The reaction mixture was diluted with ethyl acetate (100 ml) and then washed with water (50 ml). The organic layer was separated and aqueous layer was again extracted with ethyl acetate (2×100 ml). The combined organic layer was dried over sodium sulphate and evaporated under reduced pressure to obtain crude oily product which was purified by column chromatography over silica gel (100-200 mesh) using ethyl acetate in hexane as an eluent to obtain the title product (2.5 g, 46.1% yield).

¹H NMR (400 MHz, DMSO-d₆) δ 6.16-6.07 (m, 1H), 4.18-4.09 (m, 2H), 3.79-3.69 (m, 1H), 3.55-3.47 (m, 1H), 3.13-3.00 (m, 1H), 2.86-2.76 (m, 1H), 2.50-2.37 (m, 3H), 2.35-2.25 (m, 1H), 2.11-2.01 (m, 1H), 1.72-1.52 (m, 3H), 1.39 (s, 9H).

MS: m/z 289.2 (M+1).

### Step 4: (1S,4S)-2-(3-ethynylcyclopent-2-en-1-yl)-2,5-diazabicyclo[2.2.1]heptane dihydrochloride (Compound 16d)

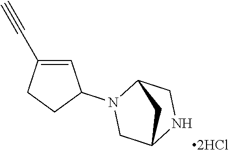

To a solution of tert-butyl (1S,4S)-5-(3-ethynylcyclopent-2-en-1-yl)-2,5-diazabicyclo[2.2.1]heptane-2-carboxylate (Compound 16c, 2.5 g, 8.67 mmol) in dichloromethane (10 ml) was added, hydrochloric acid in 1,4 dioxane (21.67 ml, 87 mmol, 4M solution in 1,4 dioxane) at 0-5° C. The reaction mixture was warmed to room temperature and stirred for 2 h. The progress of reaction was monitored by TLC. The reaction mixture was evaporated under reduced pressure to obtain solid product which was co-evaporated with diethyl ether (50 ml), followed by toluene (50 ml) to obtain the title product (2.2 g, 97% yield) as a white solid.

¹H NMR (400 MHz, DMSO-d₆) δ 6.16-6.07 (m, 1H), 4.18-4.09 (m, 2H), 3.79-3.69 (m, 1H), 3.55-3.47 (m, 1H), 3.13-3.00 (m, 1H), 2.86-2.76 (m, 1H), 2.50-2.37 (m, 3H), 2.35-2.25 (m, 1H), 2.11-2.01 (m, 1H), 1.72-1.52 (m, 3H).

MS: m/z 188.9 (M+1).

### Step 5: 4-((1S,4S)-5-(3-ethynylcyclopent-2-en-1-yl)-2,5-diazabicyclo[2.2.1]heptan-2-yl)benzonitrile (Compound 16e)

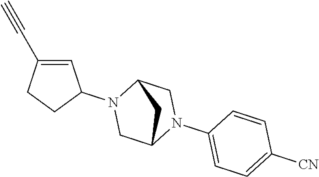 [SMILES: C#CC1=CC(N2C[C@@H]3C[C@H]2CN3c2ccc(C#N)cc2)CC1]

To a solution of (1S,4S)-2-(3-ethynylcyclopent-2-en-1-yl)-2,5-diazabicyclo [2.2.1]heptane dihydrochloride (Compound 16d, 2.2 g, 8.42 mmol) in dimethylsulfoxide (40 ml) was added, potassium carbonate (5.24 g, 37.9 mmol) followed by the addition of 4-fluorobenzonitrile (1.326 g, 10.95 mmol) at 25-30° C. The reaction mixture was warmed and stirred at 120° C. for 18 h. The progress of reaction was monitored by TLC. The reaction mixture was poured into water (25 ml) and extracted with ethyl acetate (2×100 ml) and organic layer was washed with water (50 ml) and brine solution (50 ml). The organic layer was dried over sodium sulphate and evaporated under vacuum to obtain crude oily product which was purified by column chromatography over silica gel (100-200 mesh) using ethyl acetate in hexane (35-40% ethyl acetate) as an eluent to obtain the title product (1.6 gm, 65.6% yield).

¹H NMR (400 MHz, CDCl₃) δ 7.50-7.42 (m, 2H), 6.56-6.48 (m, 2H), 6.04 (dd, J=7.3, 2.2 Hz, 1H), 4.32 (s, 1H), 3.84-3.71 (m, 2H), 3.46-3.34 (m, 2H), 3.15-2.99 (m, 2H), 2.77-2.52 (m, 2H), 2.51-2.36 (m, 1H), 2.21-1.98 (m, 2H), 1.95-1.88 (m, 1H), 1.78-1.65 (m, 1H).

MS: m/z 290.1 (M+1).

A chiral separation of 4-((1S,4S)-5-(3-ethynylcyclopent-2-en-1-yl)-2,5-diazabicyclo[2.2.1]heptan-2-yl)benzonitrile by chiral HPLC was carried out using chiral column to obtain

### 4-((1S,4S)-5-((R/S)-3-ethynylcyclopent-2-en-1-yl)-2,5-diazabicyclo [2.2.1]heptan-2-yl)benzonitrile (Compound 16e′)

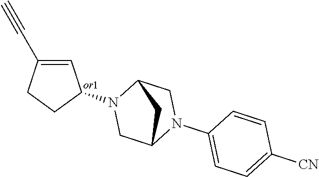 [SMILES: C#CC1=C[C@H](N2C[C@@H]3C[C@H]2CN3c2ccc(C#N)cc2)CC1]

¹H NMR (400 MHz, CDCl₃) δ 7.46 (d, J=8.5 Hz, 2H), 6.52 (d, J=8.5 Hz, 2H), 6.05 (d, J=2.5 Hz, 1H), 4.32 (s, 1H), 3.85-3.77 (m, 1H), 3.74 (s, 1H), 3.48-3.35 (m, 2H), 3.07-3.01 (m, 1H), 2.73-2.65 (m, 1H), 2.65-2.54 (m, 1H), 2.50-2.37 (m, 1H), 2.20-2.07 (m, 1H), 2.05-1.99 (m, 1H), 1.96-1.87 (m, 1H), 1.79-1.68 (m, 2H).

MS: m/z 290.1 (M+1).

and

### 4-((1S,4S)-5-((S/R)-3-ethynylcyclopent-2-en-1-yl)-2,5-diazabicyclo [2.2.1]heptan-2-yl)benzonitrile (Compound 16e″)

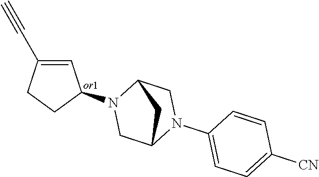 [SMILES: C#CC1=C[C@@H](N2C[C@@H]3C[C@H]2CN3c2ccc(C#N)cc2)CC1]

¹H NMR (400 MHz, CDCl₃) δ 7.46 (d, J=8.6 Hz, 2H), 6.53 (d, J=8.6 Hz, 2H), 6.03 (s, 1H), 4.33 (s, 1H), 3.83-3.72 (m, 2H), 3.46-3.34 (m, 2H), 3.15-3.09 (m, 1H), 2.78-2.71 (m, 1H), 2.65-2.54 (m, 1H), 2.51-2.39 (m, 1H), 2.21-2.09 (m, 1H), 2.06-1.99 (m, 1H), 1.96-1.90 (m, 1H), 1.83-1.71 (m, 2H).

MS: m/z 290.2 (M+1).

Both these diastereomers were processed further individually to obtain the respective title products.

### Step 6: 4-((1S,4S)-5-((R/S)-3-(5-oxo-5H-pyrano[4,3-b]pyridin-7-yl)cyclopent-2-en-1-yl)-2,5-diazabicyclo[2.2.1]heptan-2-yl)benzonitrile (Compound 16f)

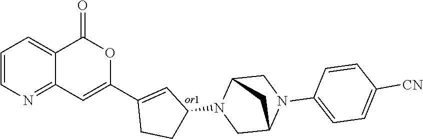 [SMILES: N#Cc1ccc(N2C[C@@H]3C[C@H]2CN3[C@H]2C=C(c3cc4ncccc4c(=O)o3)CC2)cc1]

To a stirred solution in another round bottom flask of 2-bromonicotinic acid (0.635 g, 3.14 mmol) in acetonitrile (50 ml) (degassed by N₂ purge separately) was added bis(triphenylphosphine)palladium(II) chloride (0.085 g, 0.121 mmol). The reaction mixture was heated up to 70° C. and diisopropylethyl amine (2.53 ml, 14.51 mmol) was added slowly, followed by a solution of 4-((1S,4S)-5-((R/S)-3-ethynylcyclopent-2-en-1-yl)-2,5-diazabicyclo[2.2.1]heptan-2-yl)benzonitrile (Compound 16e′, 0.700 g, 2.419 mmol) in acetonitrile (10 ml) was added slowly at same temperature. The mixture was heated and stirred at 80-85° C. for 24 hrs. The progress of the reaction was monitored by TLC. The reaction mixture was distilled under vacuum till dryness to obtain crude product which was purified by column chromatography over silica gel (100-200 mesh) using ethyl acetate in hexane (100% ethyl acetate) as an eluent to obtain the title product (150 mg, 38.0% yield).

MS: m/z 411.3 (M+1).

### Step 7: 4-((1S,4S)-5-((R/S)-3-(5-oxo-5,6-dihydro-1,6-naphthyridin-7-yl)cyclopent-2-en-1-yl)-2,5-diazabicyclo[2.2.1]heptan-2-yl)benzonitrile (Compound 16)

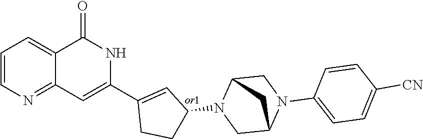 [SMILES: N#Cc1ccc(N2C[C@@H]3C[C@H]2CN3[C@H]2C=C(c3cc4ncccc4c(=O)[nH]3)CC2)cc1]

To a solution of 4-((1S,4S)-5-((R/S)-3-(5-oxo-5H-pyrano[4,3-b]pyridin-7-yl)cyclopent-2-en-1-yl)-2,5-diazabicyclo[2.2.1]heptan-2-yl)benzonitrile (Compound 16f, 0.150 g, 0.365 mmol) in anhydrous tetrahydrofuran (5 ml) was added ammonia in methanol (5.22 ml, 36.5 mmol, 7M solution in methanol) at 25° C., reaction mixture was stirred at 80-85° C. for 12 h. The progress of the reaction was monitored by TLC. The reaction mixture was distilled under vacuum. A crude product was purified by chromatography using methanol in dichloromethane. The desired compound was isolated at 3-4% of methanol in dichloromethane. (0.050 g, 33.4% yield)

MS: m/z 410.1 (M+1).

### Step 8: 4-((1S,4S)-5-((R/S)-3-(5-oxo-5,6-dihydro-1,6-naphthyridin-7-yl)cyclopent-2-en-1-yl)-2,5-diazabicyclo[2.2.1]heptan-2-yl)benzonitrile (Compound 16-hydrochloride salt)

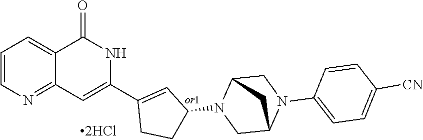 [SMILES: N#Cc1ccc(N2C[C@@H]3C[C@H]2CN3[C@H]2C=C(c3cc4ncccc4c(=O)[nH]3)CC2)cc1]

A clear solution of 4-((1S,4S)-5-((R/S)-3-(5-oxo-5,6-dihydro-1,6-naphthyridin-7-yl)cyclopent-2-en-1-yl)-2,5-diazabicyclo[2.2.1]heptan-2-yl)benzonitrile (Compound 16, 0.050 g, 0.122 mmol) in dichloromethane (5 ml) and methanol (5 ml), was warmed and stirred at 55-60° C., and hydrochloric acid in dioxane (0.244 ml, 0.977 mmol, 3M solution in dioxane) was added at the same temperature in small portions over a period of 5 minutes. The reaction mixture was stirred for 30 min at 55-60° C. The reaction mixture was cooled to room temperature, diluted with diethyl ether (10 ml), and product was collected upon filtration. The solid compound was washed with diethyl ether (10 ml) and dried under reduced pressure for 3 h at 40° C. to obtain the title compound (0.011 g, 18.67% yield).

¹H NMR (400 MHz, DMSO-d₆) δ 11.61 (brs-exchangeable with D₂O, 1H), 10.35 (brs-exchangeable with D₂O, 1H), 9.01-8.91 (m, 1H), 8.60-8.51 (m, 1H), 7.72-7.51 (m, 3H), 6.86-6.73 (m, 3H), 4.96-4.82 (m, 1H), 4.77-4.65 (m, 1H), 4.58 (s, 1H), 3.87-3.74 (m, 1H), 3.75-3.62 (m, 1H), 3.59-3.52 (m, 2H), 3.42-3.31 (m, 1H), 3.18-2.95 (m, 1H), 2.90-2.62 (m, 1H), 2.48-2.29 (m, 2H), 2.30-2.17 (m, 1H), 2.18-2.05 (m, 1H).

MS: m/z 410.2 (M+1).

The following compound was prepared using the procedure described above in Example 8 by using 4-((1S,4S)-5-((S/R)-3-ethynylcyclopent-2-en-1-yl)-2,5-diazabicyclo[2.2.1]heptan-2-yl)benzonitrile (Compound 16e″).

### 4-((1S,4S)-5-((S/R)-3-(5-oxo-5,6-dihydro-1,6-naphthyridin-7-yl)cyclopent-2-en-1-yl)-2,5-diazabicyclo[2.2.1]heptan-2-yl)benzonitrile (Compound 17—hydrochloride salt)

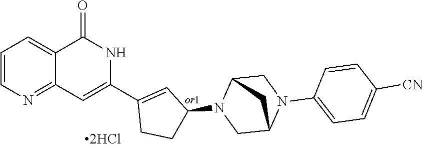 [SMILES: N#Cc1ccc(N2C[C@@H]3C[C@H]2CN3[C@@H]2C=C(c3cc4ncccc4c(=O)[nH]3)CC2)cc1]

¹H NMR (400 MHz, DMSO-d₆) δ 11.66 (brs-exchangeable with D₂O, 1H), 10.91 (brs-exchangeable with D₂O, 1H), 9.08-8.97 (m, 1H), 8.71-8.56 (m, 1H), 7.75-7.57 (m, 3H), 6.89-6.72 (m, 3H), 4.96-4.82 (m, 1H), 4.77-4.66 (m, 1H), 4.60 (s, 1H), 3.89-3.79 (m, 1H), 3.75-3.64 (m, 1H), 3.62-3.56 (m, 2H), 3.42-3.33 (m, 1H), 3.18-2.95 (m, 1H), 2.92-2.63 (m, 1H), 2.48-2.29 (m, 2H), 2.31-2.18 (m, 1H), 2.19-2.07 (m, 1H).

MS: m/z 410.2 (M+1).

### Example 9: Synthesis of 4-(4-((1R,3S/3R)-3-(5-oxo-5,6-dihydro-1,6-naphthyridin-7-yl)cyclopentyl)piperazin-1-yl)benzonitrile (Compound 11)

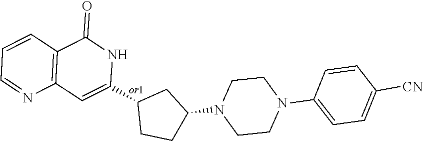 [SMILES: N#Cc1ccc(N2CCN([C@@H]3CC[C@H](c4cc5ncccc5c(=O)[nH]4)C3)CC2)cc1]

and

### 4-(4-((1R,3R/3S)-3-(5-oxo-5,6-dihydro-1,6-naphthyridin-7-yl)cyclopentyl)piperazin-1-yl)benzonitrile (Compound 12)

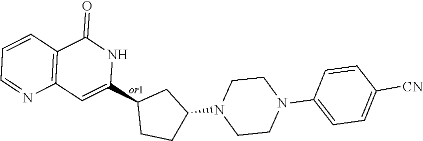 [SMILES: N#Cc1ccc(N2CCN([C@@H]3CC[C@@H](c4cc5ncccc5c(=O)[nH]4)C3)CC2)cc1]

### Step 1: tert-butyl 4-((1R)-3-cyanocyclopentyl)piperazine-1-carboxylate (Compound 11a)

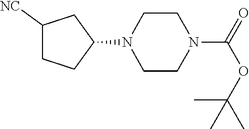 [SMILES: CC(C)(C)OC(=O)N1CCN([C@@H]2CCC(C#N)C2)CC1]

To a stirred solution of tert-butyl (R)-4-(3-cyanocyclopent-2-en-1-yl)piperazine-1-carboxylate (Compound 23b′, 13.5 g, 48.7 mmol) in methanol (150 ml) at 25° C. was added 10% Pd/C (5 g). The resulting suspension was stirred under Hydrogen Balloon pressure for 3 hrs. The progress of the reaction was monitored by TLC. The reaction mixture filtered through a bed of Celite and was washed with methanol (50 ml). The combined filtrate was concentrated under reduced pressure to obtain a crude product, which was purified by column chromatography over silica gel (100-200 mesh) using 50% ethyl acetate in hexane as an eluent to obtain the title compound (7.4 g, 54%).

¹HNMR (400 MHz, CDCl₃): δ 3.47-3.42 (m, 4H), 2.86-2.72 (m, 1H), 2.65-2.61 (m, 1H), 2.43 (d, J=5.3 Hz, 4H), 2.42-2.25 (m, 1H), 2.11-2.01 (m, 2H), 1.97-1.88 (m, 1H), 1.85-1.69 (m, 2H), 1.47 (s, 9H).

MS: m/z 280 (M+1).

### Step 2: tert-butyl 4-((1R)-3-formylcyclopentyl)piperazine-1-carboxylate (Compound 11b)

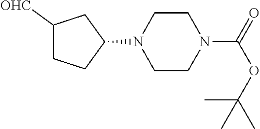 [SMILES: CC(C)(C)OC(=O)N1CCN([C@@H]2CCC(C=O)C2)CC1]

To a stirred solution of tert-butyl 4-((1R)-3-cyanocyclopentyl)piperazine-1-carboxylate (Compound 11a, 7.4 g, 26.5 mmol) in dichloromethane (400 ml) at −78° C. DIBAL-H in toluene (39.7 ml, 39.7 mmol) was added slowly. The reaction mixture was allowed to reach 25-30° C. The progress of the reaction was monitored by TLC. After completion of reaction, the mixture was cooled to 0° C. and then quenched with saturated ammonium chloride solution (30 ml). The reaction mixture was diluted with 10% methanol in dichloromethane (500 ml) and stirred for 30 min. The reaction mass was filtered through bed of Celite and washed with 10% methanol in dichloromethane (500 ml). The organic layer was concentrated under reduced pressure to obtain crude product, which was purified by flash column chromatography over silica gel (100-200 mesh) using ethyl acetate in hexane as an eluent to obtain title compound (4.1 g, 54.8% yield).

¹HNMR (400 MHz, CDCl₃): δ 9.64 (dd, J=8.8, 2.0 Hz, 1H), 3.45-3.40 (m, 4H), 2.84-2.70 (m, 1H), 2.65-2.61 (m, 1H), 2.45-2.40 (m, 4H), 2.41-2.23 (m, 1H), 2.13-2.04 (m, 2H), 1.98-1.85 (m, 1H), 1.82-1.67 (m, 2H), 1.47 (s, 9H).

MS: m/z 283 (M+1).

### Step 3: tert-butyl 4-((1R,3S/3R)-3-ethynylcyclopentyl)piperazine-1-carboxylate (Compound 11c)

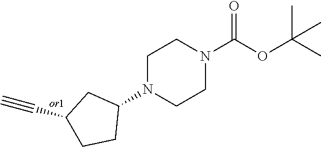 [SMILES: C#C[C@H]1CC[C@@H](N2CCN(C(=O)OC(C)(C)C)CC2)C1]

and

### tert-butyl 4-((1R,3R/3S)-3-ethynylcyclopentyl)piperazine-1-carboxylate (Compound 11c′)

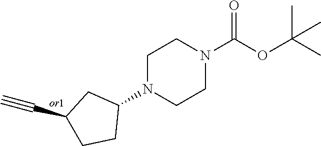 [SMILES: C#C[C@@H]1CC[C@@H](N2CCN(C(=O)OC(C)(C)C)CC2)C1]

To a stirred solution of trimethylsilyl diazomethane (11.33 ml, 22.66 mmol, 2.0 M solution in hexane) in dry tetrahydrofuran at −78° C. was added nBuLi (13.28 ml, 21.25 mmol, 1.6 M in toluene) under nitrogen atmosphere. The reaction mixture was stirred for 30 min. A solution of tert-butyl 4-((1R)-3-formylcyclopentyl)piperazine-1-carboxylate (Compound 1 b, 4.0g, 14.17 mmol) in tetrahydrofuran (50 ml) was added slowly. The reaction mixture was allowed to come to room temperature and stirred for 2 hrs. The progress of the reaction was monitored by TLC. The reaction mixture was diluted with ethyl acetate (250 ml) and water (150 ml), organic layer was separated dried over sodium sulphate, filtered and filtrate was concentrated under reduced pressure to obtain crude product which was purified by flash column chromatography over silica gel (100-200 mesh) using 45-50% ethyl acetate in hexane as an eluent to obtain the title compound assigned as tert-butyl 4-((1R,3S/3R)-3-ethynylcyclopentyl)piperazine-1-carboxylate (Compound 11c, 1.75 gm) and another polar spot was eluted using 45-50% ethyl acetate in hexane were concentrated as tert-butyl 4-((1R,3R/3S)-3-ethynylcyclopentyl)piperazine-1-carboxylate (Compound 11c′, 0.75 gm).

### tert-butyl 4-((1R,3S/3R)-3-ethynylcyclopentyl)piperazine-1-carboxylate (Compound 11c)

¹H NMR (400 MHz, CDCl₃): δ 3.52-3.45 (m, 4H), 2.72-2.56 (m, 2H), 2.49-2.46 (m, 4H), 2.31-2.22 (m, 1H), 2.09 (d, J=2.2 Hz, 1H), 2.05-1.96 (m, 1H), 1.91-1.78 (m, 2H), 1.73-1.58 (m, 2H), 1.48 (s, 9H).

MS: m/z 279 (M+1).

### tert-butyl 4-((1R,3R/3S)-3-ethynylcyclopentyl)piperazine-1-carboxylate (Compound 11c′)

¹H NMR (400 MHz, CDCl₃): δ 3.53-3.48 (m, 4H), 2.71-2.57 (m, 2H), 2.48-2.44 (m, 4H), 2.31-2.22 (m, 1H), 2.09 (d, J=2.2 Hz, 1H), 2.05-1.96 (m, 1H), 1.91-1.78 (m, 2H), 1.75-1.55 (m, 2H), 1.47 (s, 9H).

MS: m/z 279 (M+1).

Both these diastereomers were processed further individually to obtain the respective title products.

### Step 4: 1-((1R,3S/3R)-3-ethynylcyclopentyl)piperazine dihydrochloride (Compound 11d)

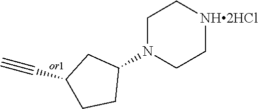

To a stirred solution of tert-butyl 4-((1R,3S/3R)-3-ethynylcyclopentyl)piperazine-1-carboxylate (Compound 11c, 1.7 g, 6.11 mmol) in Dichloromethane (40 ml) was added HCl (20.36 ml, 61.1 mmol) in 1,4 Dioxane drop wise at 0° C. After complete addition the reaction mixture was stirred at room temperature for 2 hrs. The progress of reaction was checked by TLC. The reaction mixture was concentrated under reduced pressure to remove solvent. The residue was co-evaporated with toluene to remove traces of moisture and solid was dried under vacuum to obtain compound 1-((1R,3S/3R)-3-ethynylcyclopentyl)piperazine dihydrochloride (1.25 g, 95%)

MS: m/z 180 (M+1).

### Step 5: 4-(4-((1R,3S/3R)-3-ethynylcyclopentyl)piperazin-1-yl)benzonitrile (compound 11e)

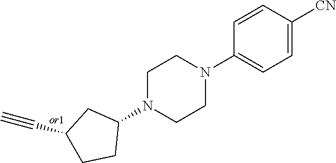 [SMILES: C#C[C@H]1CC[C@@H](N2CCN(c3ccc(C#N)cc3)CC2)C1]

To a stirred suspension of 1-((1R,3S/3R)-3-ethynylcyclopentyl)piperazine hydrochloride (Compound 11d, 1.23 g, 5.73 mmol) in dimethyl sulphoxide (20 ml) was added potassium carbonate (3.96 g, 28.6 mmol) and stirred for 30 minutes at room temperature. 4-fluorobenzonitrile (0.902 g, 7.45 mmol) was added and reaction mixture was heated at 120° C. for 15 hrs. The progress of reaction was monitored by TLC. The reaction mixture was cooled to room temperature and was diluted with ethyl acetate (120 ml) and was washed with water (2×100 ml). The separated organic layer was dried over anhydrous sodium sulfate, filtered and concentrated to yield crude compound. A crude compound was purified by flash column chromatography using 50-60% ethyl acetate in hexane to obtain compound (1.35 g, 85%).

¹H NMR (400 MHz, CDCl₃): δ 7.51 (d, J=8.6 Hz, 2H), 6.88 (d, J=8.6 Hz, 2H), 3.39-3.36 (m, 4H), 2.77-2.62 (m, 5H), 2.32-2.24 (m, 1H), 2.10 (d, J=2.2 Hz, 1H), 2.06-2.01 (m, 1H), 1.97-1.65 (m, 5H).

MS: m/z 280 (M+1).

### Step 6: methyl 2-(((1S/1R,3R)-3-(4-(4-cyanophenyl)piperazin-1-yl)cyclopentyl)ethynyl)nicotinate (Compound 11f)

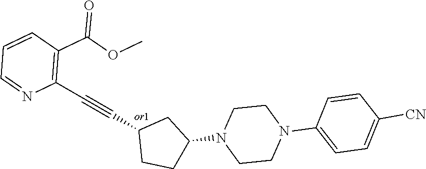 [SMILES: COC(=O)c1cccnc1C#C[C@H]1CC[C@@H](N2CCN(c3ccc(C#N)cc3)CC2)C1]

To a stirred solution of methyl 2-bromonicotinate (1.055 g, 4.89 mmol) and DIPEA (3.94 ml, 22.55 mmol) in acetonitrile (20 ml), nitrogen gas was purged for 20 minutes and bis(triphenylphosphine) palladium (II) chloride (0.264 g, 0.376 mmol) was added. The reaction mixture was heated at 85° C. and solution of 4-(4-((1R,3S/3R)-3-ethynylcyclopentyl)piperazin-1-yl)benzonitrile (Compound 11e, 1.05 g, 3.76 mmol) in acetonitrile (20 ml) was added. The reaction mixture was stirred for 18 hrs at 85° C. The progress of reaction was monitored by TLC. The reaction mixture was diluted with water (100 ml) and extracted with ethyl acetate (2×50 ml). The combined organic layer was washed with water (70 ml). The organic layer separated was washed with brine (50 ml) and dried over sodium sulphate and concentrated under reduced pressure to obtain crude compound. A crude compound was purified by Flash column chromatography using 30-40% ethyl acetate in hexane to obtain the title compound (0.51 g, 32.7%).

¹H NMR (400 MHz, CDCl₃): δ 8.77-8.66 (m, 1H), 8.23 (dd, J=8.0, 1.8 Hz, 1H), 7.52 (8.6 Hz, 2H), 7.32 (d, J=8.0 Hz, 1H), 6.88 (d, J=8.6 Hz, 2H), 3.97 (s, 3H), 3.51-3.34 (m, 4H), 3.14-3.01 (m, 1H), 2.90-2.63 (m, 5H), 2.42-1.91 (m, 6H).

MS: m/z 415 (M+1).

### Step 7: 2-(((1S/1R, 3R)-3-(4-(4-cyanophenyl)piperazin-1-yl)cyclopentyl)ethynyl)nicotinic acid (Compound 11g)

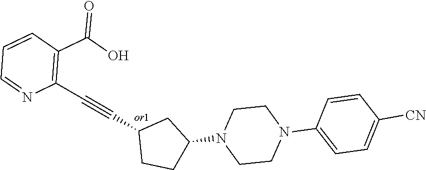 [SMILES: N#Cc1ccc(N2CCN([C@@H]3CC[C@H](C#Cc4ncccc4C(=O)O)C3)CC2)cc1]

To a stirred solution of methyl 2-(((1S/1R,3R)-3-(4-(4-cyanophenyl)piperazin-1-yl)cyclopentyl)ethynyl)nicotinate (Compound 11f, 0.5 g, 1.206 mmol) in Methanol (30 ml) was added solution of NaOH (0.193 g, 4.83 mmol) in water (10 ml) at 0° C. The reaction mixture was then stirred at 25° C. for 4 hrs. The progress of reaction was monitored by TLC. The reaction mixture was concentrated under reduced pressure to remove solvent. The residue was taken in water (15 ml) and neutralized with 2N HCl, pH was adjusted to 6-7, solid formed was filtered and co evaporated with toluene (3×20 ml) to remove moisture. The resulting solid was dried to obtain the title compound (0.460 g, 95%)

MS: m/z 401 (M+1).

### Step 8: 4-(4-((1R,3S/3R)-3-(5-oxo-5H-pyrano [4,3-b]pyridin-7-yl)cyclopentyl)piperazin-1-yl)benzonitrile (Compound 11 h)

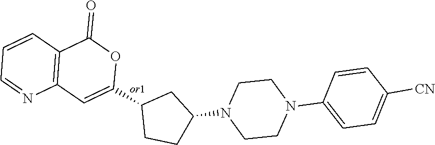 [SMILES: N#Cc1ccc(N2CCN([C@@H]3CC[C@H](c4cc5ncccc5c(=O)o4)C3)CC2)cc1]

To a stirred solution of 2-(((1S/1R,3R)-3-(4-(4-cyanophenyl)piperazin-1-yl)cyclopentyl)ethynyl)nicotinic acid (Compound 11g, 0.45 g, 1.124 mmol) in Dichloromethane (20 ml) was added trifluoromethane sulfonic acid (0.422 g, 2.81 mmol) slowly at 0° C. The reaction mixture was then stirred at 25° C. for 42 hrs. The progress of reaction was monitored by TLC. The reaction mixture was cooled in ice bath and diluted with diethyl ether (100 ml) and stirred for 30 minutes, solid obtained was filtered to yield sticky crude compound. A crude compound was purified by flash column chromatography using 4-5% methanol in dichloromethane to obtain the title compound (0.33 g, 73.3%)

¹H NMR (400 MHz, DMSO-d₆): δ 8.99 (dd, J=4.7, 1.8 Hz, 1H), 8.48 (dd, J=8.2, 1.8 Hz, 1H), 7.69 (d, J=8.4 Hz, 2H), 7.61-7.59 (m, 1H), 7.16 (d, J=8.4 Hz, 2H), 6.82 (s, 1H), 4.14-4.11 (m, 2H), 3.84-3.73 (m, 2H), 3.67-3.65 (m, 2H), 3.35-3.33 (m, 1H), 3.23-3.05 (m, 5H), 2.14-1.98 (m, 4H).

MS: m/z 401 (M+1).

### Step 9: 4-(4-((1R,3S/3R)-3-(5-oxo-5,6-dihydro-1,6-naphthyridin-7-yl)cyclopentyl)piperazin-1-yl)benzonitrile (Compound 11i)

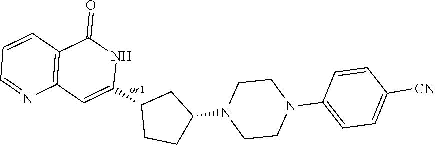 [SMILES: N#Cc1ccc(N2CCN([C@@H]3CC[C@H](c4cc5ncccc5c(=O)[nH]4)C3)CC2)cc1]

To a stirred solution of 4-(4-((1R,3S/3R)-3-(5-oxo-5H-pyrano[4,3-b]pyridin-7-yl)cyclopentyl)piperazin-1-yl)benzonitrile (Compound 11h, 0.32 g, 0.799 mmol) in methanol (2 ml) was added ammonia (7N in methanol, 10 ml). The reaction mixture in a sealed tube was then stirred at 90° C. for 15 hrs. The progress of reaction was monitored by TLC. The reaction mixture was cooled and the solid formed was filtered to yield a dark brown solid. The crude solid was purified by column chromatography over silica gel 100-200 mesh using 5-6% methanol in dichloromethane to obtain the title compound (0.13 g, 40.7%).

MS: m/z 400.2 (M+1).

### Step 10: 4-(4-((1R,3S/3R)-3-(5-oxo-5,6-dihydro-1,6-naphthyridin-7-yl)cyclopentyl)piperazin-1-yl)benzonitrile (Compound 11—hydrochloride salt)

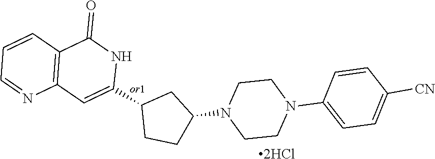 [SMILES: N#Cc1ccc(N2CCN([C@@H]3CC[C@H](c4cc5ncccc5c(=O)[nH]4)C3)CC2)cc1 ; "·2HCl"]

To a solution of 4-(4-((1R,3S/3R)-3-(5-oxo-5,6-dihydro-1,6-naphthyridin-7-yl)cyclopentyl)piperazin-1-yl)benzonitrile (Compound 11, 0.12 g, 0.30 mmol) in dichloromethane (5 ml) and ethanol (5 ml), HCl (0.74 ml, 2.403 mmol, 3 M in Dioxane) was added at 25° C. The reaction mixture was further stirred at 25° C. for 0.5 h. The reaction mixture was diluted with diethyl ether (30 ml) and stirred for 10 mins. The solid material was separated and dried under vacuum to obtain title compound (0.135 g, 95%).

¹H NMR (400 MHz, DMSO-d₆) δ 12.11 (brs-exchangeable with D₂O, 1H), 11.82 (brs-exchangeable with D₂O, 1H), 9.04 (d, J=5.2 Hz, 1H), 8.83 (d, J=8.0 Hz, 1H), 7.73-7.68 (m, 3H), 7.14 (d, J=8.0 Hz, 2H), 6.78 (s, 1H), 4.12-4.09 (m, 2H), 3.78-3.75 (m, 1H), 3.63-3.59 (m, 2H), 3.39-2.36 (m, 2H), 3.16-3.12 (m, 3H), 2.58-2.54 (m, 1H), 2.21-2.16 (m, 4H), 1.93-1.91 (m, 1H)

MS: m/z 400.2 (M+1).

The following compound of the present invention was prepared using a process analogous to Example 9 by changing the reactants to 11c′ in step 4 and following same reaction sequence.

### 4-(4-((1R, 3R/3S)-3-(5-oxo-5,6-dihydro-1,6-naphthyridin-7-yl)cyclopentyl)piperazin-1-yl)benzonitrile (Compound 12—hydrochloride salt)

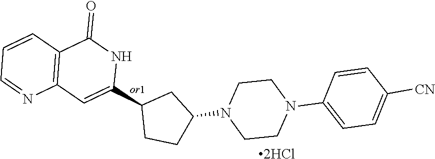 [SMILES: N#Cc1ccc(N2CCN([C@@H]3CC[C@@H](c4cc5ncccc5c(=O)[nH]4)C3)CC2)cc1 ; "·2HCl"]

¹H NMR (400 MHz, DMSO-d₆) δ 12.03 (brs-exchangeable with D₂O, 1H), 11.69 (brs-exchangeable with D₂O, 1H), 9.01 (d, J=5.2 Hz, 1H), 8.76 (d, J=8.0 Hz, 1H), 7.69-7.67 (m, 3H), 7.14 (d, J=8.6 Hz, 2H), 6.71 (s, 1H), 4.11-4.09 (m, 2H), 3.86-3.82 (m, 1H), 3.62-3.59 (m, 2H), 3.38-2.36 (m, 3H), 3.17-3.14 (m, 2H), 2.58-2.53 (m, 1H), 2.21-2.16 (m, 3H), 2.07-2.05 (m, 1H), 1.81-1.79 (m, 1H).

MS: m/z 400.2 (M+1).

### Example 10: Synthesis of (R)-4-(4-(3-(8-methyl-5-oxo-5,6-dihydro-1,6-naphthyridin-7-yl)cyclopent-2-en-1-yl)piperazin-1-yl)benzonitrile (Compound 9)

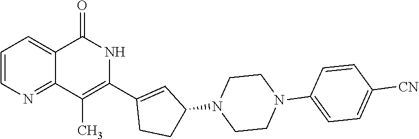 [SMILES: Cc1c(C2=C[C@H](N3CCN(c4ccc(C#N)cc4)CC3)CC2)[nH]c(=O)c2cccnc12]

and

### (S)-4-(4-(3-(8-methyl-5-oxo-5,6-dihydro-1,6-naphthyridin-7-yl)cyclopent-2-en-1-yl)piperazin-1-yl)benzonitrile (Compound 10)

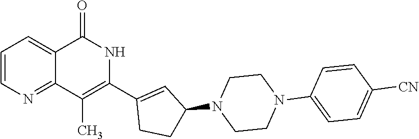 [SMILES: Cc1c(C2=C[C@@H](N3CCN(c4ccc(C#N)cc4)CC3)CC2)[nH]c(=O)c2cccnc12]

### Step 1: methyl 2-(1-cyanoethyl)nicotinate (Compound 9a)

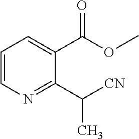

To a solution of methyl 2-(cyanomethyl)nicotinate (prepared according to the procedure reported in WO02015/200677; 15 g, 85 mmol) in dry dimethylformamide (40 ml) was added sodium hydride (3.41 g, 85 mmol) at 0-5° C. The reaction mixture was stirred at room temperature for 1 hr. To the reaction mixture, methyl iodide (12.09 g, 85 mmol) was added. The reaction mixture was stirred at room temperature for 1 hr. The progress of reaction was monitored by TLC. The reaction mixture was then concentrated under reduced pressure. The residue obtained was diluted with saturated aqueous ammonium chloride (250 ml) and extracted with ethyl acetate (3×250 ml). The combined organic layer was dried over anhydrous sodium sulphate. The organic layer was evaporated under reduced pressure to obtain a crude product. The crude product was purified by flash column chromatography over silica gel (100-200 mesh) using 30% ethyl acetate in hexane as an eluent to obtain the title compound (8 g, 49.4% yield).

¹H NMR (400 MHz, CDCl₃) δ 8.82 (dd, J=4.8, 1.8 Hz, 1H), 8.33 (dd, J=8.0, 1.8 Hz, 1H), 7.40 (dd, J=7.9, 4.8 Hz, 1H), 5.25 (q, J=7.1 Hz, 1H), 3.98 (s, 3H), 1.73 (d, J=7.1 Hz, 3H).

MS: m/z 190 (M) (GCMS).

### Step 2: 2-(1-cyanoethyl)nicotinic acid (Compound 9b)

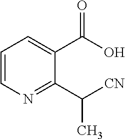

To a solution of methyl 2-(cyanomethyl)nicotinate (Compound 9a, 15 g, 85 mmol) in methanol (100 ml) was added sodium hydroxide (5.05 g, 126 mmol) in water (20 ml) at 0-25° C. The reaction mixture was stirred at room temperature for 1 hr. The progress of the reaction was monitored by TLC. The reaction mixture was then concentrated under reduced pressure. The residue obtained was diluted with water (100 ml). Aqueous phase was acidified with 2N HCl (15 ml) and extracted with ethyl acetate (4×100 ml). The combined organic layer was dried over anhydrous sodium sulphate. The organic layer was evaporated under reduced pressure to obtain crude product. The crude product was carried for next step without purification.

### Step 3: 5,7-dichloro-8-methyl-1,6-naphthyridine (Compound 9c)

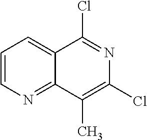

PCl₅ (9.10 g, 43.7 mmol) was dissolved in POCl₃ (60 ml) and to this solution was added 2-(1-cyanoethyl)nicotinic acid (Compound 9b, 7.0 g, 39.7 mmol) in portions. The reaction mixture was stirred at room temperature for 90 min. to form a clear solution. The reaction mixture was stirred at 70° C. for 16 hrs. The progress of the reaction was monitored by TLC. The reaction mixture was concentrated under reduced pressure. The residue obtained was poured cautiously onto 50.0 g of ice and ethyl acetate (300 ml). The phases were separated and the aqueous phase was extracted with ethyl acetate (3×100 ml). The combined organic layer was dried over anhydrous sodium sulphate. The solvent in the organic layer was evaporated under reduced pressure to obtain crude product. The crude product purified by flash column chromatography over silica gel (100-200 mesh) using 15% ethyl acetate in hexane as an eluent to obtain the title compound (4.0 g, 47.2% yield).

¹H NMR (400 MHz, CDCl₃) δ 9.17 (d, J=2.8 Hz, 1H), 8.61 (d, J=8.5 Hz, 1H), 7.63 (dd, J=8.5, 4.2 Hz, 1H), 2.83 (s, 3H).

MS: m/z 212 (M) (GCMS).

### Step 4: 7-chloro-5-methoxy-8-methyl-1,6-naphthyridine (Compound 9d)

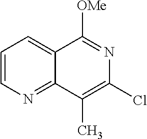

Sodium (2.158 g, 94 mmol) was dissolved in methanol (200 ml) at room temperature to form sodium methoxide. To the sodium methoxide solution was added 5,7-dichloro-8-methyl-1,6-naphthyridine (Compound 9c, 4.0 g, 18.77 mmol) in small portions. The reaction mixture was stirred at reflux temperature for 20 hr. The progress of reaction was monitored by TLC. The reaction mixture was concentrated under reduced pressure. The residue obtained was purified by flash column chromatography over silica gel (100-200 mesh) using 20% ethyl acetate in hexane as an eluent to obtain the title compound (3.4g, 87% yield).

¹H NMR (400 MHz, CDCl₃) δ 9.07 (dd, J=4.3, 1.8 Hz, 1H), 8.52 (dd, J=8.4, 1.8 Hz, 1H), 7.46 (dd, J=8.3, 4.3 Hz, 1H), 4.15 (s, 3H), 2.72 (s, 3H).

MS: m/z 208 (M) (GCMS).

### Step 5: 3-(5-methoxy-8-methyl-1,6-naphthyridin-7-yl)cyclopent-2-en-1-one (Compound 9e)

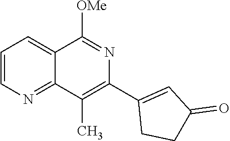 [SMILES: COc1nc(C2=CC(=O)CC2)c(C)c2ncccc12]

To a solution of 7-chloro-5-methoxy-8-methyl-1,6-naphthyridine (Compound 9d, 1.5g, 7.188 mmol) and 3-(4,4,5,5-tetramethyl-1,3,2-dioxaborolan-2-yl)cyclopent-2-enone (1.644 g, 7.92 mmol) (Synthesis reported in US2012/77814) in 1,4 Dioxane (15 ml) was added tripotassium phosphate (4.578 g, 21.57 mmol) and dicyclohexyl-[2-[2,6-di(propan-2-yloxy)phenyl]phenyl]phosphane (267 mg, 0.576 mmol) at room temperature under nitrogen purging in a microwave reaction tube for 15 minutes and Pd(OAc)₂ (65 mg, 0.30 mmol) was added to the reaction mixture. The reaction mixture was heated for 1 hr at 110° C. in microwave. The progress of reaction was monitored by TLC. The reaction mixture was diluted with water (50 ml) and ethyl acetate (50 ml). The phases were separated and the aqueous phase was extracted with ethyl acetate (2×20 ml). The combined organic layer was dried over anhydrous sodium sulphate. The solvent in the organic layer was evaporated under reduced pressure to obtain a crude product. The crude product was purified by flash column chromatography over silica gel (100-200 mesh) using 40% ethyl acetate in hexane as an eluent to obtain the title compound (1.5 g, 82% yield).

¹H NMR (400 MHz, CDCl₃) δ 9.13 (d, 1H), 8.58 (d, J=8.3 Hz, 1H), 7.54 (dd, J=8.2, 4.3 Hz, 1H), 6.61 (s, 1H), 4.16 (s, 3H), 3.36-3.28 (m, 2H), 2.82 (s, 3H), 2.66-2.59 (m, 2H).

MS: m/z 255 (M+1).

### Step 6: 3-(5-methoxy-8-methyl-1,6-naphthyridin-7-yl)cyclopent-2-en-1-ol (Compound 9f)

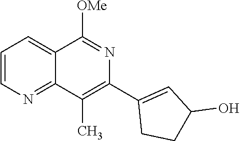 [SMILES: COc1nc(C2=CC(O)CC2)c(C)c2ncccc12]

To a solution of 3-(5-methoxy-8-methyl-1,6-naphthyridin-7-yl)cyclopent-2-enone (Compound 9e, 1.5 g, 5.90 mmol) in methanol (Volume: 50 ml) was added Cerium(III) chloride (2.93 g, 7.87 mmol). The reaction mixture was stirred at room temperature for 1 hr. The reaction mixture was cooled to 0-5° C. and sodium borohydride (0.446 g, 11.80 mmol) was added in portions. The reaction mixture was stirred at room temperature for 10 min. The progress of the reaction was monitored by TLC. The reaction mixture was diluted with water (50 ml) and ethyl acetate (25 ml). The phases were separated and the aqueous phase was extracted with ethyl acetate (3×25 ml). The combined organic layer was dried over anhydrous sodium sulphate. The solvent in the organic layer was evaporated under reduced pressure to obtain a crude product. The crude product was purified by flash column chromatography over silica gel (100-200 mesh) using 80% ethyl acetate in hexane as an eluent to obtain the title compound (1.5 gm, 99% yield).

¹H NMR (400 MHz, CDCl₃) δ 9.08 (dd, J=4.2, 1.6 Hz, 1H), 8.52 (dd, J=8.2, 1.6 Hz, 1H), 7.44 (dd, J=8.2, 4.3 Hz, 1H), 6.23 (s, 1H), 5.15 (s, 1H), 4.11 (s, 3H), 3.22-3.08 (m, 1H), 2.98-2.85 (m, 1H), 2.77 (s, 3H), 2.57-2.45 (m, 1H), 1.96-1.85 (m, 1H).

### Step 7: 3-(5-methoxy-8-methyl-1,6-naphthyridin-7-yl)cyclopent-2-en-1-yl acetate (Compound 9g)

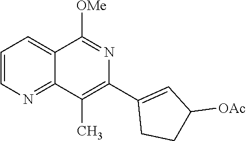 [SMILES: COc1nc(C2=CC(OC(C)=O)CC2)c(C)c2ncccc12]

To a solution of 3-(5-methoxy-8-methyl-1,6-naphthyridin-7-yl)cyclopent-2-enol (Compound 9f, 1.5 g, 5.85 mmol) in dichloromethane (25 ml) was added acetic anhydride (1.792 g, 17.56 mmol). The reaction mixture was stirred at room temperature for 1 hr. The reaction mixture was cooled to 0-5° C. and triethyl amine (1.777 g, 17.56 mmol) and DMAP (0.071 g, 0.585 mmol) were added slowly. The reaction mixture was stirred at room temperature for 5 min. The progress of the reaction was monitored by TLC. The reaction mixture was diluted with water (50 ml) and ethyl acetate (25 ml). The phases were separated and the aqueous phase was extracted with ethyl acetate (3×25 ml). The combined organic layer was dried over anhydrous sodium sulphate. The solvent in the organic layer was evaporated under reduced pressure to obtain crude product. The crude product was purified by flash column chromatography over silica gel (100-200 mesh) using 50% ethyl acetate in hexane as an eluent to obtain the title compound (1.1 g, 63% yield).

¹H NMR (400 MHz, CDCl₃) δ 9.08 (d, J=3.6 Hz, 1H), 8.52 (d, J=8.2 Hz, 1H), 7.45 (dd, J=8.2, 4.3 Hz, 1H), 6.21 (s, 1H), 5.97 (d, J=5.0 Hz, 1H), 4.12 (s, 3H), 3.23-3.11 (m, 1H), 3.02-2.92 (m, 1H), 2.77 (s, 3H), 2.58-2.46 (m, 1H), 2.14-2.09 (m, 3H), 2.08-1.99 (m, 1H).

MS: m/z 299 (M+1).

### Step 8: 4-(4-(3-(5-methoxy-8-methyl-1,6-naphthyridin-7-yl)cyclopent-2-en-1-yl)piperazin-1-yl)benzonitrile (Compound 9h)

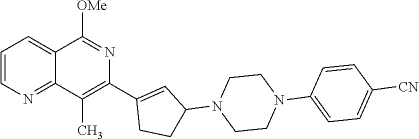 [SMILES: COc1nc(C2=CC(N3CCN(c4ccc(C#N)cc4)CC3)CC2)c(C)c2ncccc12]

To a solution of 3-(5-methoxy-8-methyl-1,6-naphthyridin-7-yl)cyclopent-2-en-1-yl acetate (Compound 9g, 1.1 g, 3.69 mmol) and 4-(piperazin-1-yl)benzonitrile (1.036 g, 5.53 mmol) in dioxane (8 ml) and water (2 ml) was added Pd(PPh₃)₄ (0.032 g, 0.028 mmol). The reaction mixture was stirred at room temperature for 15 hrs. The progress of reaction was monitored by TLC. The reaction mixture was diluted with water (100 ml) and ethyl acetate (100 ml). The phases were separated and the aqueous phase was extracted with ethyl acetate (2×100 ml). The combined organic layer was dried over anhydrous sodium sulphate. The solvent in the organic layer was evaporated under reduced pressure to obtain crude product. The crude product was purified by flash column chromatography over silica gel (100-200 mesh) using 40% ethyl acetate in hexane as an eluent to obtain the title compound (1.2 g, 76% yield).

¹H NMR (400 MHz, DMSO-d₆) δ 9.12 (dd, J=4.2, 1.6 Hz, 1H), 8.52 (dd, J=8.2, 1.7 Hz, 1H), 7.69-7.53 (m, 3H), 7.05 (d, J=8.9 Hz, 2H), 6.23 (s, 1H), 4.06 (s, 3H, overlap with m, 1H), 3.40-3.35 (m, 4H), 2.95-2.83 (m, 2H), 2.73-2.64 (m, 7H), 2.14-2.02 (m, 1H), 1.98-1.87 (m, 1H).

### Step 9: (R)-4-(4-(3-(8-methyl-5-oxo-5,6-dihydro-1,6-naphthyridin-7-yl)cyclopent-2-en-1-yl)piperazin-1-yl)benzonitrile (Compound 9)

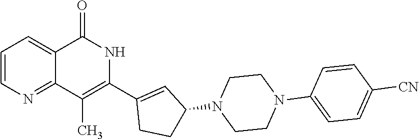 [SMILES: Cc1c(C2=C[C@H](N3CCN(c4ccc(C#N)cc4)CC3)CC2)[nH]c(=O)c2cccnc12]

and

### (S)-4-(4-(3-(8-methyl-5-oxo-5,6-dihydro-1,6-naphthyridin-7-yl)cyclopent-2-en-1-yl)piperazin-1-yl)benzonitrile (Compound 10)

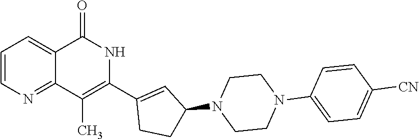 [SMILES: Cc1c(C2=C[C@@H](N3CCN(c4ccc(C#N)cc4)CC3)CC2)[nH]c(=O)c2cccnc12]

To a solution of 4-(4-(3-(5-methoxy-8-methyl-1,6-naphthyridin-7-yl)cyclopent-2-en-1-yl)piperazin-1-yl)benzonitrile (Compound 9h, 1.0 g, 2.350 mmol) and TMS-Cl (0.511 g, 0.601 mL, 4.70 mmol) in acetonitrile (30 ml) was added sodium iodide (0.705 g, 4.70 mmol). The reaction mixture was stirred at 75° C. for 8 hrs. The progress of reaction was monitored by TLC. The reaction mixture was diluted with saturated aqueous sodium bicarbonate (200 ml) and dichloromethane (200 ml). The phases were separated and the aqueous phase was extracted with dichloromethane (3×100 ml). The combined organic layer was dried over anhydrous sodium sulphate. The solvent in the organic layer was evaporated under reduced pressure to obtain crude product. The crude product was purified by flash column chromatography over silica gel (100-200 mesh) using 5% methanol in dichloromethane as an eluent to obtain the racemic title compound (0.650g, 67.2% yield).

¹H NMR (400 MHz, DMSO-d₆) δ 11.26 (brs-exchangeable with D₂O, 1H), 8.99 (dd, J=4.5, 1.9 Hz, 1H), 8.51 (dd, J=8.1, 1.9 Hz, 1H), 7.59 (d, J=8.7 Hz, 2H), 7.51 (dd, J=8.0, 4.5 Hz, 1H), 7.05 (d, J=8.8 Hz, 2H), 6.14 (d, J=2.2 Hz, 1H), 3.96 (s, 1H), 3.40-3.35 (m, 4H), 2.76-2.62 (m, 6H), 2.33 (s, 3H), 2.11-2.01 (m, 1H), 1.99-1.87 (m, 1H).

MS: m/z 412.3 (M+1).

Racemic compound was separated by CHIRALCEL OJ-H column using 0.1% DEA in Methanol as mobile phase to obtain:

### (R)-4-(4-(3-(8-methyl-5-oxo-5,6-dihydro-1,6-naphthyridin-7-yl)cyclopent-2-en-1-yl)piperazin-1-yl)benzonitrile (Compound 9)

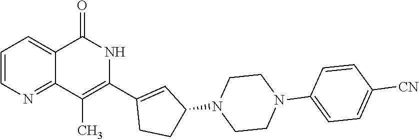 [SMILES: Cc1c(C2=C[C@H](N3CCN(c4ccc(C#N)cc4)CC3)CC2)[nH]c(=O)c2cccnc12]

and

### (S)-4-(4-(3-(8-methyl-5-oxo-5,6-dihydro-1,6-naphthyridin-7-yl)cyclopent-2-en-1-yl)piperazin-1-yl)benzonitrile (Compound 10)

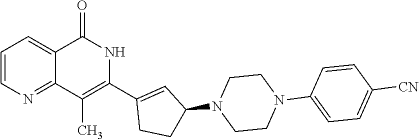 [SMILES: Cc1c(C2=C[C@@H](N3CCN(c4ccc(C#N)cc4)CC3)CC2)[nH]c(=O)c2cccnc12]

### Step 10: (R)-4-(4-(3-(8-methyl-5-oxo-5,6-dihydro-1,6-naphthyridin-7-yl)cyclopent-2-en-1-yl)piperazin-1-yl)benzonitrile (Compound 9—hydrochloride salt)

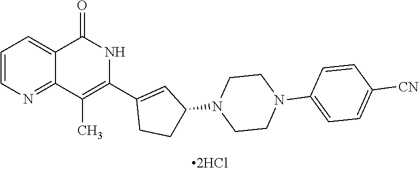 [SMILES: Cc1c(C2=C[C@H](N3CCN(c4ccc(C#N)cc4)CC3)CC2)[nH]c(=O)c2cccnc12]

To a solution of (R)-4-(4-(3-(8-methyl-5-oxo-5,6-dihydro-1,6-naphthyridin-7-yl)cyclopent-2-en-1-yl)piperazin-1-yl)benzonitrile (Compound 9, 100 mg, 0.243 mmol) in dichloromethane (10 ml) and methanol (5 ml) was added methanolic HCl (0.243 ml, 0.972 mmol). The reaction mixture was stirred at room temperature for 1 hr. After completion of reaction, solvent was distilled out under vacuum till dryness. A product was washed with diethyl ether (2×50 ml). A residue was dried under vacuum to obtain the title compound (80 mg, 68.0% yield).

¹H NMR (400 MHz, DMSO-d₆) δ 11.41 (brs-exchangeable with D₂O, 1H), 10.97 (brs-exchangeable with D₂O, 1H), 9.02 (dd, J=4.5, 1.6 Hz, 1H), 8.55 (dd, J=8.0, 1.5 Hz, 1H), 7.69 (d, J=8.9 Hz, 2H), 7.56 (dd, J=8.0, 4.6 Hz, 1H), 7.16 (d, J=8.9 Hz, 2H), 6.27 (s, 1H), 4.68 (s, 1H), 4.16 (d, J=11.5 Hz, 2H), 3.62 (d, J=11.9 Hz, 2H), 3.36-3.15 (m, 4H), 2.88 (d, J=9.0 Hz, 2H), 2.43-2.37 (m, 2H), 2.35 (s, 3H).

MS: m/z 412.3 (M+1).

### Step 11: (S)-4-(4-(3-(8-methyl-5-oxo-5,6-dihydro-1,6-naphthyridin-7-yl)cyclopent-2-en-1-yl)piperazin-1-yl)benzonitrile (Compound 10—hydrochloride salt)

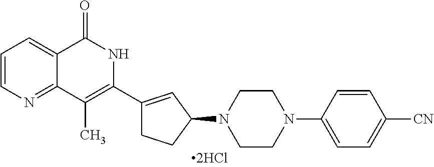 [SMILES: Cc1c(C2=C[C@@H](N3CCN(c4ccc(C#N)cc4)CC3)CC2)[nH]c(=O)c2cccnc12]

To a solution of (S)-4-(4-(3-(8-methyl-5-oxo-5,6-dihydro-1,6-naphthyridin-7-yl)cyclopent-2-en-1-yl)piperazin-1-yl)benzonitrile (Compound 10, 100 mg, 0.243 mmol) in dichloromethane (10 ml) and methanol (5 ml) was added methanolic HCl (0.243 ml, 0.972 mmol). The reaction mixture was stirred at room temperature for 1 hr. After completion of the reaction, the solvent was distilled out under vacuum till dryness. The product was washed with diethyl ether (2×50 ml). The residue was dried under vacuum to obtain the title compound (100 mg, 85% yield).

¹H NMR (400 MHz, DMSO-d₆) δ 11.42 (brs-exchangeable with D₂O, 1H), 11.14 (brs-exchangeable with D₂O, 1H), 9.06-8.98 (m, 1H), 8.55 (dd, J=8.1, 1.5 Hz, 1H), 7.69 (d, J=8.8 Hz, 2H), 7.57 (dd, J=8.0, 4.6 Hz, 1H), 7.16 (d, J=9.0 Hz, 2H), 6.28 (s, 1H), 4.68 (s, 1H), 4.15 (d, J=13.5 Hz, 2H), 3.62 (d, J=12.5 Hz, 2H), 3.38-3.14 (m, 4H), 2.88 (q, J=9.7 Hz, 2H), 2.44-2.36 (m, 2H), 2.35 (s, 3H).

MS: m/z 412.3 (M+1).

### Example 11: Synthesis of (R)-6-(4-(3-(5-oxo-5,6-dihydro-1,6-naphthyridin-7-yl)cyclopent-2-en-1-yl)piperazin-1-yl)nicotinonitrile (Compound 34)

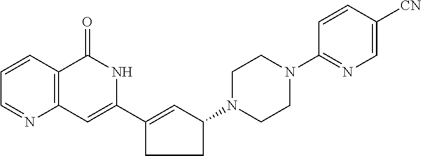 [SMILES: N#Cc1ccc(N2CCN([C@H]3C=C(c4cc5ncccc5c(=O)[nH]4)CC3)CC2)nc1]

### Step 1: (R)-tert-butyl 4-(3-(5-oxo-5H-pyrano[4,3-b]pyridin-7-yl)cyclopent-2-en-1-yl)piperazine-1-carboxylate (Compound 34a)

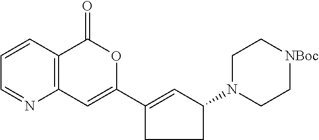 [SMILES: CC(C)(C)OC(=O)N1CCN([C@H]2C=C(c3cc4ncccc4c(=O)o3)CC2)CC1]

To a stirred solution of 2-bromonicotinic acid (5 g, 25 mmol) in acetonitrile (50 ml) (degassed by N₂ purge seperately), was added bis(triphenylphosphine)palladium(II) chloride (0.7 g, 1 mmol) and the reaction mixture was heated to 70° C. At this temperature, diisopropyl ethylamine (18.96 ml, 109 mmol) was added followed by the addition of (R)-tert-butyl 4-(3-ethynylcyclopent-2-en-1-yl)piperazine-1-carboxylate (Compound 23d, 5.3 g, 20 mmol) in acetonitrile (50 ml). The mixture was heated and stirred at 80-85° C. for 16 h. The progress of the reaction was monitored by TLC. Upon completion of the reaction, the reaction mixture was diluted with ethyl acetate (200 ml). The reaction mixture was washed with water (50 ml). The aqueous layer was extracted with ethyl acetate (2×100 ml) and the combined organic layer was dried over sodium sulphate and concentrated under reduced pressure. The crude product thus obtained was used without purification for the further reaction (yield 3.1 g, 41%).

MS: M/Z=398 (M+1).

### Step 2: (R)-tert-butyl 4-(3-(5-oxo-5,6-dihydro-1,6-naphthyridin-7-yl)cyclopent-2-en-1-yl)piperazine-1-carboxylate (Compound 34b)

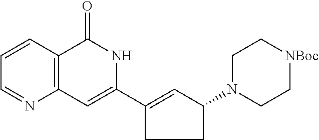 [SMILES: CC(C)(C)OC(=O)N1CCN([C@H]2C=C(c3cc4ncccc4c(=O)[nH]3)CC2)CC1]

In a steel bomb, a solution of (R)-tert-butyl 4-(3-(5-oxo-5H-pyrano[4,3-b]pyridin-7-yl)cyclopent-2-en-1-yl)piperazine-1-carboxylate (Compound 34a, 3.0 g, 7.55 mmol) in tetrahydrofuran (5 ml) and ammonia (32 ml, 150 mmol, 7M solution in methanol) was stirred at 25° C. for 5 min and the reaction was continued at 80-85° C. for 24 h. The progress of the reaction was monitored by TLC. Upon completion, the reaction mass was distilled under vacuum till dryness. The crude product thus obtained, was purified by chromatography using methanol in dichloromethane. The desired compound was isolated at 3-4% of methanol in dichloromethane. The combined fractions were concentrated to obtain the title compound as brown solid. (1.5 g, 50% yield).

¹HNMR (400 MHz, DMSO-d₆) δ 11.44 (brs-exchangeable with D₂O, 1H), 8.90 (dd, J=4.5, 1.5 Hz, 1H), 8.47 (d, J=7.8 Hz, 1H), 7.47 (dd, J=8.0, 4.6 Hz, 1H), 6.91 (s, 1H), 6.58 (s, 1H), 3.89 (s, 1H), 3.33-3.29 (m, 4H), 2.77-2.60 (m, 2H), 2.49-2.35 (m, 4H), 2.10-1.99 (m, 1H), 1.91-1.79 (m, 1H), 1.40 (s, 9H).

MS: M/Z=397 (M+1).

### Step 3: (R)-7-(3-(piperazin-1-yl)cyclopent-1-en-1-yl)-1,6-naphthyridin-5(6H)-one (Compound 34c)

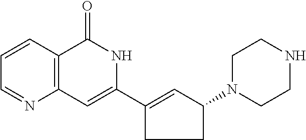 [SMILES: O=c1[nH]c(C2=C[C@H](N3CCNCC3)CC2)cc2ncccc12]

To a solution of tert-butyl (R)-4-(3-(5-oxo-5,6-dihydro-1,6-naphthyridin-7-yl)cyclopent-2-en-1-yl)piperazine-1-carboxylate (Compound 34b, 1.3 g, 5.17 mmol) in dichloromethane (10 ml), hydrochloric acid in 1,4 dioxane (12.91 ml, 51.7 mmol, 4M solution in 1,4 dioxane) was added at 0-5° C. The reaction mixture was warmed to room temperature and stirred for 2 h. The progress of the reaction was monitored by TLC. The reaction mixture was evaporated under reduced pressure to obtain solid product which was co-evaporated with diethyl ether (50 ml), followed by toluene (50 ml) to obtain hydrochloride salt. The resulting salt was neutralized with ammonia solution (30 ml, 7M in methanol) to obtain a crude product. The crude product was purified by chromatography using methanol-dichloromethane. The desired compound was eluted in 5-7% methanol in dichloromethane. The combined fractions were concentrated to yield the title compound as an off white solid (0.65 gm, 67%).

¹H NMR (400 MHz, DMSO-d₆) δ 11.49 (brs-exchangeable with D₂O, 1H), 8.90 (dd, J=4.5, 1.8 Hz, 1H), 8.47 (dd, J=8.1, 1.7 Hz, 1H), 7.48 (dd, J=8.0, 4.6 Hz, 2H), 6.87 (d, J=2.3 Hz, 1H), 6.60 (s, 1H), 3.93 (s, 1H), 3.05 (d, J=5.6 Hz, 4H), 2.80-2.59 (m, 6H), 2.14-2.00 (m, 1H), 1.91-1.77 (m, 1H).

MS: M/Z=297 (M+1).

### Step 4: (R)-6-(4-(3-(5-oxo-5,6-dihydro-1,6-naphthyridin-7-yl)cyclopent-2-en-1-yl)piperazin-1-yl)nicotinonitrile (Compound 34)

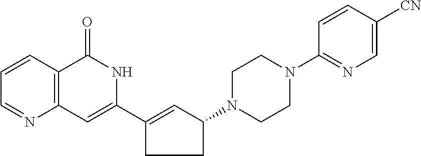 [SMILES: N#Cc1ccc(N2CCN([C@H]3C=C(c4cc5ncccc5c(=O)[nH]4)CC3)CC2)nc1]

To a solution of (R)-7-(3-(piperazin-1-yl)cyclopent-1-en-1-yl)-1,6-naphthyridin-5(6H)-one (Compound 34c, 100 mg, 0.337 mmol) in DMSO (5 ml) was added potassium carbonate (280 mg, 2.025 mmol) and 6-fluoronicotinonitrile (53.6 mg, 0.439 mmol) at 27° C. The reaction mixture was stirred at 120° C. for 18 hrs. The reaction mixture was poured into ice; the solid thus separated was filtered, washed with water (50 ml) and ether (20 ml). The solid was dissolved in methanol (2 ml) and precipitated with Diethyl ether (20 ml). It was filtered and dried to obtain the title compound (40 mg, 0.100 mmol, 29.8% yield) as light brown solid.

¹H NMR (400 MHz, DMSO-d₆) δ 11.45 (brs, exchangeable with D₂O, 1H), 8.90 (d, J=4.3 Hz, 1H), 8.47 (d, J=10.4 Hz, 2H), 7.85 (d, J=9.1 Hz, 1H), 7.48 (t, J=6.5 Hz, 1H), 6.95 (d, J=9.9 Hz, 2H), 6.59 (s, 1H), 3.97-3.88 (m, 1H), 3.78-3.58 (m, 4H), 2.79-2.65 (m, 2H), 2.65-2.55 (m, 4H), 2.17-2.01 (m, 1H), 1.97-1.82 (m, 1H).

MS: M/Z=399 (M+1).

### Step 5: (R)-6-(4-(3-(5-oxo-5,6-dihydro-1,6-naphthyridin-7-yl)cyclopent-2-en-1-yl)piperazin-1-yl)nicotinonitrile (Compound 34—hydrochloride salt)

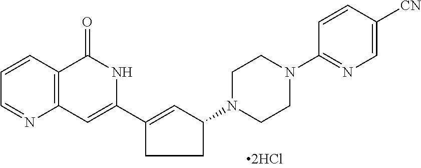 [SMILES: N#Cc1ccc(N2CCN([C@H]3C=C(c4cc5ncccc5c(=O)[nH]4)CC3)CC2)nc1]

To a solution of (R)-6-(4-(3-(5-oxo-5,6-dihydro-1,6-naphthyridin-7-yl)cyclopent-2-en-1-yl)piperazin-1-yl)nicotinonitrile (Compound 34, 40 mg, 0.1 mmol) in dichloromethane (5 ml) and methanol (5 ml), hydrochloric acid in dioxane (0.5 ml, 1 mmol, 3M solution in dioxane) was added at same temperature in small portions over a period of 2 minutes. The reaction mixture was stirred for 30 min at 55-60° C. The reaction mixture was cooled to room temperature, diluted with diethyl ether (10 ml), and product was collected upon filtration. The solid compound was washed with diethyl ether (10 ml) and dried under reduced pressure at 40° C. to obtain the title compound as brown solid (40 mg, 78% yield).

¹H NMR (400 MHz, DMSO-d₆) δ 11.73 (brs-exchangeable with D₂O, 1H), 11.59 (brs-exchangeable with D₂O, 1H), 9.00 (d, J=4.8 Hz, 1H), 8.64 (d, J=8.0 Hz, 1H), 8.58 (d, J=2.2 Hz, 1H), 7.99 (dd, J=9.1, 2.3 Hz, 1H), 7.63 (dd, J=8.0, 4.9 Hz, 1H), 7.10 (d, J=9.2 Hz, 1H), 6.85 (d, J=18.1 Hz, 2H), 4.72-4.58 (m, 3H), 3.58 (t, J=12.1 Hz, 2H), 3.48 (t, J=13.2 Hz, 2H), 3.19-3.02 (m, 2H), 2.96-2.85 (m, 2H), 2.45-2.33 (m, 2H).

MS: M/Z=399.1 (M+1).

The following compound was prepared using the procedure described above in Example 11 with appropriate changes to the reactants and reaction conditions.

### (R)-2-(4-(3-(5-oxo-5,6-dihydro-1,6-naphthyridin-7-yl)cyclopent-2-en-1-yl)piperazin-1-yl)thiazole-5-carbonitrile (Compound 35—hydrochloride salt)

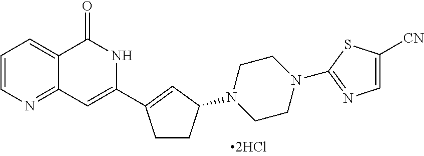 [SMILES: N#Cc1cnc(N2CCN([C@H]3C=C(c4cc5ncccc5c(=O)[nH]4)CC3)CC2)s1]

¹H NMR (400 MHz, DMSO-d₆) δ 11.69 (brs-exchangeable with D₂O, 1H), 11.65 (brs-exchangeable with D₂O, 1H), 8.99 (d, J=4.2 Hz, 1H), 8.59 (d, J=8.1 Hz, 1H), 8.13 (s, 1H), 7.60 (dd, J=7.9, 4.6 Hz, 1H), 6.84 (s, 1H), 6.81 (s, 1H), 4.69 (s, 1H), 4.26-4.12 (m, 2H), 3.71 (t, J=12.6 Hz, 2H), 3.59 (t, J=12.6 Hz, 2H), 3.32-3.13 (m, 2H), 2.96-2.82 (m, 2H), 2.43-2.31 (m, 2H).

MS: M/Z=405.3 (M+1).

### Example 12: Synthesis of (R)-4-(4-(3-(1-oxo-1,2-dihydropyrrolo[1,2-c]pyrimidin-3-yl)cyclopent-2-en-1-yl)piperazin-1-yl)benzonitrile (Compound 54)

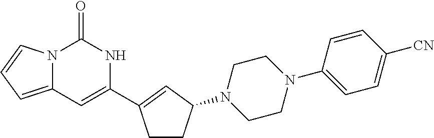 [SMILES: N#Cc1ccc(N2CCN([C@H]3C=C(c4cc5cccn5c(=O)[nH]4)CC3)CC2)cc1]

### Step 1: N-(pivaloyloxy)-1H-pyrrole-1-carboxamide (Compound 54a)

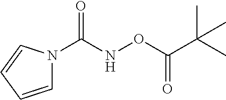

To a stirred solution of oxalyl chloride (0.945 ml, 10.80 mmol) in tetrahydrofuran (25 ml) was added dimethyl formamide (0.070 ml, 0.90 mmol) at 0° C. The reaction mixture was stirred for 10 min and 1H-pyrrole-1-carboxylic acid (1.0 g, 9.00 mmol) was added at 0° C. in two portions. The reaction mixture was stirred for 15 min at 0° C., cooling bath was removed and the reaction mixture was stirred at room temperature for 15 min. The solvent was evaporated under reduced pressure to obtain a crude acid chloride. In another round bottom flaskcontaining a stirred solution of sodium carbonate(1.90 g, 18.00 mmol) in ethyl acetate (40 ml) and water (20 ml) was added O-pivaloylhydroxylammonium trifluoromethanesulfonate (2.396 g, 9.00 mmol) at 0° C., followed by the addition of the acid chloride in ethyl acetate (5 ml). The reaction mixture was stirred at 0° C. for 2 hr, the progress of the reaction was monitored by TLC, and ethyl acetate (60 ml) was added to it. The two layers were separated and the aqueous layer was extracted with ethyl acetate (2×50 ml). The combined organic layer was dried over sodium sulphate, filtered and concentrated to obtain crude product. The crude product was purified by flash column chromatography (20-25% ethyl acetate in hexane) to obtain the title compound as a white solid (0.30 g, 16%).

¹H NMR (400 MHz, CDCl₃) δ 7.20-7.26 (m, 2H), 6.29-6.33 (m, 2H), 4.82 (bs-exchanges with D₂O, 1H), 1.37 (s, 9H).

MS: m/z 233 (M+23).

### Step 2: (R)-4-(4-(3-(1-oxo-1,2-dihydropyrrolo[1,2-c]pyrimidin-3-yl)cyclopent-2-en-1-yl)piperazin-1-yl)benzonitrile (Compound 54)

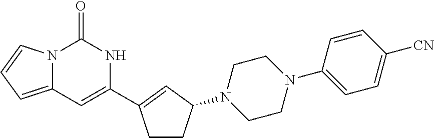 [SMILES: N#Cc1ccc(N2CCN([C@H]3C=C(c4cc5cccn5c(=O)[nH]4)CC3)CC2)cc1]

To the stirred solution of N-(pivaloyloxy)-1H-pyrrole-1-carboxamide (Compound 54a, 0.1 g, 0.476 mmol) in methanol (10 ml) were added cesium acetate (0.091 g, 0.476 mmol), Bis[(pentamethylcyclopentadienyl)dichloro-rhodium](0.029 g, 0.048 mmol) and (R)-4-(4-(3-ethynylcyclopent-2-en-1-yl)piperazin-1-yl)benzonitrile (Compound 1j, 0.1 g, 0.476 mmol). The reaction mixture was stirred at room temperature for 18 hrs. The progress of the reaction was monitored by TLC. The reaction mixture was concentrated to obtain the crude product; which was purified by flash column chromatography using 5% methanol in dichloromethane as an eluent to obtain the title compound (0.08 g, 44.0%).

¹H NMR (400 MHz, DMSO-d₆) δ10.78 (bs-exchanges with D₂O, 1H), 7.56-7.50 (m, 1H), 7.05 (t, J=8.7 Hz, 2H), 6.95 (dd, J=9.1, 4.7 Hz, 2H), 6.69 (s, 1H), 6.65 (t, J=3.3 Hz, 1H), 6.50 (s, 1H), 6.40 (d, J=3.5 Hz, 1H), 3.84 (s, 1H), 3.10 (m, 4H), 2.63-2.61 (m, 6H), 2.04-2.01 (m, 1H), 1.88 (m, 1H).

MS: m/z 386 (M+1).

### Step 3: (R)-4-(4-(3-(1-oxo-1,2-dihydropyrrolo[1,2-c]pyrimidin-3-yl)cyclopent-2-en-1-yl)piperazin-1-yl)benzonitrile (Compound 54—hydrochloride salt)

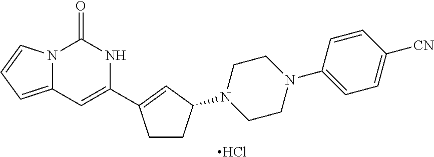 [SMILES: N#Cc1ccc(N2CCN([C@H]3C=C(c4cc5cccn5c(=O)[nH]4)CC3)CC2)cc1 ; Cl]

To the solution of (R)-4-(4-(3-(1-oxo-1,2-dihydropyrrolo[1,2-c]pyrimidin-3-yl)cyclopent-2-en-1-yl)piperazin-1-yl)benzonitrile (Compound 54, 0.06 g, 0.159 mmol) in dichloromethane (10 ml) was added hydrochloric acid (0.159 ml of 4M solution in dioxane, 0.634 mmol,) at 0° C. The reaction mixture was stirred for 1h at 25° C. The reaction mixture was diluted with diethyl ether (10 ml), and filtered through a Buchner funnel. The resulting solid was washed with diethyl ether (10 ml) and dried under reduced pressure to obtain the title compound (0.052 g, 87% yield).

¹H NMR (400 MHz, DMSO-d₆) δ 11.16 (brs-exchanges with D₂O, 1H), 10.89 (s, D₂O exchangeable, 1H), 7.68 (d, J=8.5 Hz, 2H), 7.61-7.55 (m, 1H), 7.14 (d, J=8.6 Hz, 2H), 6.71 (s, 1H), 6.69 (t, J=3.3 Hz, 1H), 6.60 (s, 1H), 6.49 (d, J=3.5 Hz, 1H), 4.62-4.60 (m, 1H), 4.18-4.09 (m, 2H), 3.61-3.50 (m, 2H), 3.34-3.26 (m, 2H), 3.18-3.05 (m, 2H), 2.92-2.69 (m, 2H), 2.41-2.31 (m, 2H).

MS: m/z 386.2 (M+1).

The following compound was prepared using a process analogous to Example 12 by appropriately changing the reactants/intermediates and reaction conditions as required.

### (R)-3-(3-(4-(4-fluorophenyl)piperazin-1-yl)cyclopent-1-en-1-yl)pyrrolo [1,2-c]pyrimidin-1(2H)-one (Compound 55—hydrochloride salt)

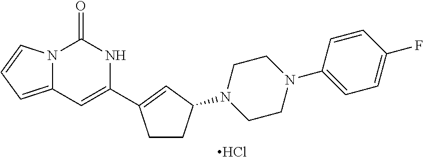 [SMILES: O=c1[nH]c(C2=C[C@H](N3CCN(c4ccc(F)cc4)CC3)CC2)cc2cccn12 ; Cl]

¹H NMR (400 MHz, DMSO-d₆) δ 11.23 (brs-exchanges with D₂O, 1H), 10.92 (bs-exchanges with D₂O, 1H), 7.58 (d, J=2.9 Hz, 1H), 7.20-7.00 (m, 4H), 6.73-6.67 (m, 2H), 6.63-6.61 (s, 1H), 6.49 (d, J=3.5 Hz, 1H), 4.61 (s, 1H), 3.83-3.73 (m, 2H), 3.57-3.47 (m, 2H), 3.19-3.10 (m, 4H), 2.89-2.71 (m, 2H), 2.39-2.32 (m, 2H).

MS: m/z 379.1 (M+1).

### Example 13: Synthesis of (R)-4-(4-(3-(1-oxo-1,2-dihydropyrrolo[1,2-a]pyrazin-3-yl)cyclopent-2-en-1-yl)piperazin-1-yl)benzonitrile (Compound 56)

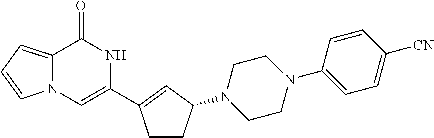 [SMILES: N#Cc1ccc(N2CCN([C@H]3C=C(c4cn5cccc5c(=O)[nH]4)CC3)CC2)cc1]

### Step 1: tert-butyl (R)-4-(3-acetylcyclopent-2-en-1-yl)piperazine-1-carboxylate (Compound 56a)

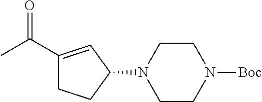 [SMILES: CC(=O)C1=C[C@H](N2CCN(C(=O)OC(C)(C)C)CC2)CC1]

A solution of methyl lithium (40 ml of 5% solution in tetrahydrofuran, 90.0 mmol) was added to a cooled methyl tert-butyl ether (200 ml) at 0° C. A solution of tert-butyl (R)-4-(3-cyanocyclopent-2-en-1-yl)piperazine-1-carboxylate (Compound 23b′, 10 g, 36.1 mmol) in methyl tert-butyl ether (70 ml) was added drop wise to the reaction mixture at 0° C. After complete addition, the reaction mixture was stirred at 0° C. for 30 min. The progress of the reaction was monitored by TLC. The reaction mixture was quenched with saturated aqueous ammonium chloride (10 ml), diluted with methyl tert-butyl ether (200 ml) and washed with water (50 ml).

The separated organic layer was dried over anhydrous sodium sulphate and concentrated to obtain a crude product. The crude product was purified by flash column chromatography using 50% ethyl acetate in hexane to obtain the title compound (5.0 g, 47.1%).

¹H NMR (400 MHz, CDCl₃) δ 6.71-6.69 (m, 1H), 3.98-3.89 (m, 1H), 3.51-3.41 (m, 4H), 2.68-2.42 (m, 6H), 2.36 (s, 3H), 2.12-2.02 (m, 1H), 1.92-1.84 (m, 1H), 1.48 (s, 9H).

MS: m/z 295 (M+1).

### Step 2: tert-butyl (R)-4-(3-(2-chloroacetyl)cyclopent-2-en-1-yl)piperazine-1-carboxylate (Compound 56b)

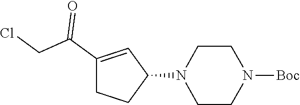 [SMILES: CC(C)(C)OC(=O)N1CCN([C@H]2C=C(C(=O)CCl)CC2)CC1]

To a stirred solution of lithium di-isopropyl amide in tetrahydrofuran (25 ml); which was prepared from diisopropylamine (1.81 ml, 12.74 mmol) and n-butyl lithium (6.90 ml of 1.6 M in hexane, 11.04 mmol) was added a solution of tert-butyl (R)-4-(3-acetylcyclopent-2-en-1-yl)piperazine-1-carboxylate (Compound 56a, 2.5 g, 8.49 mmol) in tetrahydrofuran (25 ml) under a nitrogen atmosphere at −78° C. The reaction mixture was stirred at −78° C. for lhr. A solution of N-chloro succinimide (1.58 g, 11.89 mnol) in tetrahydrofuran (12 ml) was added in 1 min at −78° C. The reaction mixture was stirred for 1 hr at −78° C. The progress of the reaction was monitored by TLC. The reaction mixture was quenched with saturated aqueous solution of ammonium chloride (10 ml) and stirred at room temperature for 15 min. The reaction mixture was diluted with ethyl acetate (100 ml). The organic layer was separated, washed with water (50 ml), brine (50 ml), dried over sodium sulphate and concentrated to obtain a crude compound. The crude compound was purified by flash column chromatography using 50% ethyl acetate in hexane to obtain the title compound (1.0 g, 35.8%).

¹H NMR (400 MHz, CDCl₃) δ 6.81-6.80 (m, 1H), 4.46-4.38 (m, 2H), 3.88-4.01 (m, 1H), 3.49-3.41 (m, 4H), 2.55-2.44 (m, 6H), 2.09-2.06 (m, 1H), 1.93-1.90 (m, 1H), 1.48 (s, 9H).

MS: m/z 329 (M+1).

### Step 3: tert-butyl (R)-4-(3-(2-(2-(ethoxycarbonyl)-1H-pyrrol-1-yl)acetyl)cyclopent-2-en-1-yl)piperazine-1-carboxylate (Compound 56c)

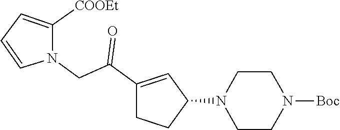 [SMILES: CCOC(=O)c1cccn1CC(=O)C1=C[C@H](N2CCN(C(=O)OC(C)(C)C)CC2)CC1]

To a stirred solution of ethyl 1H-pyrrole-2-carboxylate (0.931 g, 6.69 mmol) in dimethyl formamide (10 ml) was added cesium carbonate (3.27 g, 10.04 mmol) and stirred at 50° C. for 15 minutes. To this suspension, tert-butyl (R)-4-(3-(2-chloroacetyl)cyclopent-2-en-1-yl)piperazine-1-carboxylate (Compound 56b, 1.1 g, 3.35 mmol) in dimethyl formamide (5 ml) was added at 50° C. The reaction mixture was stirred for 1 hr. The progress of the reaction was monitored by TLC. The reaction mixture was cooled to room temperature and diluted with water (100 ml) and extracted with ethyl acetate (2×100 ml). The combined organic layer was dried over sodium sulphate, filtered and concentrated under reduced pressure to obtain the crude product. The crude compound was purified by column chromatography over silica gel (100-200 mesh) using 75% ethyl acetate in hexane as an eluent to obtain the title compound (0.80 gm, 55.4%).

¹H NMR (400 MHz, CDCl₃) δ 7.02 (dd, J=4.0, 1.8 Hz, 1H), 6.81-6.76 (m, 2H), 6.23 (dd, J=4.0, 1.8 Hz, 1H), 5.54 (d, J=17.4 Hz, 1H), 5.36 (d, J=17.4 Hz, 1H), 4.22 (q, J=7.1 Hz, 2H), 4.05-3.94 (m, 1H), 3.50-3.45 (m, 4H), 2.57-2.43 (m, 6H), 2.06-2.04 (m, 1H), 1.97-1.69 (m, 1H), 1.48 (s, 9H), 1.32 (t, J=7.1 Hz, 3H).

MS: m/z 432 (M+1).

### Step 4: tert-butyl (R)-4-(3-(1-oxo-1,2-dihydropyrrolo[1,2-a]pyrazin-3-yl)cyclopent-2-en-1-yl)piperazine-1-carboxylate (Compound 56d)

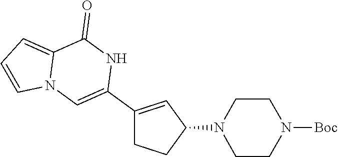 [SMILES: CC(C)(C)OC(=O)N1CCN([C@H]2C=C(c3cn4cccc4c(=O)[nH]3)CC2)CC1]

A stirred solution of tert-butyl (R)-4-(3-(2-(2-(ethoxycarbonyl)-1H-pyrrol-1-yl)acetyl)cyclopent-2-en-1-yl)piperazine-1-carboxylate (Compound 56c, 0.8 g, 1.854 mmol) in methanolic ammonia (5 ml) was heated at 90° C. for 14 hr in a sealed tube. The reaction mixture was cooled to room temperature and the progress of the reaction was monitored by TLC, the solvent was evaporated under reduced pressure to obtain a crude product, which was purified by flash column chromatography using 6% methanol in dichloromethane to obtain the title compound (0.55 g, 77.0%)

¹H NMR (400 MHz, DMSO-d₆) δ 10.44 (brs-exchanges with D₂O, 1H), 7.47-7.44 (m, 1H), 7.43 (s, 1H), 6.89 (dd, J=3.9, 1.5 Hz, 1H), 6.59 (s, 1H), 6.55-6.53 (m, 1H), 3.82-3.80 (m, 1H), 3.33-3.30 (m, 4H), 2.43-2.39 (m, 6H), 2.01-1.99 (m, 1H), 1.88-1.87 (m, 1H), 1.40 (s, 9H).

MS: m/z 385 (M+1).

### Step 5: (R)-3-(3-(piperazin-1-yl)cyclopent-1-en-1-yl)pyrrolo[1,2-a]pyrazin-1 (2H)-one dihydrochloride (Compound 56e)

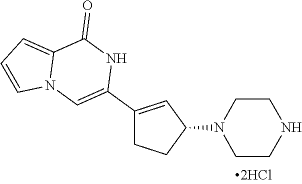 [SMILES: O=c1[nH]c(C2=C[C@H](N3CCNCC3)CC2)cn2cccc12]

To a stirred solution tert-butyl (R)-4-(3-(1-oxo-1,2-dihydropyrrolo[1,2-a]pyrazin-3-yl)cyclopent-2-en-1-yl)piperazine-1-carboxylate (Compound 56d, 0.43 g, 1.19 mmol) in dichloromethane (10 ml) at 0° C. was added hydrochloric acid (2.24 ml of 4M solution in dioxane, 8.95 mmol). The reaction mixture was allowed to come to room temperature and stirred for 2 hr. The progress of the reaction was monitored by TLC. The reaction mixture was concentrated under reduced pressure to yield a crude compound which was washed with hexane to obtain title compound (0.38 g, 95.0%)

MS: m/z 285 (M+1).

### Step 6: (R)-4-(4-(3-(1-oxo-1,2-dihydropyrrolo[1,2-a]pyrazin-3-yl)cyclopent-2-en-1-yl)piperazin-1-yl)benzonitrile (Compound 56)

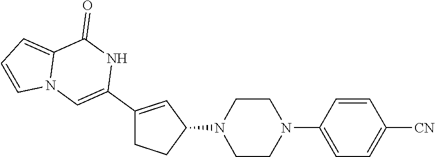 [SMILES: N#Cc1ccc(N2CCN([C@H]3C=C(c4cn5cccc5c(=O)[nH]4)CC3)CC2)cc1]

To a stirred solution of (R)-3-(3-(piperazin-1-yl)cyclopent-1-en-1-yl)pyrrolo[1,2-a]pyrazin-1(2H)-one dihydrochloride (Compound 56e, 0.20 g, 0.56 mmol) in dimethyl sulphoxide (10 ml) was added potassium carbonate (0.31 g, 2.24 mmol), and the reaction mixture was stirred at room temperature for 10 min. To this suspension, 4-fluorobenzonitrile (0.088 g, 0.730 mmol) was added and the reaction mixture was heated at 115° C. for 18 hrs. The reaction mixture was cooled to room temperature and the progress of the reaction was monitored by TLC. The reaction mixture was diluted with ethyl acetate (30 ml), filtered through Celite®, and the filtrate was washed with water (2×20 ml). The separated organic layer was washed with brine (20 ml), dried over sodium sulphate, filtered and concentrated under reduced pressure to yield a crude compound; which was purified by flash column chromatography using 70-80% ethyl acetate in hexane to obtain the title compound (0.07 g, 32.0%).

¹H NMR (400 MHz, DMSO-d₆) δ 10.46 (brs-exchanges with D₂O, 1H), 7.58 (d, J=8.6 Hz, 2H), 7.46-7.45 (m, 2H), 7.03 (d, J=8.6 Hz, 2H), 6.90 (d, J=3.9 Hz, 1H), 6.65 (s, 1H), 6.57-6.56 (m, 1H), 3.85-3.83 (m, 1H), 3.36-3.32 (m, 4H), 2.66-3.52 (m, 6H), 2.06-2.02 (m, 1H), 1.89-1.86 (m, 1H).

MS: m/z 386 (M+1).

### Step 7: (R)-4-(4-(3-(1-oxo-1,2-dihydropyrrolo[1,2-a]pyrazin-3-yl)cyclopent-2-en-1-yl)piperazin-1-yl)benzonitrile (Compound 56—hydrochloride salt)

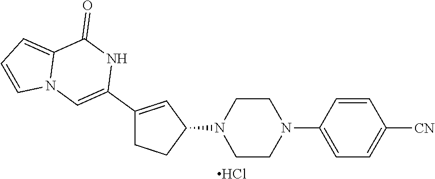 [SMILES: N#Cc1ccc(N2CCN([C@H]3C=C(c4cn5cccc5c(=O)[nH]4)CC3)CC2)cc1 ; Cl]

To a stirred solution of (R)-4-(4-(3-(1-oxo-1,2-dihydropyrrolo[1,2-a]pyrazin-3-yl)cyclopent-2-en-1-yl)piperazin-1-yl)benzonitrile (Compound 56, 0.06 g, 0.156 mmol) in dichloromethane (10 ml) was added hydrochloric acid (0.311 ml of 4.0M in dioxane, 1.245 mmol) at 0° C. The reaction mixture was warmed to room temperature and stirred for 1 hr. The reaction mixture was diluted with ether (10 ml), stirred for 10 minutes, solid was filtered and well dried under vacuum to obtain the title compound (0.052 g, 79.0%).

¹H NMR (400 MHz, DMSO-d₆) δ 11.21 (bs-exchanges with D₂O, 1H), 10.55 (bs-exchanges with D₂O, 1H), 7.69-7.64 (m, 3H), 7.51-7.47 (m, 1H), 7.14 (d, J=8.7 Hz, 2H), 6.94 (d, J=3.9 Hz, 1H), 6.62-6.56 (m, 2H), 4.59 (s, 1H), 4.18-4.12 (m, 2H), 3.62-3.46 (m, 2H), 3.42-3.24 (m, 2H), 3.18-3.02 (m, 2H), 2.88-2.76 (m, 1H), 2.74-2.64 (m, 1H), 2.46-2.33 (m, 2H).

MS: m/z 386 (M+1).

### Example 14: Synthesis of (R) 4-(4-(3-(8-nitro-5-oxo-5,6-dihydro-1,6-naphthyridin-7-yl)cyclopent-2-en-1-yl)piperazin-1-yl)benzonitrile (Compound 8)

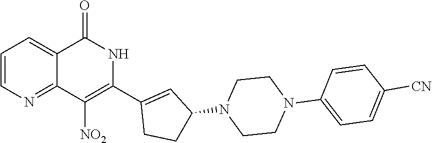 [SMILES: N#Cc1ccc(N2CCN([C@H]3C=C(c4[nH]c(=O)c5cccnc5c4[N+](=O)[O-])CC3)CC2)cc1]

### Step 1: tert-butyl (R)-4-(3-(8-nitro-5-oxo-5,6-dihydro-1,6-naphthyridin-7-yl)cyclopent-2-en-1-yl)piperazine-1-carboxylate (Compound 8a)

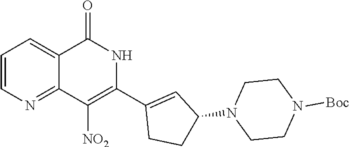 [SMILES: CC(C)(C)OC(=O)N1CCN([C@H]2C=C(c3[nH]c(=O)c4cccnc4c3[N+](=O)[O-])CC2)CC1]

To a solution of (R)-tert-butyl 4-(3-(5-oxo-5,6-dihydro-1,6-naphthyridin-7-yl)cyclopent-2-en-1-yl)piperazine-1-carboxylate (Compound 34b, 250 mg, 0.631 mmol) in trifluoroacetic acid (1 ml) was added nitric acid (0.028 ml, 0.631 mmol). The reaction mixture was stirred at 25° C. for 15 hrs. The progress of the reaction was monitored by TLC. The reaction mixture was diluted with ice cold water (10 ml) and basified with 2N sodium hydroxide (10 ml). To the resulting solution, BOC anhydride (1 ml) was added and stirred for another 2 hrs. The reaction mixture was diluted with dichloromethane (50 ml). The phases were separated and the aqueous phase was extracted with dichloromethane (3×100 ml). The combined organic layer was dried over anhydrous sodium sulphate. The solvent in the organic layer was evaporated under reduced pressure to obtain a crude product. The crude product was carried forward without purification (0.250 g, 90% yield).

### Step 2: (R)-4-(4-(3-(8-nitro-5-oxo-5,6-dihydro-1,6-naphthyridin-7-yl)cyclopent-2-en-1-yl)piperazin-1-yl)benzonitrile (Compound 8)

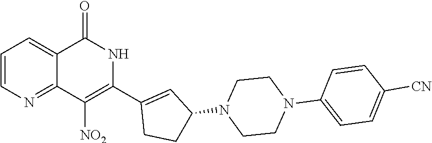 [SMILES: N#Cc1ccc(N2CCN([C@H]3C=C(c4[nH]c(=O)c5cccnc5c4[N+](=O)[O-])CC3)CC2)cc1]

To a solution of (R)-tert-butyl 4-(3-(8-nitro-5-oxo-5,6-dihydro-1,6-naphthyridin-7-yl)cyclopent-2-en-1-yl)piperazine-1-carboxylate (Compound 8a, 250 mg, 0.566 mmol) in dichloromethane (5 ml) was added trifluoroacetic acid (1 ml). The reaction mixture was stirred at 25° C. for 2 hrs. The progress of the reaction was monitored by TLC. After completion of the reaction, the solvent was removed by distillation and the product dried under vacuum. To this crude material, dimethyl sulphoxide (5 ml) was added, followed by the addition of 4-fluorobenzonitrile (274 mg, 2.265 mmol) and potassium carbonate (391 mg, 2.83 mmol). The reaction mixture was stirred at 120° C. for 15 hrs. The progress of the reaction was monitored by TLC. After completion of the reaction, water (50 ml) was added and the precipitated solid material was filtered, washed with diethyl ether (25 ml) and dried to obtain a crude product. The crude product was purified by flash column chromatography over silica gel (100-200 mesh) using 5% methanol in dichloromethane as an eluent to obtain the title compound (50 mg, 0.113 mmol, 20% yield).

MS: m/z 443 (M+1).

### Step 3: (R)-4-(4-(3-(8-nitro-5-oxo-5,6-dihydro-1,6-naphthyridin-7-yl)cyclopent-2-en-1-yl)piperazin-1-yl)benzonitrile (Compound 8—hydrochloride salt)

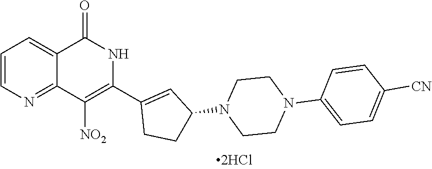 [SMILES: N#Cc1ccc(N2CCN([C@H]3C=C(c4[nH]c(=O)c5cccnc5c4[N+](=O)[O-])CC3)CC2)cc1]

To a solution of (R)-4-(4-(3-(8-nitro-5-oxo-5,6-dihydro-1,6-naphthyridin-7-yl)cyclopent-2-en-1-yl)piperazin-1-yl)benzonitrile (Compound 8, 20 mg, 0.045 mmol) in dichloromethane (5 ml) and methanol (10 ml) was added methanolic HCl (4 M, 0.045 ml, 0.181 mmol). The reaction mixture was stirred at room temperature for 1 hr. After completion of the reaction, the solvent was distilled out under vacuum till dryness. The product was washed with diethyl ether (2×50 ml). The residue was dried under vacuum to obtain the title compound (15 mg, 0.034 mmol, 75% yield).

¹H NMR (400 MHz, DMSO-d₆) δ 12.24 (brs-exchangeable with D₂O, 1H), 11.38 (brs-exchangeable with D₂O, 1H), 9.01 (s, 1H), 8.60 (d, J=8.1 Hz, 1H), 7.77-7.61 (m, 3H), 7.16 (d, J=8.3 Hz, 2H), 6.59 (s, 1H), 4.72 (s, 1H), 4.16 (d, J=12.9 Hz, 2H), 3.43-3.09 (m, 6H), 2.94-2.64 (m, 2H), 2.43-2.30 (m, 2H).

MS: m/z 443 (M+1).

### Example 15: Synthesis of (R)-4-(4-(3-(3-amino-5-oxo-5,6-dihydro-1,6-naphthyridin-7-yl)cyclopent-2-en-1-yl)piperazin-1-yl)benzonitrile (Compound 7)

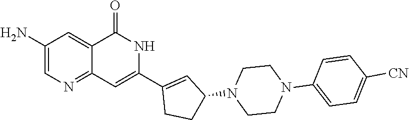 [SMILES: N#Cc1ccc(N2CCN([C@H]3C=C(c4cc5ncc(N)cc5c(=O)[nH]4)CC3)CC2)cc1]

### Step 1: (R)-4-(4-(3-(3-nitro-5-oxo-5,6-dihydro-1,6-naphthyridin-7-yl)cyclopent-2-en-1-yl)piperazin-1-yl)benzonitrile (Compound 7a)

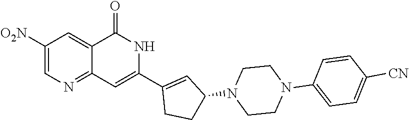 [SMILES: N#Cc1ccc(N2CCN([C@H]3C=C(c4cc5ncc([N+](=O)[O-])cc5c(=O)[nH]4)CC3)CC2)cc1]

A solution of (R)-4-(4-(3-ethynylcyclopent-2-en-1-yl)piperazin-1-yl)benzonitrile (Compound 1j-prepared according to the procedure given in Example 6, 0.70 g, 2.52 mmol) and 2-bromo-5-nitronicotinic acid (0.81 g, 3.28 mmol) in anhydrous acetonitrile was added to a mixture of bis(triphenylphosphine)palladium (II) chloride (0.177 g, 0.252 mmol) and diisopropylethyl amine (1.95 g, 15.14 mmol) in acetonitrile (70 ml) at 60-65° C. under nitrogen and the reaction mixture was heated at same temperature for 3 h. The progress of the reaction was monitored by TLC. The reaction mixture was cooled to room temperature and diluted with water (5 ml). The aqueous layer was extracted with dichloromethane (2×25 ml), combined organic layer was dried over anhydrous sodium sulphate, filtered and concentrated under reduced pressure to obtain crude product (0.750 g), which was dissolved in tetrahydrofuran (15 ml). To this crude product in tetrahydrofuran was added ammonia (11.28 ml, 79 mmol, 7N solution in methanol) and the reaction mixture was heated at 85° C. for 3 h in a sealed tube. The progress of reaction was monitored by TLC. The reaction mixture was cooled to room temperature and solvent was evaporated under reduced pressure to obtain crude product which was purified by flash column chromatography over silica gel (100-200 mesh) using 3% methanol in dichloromethane as an eluent to obtain the title compound (0.150 g, 21%).

¹H NMR (400 MHz, DMSO-d₆) δ 11.96 (brs-exchangeable with D₂O, 1H), 9.60 (d, J=2.4 Hz, 1H), 9.10-8.97 (m, 1H), 7.59 (d, J=8.8 Hz, 2H), 7.12 (s, 1H), 7.04 (d, J=8.8 Hz, 2H), 6.77 (s, 1H), 3.92-3.83 (s, 1H), 3.25-3.16 (m, 4H), 2.76-2.54 (m, 6H), 2.12-1.79 (m, 2H).

MS: m/z 443.2 (M+1).

### Step 2: (R)-4-(4-(3-(3-amino-5-oxo-5,6-dihydro-1,6-naphthyridin-7-yl)cyclopent-2-en-1-yl)piperazin-1-yl)benzonitrile (Compound 7)

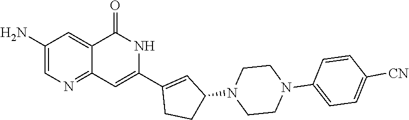 [SMILES: N#Cc1ccc(N2CCN([C@H]3C=C(c4cc5ncc(N)cc5c(=O)[nH]4)CC3)CC2)cc1]

To a solution of (R)-4-(4-(3-(3-nitro-5-oxo-5,6-dihydro-1,6-naphthyridin-7-yl)cyclopent-2-en-1-yl)piperazin-1-yl)benzonitrile (Compound 7a, 60 mg, 0.1355 mmol) in acetic acid (5 ml) and ethanol (5 ml) was added iron powder (30.0 mg, 0.542 mmol) at 25° C. The reaction mixture was heated at 80-85° C. for 1 hr under nitrogen atmosphere. The progress of the reaction was monitored by TLC. The reaction mixture was cooled to room temperature, solvents were removed under reduced pressure, and residue was dissolved in ammonium hydroxide (30%). The aqueous layer was extracted with ethyl acetate (3×30 ml). The combined organic layer was dried over anhydrous sodium sulphate, filtered and concentrated under reduced pressure to obtain crude product which was purified by flash column chromatography over silica gel (100-200 mesh) using 4% methanol in dichloromethane as an eluent to obtain the title compound (0.025 g, 44%).

¹H NMR (400 MHz, DMSO-d₆) δ 11.06 (brs-exchangeable with D₂O, 1H), 8.35 (d, J=2.8 Hz, 1H), 7.58 (d, J=8.4 Hz, 2H), 7.51 (d, J=2.8 Hz, 1H), 7.03 (d, J=8.4 Hz, 2H), 6.78 (s, 1H), 6.44 (s, 1H), 5.86 (brs-exchangeable with D₂O, 1H), 3.92-3.83 (s, 1H), 3.25-3.16 (m, 4H), 2.76-2.54 (m, 6H), 2.12-1.79 (m, 2H).

MS: m/z 413.3 (M+1).

### Step 3: (R)-4-(4-(3-(3-amino-5-oxo-5,6-dihydro-1,6-naphthyridin-7-yl)cyclopent-2-en-1-yl)piperazin-1-yl)benzonitrile (Compound 7—hydrochloride salt)

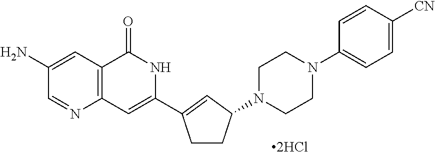 [SMILES: N#Cc1ccc(N2CCN([C@H]3C=C(c4cc5ncc(N)cc5c(=O)[nH]4)CC3)CC2)cc1]

A clear solution of (R)-4-(4-(3-(3-amino-5-oxo-5,6-dihydro-1,6-naphthyridin-7-yl)cyclopent-2-en-1-yl)piperazin-1-yl)benzonitrile (Compound 7, 25 mg, 0.061 mmol) in dichloromethane (5 ml) and methanol (5 ml) was warmed and stirred at 55-60° C., and a solution of hydrochloric acid in dioxane (0.13 ml, 0.364 mmol, 3M solution in dioxane) was added at the same temperature in small portions over a period of 5 minute. The reaction mixture was stirred for 30 min at 55-60° C. The reaction mixture was cooled to room temperature, diluted with diethyl ether (10 ml), and the product obtained was collected upon filtration. The solid compound was washed with diethyl ether (10 ml) and dried under reduced pressure for 3 hrs at 40° C. to obtain the title compound (0.015 g, 55% yield).

¹H NMR (400 MHz, DMSO-d₆) δ 11.59 (brs-exchangeable with D₂O, 1H), 8.45 (s, 1H), 7.94 (s, 1H), 7.68 (d, J=8.2 Hz, 2H), 7.14 (d, J=8.2 Hz, 2H), 6.81 (s, 1H), 6.74 (s, 1H), 4.69-4.63 (m, 1H), 4.41 (s, 2H), 4.18-4.09 (m, 2H), 3.62-3.52 (m, 2H), 3.22-3.04 (m, 3H), 2.94-2.72 (m, 2H), 2.44-2.31 (m, 3H).

MS: m/z 413.3 (M+1).

### Example 16: PARP1 Biochemical Assay

The assay was performed using BPS Bioscience kit. The 96-well strip plate was coated with 50 μl of histone mixture and incubated at 4° C. overnight. The next day, the wells were blocked by adding 100 μl of blocking buffer. The plate was washed and 25 μl of appropriate concentration of PARP1 (25-75 ng/well) was added in all of the Test and Positive control wells. In the Negative control wells, the enzyme was replaced with 25 μl of water. 5 μl each of 10× PARP assay buffer and activated DNA was added in all the wells (Test, Positive and Negative control wells). 10× concentration of the test compounds were prepared and 5 μl test compounds were added to the respective wells. The reaction volume was made up to 45 μl by adding water to all of the wells. 5 μl of 10× PARP assay mixture containing biotinilated NAD⁺ was added in each well and the plate was incubated at ambient temperature (25° C.) for 60 min. After washing the plate 50 μl of Streptavidin-HRP was added in each well, the plate was incubated at RT for 30 min. The plate was washed and the luminescence was read in PHERAStar plate reader after adding 100 μl of chemiluminescent substrate.

PARP inhibition was calculated using the following formula:

% PARP inhibition=100−[(RLU test compound treated sample−RLU negative control)/(RLU Positive control−RLU negative control)×100]

IC₅₀ values were calculated by plotting % inhibition against the respective concentrations of test compounds using GraphPad Prism 5.

PARP 1 inhibition IC₅₀ of the compounds of invention is provided in Table 1 below: Compounds with IC₅₀ between 0.5 nM and 5 nM are grouped under group A, and compounds with IC₅₀ between 5.1 nM and 50 nM are grouped under group B.

TABLE 1

| Group | Compound Nos. |
|---|---|
| A | 1, 2, 3, 6, 7, 8, 9, 10, 11, 14, 18, 19, 20, 21, 22, 23, 24, 25, |
| | 26, 28, 29, 30, 31, 32, 34, 35, 36, 37, 38, 39, 40, 42, 43, 44, |
| | 47, 48, 49, 50, 52, 53, 54, 55, and 56. |
| B | 4, 12, 13, 15, 16, 27, 33, 41, 45, and 51. |

1. A compound of the general formula (I), its tautomeric form, its stereoisomer, or its pharmaceutically acceptable salt, 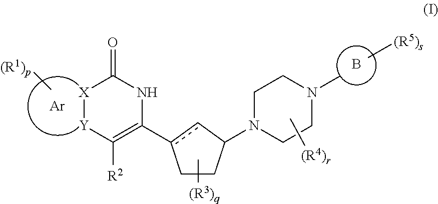
wherein, [symbol]
is either a single or a double bond;
X and Y independently represent carbon or nitrogen;
ring Ar is selected from
a) 6 membered heteroaromatic ring containing 1 to 2 nitrogen atoms, with X and Y being carbon; and
b) 5 membered heteroaromatic ring containing 1 to 2 heteroatoms selected from nitrogen, oxygen, and sulphur, wherein both X and Y are not selected as nitrogen at the same time;
R¹ is independently selected at each occurrence from halogen, nitro, cyano, perhaloalkyl, substituted- or unsubstituted-alkyl, substituted- or unsubstituted-cyclopropyl, —NH₂, —N(H)CH₃, —OH, and —OCH₃;
R² is selected from hydrogen, halogen, nitro, cyano, —NH₂, —N(H)CH₃, —OH, —OCH₃, substituted- or unsubstituted-cyclopropyl, and substituted- or unsubstituted-alkyl;
R³ is independently selected at each occurrence from halogen, and substituted- or unsubstituted-alkyl, or two R³ on the same carbon form an oxo (═O), or two R³ groups together with the carbon atom(s) to which they are attached form a substituted- or unsubstituted-carbocycle;
R⁴ is independently selected at each occurrence as substituted- or unsubstituted-alkyl, or two R⁴ on the same carbon form an oxo (═O), or two R⁴ groups together with the carbon atom(s) to which they are attached form a substituted- or unsubstituted-carbocycle or substituted- or unsubstituted-heterocycle;
ring B is selected from cycloalkyl, heterocyclyl, aryl, and heteroaryl;
R⁵ is independently selected at each occurrence from halogen, nitro, cyano, perhaloalkyl, substituted- or unsubstituted-alkyl, C(═O)R^{1a}, —C(═O)OR^{1b}, —C(═O)NR^{1b}R^{1c}, —NR^{1d}R^{1e}, and —OR^{1f};
R^{1a} is selected from substituted- or unsubstituted-alkyl, and substituted- or unsubstituted-cycloalkyl;
R^{1b} and R^{1c} are each independently selected from hydrogen, substituted- or unsubstituted-alkyl, and substituted- or unsubstituted-cycloalkyl;
R^{1d} and R^{1e} are each independently selected from hydrogen, —C(═O)alkyl, substituted- or unsubstituted-alkyl, and substituted- or unsubstituted-cycloalkyl;
R^{1f} is selected from hydrogen, —C(═O)alkyl, substituted- or unsubstituted-alkyl, perhaloalkyl, and substituted- or unsubstituted-cycloalkyl;
p is selected from 0, 1, and 2;
q is selected from 0, 1, 2, and 3;
r is selected from 0, 1, 2, and 3;
s is selected from 0, 1, 2, and 3;
when ‘alkyl’ is substituted, it is substituted with 1 to 3 substituents independently selected from oxo (═O), halogen, nitro, cyano, perhaloalkyl, cycloalkyl, cycloalkenyl, heterocyclyl, —OR^{6b}, —SO₂R^{6a}, —C(═O)OR^{6a}, —OC(═O)R^{6a}, —C(═O)N(H)R⁶, —C(═O)N(alkyl)R⁶, —N(H)C(═O)R^{6a}, —N(H)R⁶, and —N(alkyl)R⁶;
when ‘cycloalkyl’ and ‘carbocycle’ are substituted, each is substituted with 1 to 3 substituents independently selected from oxo (═O), halogen, nitro, cyano, alkyl, alkenyl, perhaloalkyl, heterocyclyl, —OR^{6b}, —SO₂R^{6a}, —C(═O)OR^{6a}, —OC(═O)R^{6a}, —C(═O)N(H)R⁶, —C(═O)N(alkyl)R⁶, —N(H)C(═O)R^{6a}, —N(H)R⁶, and —N(alkyl)R⁶;
when the ‘heterocycle’ is substituted, it is substituted either on one or more ring carbon atoms or on one or more ring hetero atoms, and when it is substituted on ring carbon atom(s), it is substituted with 1 to 3 substituents independently selected from oxo (═O), halogen, cyano, alkyl, alkenyl, perhaloalkyl, —OR⁶, —SO₂(alkyl), —C(═O)O(alkyl), —C(═O)N(H)R⁶, —C(═O)N(alkyl)R⁶, —N(H)C(═O)(alkyl), —N(H)R⁶, and —N(alkyl)₂; and when the heterocyclic group is substituted on ring nitrogen atom(s), it is substituted with a substituent or substituents independently selected from alkyl, alkenyl, cycloalkyl, cycloalkenyl, —SO₂(alkyl), —C(═O)(alkyl), C(═O)O(alkyl), —C(═O)N(H)R⁶, and —C(═O)N(alkyl)R⁶;
each R⁶ is independently selected from hydrogen, alkyl, alkenyl, cycloalkyl, cycloalkenyl, and heterocyclyl;
each R^{6a} is independently selected from alkyl, alkenyl, perhaloalkyl, cycloalkyl, cycloalkenyl, and heterocyclyl; and
R^{6b} is selected from hydrogen, alkyl, alkenyl, perhaloalkyl, cycloalkyl, cycloalkenyl, and heterocyclyl.

2. The compound of formula (I), its tautomeric form, its stereoisomer, or its pharmaceutically acceptable salt, as claimed in claim 1, wherein ring Ar is 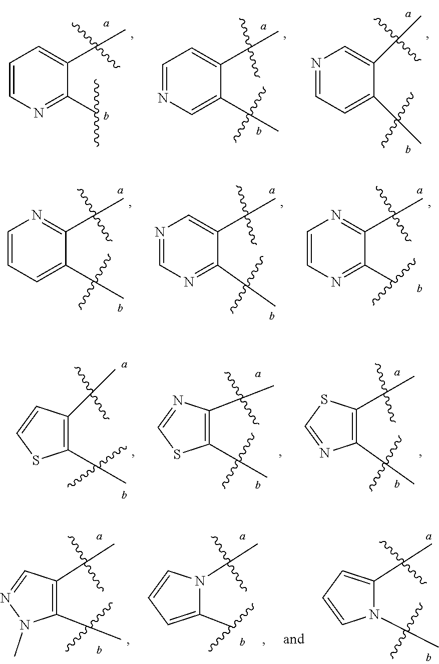
wherein a and b represent the points of attachment of the C═O and CR² moieties of the adjoining dihydropyridinone ring.

3. The compound of formula (I), its tautomeric form, its stereoisomer, or its pharmaceutically acceptable salt, as claimed in claim 1, wherein R¹ is independently selected at each occurrence from halogen, substituted- or unsubstituted-alkyl, and —NH₂.

4. The compound of formula (I), its tautomeric form, its stereoisomer, or its pharmaceutically acceptable salt, as claimed in claim 1, wherein R¹ is independently selected at each occurrence from fluorine, methyl, and amino.

5. The compound of formula (I), its tautomeric form, its stereoisomer, or its pharmaceutically acceptable salt, as claimed in claim 1, wherein p is 0 or 1.

6. The compound of formula (I), its tautomeric form, its stereoisomer, or its pharmaceutically acceptable salt, as claimed in claim 1, wherein R² is selected from hydrogen, nitro, and substituted- or unsubstituted-alkyl.

7. The compound of formula (I), its tautomeric form, its stereoisomer, or its pharmaceutically acceptable salt, as claimed in claim 1, wherein R² is selected from hydrogen, nitro, and methyl.

8. The compound of formula (I), its tautomeric form, its stereoisomer, or its pharmaceutically acceptable salt, as claimed in claim 1, wherein q is 0.

9. The compound of formula (I), its tautomeric form, its stereoisomer, or its pharmaceutically acceptable salt, as claimed in claim 1, wherein R⁴ is independently selected at each occurrence as substituted- or unsubstituted-alkyl, or two R⁴ on the same carbon form an oxo (═O), or two R⁴ groups together with the carbon atoms to which they are attached form a substituted- or unsubstituted-heterocycle.

10. The compound of formula (I), its tautomeric form, its stereoisomer, or its pharmaceutically acceptable salt, as claimed in claim 1, wherein R⁴ is independently selected at each occurrence as methyl, or two R⁴ on the same carbon form an oxo (═O), or two R⁴ groups together with the carbon atoms to which they are attached form a 2,5-diazabicyclo[2.2.1]heptane.

11. The compound of formula (I), its tautomeric form, its stereoisomer, or its pharmaceutically acceptable salt, as claimed in claim 1, wherein r is selected from 0, 1, and 2.

12. The compound of formula (I), its tautomeric form, its stereoisomer, or its pharmaceutically acceptable salt, as claimed in claim 1, wherein ring B is selected from aryl and heteroaryl.

13. The compound of formula (I), its tautomeric form, its stereoisomer, or its pharmaceutically acceptable salt, as claimed in claim 1, wherein ring B is selected from phenyl, pyridinyl, thiazolyl, 2,3-dihydro-indene-5-yl, 2,3-dihydro-1-indenone-5-yl, 1-isoindolinone-5-yl, and 2,3-dihydro-1-isobenzofuranone-5-yl.

14. The compound of formula (I), its tautomeric form, its stereoisomer, or its pharmaceutically acceptable salt, as claimed in claim 1, wherein R⁵ is independently selected at each occurrence from halogen, cyano, perhaloalkyl, substituted- or unsubstituted-alkyl, C(═O)R^{1a}, —C(═O)OR^{1b}, —C(═O)NR^{1b}R^{1c}, —NR^{1d}R^{1e}, and —OR^{1f}, wherein R^{1a} is substituted- or unsubstituted-alkyl; R^{1b} and R^{1c} are each independently selected from hydrogen, and substituted- or unsubstituted-alkyl; R^{1d} and R^{1e} are each independently selected from hydrogen and substituted- or unsubstituted-alkyl; and R^{1f} is substituted- or unsubstituted-alkyl.

15. The compound of formula (I), its tautomeric form, its stereoisomer, or its pharmaceutically acceptable salt, as claimed in claim 1, wherein R⁵ is independently selected at each occurrence from fluorine, chlorine, cyano, trifluoromethyl, methyl, —C(═O)CH₃, —C(═O)OCH₂CH₃, —C(═O)NHCH₃, —C(═O)NH₂, —NHCH₃, and —OCH₃.

16. The compound of formula (I), its tautomeric form, its stereoisomer, or its pharmaceutically acceptable salt, as claimed in claim 1, wherein s is selected from 0, 1, and 2.

17. The compound of formula (I), its tautomeric form, its stereoisomer, or its pharmaceutically acceptable salt, as claimed in claim 1, ring Ar is 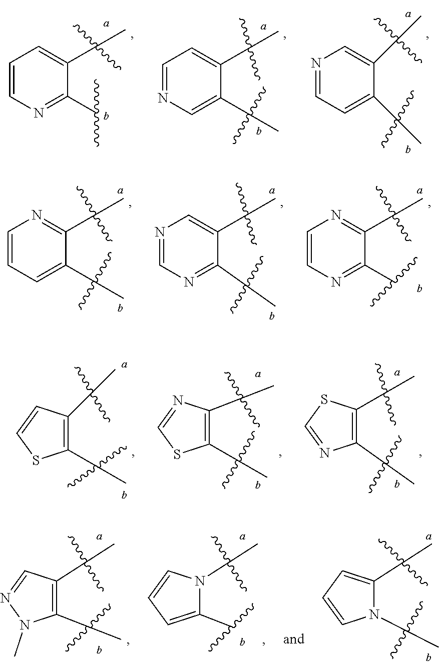
wherein a and b represent the points of attachment of the C═O and CR² moieties of the adjoining dihydropyridinone ring;
R¹ is independently selected at each occurrence from halogen, substituted- or unsubstituted-alkyl, and —NH₂;
R² is selected from hydrogen, nitro, and substituted- or unsubstituted-alkyl;
R⁴ is independently selected at each occurrence as substituted- or unsubstituted-alkyl, or two R⁴ on the same carbon form an oxo (═O), or two R⁴ groups together with the carbon atoms to which they are attached form a substituted- or unsubstituted-heterocycle;
ring B is selected from aryl and heteroaryl;
R⁵ is independently selected at each occurrence from halogen, cyano, perhaloalkyl, substituted- or unsubstituted-alkyl, C(═O)R^{1a}, —C(═O)OR^{1b}, —C(═O)NR^{1b}R^{1c}, —NR^{1d}R^{1e}, and —OR^{1f}, wherein R^{1a} is substituted- or unsubstituted-alkyl; R^{1b} and R^{1c} are each independently selected from hydrogen and substituted- or unsubstituted-alkyl; R^{1d} and R^{1e} are each independently selected from hydrogen and substituted- or unsubstituted-alkyl; and R^{1f} is substituted- or unsubstituted-alkyl;
p is 0 or 1;
q is 0;
r is selected from 0, 1, and 2; and
s is selected from 0, 1, and 2.

18. The compound of formula (I), its tautomeric form, its stereoisomer, or its pharmaceutically acceptable salt, as claimed in claim 1, wherein ring Ar is 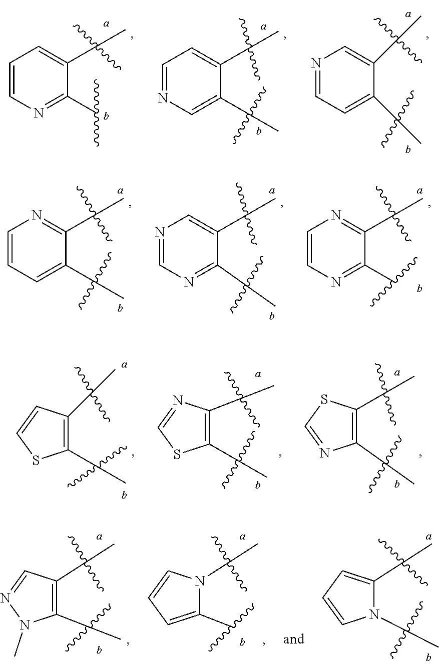
wherein a and b represent the points of attachment of the C═O and CR² moieties of the adjoining dihydropyridinone ring;
R¹ is independently selected at each occurrence from fluorine, methyl, and amino;
R² is selected from hydrogen, nitro, and methyl;
R⁴ is independently selected at each occurrence as methyl, or two R⁴ on the same carbon form an oxo (═O), or two R⁴ groups together with the carbon atoms to which they are attached form a 2,5-diazabicyclo[2.2.1]heptane;
ring B is selected from phenyl, pyridinyl, thiazolyl, 2,3-dihydro-indene-5-yl, 2,3-dihydro-1-indenone-5-yl, 2,3-dihydro-1-isobenzofuranone-5-yl, and 1-isoindolinone-5-yl;
R⁵ is independently selected at each occurrence from fluorine, chlorine, cyano, trifluoromethyl, methyl, —C(═O)CH₃, —C(═O)OCH₂CH₃, —C(═O)NHCH₃, —C(═O)NH₂, —NH(CH₃), and —OCH₃;
p is 0 or 1;
q is 0;
r is selected from 0, 1, and 2; and
s is selected from 0, 1, and 2.

19. The compound of formula (I), its tautomeric form, its stereoisomer, racemates or its pharmaceutically acceptable salt, as claimed in claim 1, wherein the compound is selected from:
(R)-4-(4-(3-(5-oxo-5,6-dihydro-1,6-naphthyridin-7-yl)cyclopent-2-en-1-yl)piperazin-1-yl)benzonitrile (Compound 1);
(R)-4-(4-(3-(3-fluoro-5-oxo-5,6-dihydro-1,6-naphthyridin-7-yl)cyclopent-2-en-1-yl)piperazin-1-yl)benzonitrile (Compound 2);
(R)-7-(3-(4-(o-tolyl)piperazin-1-yl)cyclopent-1-en-1-yl)-1,6-naphthyridin-5 (6H)-one (Compound 3);
(S)-4-(4-(3-(5-oxo-5,6-dihydro-1,6-naphthyridin-7-yl)cyclopent-2-en-1-yl)piperazin-1-yl)benzonitrile (Compound 4);
(S)-4-(4-(3-(3-fluoro-5-oxo-5,6-dihydro-1,6-naphthyridin-7-yl)cyclopent-2-en-1-yl)piperazin-1-yl)benzonitrile (Compound 5);
(R)-4-(4-(3-(2-methyl-5-oxo-5,6-dihydro-1,6-naphthyridin-7-yl)cyclopent-2-en-1-yl)piperazin-1-yl)benzonitrile (Compound 6);
(R)-4-(4-(3-(3-amino-5-oxo-5,6-dihydro-1,6-naphthyridin-7-yl)cyclopent-2-en-1-yl)piperazin-1-yl)benzonitrile (Compound 7);
(R)-4-(4-(3-(8-nitro-5-oxo-5,6-dihydro-1,6-naphthyridin-7-yl)cyclopent-2-en-1-yl)piperazin-1-yl)benzonitrile (Compound 8);
(R)-4-(4-(3-(8-methyl-5-oxo-5,6-dihydro-1,6-naphthyridin-7-yl)cyclopent-2-en-1-yl)piperazin-1-yl)benzonitrile (Compound 9);
(S)-4-(4-(3-(8-methyl-5-oxo-5,6-dihydro-1,6-naphthyridin-7-yl)cyclopent-2-en-1-yl)piperazin-1-yl)benzonitrile (Compound 10);
4-(4-((1R,3S/3R)-3-(5-oxo-5,6-dihydro-1,6-naphthyridin-7-yl)cyclopentyl)piperazin-1-yl)benzonitrile (Compound 11);
4-(4-((1R,3R/3S)-3-(5-oxo-5,6-dihydro-1,6-naphthyridin-7-yl)cyclopentyl)piperazin-1-yl)benzonitrile (Compound 12);
(R)-4-(2-oxo-4-(3-(5-oxo-5,6-dihydro-1,6-naphthyridin-7-yl)cyclopent-2-en-1-yl)piperazin-1-yl)benzonitrile (Compound 13);
4-((R)-3-methyl-4-((R/S)-3-(5-oxo-5,6-dihydro-1,6-naphthyridin-7-yl)cyclopent-2-en-1-yl)piperazin-1-yl)benzonitrile (Compound 14);
4-((R)-3-methyl-4-((S/R)-3-(5-oxo-5,6-dihydro-1,6-naphthyridin-7-yl)cyclopent-2-en-1-yl)piperazin-1-yl)benzonitrile (Compound 15);
4-((1S,4S)-5-((R/S)-3-(5-oxo-5,6-dihydro-1,6-naphthyridin-7-yl)cyclopent-2-en-1-yl)-2,5-diazabicyclo[2.2.1]heptan-2-yl)benzonitrile (Compound 16);
4-((1S,4S)-5-((S/R)-3-(5-oxo-5,6-dihydro-1,6-naphthyridin-7-yl)cyclopent-2-en-1-yl)-2,5-diazabicyclo[2.2.1]heptan-2-yl)benzonitrile (Compound 17);
(R)—N-methyl-4-(4-(3-(5-oxo-5,6-dihydro-1,6-naphthyridin-7-yl)cyclopent-2-en-1-yl)piperazin-1-yl)benzamide (Compound 18);
(R)-4-(4-(3-(5-oxo-5,6-dihydro-1,6-naphthyridin-7-yl)cyclopent-2-en-1-yl)piperazin-1-yl)benzamide (Compound 19);
Ethyl(R)-4-(4-(3-(5-oxo-5,6-dihydro-1,6-naphthyridin-7-yl)cyclopent-2-en-1-yl)piperazin-1-yl)benzoate (Compound 20);
(R)-7-(3-(4-phenylpiperazin-1-yl)cyclopent-1-en-1-yl)-1,6-naphthyridin-5 (6H)-one (Compound 21);
(R)-7-(3-(4-(4-fluorophenyl)piperazin-1-yl)cyclopent-1-en-1-yl)-1,6-naphthyridin-5(6H)-one (Compound 22);
(R)-3-fluoro-4-(4-(3-(5-oxo-5,6-dihydro-1,6-naphthyridin-7-yl)cyclopent-2-en-1-yl)piperazin-1-yl)benzonitrile (Compound 23);
(R)-7-(3-(4-(4-chlorophenyl)piperazin-1-yl)cyclopent-1-en-1-yl)-1,6-naphthyridin-5(6H)-one (Compound 24);
(R)-7-(3-(4-(4-methoxyphenyl)piperazin-1-yl)cyclopent-1-en-1-yl)-1,6-naphthyridin-5(6H)-one (Compound 25);
(R)-7-(3-(4-(p-tolyl)piperazin-1-yl)cyclopent-1-en-1-yl)-1,6-naphthyridin-5 (6H)-one (Compound 26);
(R)-7-(3-(4-(4-(methylamino)phenyl)piperazin-1-yl)cyclopent-1-en-1-yl)-1,6-naphthyridin-5(6H)-one (Compound 27);
(R)-7-(3-(4-(4-acetylphenyl)piperazin-1-yl)cyclopent-1-en-1-yl)-1,6-naphthyridin-5(6H)-one (Compound 28);
(R)-7-(3-(4-(1-oxo-2,3-dihydro-1H-inden-5-yl)piperazin-1-yl)cyclopent-1-en-1-yl)-1,6-naphthyridin-5(6H)-one (Compound 29);
(R)-7-(3-(4-(2,3-dihydro-1H-inden-5-yl)piperazin-1-yl)cyclopent-1-en-1-yl)-1,6-naphthyridin-5(6H)-one (Compound 30);
(R)-7-(3-(4-(1-oxo-1,3-dihydroisobenzofuran-5-yl)piperazin-1-yl)cyclopent-1-en-1-yl)-1,6-naphthyridin-5(6H)-one (Compound 31);
(R)-7-(3-(4-(1-oxoisoindolin-5-yl)piperazin-1-yl)cyclopent-1-en-1-yl)-1,6-naphthyridin-5(6H)-one (Compound 32);
(R)-7-(3-(4-(4-(trifluoromethyl)phenyl)piperazin-1-yl)cyclopent-1-en-1-yl)-1,6-naphthyridin-5(6H)-one (Compound 33);
(R)-6-(4-(3-(5-oxo-5,6-dihydro-1,6-naphthyridin-7-yl)cyclopent-2-en-1-yl)piperazin-1-yl)nicotinonitrile (Compound 34);
(R)-2-(4-(3-(5-oxo-5,6-dihydro-1,6-naphthyridin-7-yl)cyclopent-2-en-1-yl)piperazin-1-yl)thiazole-5-carbonitrile (Compound 35);
(R)-4-(4-(3-(1-oxo-1,2-dihydro-2,6-naphthyridin-3-yl)cyclopent-2-en-1-yl)piperazin-1-yl)benzonitrile (Compound 36);
(R)-4-(4-(3-(8-oxo-7,8-dihydro-1,7-naphthyridin-6-yl)cyclopent-2-en-1-yl)piperazin-1-yl)benzonitrile (Compound 37);
(R)-4-(4-(3-(1-oxo-1,2-dihydro-2,7-naphthyridin-3-yl)cyclopent-2-en-1-yl)piperazin-1-yl)benzonitrile (Compound 38);
(R)-7-(3-(4-(2,4-difluorophenyl)piperazin-1-yl)cyclopent-1-en-1-yl)-1,6-naphthyridin-5(6H)-one (Compound 39);
(R)-4-(4-(3-(5-oxo-5,6-dihydropyrido[4,3-d]pyrimidin-7-yl)cyclopent-2-en-1-yl)piperazin-1-yl)benzonitrile (Compound 40);
(R)-4-(4-(3-(5-oxo-5,6-dihydropyrido[3,4-b]pyrazin-7-yl)cyclopent-2-en-1-yl)piperazin-1-yl)benzonitrile (Compound 41);
(R)-4-(4-(3-(4-oxo-4,5-dihydrothieno[3,2-c]pyridin-6-yl)cyclopent-2-en-1-yl)piperazin-1-yl)benzonitrile (Compound 42);
(R)-4-(4-(3-(4-oxo-4,5-dihydrothiazolo[5,4-c]pyridin-6-yl)cyclopent-2-en-1-yl)piperazin-1-yl)benzonitrile (Compound 43);
(R)-4-(4-(3-(4-oxo-4,5-dihydrothiazolo[4,5-c]pyridin-6-yl)cyclopent-2-en-1-yl)piperazin-1-yl)benzonitrile (Compound 44);
(S)-4-(4-(3-(4-oxo-4,5-dihydrothieno[3,2-c]pyridin-6-yl)cyclopent-2-en-1-yl)piperazin-1-yl)benzonitrile (Compound 45);
(S)-4-(4-(3-(4-oxo-4,5-dihydrothiazolo[5,4-c]pyridin-6-yl)cyclopent-2-en-1-yl)piperazin-1-yl)benzonitrile (Compound 46);
(R)-6-(3-(4-(4-fluorophenyl)piperazin-1-yl)cyclopent-1-en-1-yl)thieno[3,2-c]pyridin-4(5H)-one (Compound 47);
(R)-6-(3-(4-phenylpiperazin-1-yl)cyclopent-1-en-1-yl)thieno[3,2-c]pyridin-4(5H)-one (Compound 48);
(R)—N-methyl-4-(4-(3-(4-oxo-4,5-dihydrothieno[3,2-c]pyridin-6-yl)cyclopent-2-en-1-yl)piperazin-1-yl)benzamide (Compound 49);
(R)-6-(4-(3-(4-oxo-4,5-dihydrothieno[3,2-c]pyridin-6-yl)cyclopent-2-en-1-yl)piperazin-1-yl)nicotinonitrile (Compound 50);
(R)-6-(3-(4-(thiazol-2-yl)piperazin-1-yl)cyclopent-1-en-1-yl)thieno[3,2-c]pyridin-4(5H)-one (Compound 51);
(R)-3-fluoro-4-(4-(3-(4-oxo-4,5-dihydrothiazolo[5,4-c]pyridin-6-yl)cyclopent-2-en-1-yl)piperazin-1-yl)benzonitrile (Compound 52);
(R)-4-(4-(3-(1-methyl-4-oxo-4,5-dihydro-1H-pyrazolo[4,3-c]pyridin-6-yl)cyclopent-2-en-1-yl)piperazin-1-yl)benzonitrile (Compound 53);
(R)-4-(4-(3-(1-oxo-1,2-dihydropyrrolo[1,2-c]pyrimidin-3-yl)cyclopent-2-en-1-yl)piperazin-1-yl)benzonitrile (Compound 54);
(R)-3-(3-(4-(4-fluorophenyl)piperazin-1-yl)cyclopent-1-en-1-yl)pyrrolo[1,2-c]pyrimidin-1(2H)-one (Compound 55); and
(R)-4-(4-(3-(1-oxo-1,2-dihydropyrrolo[1,2-a]pyrazin-3-yl)cyclopent-2-en-1-yl)piperazin-1-yl)benzonitrile (Compound 56).

20. A pharmaceutical composition comprising the compound of claim 1, its tautomeric form, its stereoisomer, or its pharmaceutically acceptable salt, and a pharmaceutically acceptable carrier.

21. The pharmaceutical composition of claim 20, further comprising at least one anticancer agent, or a pharmaceutically acceptable salt of said anticancer agent.

22. The pharmaceutical composition of claim 21, wherein the anticancer agent is selected from busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, elliptinium, etoposide, 5-azacytidine, gemcitabine, 5-fluorouracil, methotrexate, 5-fluoro-2′-deoxy-uridine, fludarabine, nelarabine, ara-C, alanosine, pralatrexate, pemetrexed, hydroxyurea, thioguanine, colchicine, vinblastine, vincristine, vinorelbine, paclitaxel, ixabepilone, cabazitaxel, docetaxel, campath, imatinib, gefitinib, erlotinib, lapatinib, sorafenib, sunitinib, nilotinib, dasatinib, pazopanib, temsirolimus, everolimus, vorinostat, romidepsin, tamoxifen, letrozole, fulvestrant, mitoguazone, octreotide, retinoic acid, arsenic trioxide, zoledronic acid, bortezomib, thalidomide and lenalidomide.

23. A method of treating or preventing a disorder responsive to the inhibition of PARP activity in a mammal suffering therefrom, comprising administering to the mammal in need of such treatment a therapeutically effective amount of a compound, its tautomeric form, its stereoisomer, or its pharmaceutically acceptable salt, of claim 1 or the pharmaceutical composition of claim 20.

24. The method of claim 23, wherein said disorder is cancer.

25. The method according to claim 24, wherein said cancer is liver cancer, melanoma, Hodgkin's disease, non-Hodgkin's lymphomas, acute or chronic lymphocytic leukaemia, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukaemia, primary brain carcinoma, malignant melanoma, small-cell lung carcinoma, stomach carcinoma, colon carcinoma, malignant pancreatic insulinoma, malignant carcinoid carcinoma, malignant melanoma, chorio carcinoma, mycosis fungoide, head or neck carcinoma, osteogenic sarcoma, pancreatic carcinoma, acute granulocytic leukaemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, or prostatic carcinoma.

26. A method of potentiating the efficacy of chemotherapeutic regimen for a patient undergoing chemotherapeutic treatment comprising co-administering to the patient an effective amount of a compound, tautomer, stereoisomer, or salt of claim 1.

27. The method of claim 26, wherein the compound, tautomer, stereoisomer, or salt is co-administered simultaneously, sequentially, or cyclically with the anticancer agent.

28. The method of claim 27, wherein the anticancer agent is selected from busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, elliptinium, etoposide, 5-azacytidine, gemcitabine, 5-fluorouracil, methotrexate, 5-fluoro-2′-deoxy-uridine, fludarabine, nelarabine, ara-C, alanosine, pralatrexate, pemetrexed, hydroxyurea, thioguanine, colchicine, vinblastine, vincristine, vinorelbine, paclitaxel, ixabepilone, cabazitaxel, docetaxel, campath, panitumumab, ofatumumab, bevacizumab, trastuzumab, adalimumab, imatinib, gefitinib, erlotinib, lapatinib, sorafenib, sunitinib, nilotinib, dasatinib, pazopanib, temsirolimus, everolimus, vorinostat, romidepsin, tamoxifen, letrozole, fulvestrant, mitoguazone, octreotide, retinoic acid, arsenic trioxide, zoledronic acid, bortezomib, thalidomide and lenalidomide.

29. A method for sensitizing a patient who has developed or who is likely to develop resistance to chemotherapic agents comprising administering an effective amount of a compound, its tautomeric form, its stereoisomer, or its pharmaceutically acceptable salt, of claim 1.

30-32. (canceled)
